SEQUENCE LISTING (185 sequences)

SEQ ID NO: 1 - DNA, 3393 bp, Artificial Sequence
  L1 Construct
atgcgagttcctgctcagctgcttggtctgttactgctgtggttgccgggcgcacgatgtgctatctgggaattaaagaaagatgtgtacgtggtggaattggattggtacccagatgctccgggcgaaatggttgtactcacatgcgatactccggaggaagacggtatcacttggacattggatcagtcgagtgaggttctcggtagtggtaaaacactaacgatccaagtcaaagaattcggtgatgcggggcaatatacctgtcataaaggcggcgaagtactatctcatagcctcctgctgttacacaagaaggaagatggcatatggtccaccgacatccttaaggatcagaaagaacccaagaacaaaactttcttgcgttgcgaagctaagaactactccggccgcttcacatgctggtggttgacaacgatcagtacggatctaaccttctctgttaagtccagtcgggggagttcggacccccaaggcgtcacgtgtggagctgccacactttccgctgagcgcgtacgtggagataataaagagtatgaatactccgttgagtgccaggaggactccgcgtgccccgctgccgaggagagtctccccatagaggtgatggtcgacgctgttcacaaactgaaatatgagaactatacctcatccttctttatacgtgacataattaagccagatcccccgaagaacttacaattgaaaccattgaagaattcacgtcaagtcgaggtatcctgggagtatcccgacacctggtccacgccacactcatatttctctctgaccttctgtgtgcaggtacaaggcaagagcaaacgagaaaaaaaggacagagttttcacggataagactagcgccacagtgatatgtaggaaaaacgcatcgatctcagtccgcgcgcaagatcggtattactcaagcagttggtcagagtgggcatcggtgccctgctcggggggttctggtgggggcagtggaggaggatcaggtggcggcagtcgcaatctacccgtggcaacaccagacccgggaatgttcccatgtctgcaccacagtcaaaacctcttaagggccgtgtcaaatatgctacagaaggcgagacagactttagaattctacccttgtacaagcgaggagattgaccacgaggacatcaccaaagataagacgagcaccgtcgaggcttgcctgcctctagaactaacaaaaaatgaatcatgcttgaactcgagggagaccagtttcattactaacggttcatgtcttgcatcgaggaagacctcattcatgatggccctgtgcctctcgtccatttatgaagacctaaagatgtaccaggtagagtttaagaccatgaacgccaagctcctcatggatccaaaacggcaaatattcttagatcagaatatgctcgctgttatcgacgaactcatgcaggcgcttaacttcaactcagaaaccgttccccaaaagtcgagtctagaagaaccggacttttataagaccaaaattaaactgtgtatactacttcacgccttcaggataagagcagtgacgattgacagggtgatgtcctacttgaatgcatcaggatcagggggaggctcggacgcccataagagcgaagtcgcccaccgcttcaaggatttgggtgaggaaaacttcaaagccctggtcctgatagcgtttgcccaatatttgcagcagtgtccattcgaagatcacgtgaaattggtgaacgaggtaacagaatttgctaagacttgtgtggctgacgagtcggccgaaaactgtgataagagtcttcatacactgtttggcgataagctatgtactgtcgctacacttagggagacttacggtgagatggccgactgctgcgccaagcaagagccagaacgaaacgagtgttttctgcaacataaggacgacaatcccaacctgcccagattggttcgccctgaagttgatgttatgtgcaccgcatttcacgacaacgaagaaacctttcttaaaaagtatctgtacgagatagctcgacgtcacccttacttctacgcgcccgaacttctgtttttcgccaagcgatacaaagccgctttcacagagtgttgccaagctgccgacaaagccgcttgccttctaccaaagcttgacgagctcagagatgaagggaaagctagttcggcaaagcaacgattaaagtgtgcatcactgcaaaaattcggcgaacgagcctttaaagcatgggcagttgccaggttatcccaaaggttcccgaaagctgaattcgctgaggtgagcaagttagtcacggaccttacgaaggtacataccgaatgctgccacggggacctcttggagtgcgctgacgacagggcggacttagctaaatacatttgcgagaatcaggactcaatcagttctaaacttaaagaatgctgcgagaaaccgctcctggaaaaatcacattgcatcgccgaggtggaaaacgatgagatgccagcagatttaccatctctagccgccgacttcgtggaaagtaaggatgtgtgtaaaaactatgcggaagcaaaagacgtgttcctcggaatgtttctatacgaatacgctagaaggcatcctgactattctgtcgttttactgcttagactagcgaagacatatgaaacgacgttagagaaatgctgcgcggccgctgacccccacgaatgttacgcgaaggtctttgatgagttcaagcccctggttgaggagccgcaaaaccttattaaacagaattgtgagctatttgagcagttaggcgaatataaattccagaatgcacttctagtacgatacaccaaaaaggtccctcaagtgagcacccccactcttgtggaggtatccagaaatctaggaaaggtaggctctaaatgctgcaagcatcccgaagccaagagaatgccatgcgctgaagactaccttagcgttgttctgaatcagttgtgtgtccttcacgaaaagacgccggtgagtgatcgtgtcacgaagtgctgtacagagagcctcgtcaaccgtaggccatgtttctccgctctcgaggtggatgaaacatatgtacctaaggaatttaatgcggaaactttcacctttcacgcggacatctgtaccctgagcgagaaggagaggcagataaaaaagcagacggctcttgtagagttggtcaaacataagcctaaggccactaaagagcagctaaaggcagtaatggacgactttgcggctttcgttgagaagtgctgcaaggccgacgataaagagacctgtttcgcggaagaaggtaaaaagttagtggccgcctcccaggcggccctgggcctgtag

SEQ ID NO: 2 - DNA, 3393 bp, Artificial Sequence
  L2 Construct
atgcgcgtgcccgcacaattgctcggactgctgctactgtggctgcccggggctcgctgcgcaatttgggaacttaagaaggacgtatacgttgtggaacttgactggtaccctgatgctccaggggagatggtggttttgacgtgtgacaccccggaagaagatggaattacatggaccttagaccaatcctccgaggttcttggctcgggcaaaaccttgaccattcaggtcaaggaatttggcgatgctggccaatacacctgccataaaggtggtgaagttttatctcactccctactgttgctccataagaaagaagacggcatttggtcgacagacatattgaaggatcagaaggaacctaagaataagaccttcttacgatgtgaggccaagaattattccggacgttttacgtgttggtggttgaccacgatctccactgacttaaccttctcagtgaaatcctcacgaggtagttccgatccccagggtgtgacgtgcggtgcggccacgttaagtgctgagagagtacggggcgacaataaggaatacgagtactcagttgaatgccaagaggactcggcctgtcccgcggcagaggagagtctccctatcgaagtgatggtggatgcggtgcacaagctcaagtatgaaaattacacatcatcttttttcatcagagacattataaagcccgacccaccaaagaacctccagttaaagcccttaaagaacagtagacaggttgaagtatcatgggaatacccagacacctggtccacaccccattcgtatttttccttgacgttttgcgtacaagttcagggaaagtccaaacgggaaaagaaagaccgcgtttttacagacaaaacttctgccactgtcatctgtagaaagaacgcatcaattagcgtgcgagcgcaagacagatactattcaagtagctggagcgagtgggccagtgttccatgttctggcggttcgggcggagggtccggcggtggtagcggaggcgggagcaggaacttgcccgtggctactccagaccctggcatgttcccctgtttacaccactcccagaacttattacgtgctgttagcaacatgttgcaaaaggcccgtcaaaccctcgagttttacccctgtactagtgaagaaatcgaccacgaagacataacaaaagacaagacaagcacagttgaggcatgcttacccctggagttaacaaagaacgagagctgtctgaactctcgagagacgagcttcatcaccaatgggagttgccttgcttctcgaaaaacgtcgttcatgatggccctgtgcctgtcgtctatctatgaggatctgaaaatgtatcaggttgaattcaagacaatgaatgccaaactactcatggatccaaaacggcagatattcctcgatcagaatatgctcgcagttattgacgaactaatgcaggctctgaattttaacagcgagaccgttcctcagaagtcaagtttggaagaacctgacttctacaaaactaaaataaaattatgcatcttactgcatgctttcagaattagagctgtcactattgatcgagtgatgtcatacttgaatgcttccggatcaggaggtgggagcgacgcgcacaaaagcgaggtagcgcatcgctttaaagacttaggagaggaaaactttaaggcgctggtgctcatcgcatttgcccaatacttacagcagtgtccttttgaggaccacgtaaagcttgtaaacgaagtcactgaattcgccaagacatgtgttgctgacgaaagcgcagagaactgtgacaaaagccttcataccctctttggtgacaaactctgcaccgtggcaactctaagagaaacctacggggaaatggcagactgctgcgcaaagcaggaacccgaacgcaatgagtgtttcctgcagcacaaggatgataatcccaatctgccacgacttgtacggccggaggtagatgttatgtgcactgcttttcatgacaacgaggaaactttccttaagaaatatctgtatgagatcgcaaggcggcacccttacttctacgcacccgaactgttgtttttcgcaaagaggtataaggccgcatttaccgagtgttgtcaggccgctgataaagccgcctgtcttttgccaaaattagatgaactaagggacgaaggcaaagcgagtagcgccaaacaaagattaaaatgtgcaagcctccaaaaattcggtgaaagagcatttaaggcgtgggctgtcgcccgactttcacaacgcttccccaaagctgaattcgctgaggtttcgaagctggttaccgacctaactaaagtgcatacagagtgctgtcatggggatctcttagagtgcgcggatgaccgggcagacctggctaagtacatatgtgagaaccaggacagtatatcatcaaagctgaaagagtgttgtgagaagccactactcgagaagagtcactgtattgccgaggtggaaaatgatgagatgccagccgatcttccttctttggccgctgactttgtagagagcaaggatgtctgtaagaactacgctgaggccaaggatgtctttttggggatgttcctctatgagtacgcccgacgacaccctgactatagtgtagtacttttgcttagactggctaaaacatatgagacgactctcgaaaagtgctgtgccgctgccgatccacacgagtgctacgctaaggtgtttgatgagtttaagccgctggtggaggaaccccagaacctgatcaagcagaactgtgaactattcgagcaactaggggagtacaaattccagaacgcacttttagtgcggtacaccaaaaaagtgccacaggtcagtacaccaacattagtggaagtatccaggaacctgggcaaagtgggcagcaaatgctgcaaacatccggaggctaagcggatgccctgtgcagaggactacctgtccgtggtgcttaaccagctgtgtgtgcttcacgagaaaacgcctgtgtccgaccgggtgaccaagtgctgtacggagtcactggtaaatcgacgaccgtgtttttcagcactagaagttgatgaaacttatgtaccgaaagagtttaacgcagagacctttacattccacgccgacatctgcacgctgtccgagaaggaaagacagattaaaaagcagactgccctagtcgagcttgtcaaacacaaaccgaaggcaaccaaggaacagttaaaagcagtgatggatgattttgctgcgttcgtcgaaaaatgttgcaaagcggacgacaaggagacttgcttcgcagaggaagggaagaaattggttgcggcgtcccaagcggccttagggctatag

SEQ ID NO: 3 - DNA, 3393 bp, Artificial Sequence
  L3 Construct
atgagagttcctgctcaactactagggctgctgttgttgtggttgcccggcgcgcgatgcgcaatatgggagctcaagaaggacgtttatgtcgttgagctggattggtaccctgatgccccgggcgaaatggttgtgcttacgtgcgatacccccgaggaggatggcataacatggacgttagatcagtcttccgaggtccttggttccggtaagactcttactatccaggtgaaggagttcggcgatgccggccagtacacttgccataaaggcggtgaagttctaagccactctctactgcttttgcacaagaaggaagatggaatatggtccaccgacatcttgaaggaccagaaagaaccaaaaaataagacatttttgaggtgtgaggcaaaaaattattcgggacgcttcacctgctggtggttgacgacgatttcaaccgacctcaccttctcagtaaagagttcgagaggtagttccgatccccaaggtgtgacatgtggcgctgcgactctaagcgctgaacgcgtaagaggtgataacaaagagtacgaatacagtgtggaatgccaagaagatagcgcgtgtccagccgcagaagaatctttaccaatagaggttatggttgatgccgttcacaaattgaaatatgagaattacacctcaagctttttcattcgagacataataaagcccgaccctcctaagaatcttcagttaaaaccgctgaagaacagtagacaagttgaggttagctgggaatatcctgatacctggtcaacgccgcactcgtatttctccctgactttctgtgttcaggttcaaggaaaatctaaaagggagaagaaagaccgtgttttcaccgacaagacatctgccacagtcatatgtaggaagaacgcttcaatcagcgtgcgagcgcaggaccgatactacagctctagttggtccgaatgggccagcgtaccatgctctggcggttccggcgggggctcggggggcgggagcggtggaggcagcaggaatcttcctgtcgcgacccctgatcccggcatgtttccttgcctgcaccacagtcagaacttattacgcgccgtttccaatatgttacagaaggccagacagacattagagttttatccttgcacatcggaggagatcgaccatgaagatatcacaaaagataaaacatccaccgttgaggcctgcctgccacttgaacttactaaaaacgagagctgcctgaatagccgggaaacttctttcatcactaatggatcgtgtctagcaagccgaaaaaccagcttcatgatggctttgtgcctctcgtccatctatgaagacctgaaaatgtatcaagtagaatttaaaacgatgaatgccaaactgcttatggatcccaaacgccagatatttctagaccagaatatgctggccgtcattgatgagctaatgcaggctctcaattttaatagcgaaacagtgccccaaaaaagctctttggaagagccggatttttacaaaaccaagattaagctatgtatcctgctgcatgctttcagaattcgagctgttacaattgatcgggttatgagctacttaaacgcctcgggtagtggcggggggagcgacgcccacaagtctgaagtggcacaccggttcaaggacctcggggaggagaattttaaagccctcgtgctgatcgctttcgcgcagtacttgcagcagtgcccttttgaggaccatgtcaaattagtaaacgaagtgacggaattcgcaaagacttgcgtagcggatgagtcagcagagaattgcgacaagtcgctacacactctgttcggggataagttgtgcacagttgctaccttacgagagacctatggagagatggctgactgctgcgccaaacaagagcctgaaagaaacgagtgcttcttacaacacaaagacgataacccgaatttgccaaggcttgtaagacccgaagtagatgttatgtgcacagcttttcacgacaacgaggagacgttccttaagaaatatctgtatgaaatagcccgtcggcacccctatttttatgctcccgaactattgttcttcgccaaacgatacaaggctgcgttcactgagtgctgtcaggctgcagacaaagcagcctgcttgcttcccaaattggacgaactacgggacgaaggtaaggcgagctccgcaaaacagcggttgaaatgcgcgtcactacagaagtttggggaaagagcgttcaaagcttgggctgtggcacgattgagccaacgcttccccaaagcagagtttgcggaagtttcgaaactcgtgacagacttaacaaaggttcacaccgagtgctgtcacggcgatctgctcgaatgcgctgatgaccgcgctgacttggctaaatacatttgtgagaaccaggactctatatcgagcaaactcaaggaatgctgcgaaaagcccctgcttgagaagtcccactgcatcgctgaggtagagaatgatgagatgcctgcggatcttccgagtttagcagctgatttcgtcgagagcaaagatgtgtgcaaaaattatgccgaagcaaaagacgtatttcttgggatgttcttatacgagtatgcacggcgccacccagattactccgtagtgctactgttaagattggcgaagacctatgaaaccacattggaaaagtgctgcgccgccgccgacccccacgagtgttacgcgaaggtgttcgatgaatttaaaccgcttgttgaggagccgcaaaacctaataaagcaaaactgtgagctctttgagcaactaggtgaatacaagtttcagaatgcactcctagttcggtacaccaaaaaagtacctcaggtatctacgccaacgttagtcgaggtctcgcggaatttgggtaaagtaggttccaagtgttgcaaacacccggaagctaaacgtatgccgtgtgctgaggactatctcagcgttgtgttaaaccaactttgcgtactccacgagaaaacacctgtctcagatcgggtaaccaaatgctgcacggagtcgctagtaaatcgtcgcccatgcttttctgcgttagaggtggacgaaacttatgtaccgaaagaatttaacgcggaaacctttacattccatgcagatatctgtacactgtccgagaaagagagacagattaagaaacagacggcgctggtggagcttgtgaagcacaagcctaaagctacgaaggagcaactgaaggcagtcatggatgactttgcggcgtttgtggagaagtgctgtaaagcggacgataaggaaacatgcttcgcagaagaaggaaagaagctggtcgccgctagccaagcggctctgggcctgtag

SEQ ID NO: 4 - DNA, 3393 bp, Artificial Sequence
  M1 Construct
atgagagtccccgcccagctgctggggcttcttttgctttggcttcctggcgcaagatgcgctatctgggagctgaaaaaggacgtgtacgtggtggaacttgactggtaccccgacgcccccggcgagatggtggtactgacctgcgacactcccgaggaagacggcattacctggaccttggaccagagcagcgaggttctgggctccggaaaaaccttgacaatccaagtgaaagaattcggcgacgctggccagtacacctgccacaagggcggcgaggtgctgtcccacagcctgctgctgctgcataagaaagaagacgggatttggagcaccgatatactgaaggatcagaaggagcccaagaacaagaccttcctgaggtgcgaggccaaaaattacagcggcagattcacctgctggtggctgaccaccattagcacagacctgactttcagcgtaaagtcttcaaggggcagctcagacccccagggagtaacttgcggagcggcaacgttgtctgccgagcgggtcagaggcgacaataaggagtacgagtattcagtagagtgtcaggaagatagcgcctgtcccgccgcggaggagagcctccccatcgaggtgatggtggacgccgtgcacaagttaaagtacgagaattacaccagctcattttttatcagagacattatcaagccggaccccccgaagaacttacagcttaaacccctaaagaacagcaggcaggttgaggtcagctgggaatatcctgacacctggtcaaccccccacagctacttctcccttactttctgtgtgcaagtgcagggcaagagcaagagagaaaagaaggaccgggtgtttaccgacaagactagcgccaccgtgatttgcagaaagaacgccagcattagtgtgagagcccaggacaggtattactccagctcatggtctgagtgggctagtgtgccttgctctggcggcagcggcggcgggagcggcggaggctccgggggaggtagccggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaagagcagcctcgaggagcccgacttctacaaaacaaagatcaaactctgcatccttctgcacgccttcagaattagagccgtgaccatcgacagagttatgagctacctgaatgccagcggcagcggcggcggatccgatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctctag

SEQ ID NO: 5 - DNA, 3393 bp, Artificial Sequence
  M2 Construct
atgagagtccccgcccagctgctagggctgctgctgctgtggttacccggcgcccggtgtgcaatttgggagttgaagaaggacgtgtacgtggtggagctggactggtacccggatgctcccggcgagatggtggtactcacctgcgacacacctgaggaagacggcatcacctggaccctcgatcagagcagcgaggttctgggaagcggcaaaaccctgaccatccaagtgaaagagtttggcgacgccggtcagtacacctgccacaagggaggcgaggtcctgtctcactctctgctgctgctccataagaaggaggacggtatttggagcactgacatcttgaaggatcaaaaagagccaaagaataaaacgttcctgaggtgcgaagctaagaattactccgggcgttttacgtgctggtggctgaccacgatcagcaccgatctgaccttcagcgtgaagagcagccggggcagcagcgacccccaaggcgtgacttgcggcgctgcgaccctgagcgctgagcgtgtgcgcggcgacaacaaggagtatgagtattcagtggagtgtcaggaggactccgcctgtcccgcggccgaagagagtctgcctattgaggtgatggtggacgccgtgcacaagctgaagtacgagaactacacatcgtcattctttatccgcgacatcataaagcccgacccccccaagaacctgcagctgaagcctctcaagaattcccggcaagtggaggtgagctgggagtaccctgatacctggtctacccctcacagctactttagcctgaccttctgcgtccaggtgcaaggaaagtcgaagcgcgagaagaaagatagagtcttcaccgataaaaccagtgccaccgtgatttgccgcaaaaacgcctccatcagcgtgcgggctcaggatagatactactctagcagctggagcgaatgggcctcagttccttgcagcggcggcagcggtggaggaagcggcggtggcagtgggggcgggagcagaaatctgcccgtcgccactccagatcctggcatgttcccgtgcctgcatcacagccaaaacctgctgcgggcggtgtctaacatgctgcagaaggctaggcagaccttggaattctatccctgcacaagcgaggaaatagaccatgaggacatcaccaaggataagaccagcacggtcgaagcttgcctgccactggaactgacaaaaaacgagagttgcctgaactcccgcgagacatccttcatcacaaacggcagctgcctggctagcaggaagaccagcttcatgatggccctgtgcctgtcttccatctacgaggacctgaaaatgtaccaagtggagttcaagactatgaacgccaagctgctaatggatcccaagcgacagatctttctagaccagaacatgctggccgtcattgacgagctgatgcaggcactcaattttaactcagagaccgtgccacagaagtccagcctggaggagcctgacttctataagaccaagattaagctgtgcatcctgctgcatgccttccgaataagagccgtgaccattgaccgagtgatgtcatacttgaacgcaagcggctcaggcggagggagtgacgcccacaagtctgaagtggctcaccggtttaaggaccttggcgaggagaactttaaagccctggtgctgattgcctttgcccagtatttacaacaatgccctttcgaagaccacgtgaagctcgtcaatgaggtcaccgagttcgctaagacctgcgtagccgacgaaagtgccgagaactgcgacaagagcctgcacaccctgttcggggacaaactctgtaccgtggccaccctacgggagacatatggggagatggccgactgctgcgcaaaacaggagcccgagagaaatgagtgcttcctgcagcacaaggatgacaaccccaatctgcccagactggtgcgccccgaggtagacgttatgtgcaccgccttccatgacaatgaggagacgttcctgaagaaatacctgtacgagatcgcaagacgtcacccctatttctatgcacctgagctgcttttcttcgccaagagatataaggccgccttcaccgaatgctgccaggcagccgataaggcagcttgcctcctgccaaagctggacgagctgagagatgagggcaaggcctccagcgcgaagcagagactcaaatgcgcaagccttcagaagttcggagaacgcgcctttaaagcctgggccgtcgccagactgagccagcgcttccctaaagccgaattcgcagaagtgagcaagctggtaacggacctgacaaaggtgcatactgagtgctgccatggcgatctgctggagtgcgctgatgacagagcagatttggcgaaatatatttgcgaaaatcaggatagcatcagctctaagctcaaggagtgttgtgagaagcccctgctggaaaaaagccactgcattgcagaggttgagaacgatgaaatgccagccgaccttccatcattggccgccgatttcgtggagtcgaaggatgtgtgtaagaactacgccgaggccaaggacgtgttcctgggcatgttcctgtacgagtatgctagaagacatcccgattacagtgtggtgctgctattgagactggccaagacctacgaaaccaccctggagaaatgttgcgccgcggcagatcctcacgaatgttacgccaaagtgtttgacgaattcaagccactggtagaggagccccagaacttaataaagcagaattgcgagctattcgagcagttgggcgagtacaaattccagaacgcccttctggtgaggtataccaaaaaggtgccccaggtgtctacccctaccctggtggaggtcagccgaaatctgggaaaggtcggatccaagtgctgcaagcacccggaggccaagaggatgccttgcgctgaggactatctcagtgtcgtcctgaatcagctatgcgtgttgcacgagaaaaccccagtgagtgaccgcgtgactaaatgctgcaccgaaagcttggtgaatcggaggccctgtttctctgcactggaagttgacgagacttacgtcccgaaggaattcaacgccgagacattcaccttccatgctgacatatgtactctgtcagaaaaggagcgtcagatcaagaagcagacagccctggtggaactggttaagcataagcctaaagcgaccaaagagcagctgaaagccgtgatggacgattttgccgccttcgtggagaaatgttgtaaggcagacgacaaagagacatgtttcgccgaagaggggaagaaactggtggccgcaagccaggccgctctgggtctgtag

SEQ ID NO: 6 - DNA, 3393 bp, Artificial Sequence
  M3 Construct
atgagagtcccagctcagctgctgggcctactgctgctatggctccccggcgcgcggtgcgccatttgggagctcaagaaggacgtgtacgtggtggaacttgactggtacccggacgcgcccggggaaatggtggtgctaacctgtgacacccccgaagaggacggcatcacctggaccctggaccagagcagtgaggtgctaggtagtggcaaaacgttaaccatccaggtcaaggagttcggcgacgccgggcaatacacctgtcacaaggggggggaggtactatcccactccctgctgctcctgcacaagaaagaggacgggatctggagcaccgacattctgaaagaccaaaaggagcccaaaaacaaaaccttccttagatgtgaagccaagaactacagcggccgtttcacctgctggtggctgaccaccatatctacggaccttaccttttcggtgaagagcagcagggggagttccgacccgcaaggcgtaacttgcggagccgcaaccctgagcgccgagagagtgcgcggcgacaacaaggagtacgagtatagcgtggagtgccaagaggacagcgcatgcccagccgccgaagagagcctgccaatagaggtcatggtagacgccgtgcacaagctaaaatatgaaaactacaccagcagctttttcatcagggatatcatcaaacccgacccaccaaaaaacttacagcttaagcctctgaaaaacagcagacaagttgaggtcagctgggagtaccccgacacttggagcacaccccactcctatttcagtttgacattctgcgtgcaggtgcagggtaaaagcaagagagaaaagaaggacagagtgttcacagataagacctcagccacagtgatctgccgtaagaatgccagcatcagcgtccgggctcaggacaggtactactcttcctcatggagcgagtgggcctctgtcccctgcagcggcggcagcggcggtggcagcggcggcggttctggcggcggttcaagaaacctcccagtcgctacccccgatcccggaatgttcccctgcctgcaccactcccagaatctgctccgagccgttagcaacatgctgcaaaaggcccggcagaccctggagttctacccatgcacctcggaagaaatcgatcacgaggacatcaccaaggacaagactagcaccgtcgaggcctgcctgccgctggaactaaccaagaatgaaagctgcctcaactcgcgggagacctctttcataaccaacggctcatgcctggccagccggaaaactagctttatgatggctctgtgcttaagcagcatctacgaggatctgaagatgtaccaggtagagttcaagaccatgaatgccaagctgctgatggaccccaagagacaaatcttcctggaccagaacatgctggccgtaattgatgaactgatgcaggccctgaatttcaacagcgagaccgtaccccagaaaagctcactggaggagcccgacttttataagacgaaaataaagttgtgcatccttcttcacgctttccggattagagccgtgaccatcgatagagtgatgtcatacctgaacgcatcggggagtggcggtggcagcgatgcccacaaaagcgaagtcgcacacagattcaaggacttgggtgaggagaactttaaagccctggtgctgatcgccttcgcgcagtatctccagcagtgccccttcgaagatcatgtgaaactggtgaacgaggtaaccgagttcgcgaagacatgcgttgctgatgagagcgccgaaaattgcgacaaaagcctgcatactctgttcggggacaagctgtgcacggtcgcaaccctgagagaaacctacggcgagatggcagactgctgcgccaagcaggagcctgagaggaacgagtgttttctgcagcacaaggacgataatcctaaccttcctcgtctagtgagacccgaagtggacgttatgtgtaccgcctttcacgacaatgaggaaacattcctgaaaaagtacctgtacgagatcgccagacggcacccatatttctacgcccccgagctgctcttcttcgcaaagaggtacaaggctgccttcaccgagtgctgccaggcggccgacaaggcggcgtgtttgctgcctaagctggacgaactacgtgacgaaggaaaagctagcagcgccaagcagagacttaagtgcgcgtccttacagaagtttggcgaaagagcgtttaaggcctgggccgtggcaaggctgtctcaaagattccccaaggcggagttcgccgaggtgtcaaaactggtgaccgacttaaccaaggtgcacaccgaatgctgccacggcgatctgctcgagtgcgccgacgacagagccgatctggcaaaatacatctgcgaaaaccaggatagcatcagctccaaactgaaggagtgctgtgaaaaaccactgcttgaaaaatcgcattgtatagcggaggtggagaatgacgagatgcccgccgacctgccaagcctggccgccgatttcgttgaatccaaggacgtttgcaagaactatgcagaagcgaaggacgtgttcttaggaatgttcctatacgagtacgcgagaagacatcccgactacagcgtggttctgctgttgagattagccaagacgtatgagacaaccctcgaaaagtgctgcgccgccgccgacccccacgagtgttacgcaaaggtgttcgatgagtttaaaccgctggttgaggaaccgcaaaacctgatcaagcagaactgcgagctgttcgagcagctgggtgaatacaagtttcagaatgcactgttggtgcgatataccaagaaggtgcctcaggtgagcacccctacccttgttgaggtgtcccgcaatctgggtaaggttgggagcaagtgttgcaaacaccccgaggccaagagaatgccctgcgcggaagattatctcagtgtcgtgcttaatcagttatgtgtcctgcatgagaagacccccgtgagcgacagagtgaccaagtgctgtaccgaatctctcgtgaacagacgcccgtgcttcagcgccttggaggtagacgagacctacgtgcccaaggagttcaacgcagagaccttcacctttcacgccgatatctgcaccctgtccgagaaggagagacaaatcaagaaacaaacggccctcgtggagctggtcaagcacaaacccaaggccacaaaagagcagctgaaggccgtgatggacgacttcgcagcctttgtggagaaatgctgcaaggctgacgacaaggagacatgcttcgccgaggaaggcaagaagttggtggccgccagccaggcggccctgggcctgtag

SEQ ID NO: 7 - DNA, 3393 bp, Artificial Sequence
  H1 Construct
atgagagtgcccgcccagttgcttggcctgctgctcctatggcttcccggcgccagatgcgccatctgggagctgaagaaagacgtgtacgtggtggagctggactggtaccccgacgcccccggtgaaatggtggttctgacctgcgacacaccagaggaggacggcatcacctggaccctggaccagtccagcgaggtcctgggctctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggcagcggcggcggctccggcggcgggagtggcggcggcagcagaaatctgcccgtagccacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaagagcagcctggaggaacccgacttctacaaaaccaagatcaagctgtgcattctgctgcatgccttccgcattagagccgtgaccatcgatagggtgatgagctacctgaacgccagcggctctggcggcggcagtgacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgagtgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaagagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctttag

SEQ ID NO: 8 - DNA, 3393 bp, Artificial Sequence
  H2 Construct
atgagagtgcccgcccagctgctgggcctgctgctcttatggctgcccggcgcccgctgtgctatttgggagctgaagaaggacgtgtacgtggtggagctggattggtatccagacgcccccggcgagatggtcgtgttgacctgcgatactcccgaggaggacggaatcacctggacacttgaccagagcagcgaggtgctgggcagcgggaagaccctgactatccaggtgaaggagtttggcgatgccggacagtacacctgccacaagggcggcgaggtgttatctcatagcctgctgctgctgcacaaaaaggaggacgggatctggagcactgacatcctgaaggaccagaaggagcccaaaaacaagaccttcctgcgttgcgaggccaagaactacagcgggagattcacctgttggtggctgaccactatcagcactgacctaaccttcagcgtgaagagcagccggggaagctctgacccccagggggtgacatgcggcgccgccacactgagcgccgagagggtgagaggggacaataaggaatacgagtatagcgtggagtgtcaggaagattccgcctgccccgccgccgaggagagcctgcctatcgaggtcatggtggacgccgtccataaactgaagtacgaaaactatacttcaagcttcttcatcagagacatcataaagcccgacccccccaagaacctgcagctaaagcccctgaagaacagcagacaggtcgaagtgagctgggagtaccccgatacctggagcaccccgcacagctacttcagcctgaccttttgcgtccaggtgcagggcaagagcaagagagagaagaaggacagggtgttcactgacaagacaagcgccactgttatctgcagaaagaacgccagtatcagcgtgcgcgcccaagacaggtattactccagcagctggtctgaatgggccagcgtgccttgcagcggggggagcggcggtggcagcgggggcggcagcggcgggggcagcagaaacctgcccgtggccacacccgatcccggcatgttcccctgtctgcaccacagccaaaacctgctgcgtgccgtgagcaacatgctgcagaaggcgcggcagaccctggagttctatccctgcaccagtgaggagattgaccacgaggatatcaccaaagacaagaccagcaccgtggaggcctgcctccccctggagctgaccaagaacgagtcctgcttgaattcaagagagaccagcttcatcaccaacggctcctgcttagccagcagaaagactagcttcatgatggccttgtgcttgtctagcatctatgaggatctgaagatgtaccaggtcgagttcaagactatgaacgccaagctgctgatggaccccaaaagacagatcttcctggaccagaacatgctggccgtcatcgacgagctgatgcaggcccttaatttcaatagcgagacagtgccccagaaatcttctctggaggagcccgatttttacaaaaccaagatcaaactatgcatcctgttgcacgccttccggatccgcgccgtgaccatcgacagagtaatgtcctacctgaacgccagcggcagcggcggcggtagcgacgcccacaagagcgaagtggcccatagattcaaggacctgggcgaggagaacttcaaggccctcgtgctgatcgccttcgcccagtacctgcagcagtgccctttcgaggaccacgttaaactggtgaatgaagtgaccgagttcgccaaaacctgcgtggccgacgagtctgccgaaaattgcgacaaaagcttacacaccctgttcggcgacaagctgtgcaccgtggccaccttaagagaaacctacggcgagatggccgactgctgcgctaagcaggagcccgagagaaacgaatgcttcctgcagcacaaggacgataacccaaatctgcctagactggtgagacccgaggtggacgtgatgtgcacagccttccacgataacgaagagacattcctgaaaaagtacctgtacgagatcgccagaagacatccttacttctatgcccccgagcttctgttcttcgccaagagatataaggccgccttcaccgagtgctgccaagccgccgacaaggcagcctgcctgctgccaaagcttgacgagctgagagacgagggcaaagccagcagcgccaagcagagactgaagtgcgcctccctgcagaagttcggcgagagagcctttaaggcctgggccgtggccagattgagtcagagattccccaaggccgagttcgccgaggtgagcaaactggtgaccgacctcactaaagtgcacacagaatgttgccatggcgatctcctggaatgcgccgatgacagggccgacctggccaagtacatctgtgagaaccaggacagcatttcgagcaagctgaaggagtgctgcgagaaacccctgctggagaagtcccactgcatcgccgaggtggagaacgacgagatgcccgccgacctgcccagcctggccgcagatttcgtggagagcaaggacgtatgcaagaactacgcagaggccaaggatgtgttcctgggcatgttcctgtacgaatacgccagaaggcaccccgactacagcgtcgtgctgttactgagactggccaagacctacgagacaaccttagagaagtgctgcgcggccgcagacccgcacgagtgctacgccaaggtgttcgacgagttcaagcccctggtggaggaacctcagaatctgattaagcagaattgtgagctgttcgagcagctgggagagtacaagttccagaacgcgctgctggtgagatataccaagaaggtgccccaggtgagcacccccaccctggtggaggtgagtcgcaacctgggcaaggtggggagcaagtgttgcaagcatcccgaggccaaaagaatgccctgcgcagaggactatctgagcgttgtgcttaaccagctgtgcgtgctgcacgagaagacccccgtgagcgacagagtgaccaagtgttgcaccgagagcctggtaaacagaagaccctgcttcagcgccctggaggtggacgagacctacgtgcccaaggagttcaacgccgagacctttaccttccacgcagacatttgcaccctgagcgagaaagagaggcagatcaagaaacagaccgctctggtcgagcttgtgaagcacaagcccaaagctaccaaggagcagctgaaggccgtgatggatgatttcgccgccttcgtagagaaatgctgcaaggccgacgataaagagacctgctttgccgaggagggcaagaaactggtggccgccagccaggcggccctgggcctgtag

SEQ ID NO: 9 - DNA, 3393 bp, Artificial Sequence
  H3 Construct
atgagagtgcccgcccagctgctgggcctgctgttgctgtggctgcccggcgccagatgcgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaagagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggcggtggcagtggcgggggcagcagaaacctgcccgtggccacccccgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaagagcagcctggaggagcccgacttttacaagaccaagatcaagttgtgcatcctcctgcacgccttcagaatcagagcggtgacgatcgacagagtcatgagctacctcaacgctagcggcagcggtggcggcagcgacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctcttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggagaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctgtag

SEQ ID NO: 10 - DNA, 3393 bp, Artificial Sequence
  CO Construct
atgagagtgcccgcccagctgctgggcctgctgctgctgtggctgcccggcgccagatgcgccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaagagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagcggcagcggcggcggcagcgacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 11 - DNA, 3390 bp, Artificial Sequence
  CP Construct
atgagggtccccgctcagctcctggggctcctgctgctctggctcccaggtgcacgatgtgccatctgggagctgaagaaggatgtgtatgtggtggagctggactggtacccagatgcccctggagagatggtggtgctgacctgtgacaccccagaggaggatggcatcacctggaccctggaccagagcagcgaggtgctgggcagcggcaagaccctgaccatccaggtgaaggagtttggagatgctggccagtacacctgccacaagggcggggaggtgctgagccacagcctgctgctgctgcacaagaaggaggatggcatctggagcacagacatcctgaaggaccagaaggagcccaagaacaagaccttcctgcgctgtgaggccaagaactacagcggccgcttcacctgctggtggctgaccaccatcagcacagacctgaccttctctgtgaaaagcagccggggcagcagtgacccccagggcgtgacctgtggggccgccaccctgtctgctgagcgggtgcggggggacaacaaggagtatgagtacagcgtggagtgccaggaggacagcgcctgcccagctgctgaggagagcctgcccatcgaggtgatggtggatgctgtgcacaagctgaagtatgagaactacaccagcagcttcttcatccgggacatcatcaagccagacccccccaagaacctgcagctgaagcccctgaagaacagccggcaggtggaggtgtcctgggagtacccagacacctggagcaccccccacagctacttcagcctgaccttctgtgtgcaggtgcagggcaagagcaagcgggagaagaaggacagagtcttcacagacaagaccagcgccaccgtcatctgcaggaagaatgccagcatctctgtgcgggcccaggaccgctactacagcagctcctggagcgagtgggcctctgtgccctgcagcggcggcagcggcggcggcagcggcggcggcagcggcggcggcagcaggaacctgcctgtggccaccccagaccccggcatgttcccctgcctgcaccacagccagaacctgctgcgggctgtgagcaacatgctgcagaaggcccggcagaccctggagttctacccctgcaccagcgaggagattgaccacgaggacatcaccaaggacaagaccagcacagtggaggcctgcctgcccctggagctgaccaagaatgaaagctgcctgaacagccgggagaccagcttcatcaccaacggcagctgcctggccagcaggaagaccagcttcatgatggccctgtgcctgagcagcatctatgaggacctgaagatgtaccaggtggagttcaagaccatgaatgccaagctgctgatggaccccaagaggcagatcttcctggaccagaacatgctggccgtgattgatgagctgatgcaggccctgaacttcaacagcgagaccgtgccccagaaaagcagcctggaggagccagacttctacaagaccaagatcaagctgtgcatcctgctgcacgccttccgcatccgggctgtgaccatcgacagagtgatgagctacctgaatgccagcggcagcggcggcggcagtgatgcccacaagtctgaggtggcccaccgcttcaaggacctgggggaggagaacttcaaggccctggtgctgattgcctttgcccagtacctgcagcagtgcccctttgaggaccacgtgaagctggtgaatgaggtgacagaatttgccaagacctgtgtggctgatgaatctgctgagaactgtgacaagagcctgcacaccctgtttggagacaagctgtgcaccgtggccaccctgcgggagacctatggagagatggctgactgctgtgccaagcaggagcctgagagaaatgaatgcttcctgcagcacaaggatgacaaccccaacctgccccggctggtgcggcctgaggtggatgtgatgtgcacagccttccatgacaatgaggagaccttcctgaagaagtacctgtatgaaattgcccggcggcacccctacttctacgcccctgagctgctgttctttgccaagcgctacaaggccgccttcacagagtgctgccaggccgctgacaaggccgcctgcctgctgcccaagctggatgagctgagagatgagggcaaggccagcagcgccaagcagaggctgaagtgtgccagcctgcagaagtttggagagcgggccttcaaggcctgggccgtggcccggctgagccagcgcttccccaaggccgagtttgctgaggtgtccaagctggtgacagacctgaccaaggtgcacacagagtgctgccacggggacctgctggagtgtgctgatgacagagctgacctggccaagtacatctgtgagaaccaggacagcatcagcagcaagctgaaggagtgctgtgagaagcccctgctggaaaagagccactgcatcgccgaggtggagaatgatgagatgcctgctgacctgcccagcctggccgctgactttgtggagagcaaggatgtgtgcaagaactatgcagaggccaaggatgtgttcctgggcatgttcctgtatgaatatgcccggcggcacccagactacagcgtggtgctgctgctgcggctggccaagacctatgagaccaccctggagaagtgctgtgccgctgctgacccccatgaatgttatgccaaggtgtttgatgagttcaagcccctggtggaggagccccagaacctgatcaagcagaactgtgagctgtttgagcagctgggggagtacaagttccagaatgccctgctggtgcgctacaccaagaaggtgccccaggtgtccacccccaccctggtggaggtgtccaggaacctgggcaaggtgggcagcaagtgctgcaagcaccctgaggccaagaggatgccctgtgccgaggactacctgtctgtggtgctgaaccagctgtgtgtgctgcacgagaagacccctgtgtctgacagagtgaccaagtgctgcacagagagcctggtgaacagaagaccctgcttcagcgccctggaggtggatgagacctacgtgcccaaggagttcaatgctgagaccttcaccttccacgccgacatctgcaccctgtctgagaaggagcggcagatcaagaagcagacagccctggtggagctggtgaagcacaagcccaaggccaccaaggagcagctgaaggctgtgatggatgactttgctgcctttgtggagaagtgctgcaaggcagatgacaaggagacctgctttgctgaggagggcaagaagctggtggccgccagccaggccgccctgggcctg

SEQ ID NO: 12 - DNA, 3393 bp, Artificial Sequence
  vH1 Construct
atgagagtgcccgcccagctgctgggcctgctgctgctgtggctgcccggcgccagatgcgccatctgggagctgaagaaggacgtgtacgtggtggagctggactggtaccccgacgcccccggcgagatggtggtgctgacctgcgacacccccgaggaggacgggatcacctggaccctggatcagagcagcgaggtgctgggcagcggcaagaccctgaccatccaggtgaaggagtttggtgacgccggccagtacacctgccacaagggcggcgaggtgctgagccactcactgctgctgctgcacaagaaggaggacggcatctggagcaccgacattctgaaggatcagaaggagcccaagaacaagaccttcctgagatgcgaggccaagaactacagcggcagattcacctgttggtggctgactactatcagcactgatctgaccttcagcgtgaagagctcaagaggcagcagcgatccccagggcgtgacctgcggcgccgccaccctgagcgccgagagagtgagaggcgacaacaaggagtacgagtacagcgtggagtgccaggaagacagcgcctgccccgctgcagaggagtctctgcccatcgaggtgatggtggacgccgtgcacaagcttaagtacgagaactacaccagctccttcttcattagagacatcatcaagcccgacccgcccaaaaacctgcagctgaagcccctgaagaacagcagacaggtggaggtcagctgggagtaccccgacacctggagcaccccccacagctacttcagcctgaccttctgcgtgcaggtgcagggcaagagcaagagagagaagaaggacagagtgttcaccgacaaaaccagcgccaccgtgatctgcagaaaaaacgccagcattagcgtgagagctcaggatagatactacagcagcagctggagtgagtgggccagcgtgccctgcagcggcggctcaggcggcggctcaggcggcggcagcggcggcggcagcagaaacctgcccgtggccacgcccgaccccggcatgtttccctgcctgcaccatagccagaatctgctgagagccgtgagcaacatgctgcagaaggccagacagacgctcgagttctacccctgcaccagcgaggagattgaccacgaggacatcaccaaggacaagaccagcaccgtggaggcctgcctgcccctcgagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagacaatgaacgccaagctgctgatggaccccaagaggcagatttttctggaccagaacatgctggccgttatcgacgagctgatgcaggccctgaatttcaatagcgaaaccgtgccccagaagagcagcctggaggagcctgacttctacaaaaccaagatcaagctttgcatcctgctgcacgccttcagaatcagagccgtgaccatcgacagagtgatgagctacctgaacgccagcggcagcggcggcggcagcgacgcccacaagagcgaggtggcccataggttcaaggacctgggcgaggagaacttcaaggccctggtactcatcgccttcgcccaatacctgcaacagtgccccttcgaggaccatgttaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgaacgtaacgagtgcttcctgcagcacaaggacgacaaccccaacctgccccgactggtcagacccgaggtggacgtgatgtgcacagccttccacgacaacgaggagaccttcctgaagaagtacctctacgagatcgccagaagacatccatacttctacgcccccgagctgctgttcttcgccaagaggtacaaggccgccttcacagagtgctgccaggccgccgacaaggccgcttgcctgctgcctaagttggacgagctgagagacgagggcaaggccagcagcgccaagcagagactgaagtgcgcaagcctgcagaaattcggcgagagagcctttaaggcctgggccgtggccagactgagccagcgcttccccaaggccgagttcgccgaggtgagcaagctggtgaccgacctgaccaaagtgcacaccgagtgctgccacggcgacctgctggagtgcgccgacgacagagccgacctggccaagtacatctgcgagaaccaggacagcatcagcagcaagttgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggagacaccccgactacagcgtggtgctgctgctgagactggccaagacctacgagaccaccctggagaagtgctgtgccgccgccgacccccacgagtgctacgccaaggtgttcgacgagttcaagcccctggtggaggagccccagaacctgatcaagcagaactgcgagctgttcgagcagctgggcgagtataagttccagaacgccctgctggtgagatacaccaagaaggtgccccaggtcagcacccccaccctggtggaggtgagcagaaatctgggcaaggtgggcagcaaatgctgcaagcaccccgaggccaagagaatgccctgcgccgaggactacctgtcagtggtgctgaaccagctgtgtgtgctgcacgagaagacccccgtgagcgacagagtgaccaagtgctgcaccgagagtctcgtgaacagacggccctgcttcagcgccctggaggtggacgaaacatacgtgcccaaggagttcaacgcagagaccttcaccttccacgcagacatctgcaccctgagcgagaaggagagacagatcaagaagcagaccgccctggttgagcttgtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 13 - DNA, 3393 bp, Artificial Sequence
  vH2 Construct
atgagagtgcccgcccagctgctgggcctgctgctgctgtggctgcccggcgccagatgtgccatctgggagctgaagaaggacgtgtacgtggtggagctggactggtaccccgacgcccccggagagatggtggtgctgacctgcgacacccccgaagaggacggcatcacctggaccctggaccagagcagcgaggtgctgggcagcgggaagaccctgaccatccaggtgaaggagttcggcgacgccggccagtacacctgtcacaagggcggcgaggtgctgagccacagcctgctgctgctgcacaagaaggaggacggcatatggagcaccgacatcctgaaggaccagaaggagcccaagaacaagacctttctgagatgcgaggctaagaattacagcggcagattcacctgctggtggctgaccaccatcagcaccgacctgaccttcagcgtgaagagcagcagaggcagcagcgacccccagggcgtgacatgcggcgccgccaccctgagcgccgagagagtgagaggcgacaacaaggagtacgagtacagcgtggagtgccaggaggactccgcttgccccgccgccgaggagagcttgcccatcgaggtgatggtggacgccgtgcacaagctcaagtacgaaaactacaccagcagcttcttcatcagagacatcatcaagcccgacccccccaagaacctgcagctgaagcccctgaaaaacagcagacaggtggaggtgagctgggagtaccccgacacctggagcaccccccacagctacttcagcctgaccttctgcgtgcaggtgcagggaaaaagcaagagagagaagaaggacagagtgttcaccgacaagaccagcgccaccgtgatctgcagaaagaacgccagcatctccgtgagagcccaggacagatactacagcagcagctggagcgagtgggccagcgtgccctgtagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccatgaggacatcaccaaggacaagaccagcaccgtggaggcctgcctgcccctggagctgaccaagaacgaaagctgcctgaacagcagagagaccagcttcatcaccaacggcagctgtctggcctcaagaaagaccagctttatgatggccctgtgcctgtctagcatctacgaggatctgaagatgtaccaggtggagttcaagaccatgaacgccaagctgctgatggaccccaagagacagatcttcctggaccagaacatgttagccgtgattgacgagctgatgcaggccctgaacttcaatagcgagaccgtgccccagaaaagcagcctggaggagcccgacttctacaagaccaagatcaagctgtgcatcctgctgcacgccttcagaatcagagccgtaaccatcgacagagtgatgagctacctgaacgccagtggcagcggcggtggcagcgacgctcacaagagcgaggtggcccacagattcaaggacctgggcgaggagaacttcaaggccctggtcctgatcgccttcgcccagtacctgcagcagtgccccttcgaggaccacgtgaagctggtgaacgaggtgaccgagttcgccaagacctgcgtggccgacgagtcggccgagaactgcgacaagagcctgcacaccctgttcggcgacaagctgtgcaccgtggccaccctgagagaaacctacggggagatggccgactgctgcgcaaagcaggagcccgagcgaaacgagtgcttcctgcagcacaaggacgacaaccccaacttgcccagactggtaagacccgaggtggacgtcatgtgcaccgctttccacgacaacgaggagaccttcctgaagaagtacctgtacgagatcgccagaagacacccctatttctatgcccctgagctgctgttcttcgccaagcgctacaaggccgccttcaccgagtgctgccaggccgccgacaaggccgcctgcctgctccccaagctggacgagctgagagacgaaggcaaggccagcagcgccaagcagagactgaagtgcgccagcctgcagaagttcggcgagagagccttcaaggcctgggccgtggccagactgtcgcagagattccccaaggccgagttcgccgaggtgagcaagctggttaccgacctgactaaggtgcacaccgagtgctgccacggcgacctgctggagtgcgccgacgacagagccgacctggccaagtacatctgcgagaaccaggatagcatcagcagcaagctgaaggagtgctgcgagaagccccttctggagaagtcccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagactacccttgagaagtgctgcgccgccgccgacccacatgagtgctacgccaaggtgttcgacgagttcaagcccctggtggaagagccccagaacctgatcaagcagaactgcgagctgttcgagcagctgggcgagtacaagttccagaacgcccttctggtgagatacaccaagaaggtgcctcaggtgagcacccccacccttgtggaggtgagcagaaacctcggcaaggtgggcagcaagtgctgcaagcatccagaggccaagagaatgccctgcgccgaggactacctgagcgtggtgctgaaccagctgtgcgtgctgcacgagaaaactcccgtgagcgacagagtgaccaagtgctgcaccgagagtctggtgaacagaagaccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctcaaggccgtgatggacgacttcgccgcgttcgtggagaagtgctgcaaggccgacgacaaagagacctgcttcgccgaggagggcaagaagcttgtggccgccagccaggccgccctgggcctgtag

SEQ ID NO: 14 - DNA, 3393 bp, Artificial Sequence
  vH3 Construct
atgagagtgcccgcccagcttctgggcctgctgctgctgtggctgcccggcgccagatgcgccatctgggagctgaagaaggacgtgtacgtggtggagctggactggtacccggacgccccgggcgagatggtcgtgctgacctgcgacacccccgaggaggacggcatcacctggaccctggaccagtctagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcctaagaacaagaccttcctgagatgtgaggccaagaactacagcggcagattcacctgctggtggctgaccaccatcagcaccgatctgaccttcagcgtgaagagcagcagaggcagctcagacccccagggcgtgacctgcggcgccgcgaccctgagcgccgagagagtgagaggcgacaacaaggagtacgagtacagcgtggagtgccaggaggacagcgcctgccccgcggccgaggagagcctgcccatcgaggtgatggtggacgccgtgcataagctgaagtacgagaactacaccagcagcttcttcatcagagacatcatcaagcccgatccccccaagaacctgcagctgaagccactgaagaacagcagacaggtggaggtgagctgggagtaccccgacacctggagcaccccccacagctacttcagcctgaccttctgcgtgcaggtgcagggcaagagcaagagagagaagaaggaccgggtgttcaccgacaagaccagcgccactgtgatctgcagaaagaacgccagcatcagcgtgagagcccaggacagatactacagctccagctggtcagagtgggccagcgtgccctgcagcggcggcagcggtggcgggagcggcggcgggagcggcggcggaagcagaaacctgcccgtggccacccctgaccccggcatgttcccctgcctgcaccacagccagaacctgctgagagccgtaagcaacatgctgcagaaggccagacagactctggagttctacccctgcaccagcgaggagatcgatcacgaggacatcaccaaggacaagaccagtaccgtggaggcctgcctgcccctggagctgaccaagaacgagagctgcctgaacagcagagagaccagctttataaccaacggcagctgtctggctagcagaaagaccagcttcatgatggccctgtgcctgagcagcatctacgaggacttgaagatgtaccaggtggagttcaagaccatgaacgccaagcttctgatggaccccaagagacagatctttctggaccagaacatgctggccgtgatcgatgaactgatgcaggccctgaacttcaacagcgagaccgtgccccagaagagcagtctggaggagcccgacttctacaagactaagatcaagctgtgcatcctgctgcacgccttcagaatcagagccgtgaccatcgacagagtgatgagctacctgaacgcctcaggcagcggcggcgggagcgacgcccacaagagcgaggtggcccacagattcaaggacctcggcgaggagaacttcaaggccctggtgctgatcgctttcgcccagtacctgcagcagtgccccttcgaggaccacgtgaaactggtgaacgaggtgaccgagttcgccaagacctgcgtggccgacgagagcgccgagaactgcgacaagagcctgcacacgttgttcggcgacaagctgtgcaccgtggccaccctgagagagacctacggcgagatggccgactgctgcgccaagcaggagcccgagagaaacgaatgcttcctgcagcacaaggacgacaaccccaacctgccccgcctggtgagacccgaggtggacgtgatgtgcaccgccttccacgacaacgaggagaccttcctgaagaagtacttgtacgagatcgcaagaagacacccgtacttctacgcccccgagctgctgttcttcgccaagagatacaaggccgccttcaccgagtgctgccaggccgccgacaaggccgcctgcctgctgcccaagctggacgagctcagagacgagggcaaggccagcagcgccaagcagagactgaagtgcgccagcctgcagaagttcggcgagagagcctttaaggcctgggccgtggccagactgagccagagattccccaaggccgagttcgccgaagtgagcaagctggtgaccgacctgacgaaggtgcacaccgagtgctgccacggcgacctgctggagtgcgccgacgacagagcagacctggccaagtacatctgcgagaaccaggacagcatcagcagcaagctgaaggagtgctgcgagaagcccctgctggagaagagccactgcatcgccgaggtggagaacgacgagatgcccgccgacctgcccagtctggccgcagacttcgtggagagcaaggatgtgtgcaagaactacgccgaggccaaggacgtgttcctgggcatgttcctgtacgagtacgcaagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgaactgttcgagcagcttggcgagtacaagtttcagaacgccctgctggtcagatacaccaagaaggtgccccaggtgagcacccccaccctggtggaggtgtccagaaacctgggcaaggtgggcagcaagtgctgcaagcaccccgaggcaaagagaatgccctgcgccgaggactatctgagcgtggtgctgaaccagctgtgcgtgctgcacgagaagacccccgtgagcgacagagtgaccaagtgctgcaccgagagcctggtgaatagaagaccctgcttcagcgcactggaggtggacgagacctacgtgcccaaggagttcaacgccgagacattcaccttccacgccgatatctgcaccctgagcgagaaggagagacagatcaagaagcagaccgccctggtggagctggtgaagcacaagcccaaggccaccaaggagcagttgaaggccgtgatggacgacttcgccgccttcgtggagaaatgctgcaaggccgacgacaaggagacctgcttcgccgaggagggcaagaagctggtggccgccagccaggccgccctgggcctgtag

SEQ ID NO: 15 - DNA, 1617 bp, Artificial Sequence
  A1 Construct
atgagggtccccgctcagctcctggggctcctgctgctctggctcccaggtgcacgatgtgccatctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggaagcggcggaggctctggcggagggagtggcggaggcagcagaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagc

SEQ ID NO: 16 - DNA, 1614 bp, Artificial Sequence
  A2 Construct
atgagggtccccgctcagctcctggggctcctgctgctctggctcccaggtgcacgatgtgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggaggtggcagtggcggaggcagcagaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgct

SEQ ID NO: 17 - DNA, 1617 bp, Artificial Sequence
  A3 Construct
atgagggtccccgctcagctcctggggctcctgctgctctggctcccaggtgcacgatgtgccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaaaagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaaaagcagcctggaggagcccgacttctacaagaccaagatcaagctgtgcatcctgctgcacgccttcagaatcagagccgtgaccatcgacagagtgatgagctacctgaacgccagc

SEQ ID NO: 18 - DNA, 1617 bp, Artificial Sequence
  A4 Construct
atgagggtccccgctcagctcctggggctcctgctgctctggctcccaggtgcacgatgtgccatctgggagctgaagaaggatgtgtatgtggtggagctggactggtacccagatgcccctggagagatggtggtgctgacctgtgacaccccagaggaggatggcatcacctggaccctggaccagagcagcgaggtgctgggcagcggcaagaccctgaccatccaggtgaaggagtttggagatgctggccagtacacctgccacaagggcggggaggtgctgagccacagcctgctgctgctgcacaagaaggaggatggcatctggagcacagacatcctgaaggaccagaaggagcccaagaacaagaccttcctgcgctgtgaggccaagaactacagcggccgcttcacctgctggtggctgaccaccatcagcacagacctgaccttctctgtgaaaagcagccggggcagcagtgacccccagggcgtgacctgtggggccgccaccctgtctgctgagcgggtgcggggggacaacaaggagtatgagtacagcgtggagtgccaggaggacagcgcctgcccagctgctgaggagagcctgcccatcgaggtgatggtggatgctgtgcacaagctgaagtatgagaactacaccagcagcttcttcatccgggacatcatcaagccagacccccccaagaacctgcagctgaagcccctgaagaacagccggcaggtggaggtgtcctgggagtacccagacacctggagcaccccccacagctacttcagcctgaccttctgtgtgcaggtgcagggcaagagcaagcgggagaagaaggacagagtcttcacagacaagaccagcgccaccgtcatctgcaggaagaatgccagcatctctgtgcgggcccaggaccgctactacagcagctcctggagcgagtgggcctctgtgccctgcagcggcggcagcggcggcggcagcggcggcggcagcggcggcggcagcaggaacctgcctgtggccaccccagaccccggcatgttcccctgcctgcaccacagccagaacctgctgcgggctgtgagcaacatgctgcagaaggcccggcagaccctggagttctacccctgcaccagcgaggagattgaccacgaggacatcaccaaggacaagaccagcacagtggaggcctgcctgcccctggagctgaccaagaatgaaagctgcctgaacagccgggagaccagcttcatcaccaacggcagctgcctggccagcaggaagaccagcttcatgatggccctgtgcctgagcagcatctatgaggacctgaagatgtaccaggtggagttcaagaccatgaatgccaagctgctgatggaccccaagaggcagatcttcctggaccagaacatgctggccgtgattgatgagctgatgcaggccctgaacttcaacagcgagaccgtgccccagaaaagcagcctggaggagccagacttctacaagaccaagatcaagctgtgcatcctgctgcacgccttccgcatccgggctgtgaccatcgacagagtgatgagctacctgaatgccagc

SEQ ID NO: 19 - DNA, 3390 bp, Artificial Sequence
  B1 Construct
atgagggtccccgctcagctcctggggctcctgctgctctggctcccaggtgcacgatgtgccatctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggaagcggcggaggctctggcggagggagtggcggaggcagcagaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagcggctctggcggcggcagtgacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgaatgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaaaagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctt

SEQ ID NO: 20 - DNA, 3390 bp, Artificial Sequence
  B2 Construct
atgagggtccccgctcagctcctggggctcctgctgctctggctcccaggtgcacgatgtgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggaggtggcagtggcggaggcagcagaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgctagcggcagcggtggcggcagcgacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctgttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggaaaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctg

SEQ ID NO: 21 - DNA, 3390 bp, Artificial Sequence
  B3 Construct
atgagggtccccgctcagctcctggggctcctgctgctctggctcccaggtgcacgatgtgccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaaaagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaaaagcagcctggaggagcccgacttctacaagaccaagatcaagctgtgcatcctgctgcacgccttcagaatcagagccgtgaccatcgacagagtgatgagctacctgaacgccagcggcagcggcggcggcagcgacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaaaagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctg

SEQ ID NO: 22 - DNA, 3390 bp, Artificial Sequence; identical to SEQ ID NO: 11
  B4 Construct

SEQ ID NO: 23 - DNA, 4374 bp, Artificial Sequence
  C1 Construct
atgagggtccccgctcagctcctggggctcctgctgctctggctcccaggtgcacgatgtgccatctgggagctgaaaaaggacgtgtacgtggtggaacttgactggtaccccgacgcccccggcgagatggtggtactgacctgcgacactcccgaggaagacggcattacctggaccttggaccagagcagcgaggttctgggctccggaaaaaccttgacaatccaagtgaaagaattcggcgacgctggccagtacacctgccacaagggcggcgaggtgctgtcccacagcctgctgctgctgcataagaaagaagacgggatttggagcaccgatatactgaaggatcagaaggagcccaagaacaagaccttcctgaggtgcgaggccaaaaattacagcggcagattcacctgctggtggctgaccaccattagcacagacctgactttcagcgtaaagtcttcaaggggcagctcagacccccagggagtaacttgcggagcggcaacgttgtctgccgagcgggtcagaggcgacaataaggagtacgagtattcagtagagtgtcaggaagatagcgcctgtcccgccgcggaggagagcctccccatcgaggtgatggtggacgccgtgcacaagttaaagtacgagaattacaccagctcattttttatcagagacattatcaagccggaccccccgaagaacttacagcttaaacccctaaagaacagcaggcaggttgaggtcagctgggaatatcctgacacctggtcaaccccccacagctacttctcccttactttctgtgtgcaagtgcagggcaagagcaagagagaaaagaaggaccgggtgtttaccgacaagactagcgccaccgtgatttgcagaaagaacgccagcattagtgtgagagcccaggacaggtattactccagctcatggtctgagtgggctagtgtgccttgctctggaggcagcggtggcgggagcggcggaggctccgggggaggtagccggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaaaagcagcctcgaggagcccgacttctacaaaacaaagatcaaactctgcatccttctgcacgccttcagaattagagccgtgaccatcgacagagttatgagctacctgaatgccagcggcagcggcggcggatccgatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctcggtggcgggtctggtggcggttctcagtactacgattacgacttccccctgagtatctacggtcagtcctcacctaactgcgccccagagtgtaactgccccgaaagctacccgagcgccatgtactgcgacgagctgaagttgaagtccgtgcccatggtgcccccaggcatcaagtacttatacttgaggaacaaccaaatcgatcatatcgacgagaaggccttcgaaaacgtaaccgacctgcagtggctgatactggatcacaatctgctagagaattccaagatcaagggcagagtgttctcgaagctgaaacaactgaagaagctgcacatcaaccataacaatctcaccgagagcgtgggtcccctgcccaagtcgctggaggacctgcagctgacccacaacaaaataaccaaactaggcagcttcgaggggttggtgaatctgaccttcatccatttgcagcataacagactaaaggaggatgccgtgagcgccgccttcaaaggcctcaagagccttgagtacctggacctgagcttcaaccagatcgcccggctgcccagtgggctgcccgtgagcctgctgacgctgtatctggacaataacaaaatcagcaacatccccgacgaatacttcaaaagattcaacgccttacagtacctgcgactcagccacaatgagctcgctgacagcggcatccctggcaacagcttcaacgtgtcatccctggtggagctggacctgagttacaataagctgaagaacatcccaactgtcaatgagaatttggaaaactactacctggaggtgaaccagctggagaagttcgacattaagagcttttgcaaaatcctgggcccactgtcatatagcaagatcaagcacctgcgactggacggcaaccgaatcagtgaaacttccctaccccctgacatgtacgagtgcctgagagtagcaaatgaggtgaccctgaac

SEQ ID NO: 24 - DNA, 4374 bp, Artificial Sequence
  C2 Construct
atgagggtccccgctcagctcctggggctcctgctgctctggctcccaggtgcacgatgtgccatctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggaagcggcggaggctctggcggagggagtggcggaggcagcagaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagcggctctggcggcggcagtgacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgaatgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaaaagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggccttggtggcgggtctggtggcggttctcagtattacgactacgatttccccctgagcatctatggccagagcagccctaactgcgccccggagtgcaactgccccgaaagctacccaagcgccatgtactgcgatgagctgaagctgaagtctgtgcctatggtgcctcccggcatcaagtacctgtacctgagaaacaaccagatagaccacatcgatgagaaagccttcgagaacgtcaccgacctgcagtggctgattctggaccacaatttactggagaactccaagatcaagggcagagtgttctccaagttaaagcagctgaagaaactgcacatcaatcacaacaacctgaccgagagcgtgggcccactgcccaagagcctagaggatctgcagctcacccacaacaagatcactaagttgggcagcttcgagggcctcgtaaacttgacattcatacatctgcagcacaacagacttaaggaagacgccgtgagtgcggcctttaagggtctgaaaagcctggagtacttagacctgagcttcaaccagatcgcaaggctgcccagcggccttccggtcagtctgctgaccctgtatctggacaacaacaagatcagcaacatccccgacgagtacttcaagcggtttaacgccctccagtacctgagactgagccacaacgagttagctgactcgggcatacccggtaacagcttcaatgttagcagcctagttgagcttgacttgagctacaacaagcttaagaacatcccaaccgtgaacgagaacctcgagaattactacctggaagtcaaccagctggagaagttcgacattaagagcttctgcaaaatcctgggcccactgtcctatagcaagatcaagcacctgcgccttgacggaaacagaattagcgagaccagccttccaccagacatgtacgagtgcctgagggtggccaacgaggtgaccctgaac

SEQ ID NO: 25 - DNA, 4374 bp, Artificial Sequence
  C3 Construct
atgagggtccccgctcagctcctggggctcctgctgctctggctcccaggtgcacgatgtgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggaggtggcagtggcggaggcagcagaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgctagcggcagcggtggcggcagcgacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctgttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggaaaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctgggtggcgggtctggtggcggttctcagtactacgactacgatttccccctatccatctacgggcagagctcgcctaactgcgcccccgagtgtaactgccccgagtcgtaccccagcgccatgtactgtgacgagctgaagctgaaaagcgtgcccatggtgccccccggcatcaagtacctgtacttgagaaacaaccagatcgaccacattgacgaaaaggccttcgagaacgtaaccgacctgcagtggctgatcctggaccacaacctgcttgagaacagcaagatcaagggccgcgtgttcagcaagctgaagcagctgaagaagctgcacatcaaccacaacaacttgactgagtctgttggccccctaccaaagagcctggaggacctgcagctgacccacaataagataaccaagctgggctcattcgagggcctggtgaacttgacctttattcacctgcagcataacagactgaaggaggacgccgtgagcgccgcctttaaggggctgaaaagcctggagtacctggacctgagttttaaccagatcgccagactgccctcaggcctgcccgtgagtttgctgactctgtacctggacaacaataagatcagcaacattcctgacgagtatttcaaaagattcaatgctctgcagtacctgagactaagccacaacgagctggccgacagcggaatccccggcaacagcttcaacgtgagcagcttggtggagttggacctgagctacaacaaactgaagaacatccccaccgtcaatgagaacttggagaattactacctcgaggttaaccagcttgagaagttcgacatcaagagcttctgcaagatcctgggccccctcagctacagcaagatcaagcacttgagactggacgggaacagaatcagcgaaaccagccttcctcccgacatgtacgagtgccttagagtggcaaatgaggtgaccctgaac

SEQ ID NO: 26 - DNA, 63 bp, Artificial Sequence
  Leader Sequence of L1
atgcgagttcctgctcagctgcttggtctgttactgctgtggttgccgggcgcacgatgtgct

SEQ ID NO: 27 - DNA, 63 bp, Artificial Sequence
  Leader Sequence of L2
atgcgcgtgcccgcacaattgctcggactgctgctactgtggctgcccggggctcgctgcgca

SEQ ID NO: 28 - DNA, 63 bp, Artificial Sequence
  Leader Sequence of L3
atgagagttcctgctcaactactagggctgctgttgttgtggttgcccggcgcgcgatgcgca

SEQ ID NO: 29 - DNA, 63 bp, Artificial Sequence
  Leader Sequence of M1
atgagagtccccgcccagctgctggggcttcttttgctttggcttcctggcgcaagatgcgct

SEQ ID NO: 30 - DNA, 63 bp, Artificial Sequence
  Leader Sequence of M2
atgagagtccccgcccagctgctagggctgctgctgctgtggttacccggcgcccggtgtgca

SEQ ID NO: 31 - DNA, 63 bp, Artificial Sequence
  Leader Sequence of M3
atgagagtcccagctcagctgctgggcctactgctgctatggctccccggcgcgcggtgcgcc

SEQ ID NO: 32 - DNA, 65 bp, Artificial Sequence
  Leader Sequence of H1
cvatgagagtgcccgcccagttgcttggcctgctgctcctatggcttcccggcgccagatgcgcc

SEQ ID NO: 33 - DNA, 63 bp, Artificial Sequence
  Leader Sequence of H2
atgagagtgcccgcccagctgctgggcctgctgctcttatggctgcccggcgcccgctgtgct

SEQ ID NO: 34 - DNA, 63 bp, Artificial Sequence
  Leader Sequence of H3
atgagagtgcccgcccagctgctgggcctgctgttgctgtggctgcccggcgccagatgcgcc

SEQ ID NO: 35 - DNA, 63 bp, Artificial Sequence
  Leader Sequence of CO
atgagagtgcccgcccagctgctgggcctgctgctgctgtggctgcccggcgccagatgcgcc

SEQ ID NO: 36 - DNA, 63 bp, Artificial Sequence
  Leader Sequence of CP
atgagggtccccgctcagctcctggggctcctgctgctctggctcccaggtgcacgatgtgcc

SEQ ID NO: 37 - DNA, 63 bp, Artificial Sequence; identical to SEQ ID NO: 35
  Leader Sequence of vH1

SEQ ID NO: 38 - DNA, 63 bp, Artificial Sequence
  Leader Sequence of vH2
atgagagtgcccgcccagctgctgggcctgctgctgctgtggctgcccggcgccagatgtgcc

SEQ ID NO: 39 - DNA, 63 bp, Artificial Sequence
  Leader Sequence of vH3
atgagagtgcccgcccagcttctgggcctgctgctgctgtggctgcccggcgccagatgcgcc

SEQ ID NO: 40 - DNA, 63 bp, Artificial Sequence; identical to SEQ ID NO: 36
  Leader Sequence of A1

SEQ ID NO: 41 - DNA, 63 bp, Artificial Sequence; identical to SEQ ID NO: 36
  Leader Sequence of A2

SEQ ID NO: 42 - DNA, 63 bp, Artificial Sequence; identical to SEQ ID NO: 36
  Leader Sequence of A3

SEQ ID NO: 43 - DNA, 63 bp, Artificial Sequence; identical to SEQ ID NO: 36
  Leader Sequence of A4

SEQ ID NO: 44 - DNA, 63 bp, Artificial Sequence; identical to SEQ ID NO: 36
  Leader Sequence of B1

SEQ ID NO: 45 - DNA, 63 bp, Artificial Sequence; identical to SEQ ID NO: 36
  Leader Sequence of B2

SEQ ID NO: 46 - DNA, 63 bp, Artificial Sequence; identical to SEQ ID NO: 36
  Leader Sequence of B3

SEQ ID NO: 47 - DNA, 63 bp, Artificial Sequence; identical to SEQ ID NO: 36
  Leader Sequence of B4

SEQ ID NO: 48 - DNA, 63 bp, Artificial Sequence; identical to SEQ ID NO: 36
  Leader Sequence of C1

SEQ ID NO: 49 - DNA, 63 bp, Artificial Sequence; identical to SEQ ID NO: 36
  Leader Sequence of C2

SEQ ID NO: 50 - DNA, 63 bp, Artificial Sequence; identical to SEQ ID NO: 36
  Leader Sequene of C3

SEQ ID NO: 51 - DNA, 918 bp, Artificial Sequence
  IL-12b of L1
atctgggaattaaagaaagatgtgtacgtggtggaattggattggtacccagatgctccgggcgaaatggttgtactcacatgcgatactccggaggaagacggtatcacttggacattggatcagtcgagtgaggttctcggtagtggtaaaacactaacgatccaagtcaaagaattcggtgatgcggggcaatatacctgtcataaaggcggcgaagtactatctcatagcctcctgctgttacacaagaaggaagatggcatatggtccaccgacatccttaaggatcagaaagaacccaagaacaaaactttcttgcgttgcgaagctaagaactactccggccgcttcacatgctggtggttgacaacgatcagtacggatctaaccttctctgttaagtccagtcgggggagttcggacccccaaggcgtcacgtgtggagctgccacactttccgctgagcgcgtacgtggagataataaagagtatgaatactccgttgagtgccaggaggactccgcgtgccccgctgccgaggagagtctccccatagaggtgatggtcgacgctgttcacaaactgaaatatgagaactatacctcatccttctttatacgtgacataattaagccagatcccccgaagaacttacaattgaaaccattgaagaattcacgtcaagtcgaggtatcctgggagtatcccgacacctggtccacgccacactcatatttctctctgaccttctgtgtgcaggtacaaggcaagagcaaacgagaaaaaaaggacagagttttcacggataagactagcgccacagtgatatgtaggaaaaacgcatcgatctcagtccgcgcgcaagatcggtattactcaagcagttggtcagagtgggcatcggtgccctgctcg

SEQ ID NO: 52 - DNA, 918 bp, Artificial Sequence
  IL-12b of L2
atttgggaacttaagaaggacgtatacgttgtggaacttgactggtaccctgatgctccaggggagatggtggttttgacgtgtgacaccccggaagaagatggaattacatggaccttagaccaatcctccgaggttcttggctcgggcaaaaccttgaccattcaggtcaaggaatttggcgatgctggccaatacacctgccataaaggtggtgaagttttatctcactccctactgttgctccataagaaagaagacggcatttggtcgacagacatattgaaggatcagaaggaacctaagaataagaccttcttacgatgtgaggccaagaattattccggacgttttacgtgttggtggttgaccacgatctccactgacttaaccttctcagtgaaatcctcacgaggtagttccgatccccagggtgtgacgtgcggtgcggccacgttaagtgctgagagagtacggggcgacaataaggaatacgagtactcagttgaatgccaagaggactcggcctgtcccgcggcagaggagagtctccctatcgaagtgatggtggatgcggtgcacaagctcaagtatgaaaattacacatcatcttttttcatcagagacattataaagcccgacccaccaaagaacctccagttaaagcccttaaagaacagtagacaggttgaagtatcatgggaatacccagacacctggtccacaccccattcgtatttttccttgacgttttgcgtacaagttcagggaaagtccaaacgggaaaagaaagaccgcgtttttacagacaaaacttctgccactgtcatctgtagaaagaacgcatcaattagcgtgcgagcgcaagacagatactattcaagtagctggagcgagtgggccagtgttccatgttct

SEQ ID NO: 53 - DNA, 918 bp, Artificial Sequence
  IL-12b of L3
atatgggagctcaagaaggacgtttatgtcgttgagctggattggtaccctgatgccccgggcgaaatggttgtgcttacgtgcgatacccccgaggaggatggcataacatggacgttagatcagtcttccgaggtccttggttccggtaagactcttactatccaggtgaaggagttcggcgatgccggccagtacacttgccataaaggcggtgaagttctaagccactctctactgcttttgcacaagaaggaagatggaatatggtccaccgacatcttgaaggaccagaaagaaccaaaaaataagacatttttgaggtgtgaggcaaaaaattattcgggacgcttcacctgctggtggttgacgacgatttcaaccgacctcaccttctcagtaaagagttcgagaggtagttccgatccccaaggtgtgacatgtggcgctgcgactctaagcgctgaacgcgtaagaggtgataacaaagagtacgaatacagtgtggaatgccaagaagatagcgcgtgtccagccgcagaagaatctttaccaatagaggttatggttgatgccgttcacaaattgaaatatgagaattacacctcaagctttttcattcgagacataataaagcccgaccctcctaagaatcttcagttaaaaccgctgaagaacagtagacaagttgaggttagctgggaatatcctgatacctggtcaacgccgcactcgtatttctccctgactttctgtgttcaggttcaaggaaaatctaaaagggagaagaaagaccgtgttttcaccgacaagacatctgccacagtcatatgtaggaagaacgcttcaatcagcgtgcgagcgcaggaccgatactacagctctagttggtccgaatgggccagcgtaccatgctct

SEQ ID NO: 54 - DNA, 918 bp, Artificial Sequence
  IL12b of M1
atctgggagctgaaaaaggacgtgtacgtggtggaacttgactggtaccccgacgcccccggcgagatggtggtactgacctgcgacactcccgaggaagacggcattacctggaccttggaccagagcagcgaggttctgggctccggaaaaaccttgacaatccaagtgaaagaattcggcgacgctggccagtacacctgccacaagggcggcgaggtgctgtcccacagcctgctgctgctgcataagaaagaagacgggatttggagcaccgatatactgaaggatcagaaggagcccaagaacaagaccttcctgaggtgcgaggccaaaaattacagcggcagattcacctgctggtggctgaccaccattagcacagacctgactttcagcgtaaagtcttcaaggggcagctcagacccccagggagtaacttgcggagcggcaacgttgtctgccgagcgggtcagaggcgacaataaggagtacgagtattcagtagagtgtcaggaagatagcgcctgtcccgccgcggaggagagcctccccatcgaggtgatggtggacgccgtgcacaagttaaagtacgagaattacaccagctcattttttatcagagacattatcaagccggaccccccgaagaacttacagcttaaacccctaaagaacagcaggcaggttgaggtcagctgggaatatcctgacacctggtcaaccccccacagctacttctcccttactttctgtgtgcaagtgcagggcaagagcaagagagaaaagaaggaccgggtgtttaccgacaagactagcgccaccgtgatttgcagaaagaacgccagcattagtgtgagagcccaggacaggtattactccagctcatggtctgagtgggctagtgtgccttgctct

SEQ ID NO: 55 - DNA, 918 bp, Artificial Sequence
  IL12b of M2
atttgggagttgaagaaggacgtgtacgtggtggagctggactggtacccggatgctcccggcgagatggtggtactcacctgcgacacacctgaggaagacggcatcacctggaccctcgatcagagcagcgaggttctgggaagcggcaaaaccctgaccatccaagtgaaagagtttggcgacgccggtcagtacacctgccacaagggaggcgaggtcctgtctcactctctgctgctgctccataagaaggaggacggtatttggagcactgacatcttgaaggatcaaaaagagccaaagaataaaacgttcctgaggtgcgaagctaagaattactccgggcgttttacgtgctggtggctgaccacgatcagcaccgatctgaccttcagcgtgaagagcagccggggcagcagcgacccccaaggcgtgacttgcggcgctgcgaccctgagcgctgagcgtgtgcgcggcgacaacaaggagtatgagtattcagtggagtgtcaggaggactccgcctgtcccgcggccgaagagagtctgcctattgaggtgatggtggacgccgtgcacaagctgaagtacgagaactacacatcgtcattctttatccgcgacatcataaagcccgacccccccaagaacctgcagctgaagcctctcaagaattcccggcaagtggaggtgagctgggagtaccctgatacctggtctacccctcacagctactttagcctgaccttctgcgtccaggtgcaaggaaagtcgaagcgcgagaagaaagatagagtcttcaccgataaaaccagtgccaccgtgatttgccgcaaaaacgcctccatcagcgtgcgggctcaggatagatactactctagcagctggagcgaatgggcctcagttccttgcagc

SEQ ID NO: 56 - DNA, 918 bp, Artificial Sequence
  IL12b of M3
atttgggagctcaagaaggacgtgtacgtggtggaacttgactggtacccggacgcgcccggggaaatggtggtgctaacctgtgacacccccgaagaggacggcatcacctggaccctggaccagagcagtgaggtgctaggtagtggcaaaacgttaaccatccaggtcaaggagttcggcgacgccgggcaatacacctgtcacaaggggggggaggtactatcccactccctgctgctcctgcacaagaaagaggacgggatctggagcaccgacattctgaaagaccaaaaggagcccaaaaacaaaaccttccttagatgtgaagccaagaactacagcggccgtttcacctgctggtggctgaccaccatatctacggaccttaccttttcggtgaagagcagcagggggagttccgacccgcaaggcgtaacttgcggagccgcaaccctgagcgccgagagagtgcgcggcgacaacaaggagtacgagtatagcgtggagtgccaagaggacagcgcatgcccagccgccgaagagagcctgccaatagaggtcatggtagacgccgtgcacaagctaaaatatgaaaactacaccagcagctttttcatcagggatatcatcaaacccgacccaccaaaaaacttacagcttaagcctctgaaaaacagcagacaagttgaggtcagctgggagtaccccgacacttggagcacaccccactcctatttcagtttgacattctgcgtgcaggtgcagggtaaaagcaagagagaaaagaaggacagagtgttcacagataagacctcagccacagtgatctgccgtaagaatgccagcatcagcgtccgggctcaggacaggtactactcttcctcatggagcgagtgggcctctgtcccctgcagc

SEQ ID NO: 57 - DNA, 918 bp, Artificial Sequence
  IL12b of H1
atctgggagctgaagaaagacgtgtacgtggtggagctggactggtaccccgacgcccccggtgaaatggtggttctgacctgcgacacaccagaggaggacggcatcacctggaccctggaccagtccagcgaggtcctgggctctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagc

SEQ ID NO: 58 - DNA, 918 bp, Artificial Sequence
  IL12b of H2
atttgggagctgaagaaggacgtgtacgtggtggagctggattggtatccagacgcccccggcgagatggtcgtgttgacctgcgatactcccgaggaggacggaatcacctggacacttgaccagagcagcgaggtgctgggcagcgggaagaccctgactatccaggtgaaggagtttggcgatgccggacagtacacctgccacaagggcggcgaggtgttatctcatagcctgctgctgctgcacaaaaaggaggacgggatctggagcactgacatcctgaaggaccagaaggagcccaaaaacaagaccttcctgcgttgcgaggccaagaactacagcgggagattcacctgttggtggctgaccactatcagcactgacctaaccttcagcgtgaagagcagccggggaagctctgacccccagggggtgacatgcggcgccgccacactgagcgccgagagggtgagaggggacaataaggaatacgagtatagcgtggagtgtcaggaagattccgcctgccccgccgccgaggagagcctgcctatcgaggtcatggtggacgccgtccataaactgaagtacgaaaactatacttcaagcttcttcatcagagacatcataaagcccgacccccccaagaacctgcagctaaagcccctgaagaacagcagacaggtcgaagtgagctgggagtaccccgatacctggagcaccccgcacagctacttcagcctgaccttttgcgtccaggtgcagggcaagagcaagagagagaagaaggacagggtgttcactgacaagacaagcgccactgttatctgcagaaagaacgccagtatcagcgtgcgcgcccaagacaggtattactccagcagctggtctgaatgggccagcgtgccttgcagc

SEQ ID NO: 59 - DNA, 918 bp, Artificial Sequence
  IL12b of H3
atctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaagagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagc

SEQ ID NO: 60 - DNA, 918 bp, Artificial Sequence
  IL12b of CO
atctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaagagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagc

SEQ ID NO: 61 - DNA, 918 bp, Artificial Sequence
  IL12b of CP
atctgggagctgaagaaggatgtgtatgtggtggagctggactggtacccagatgcccctggagagatggtggtgctgacctgtgacaccccagaggaggatggcatcacctggaccctggaccagagcagcgaggtgctgggcagcggcaagaccctgaccatccaggtgaaggagtttggagatgctggccagtacacctgccacaagggcggggaggtgctgagccacagcctgctgctgctgcacaagaaggaggatggcatctggagcacagacatcctgaaggaccagaaggagcccaagaacaagaccttcctgcgctgtgaggccaagaactacagcggccgcttcacctgctggtggctgaccaccatcagcacagacctgaccttctctgtgaaaagcagccggggcagcagtgacccccagggcgtgacctgtggggccgccaccctgtctgctgagcgggtgcggggggacaacaaggagtatgagtacagcgtggagtgccaggaggacagcgcctgcccagctgctgaggagagcctgcccatcgaggtgatggtggatgctgtgcacaagctgaagtatgagaactacaccagcagcttcttcatccgggacatcatcaagccagacccccccaagaacctgcagctgaagcccctgaagaacagccggcaggtggaggtgtcctgggagtacccagacacctggagcaccccccacagctacttcagcctgaccttctgtgtgcaggtgcagggcaagagcaagcgggagaagaaggacagagtcttcacagacaagaccagcgccaccgtcatctgcaggaagaatgccagcatctctgtgcgggcccaggaccgctactacagcagctcctggagcgagtgggcctctgtgccctgcagc

SEQ ID NO: 62 - DNA, 918 bp, Artificial Sequence
  IL12b of vH1
atctgggagctgaagaaggacgtgtacgtggtggagctggactggtaccccgacgcccccggcgagatggtggtgctgacctgcgacacccccgaggaggacgggatcacctggaccctggatcagagcagcgaggtgctgggcagcggcaagaccctgaccatccaggtgaaggagtttggtgacgccggccagtacacctgccacaagggcggcgaggtgctgagccactcactgctgctgctgcacaagaaggaggacggcatctggagcaccgacattctgaaggatcagaaggagcccaagaacaagaccttcctgagatgcgaggccaagaactacagcggcagattcacctgttggtggctgactactatcagcactgatctgaccttcagcgtgaagagctcaagaggcagcagcgatccccagggcgtgacctgcggcgccgccaccctgagcgccgagagagtgagaggcgacaacaaggagtacgagtacagcgtggagtgccaggaagacagcgcctgccccgctgcagaggagtctctgcccatcgaggtgatggtggacgccgtgcacaagcttaagtacgagaactacaccagctccttcttcattagagacatcatcaagcccgacccgcccaaaaacctgcagctgaagcccctgaagaacagcagacaggtggaggtcagctgggagtaccccgacacctggagcaccccccacagctacttcagcctgaccttctgcgtgcaggtgcagggcaagagcaagagagagaagaaggacagagtgttcaccgacaaaaccagcgccaccgtgatctgcagaaaaaacgccagcattagcgtgagagctcaggatagatactacagcagcagctggagtgagtgggccagcgtgccctgcagc

SEQ ID NO: 63 - DNA, 918 bp, Artificial Sequence
  IL12b of vH2
atctgggagctgaagaaggacgtgtacgtggtggagctggactggtaccccgacgcccccggagagatggtggtgctgacctgcgacacccccgaagaggacggcatcacctggaccctggaccagagcagcgaggtgctgggcagcgggaagaccctgaccatccaggtgaaggagttcggcgacgccggccagtacacctgtcacaagggcggcgaggtgctgagccacagcctgctgctgctgcacaagaaggaggacggcatatggagcaccgacatcctgaaggaccagaaggagcccaagaacaagacctttctgagatgcgaggctaagaattacagcggcagattcacctgctggtggctgaccaccatcagcaccgacctgaccttcagcgtgaagagcagcagaggcagcagcgacccccagggcgtgacatgcggcgccgccaccctgagcgccgagagagtgagaggcgacaacaaggagtacgagtacagcgtggagtgccaggaggactccgcttgccccgccgccgaggagagcttgcccatcgaggtgatggtggacgccgtgcacaagctcaagtacgaaaactacaccagcagcttcttcatcagagacatcatcaagcccgacccccccaagaacctgcagctgaagcccctgaaaaacagcagacaggtggaggtgagctgggagtaccccgacacctggagcaccccccacagctacttcagcctgaccttctgcgtgcaggtgcagggaaaaagcaagagagagaagaaggacagagtgttcaccgacaagaccagcgccaccgtgatctgcagaaagaacgccagcatctccgtgagagcccaggacagatactacagcagcagctggagcgagtgggccagcgtgccctgtagc

SEQ ID NO: 64 - DNA, 918 bp, Artificial Sequence
  IL12b of vH3
atctgggagctgaagaaggacgtgtacgtggtggagctggactggtacccggacgccccgggcgagatggtcgtgctgacctgcgacacccccgaggaggacggcatcacctggaccctggaccagtctagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcctaagaacaagaccttcctgagatgtgaggccaagaactacagcggcagattcacctgctggtggctgaccaccatcagcaccgatctgaccttcagcgtgaagagcagcagaggcagctcagacccccagggcgtgacctgcggcgccgcgaccctgagcgccgagagagtgagaggcgacaacaaggagtacgagtacagcgtggagtgccaggaggacagcgcctgccccgcggccgaggagagcctgcccatcgaggtgatggtggacgccgtgcataagctgaagtacgagaactacaccagcagcttcttcatcagagacatcatcaagcccgatccccccaagaacctgcagctgaagccactgaagaacagcagacaggtggaggtgagctgggagtaccccgacacctggagcaccccccacagctacttcagcctgaccttctgcgtgcaggtgcagggcaagagcaagagagagaagaaggaccgggtgttcaccgacaagaccagcgccactgtgatctgcagaaagaacgccagcatcagcgtgagagcccaggacagatactacagctccagctggtcagagtgggccagcgtgccctgcagc

SEQ ID NO: 65 - DNA, 918 bp, Artificial Sequence
  IL12b of A1
atctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagc

SEQ ID NO: 66 - DNA, 918 bp, Artificial Sequence
  IL12b of A2
atctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagc

SEQ ID NO: 67 - DNA, 918 bp, Artificial Sequence
  IL12b of A3
atctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaaaagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagc

SEQ ID NO: 68 - DNA, 918 bp, Artificial Sequence; identical to SEQ ID NO: 61
  IL12b of A4

SEQ ID NO: 69 - DNA, 918 bp, Artificial Sequence; identical to SEQ ID NO: 65
  IL12b of B1

SEQ ID NO: 70 - DNA, 918 bp, Artificial Sequence; identical to SEQ ID NO: 66
  IL12b of B2

SEQ ID NO: 71 - DNA, 918 bp, Artificial Sequence; identical to SEQ ID NO: 67
  IL12b of B3

SEQ ID NO: 72 - DNA, 918 bp, Artificial Sequence; identical to SEQ ID NO: 61
  IL12b of B4

SEQ ID NO: 73 - DNA, 918 bp, Artificial Sequence; identical to SEQ ID NO: 54
  IL12b of C1

SEQ ID NO: 74 - DNA, 918 bp, Artificial Sequence; identical to SEQ ID NO: 65
  IL12b of C2

SEQ ID NO: 75 - DNA, 918 bp, Artificial Sequence; identical to SEQ ID NO: 66
  IL12b of C3

SEQ ID NO: 76 - DNA, 45 bp, Artificial Sequence
  GS Linker of L1
gggggttctggtgggggcagtggaggaggatcaggtggcggcagt

SEQ ID NO: 77 - DNA, 45 bp, Artificial Sequence
  GS Linker of L2
ggcggttcgggcggagggtccggcggtggtagcggaggcgggagc

SEQ ID NO: 78 - DNA, 45 bp, Artificial Sequence
  GS Linker of L3
ggcggttccggcgggggctcggggggcgggagcggtggaggcagc

SEQ ID NO: 79 - DNA, 45 bp, Artificial Sequence
  GS Linker of M1
ggcggcagcggcggcgggagcggcggaggctccgggggaggtagc

SEQ ID NO: 80 - DNA, 45 bp, Artificial Sequence
  GS Linker of M2
ggcggcagcggtggaggaagcggcggtggcagtgggggcgggagc

SEQ ID NO: 81 - DNA, 45 bp, Artificial Sequence
  GS Linker of M3
ggcggcagcggcggtggcagcggcggcggttctggcggcggttca

SEQ ID NO: 82 - DNA, 45 bp, Artificial Sequence
  GS Linker of H1
ggtggcagcggcggcggctccggcggcgggagtggcggcggcagc

SEQ ID NO: 83 - DNA, 45 bp, Artificial Sequence
  GS Linker of H2
ggggggagcggcggtggcagcgggggcggcagcggcgggggcagc

SEQ ID NO: 84 - DNA, 45 bp, Artificial Sequence
  GS Linker of H3
ggcgggagcggcggcggcagcggcggtggcagtggcgggggcagc

SEQ ID NO: 85 - DNA, 45 bp, Artificial Sequence
  GS Linker of CO
ggcggcagcggcggcggcagcggcggcggcagcggcggcggcagc

SEQ ID NO: 86 - DNA, 45 bp, Artificial Sequence; identical to SEQ ID NO: 85
  GS Linker of CP

SEQ ID NO: 87 - DNA, 45 bp, Artificial Sequence
  GS Linker of vH1
ggcggctcaggcggcggctcaggcggcggcagcggcggcggcagc

SEQ ID NO: 88 - DNA, 45 bp, Artificial Sequence; identical to SEQ ID NO: 85
  GS Linker of vH2

SEQ ID NO: 89 - DNA, 45 bp, Artificial Sequence
  GS Linker of vH3
ggcggcagcggtggcgggagcggcggcgggagcggcggcggaagc

SEQ ID NO: 90 - DNA, 45 bp, Artificial Sequence
  GS Linker of A1
ggtggaagcggcggaggctctggcggagggagtggcggaggcagc

SEQ ID NO: 91 - DNA, 45 bp, Artificial Sequence
  GS Linker of A2
ggcgggagcggcggcggcagcggaggtggcagtggcggaggcagc

SEQ ID NO: 92 - DNA, 45 bp, Artificial Sequence; identical to SEQ ID NO: 85
  GS Linker of A3

SEQ ID NO: 93 - DNA, 45 bp, Artificial Sequence; identical to SEQ ID NO: 85
  GS Linker of A4

SEQ ID NO: 94 - DNA, 45 bp, Artificial Sequence; identical to SEQ ID NO: 90
  GS Linker of B1

SEQ ID NO: 95 - DNA, 45 bp, Artificial Sequence; identical to SEQ ID NO: 91
  GS Linker of B2

SEQ ID NO: 96 - DNA, 45 bp, Artificial Sequence; identical to SEQ ID NO: 85
  GS Linker of B3

SEQ ID NO: 97 - DNA, 45 bp, Artificial Sequence; identical to SEQ ID NO: 85
  GS Linker of B4

SEQ ID NO: 98 - DNA, 45 bp, Artificial Sequence
  GS Linker of C1
ggaggcagcggtggcgggagcggcggaggctccgggggaggtagc

SEQ ID NO: 99 - DNA, 45 bp, Artificial Sequence; identical to SEQ ID NO: 90
  GS Linker of C2

SEQ ID NO: 100 - DNA, 45 bp, Artificial Sequence; identical to SEQ ID NO: 91
  GS Linker of C3

SEQ ID NO: 101 - DNA, 591 bp, Artificial Sequence
  IL12a of L1
cgcaatctacccgtggcaacaccagacccgggaatgttcccatgtctgcaccacagtcaaaacctcttaagggccgtgtcaaatatgctacagaaggcgagacagactttagaattctacccttgtacaagcgaggagattgaccacgaggacatcaccaaagataagacgagcaccgtcgaggcttgcctgcctctagaactaacaaaaaatgaatcatgcttgaactcgagggagaccagtttcattactaacggttcatgtcttgcatcgaggaagacctcattcatgatggccctgtgcctctcgtccatttatgaagacctaaagatgtaccaggtagagtttaagaccatgaacgccaagctcctcatggatccaaaacggcaaatattcttagatcagaatatgctcgctgttatcgacgaactcatgcaggcgcttaacttcaactcagaaaccgttccccaaaagtcgagtctagaagaaccggacttttataagaccaaaattaaactgtgtatactacttcacgccttcaggataagagcagtgacgattgacagggtgatgtcctacttgaatgcatca

SEQ ID NO: 102 - DNA, 591 bp, Artificial Sequence
  IL12a of L2
aggaacttgcccgtggctactccagaccctggcatgttcccctgtttacaccactcccagaacttattacgtgctgttagcaacatgttgcaaaaggcccgtcaaaccctcgagttttacccctgtactagtgaagaaatcgaccacgaagacataacaaaagacaagacaagcacagttgaggcatgcttacccctggagttaacaaagaacgagagctgtctgaactctcgagagacgagcttcatcaccaatgggagttgccttgcttctcgaaaaacgtcgttcatgatggccctgtgcctgtcgtctatctatgaggatctgaaaatgtatcaggttgaattcaagacaatgaatgccaaactactcatggatccaaaacggcagatattcctcgatcagaatatgctcgcagttattgacgaactaatgcaggctctgaattttaacagcgagaccgttcctcagaagtcaagtttggaagaacctgacttctacaaaactaaaataaaattatgcatcttactgcatgctttcagaattagagctgtcactattgatcgagtgatgtcatacttgaatgcttcc

SEQ ID NO: 103 - DNA, 591 bp, Artificial Sequence
  IL12a of L3
aggaatcttcctgtcgcgacccctgatcccggcatgtttccttgcctgcaccacagtcagaacttattacgcgccgtttccaatatgttacagaaggccagacagacattagagttttatccttgcacatcggaggagatcgaccatgaagatatcacaaaagataaaacatccaccgttgaggcctgcctgccacttgaacttactaaaaacgagagctgcctgaatagccgggaaacttctttcatcactaatggatcgtgtctagcaagccgaaaaaccagcttcatgatggctttgtgcctctcgtccatctatgaagacctgaaaatgtatcaagtagaatttaaaacgatgaatgccaaactgcttatggatcccaaacgccagatatttctagaccagaatatgctggccgtcattgatgagctaatgcaggctctcaattttaatagcgaaacagtgccccaaaaaagctctttggaagagccggatttttacaaaaccaagattaagctatgtatcctgctgcatgctttcagaattcgagctgttacaattgatcgggttatgagctacttaaacgcctcg

SEQ ID NO: 104 - DNA, 591 bp, Artificial Sequence
  IL12a of M1
cggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaagagcagcctcgaggagcccgacttctacaaaacaaagatcaaactctgcatccttctgcacgccttcagaattagagccgtgaccatcgacagagttatgagctacctgaatgccagc

SEQ ID NO: 105 - DNA, 591 bp, Artificial Sequence
  IL12a of M2
agaaatctgcccgtcgccactccagatcctggcatgttcccgtgcctgcatcacagccaaaacctgctgcgggcggtgtctaacatgctgcagaaggctaggcagaccttggaattctatccctgcacaagcgaggaaatagaccatgaggacatcaccaaggataagaccagcacggtcgaagcttgcctgccactggaactgacaaaaaacgagagttgcctgaactcccgcgagacatccttcatcacaaacggcagctgcctggctagcaggaagaccagcttcatgatggccctgtgcctgtcttccatctacgaggacctgaaaatgtaccaagtggagttcaagactatgaacgccaagctgctaatggatcccaagcgacagatctttctagaccagaacatgctggccgtcattgacgagctgatgcaggcactcaattttaactcagagaccgtgccacagaagtccagcctggaggagcctgacttctataagaccaagattaagctgtgcatcctgctgcatgccttccgaataagagccgtgaccattgaccgagtgatgtcatacttgaacgcaagc

SEQ ID NO: 106 - DNA, 591 bp, Artificial Sequence
  IL12a of M3
agaaacctcccagtcgctacccccgatcccggaatgttcccctgcctgcaccactcccagaatctgctccgagccgttagcaacatgctgcaaaaggcccggcagaccctggagttctacccatgcacctcggaagaaatcgatcacgaggacatcaccaaggacaagactagcaccgtcgaggcctgcctgccgctggaactaaccaagaatgaaagctgcctcaactcgcgggagacctctttcataaccaacggctcatgcctggccagccggaaaactagctttatgatggctctgtgcttaagcagcatctacgaggatctgaagatgtaccaggtagagttcaagaccatgaatgccaagctgctgatggaccccaagagacaaatcttcctggaccagaacatgctggccgtaattgatgaactgatgcaggccctgaatttcaacagcgagaccgtaccccagaaaagctcactggaggagcccgacttttataagacgaaaataaagttgtgcatccttcttcacgctttccggattagagccgtgaccatcgatagagtgatgtcatacctgaacgcatcg

SEQ ID NO: 107 - DNA, 591 bp, Artificial Sequence
  IL12a of H1
agaaatctgcccgtagccacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaagagcagcctggaggaacccgacttctacaaaaccaagatcaagctgtgcattctgctgcatgccttccgcattagagccgtgaccatcgatagggtgatgagctacctgaacgccagc

SEQ ID NO: 108 - DNA, 591 bp, Artificial Sequence
  IL12a of H2
agaaacctgcccgtggccacacccgatcccggcatgttcccctgtctgcaccacagccaaaacctgctgcgtgccgtgagcaacatgctgcagaaggcgcggcagaccctggagttctatccctgcaccagtgaggagattgaccacgaggatatcaccaaagacaagaccagcaccgtggaggcctgcctccccctggagctgaccaagaacgagtcctgcttgaattcaagagagaccagcttcatcaccaacggctcctgcttagccagcagaaagactagcttcatgatggccttgtgcttgtctagcatctatgaggatctgaagatgtaccaggtcgagttcaagactatgaacgccaagctgctgatggaccccaaaagacagatcttcctggaccagaacatgctggccgtcatcgacgagctgatgcaggcccttaatttcaatagcgagacagtgccccagaaatcttctctggaggagcccgatttttacaaaaccaagatcaaactatgcatcctgttgcacgccttccggatccgcgccgtgaccatcgacagagtaatgtcctacctgaacgccagc

SEQ ID NO: 109 - DNA, 591 bp, Artificial Sequence
  IL12a of H3
agaaacctgcccgtggccacccccgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaagagcagcctggaggagcccgacttttacaagaccaagatcaagttgtgcatcctcctgcacgccttcagaatcagagcggtgacgatcgacagagtcatgagctacctcaacgctagc

SEQ ID NO: 110 - DNA, 591 bp, Artificial Sequence
  IL12a of CO
agaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagc

SEQ ID NO: 111 - DNA, 591 bp, Artificial Sequence
  IL12a of CP
aggaacctgcctgtggccaccccagaccccggcatgttcccctgcctgcaccacagccagaacctgctgcgggctgtgagcaacatgctgcagaaggcccggcagaccctggagttctacccctgcaccagcgaggagattgaccacgaggacatcaccaaggacaagaccagcacagtggaggcctgcctgcccctggagctgaccaagaatgaaagctgcctgaacagccgggagaccagcttcatcaccaacggcagctgcctggccagcaggaagaccagcttcatgatggccctgtgcctgagcagcatctatgaggacctgaagatgtaccaggtggagttcaagaccatgaatgccaagctgctgatggaccccaagaggcagatcttcctggaccagaacatgctggccgtgattgatgagctgatgcaggccctgaacttcaacagcgagaccgtgccccagaaaagcagcctggaggagccagacttctacaagaccaagatcaagctgtgcatcctgctgcacgccttccgcatccgggctgtgaccatcgacagagtgatgagctacctgaatgccagc

SEQ ID NO: 112 - DNA, 591 bp, Artificial Sequence
  IL12a of vH1
agaaacctgcccgtggccacgcccgaccccggcatgtttccctgcctgcaccatagccagaatctgctgagagccgtgagcaacatgctgcagaaggccagacagacgctcgagttctacccctgcaccagcgaggagattgaccacgaggacatcaccaaggacaagaccagcaccgtggaggcctgcctgcccctcgagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagacaatgaacgccaagctgctgatggaccccaagaggcagatttttctggaccagaacatgctggccgttatcgacgagctgatgcaggccctgaatttcaatagcgaaaccgtgccccagaagagcagcctggaggagcctgacttctacaaaaccaagatcaagctttgcatcctgctgcacgccttcagaatcagagccgtgaccatcgacagagtgatgagctacctgaacgccagc

SEQ ID NO: 113 - DNA, 591 bp, Artificial Sequence
  IL12a of vH2
agaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccatgaggacatcaccaaggacaagaccagcaccgtggaggcctgcctgcccctggagctgaccaagaacgaaagctgcctgaacagcagagagaccagcttcatcaccaacggcagctgtctggcctcaagaaagaccagctttatgatggccctgtgcctgtctagcatctacgaggatctgaagatgtaccaggtggagttcaagaccatgaacgccaagctgctgatggaccccaagagacagatcttcctggaccagaacatgttagccgtgattgacgagctgatgcaggccctgaacttcaatagcgagaccgtgccccagaaaagcagcctggaggagcccgacttctacaagaccaagatcaagctgtgcatcctgctgcacgccttcagaatcagagccgtaaccatcgacagagtgatgagctacctgaacgccagt

SEQ ID NO: 114 - DNA, 591 bp, Artificial Sequence
  IL12a of vH3
agaaacctgcccgtggccacccctgaccccggcatgttcccctgcctgcaccacagccagaacctgctgagagccgtaagcaacatgctgcagaaggccagacagactctggagttctacccctgcaccagcgaggagatcgatcacgaggacatcaccaaggacaagaccagtaccgtggaggcctgcctgcccctggagctgaccaagaacgagagctgcctgaacagcagagagaccagctttataaccaacggcagctgtctggctagcagaaagaccagcttcatgatggccctgtgcctgagcagcatctacgaggacttgaagatgtaccaggtggagttcaagaccatgaacgccaagcttctgatggaccccaagagacagatctttctggaccagaacatgctggccgtgatcgatgaactgatgcaggccctgaacttcaacagcgagaccgtgccccagaagagcagtctggaggagcccgacttctacaagactaagatcaagctgtgcatcctgctgcacgccttcagaatcagagccgtgaccatcgacagagtgatgagctacctgaacgcctca

SEQ ID NO: 115 - DNA, 591 bp, Artificial Sequence
  IL12a of A1
agaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagc

SEQ ID NO: 116 - DNA, 588 bp, Artificial Sequence
  IL12a of A2
agaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgct

SEQ ID NO: 117 - DNA, 591 bp, Artificial Sequence
  IL12a of A3
agaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaaaagcagcctggaggagcccgacttctacaagaccaagatcaagctgtgcatcctgctgcacgccttcagaatcagagccgtgaccatcgacagagtgatgagctacctgaacgccagc

SEQ ID NO: 118 - DNA, 591 bp, Artificial Sequence; identical to SEQ ID NO: 111
  IL12a of A4

SEQ ID NO: 119 - DNA, 591 bp, Artificial Sequence; identical to SEQ ID NO: 115
  IL12a of B1

SEQ ID NO: 120 - DNA, 591 bp, Artificial Sequence
  IL12a of B2
agaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgctagc

SEQ ID NO: 121 - DNA, 591 bp, Artificial Sequence; identical to SEQ ID NO: 117
  IL12a of B3

SEQ ID NO: 122 - DNA, 591 bp, Artificial Sequence; identical to SEQ ID NO: 111
  IL12a of B4

SEQ ID NO: 123 - DNA, 591 bp, Artificial Sequence
  IL12a of C1
cggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaaaagcagcctcgaggagcccgacttctacaaaacaaagatcaaactctgcatccttctgcacgccttcagaattagagccgtgaccatcgacagagttatgagctacctgaatgccagc

SEQ ID NO: 124 - DNA, 591 bp, Artificial Sequence; identical to SEQ ID NO: 115
  IL12a of C2

SEQ ID NO: 125 - DNA, 591 bp, Artificial Sequence; identical to SEQ ID NO: 120
  IL12a of C3

SEQ ID NO: 126 - DNA, 18 bp, Artificial Sequence
  GS Linker of L1
ggatcagggggaggctcg

SEQ ID NO: 127 - DNA, 18 bp, Artificial Sequence
  GS Linker of L2
ggatcaggaggtgggagc

SEQ ID NO: 128 - DNA, 18 bp, Artificial Sequence
  GS Linker of L3
ggtagtggcggggggagc

SEQ ID NO: 129 - DNA, 18 bp, Artificial Sequence
  GS Linker of M1
ggcagcggcggcggatcc

SEQ ID NO: 130 - DNA, 18 bp, Artificial Sequence
  GS Linker of M2
ggctcaggcggagggagt

SEQ ID NO: 131 - DNA, 18 bp, Artificial Sequence
  GS Linker of M3
gggagtggcggtggcagc

SEQ ID NO: 132 - DNA, 18 bp, Artificial Sequence
  GS Linker of H1
ggctctggcggcggcagt

SEQ ID NO: 133 - DNA, 18 bp, Artificial Sequence
  GS Linker of H2
ggcagcggcggcggtagc

SEQ ID NO: 134 - DNA, 18 bp, Artificial Sequence
  GS Linker of H3
ggcagcggtggcggcagc

SEQ ID NO: 135 - DNA, 18 bp, Artificial Sequence
  GS Linker of CO
ggcagcggcggcggcagc

SEQ ID NO: 136 - DNA, 18 bp, Artificial Sequence
  GS Linker of CP
ggcagcggcggcggcagt

SEQ ID NO: 137 - DNA, 18 bp, Artificial Sequence
  GS Linker of vH1
ggcagcggcggcggcagc

SEQ ID NO: 138 - DNA, 18 bp, Artificial Sequence
  GS Linker of vH2
ggcagcggcggtggcagc

SEQ ID NO: 139 - DNA, 18 bp, Artificial Sequence
  GS Linker of vH3
ggcagcggcggcgggagc

SEQ ID NO: 140 - DNA, 18 bp, Artificial Sequence
  GS Linker of B1
ggctctggcggcggcagt

SEQ ID NO: 141 - DNA, 18 bp, Artificial Sequence
  GS Linker of B2
ggcagcggtggcggcagc

SEQ ID NO: 142 - DNA, 18 bp, Artificial Sequence
  GS Linker of B3
ggcagcggcggcggcagc

SEQ ID NO: 143 - DNA, 18 bp, Artificial Sequence
  GS Linker of B4
ggcagcggcggcggcagt

SEQ ID NO: 144 - DNA, 18 bp, Artificial Sequence
  GS Linker of C1
ggcagcggcggcggatcc

SEQ ID NO: 145 - DNA, 18 bp, Artificial Sequence
  GS Linker of C2
ggctctggcggcggcagt

SEQ ID NO: 146 - DNA, 18 bp, Artificial Sequence
  GS Linker of C3
ggcagcggtggcggcagc

SEQ ID NO: 147 - DNA, 1758 bp, Artificial Sequence
  Albumin of L1
gacgcccataagagcgaagtcgcccaccgcttcaaggatttgggtgaggaaaacttcaaagccctggtcctgatagcgtttgcccaatatttgcagcagtgtccattcgaagatcacgtgaaattggtgaacgaggtaacagaatttgctaagacttgtgtggctgacgagtcggccgaaaactgtgataagagtcttcatacactgtttggcgataagctatgtactgtcgctacacttagggagacttacggtgagatggccgactgctgcgccaagcaagagccagaacgaaacgagtgttttctgcaacataaggacgacaatcccaacctgcccagattggttcgccctgaagttgatgttatgtgcaccgcatttcacgacaacgaagaaacctttcttaaaaagtatctgtacgagatagctcgacgtcacccttacttctacgcgcccgaacttctgtttttcgccaagcgatacaaagccgctttcacagagtgttgccaagctgccgacaaagccgcttgccttctaccaaagcttgacgagctcagagatgaagggaaagctagttcggcaaagcaacgattaaagtgtgcatcactgcaaaaattcggcgaacgagcctttaaagcatgggcagttgccaggttatcccaaaggttcccgaaagctgaattcgctgaggtgagcaagttagtcacggaccttacgaaggtacataccgaatgctgccacggggacctcttggagtgcgctgacgacagggcggacttagctaaatacatttgcgagaatcaggactcaatcagttctaaacttaaagaatgctgcgagaaaccgctcctggaaaaatcacattgcatcgccgaggtggaaaacgatgagatgccagcagatttaccatctctagccgccgacttcgtggaaagtaaggatgtgtgtaaaaactatgcggaagcaaaagacgtgttcctcggaatgtttctatacgaatacgctagaaggcatcctgactattctgtcgttttactgcttagactagcgaagacatatgaaacgacgttagagaaatgctgcgcggccgctgacccccacgaatgttacgcgaaggtctttgatgagttcaagcccctggttgaggagccgcaaaaccttattaaacagaattgtgagctatttgagcagttaggcgaatataaattccagaatgcacttctagtacgatacaccaaaaaggtccctcaagtgagcacccccactcttgtggaggtatccagaaatctaggaaaggtaggctctaaatgctgcaagcatcccgaagccaagagaatgccatgcgctgaagactaccttagcgttgttctgaatcagttgtgtgtccttcacgaaaagacgccggtgagtgatcgtgtcacgaagtgctgtacagagagcctcgtcaaccgtaggccatgtttctccgctctcgaggtggatgaaacatatgtacctaaggaatttaatgcggaaactttcacctttcacgcggacatctgtaccctgagcgagaaggagaggcagataaaaaagcagacggctcttgtagagttggtcaaacataagcctaaggccactaaagagcagctaaaggcagtaatggacgactttgcggctttcgttgagaagtgctgcaaggccgacgataaagagacctgtttcgcggaagaaggtaaaaagttagtggccgcctcccaggcggccctgggcctgtag

SEQ ID NO: 148 - DNA, 1758 bp, Artificial Sequence
  Albumin of L2
gacgcgcacaaaagcgaggtagcgcatcgctttaaagacttaggagaggaaaactttaaggcgctggtgctcatcgcatttgcccaatacttacagcagtgtccttttgaggaccacgtaaagcttgtaaacgaagtcactgaattcgccaagacatgtgttgctgacgaaagcgcagagaactgtgacaaaagccttcataccctctttggtgacaaactctgcaccgtggcaactctaagagaaacctacggggaaatggcagactgctgcgcaaagcaggaacccgaacgcaatgagtgtttcctgcagcacaaggatgataatcccaatctgccacgacttgtacggccggaggtagatgttatgtgcactgcttttcatgacaacgaggaaactttccttaagaaatatctgtatgagatcgcaaggcggcacccttacttctacgcacccgaactgttgtttttcgcaaagaggtataaggccgcatttaccgagtgttgtcaggccgctgataaagccgcctgtcttttgccaaaattagatgaactaagggacgaaggcaaagcgagtagcgccaaacaaagattaaaatgtgcaagcctccaaaaattcggtgaaagagcatttaaggcgtgggctgtcgcccgactttcacaacgcttccccaaagctgaattcgctgaggtttcgaagctggttaccgacctaactaaagtgcatacagagtgctgtcatggggatctcttagagtgcgcggatgaccgggcagacctggctaagtacatatgtgagaaccaggacagtatatcatcaaagctgaaagagtgttgtgagaagccactactcgagaagagtcactgtattgccgaggtggaaaatgatgagatgccagccgatcttccttctttggccgctgactttgtagagagcaaggatgtctgtaagaactacgctgaggccaaggatgtctttttggggatgttcctctatgagtacgcccgacgacaccctgactatagtgtagtacttttgcttagactggctaaaacatatgagacgactctcgaaaagtgctgtgccgctgccgatccacacgagtgctacgctaaggtgtttgatgagtttaagccgctggtggaggaaccccagaacctgatcaagcagaactgtgaactattcgagcaactaggggagtacaaattccagaacgcacttttagtgcggtacaccaaaaaagtgccacaggtcagtacaccaacattagtggaagtatccaggaacctgggcaaagtgggcagcaaatgctgcaaacatccggaggctaagcggatgccctgtgcagaggactacctgtccgtggtgcttaaccagctgtgtgtgcttcacgagaaaacgcctgtgtccgaccgggtgaccaagtgctgtacggagtcactggtaaatcgacgaccgtgtttttcagcactagaagttgatgaaacttatgtaccgaaagagtttaacgcagagacctttacattccacgccgacatctgcacgctgtccgagaaggaaagacagattaaaaagcagactgccctagtcgagcttgtcaaacacaaaccgaaggcaaccaaggaacagttaaaagcagtgatggatgattttgctgcgttcgtcgaaaaatgttgcaaagcggacgacaaggagacttgcttcgcagaggaagggaagaaattggttgcggcgtcccaagcggccttagggctatag

SEQ ID NO: 149 - DNA, 1758 bp, Artificial Sequence
  Albumin of L3
gacgcccacaagtctgaagtggcacaccggttcaaggacctcggggaggagaattttaaagccctcgtgctgatcgctttcgcgcagtacttgcagcagtgcccttttgaggaccatgtcaaattagtaaacgaagtgacggaattcgcaaagacttgcgtagcggatgagtcagcagagaattgcgacaagtcgctacacactctgttcggggataagttgtgcacagttgctaccttacgagagacctatggagagatggctgactgctgcgccaaacaagagcctgaaagaaacgagtgcttcttacaacacaaagacgataacccgaatttgccaaggcttgtaagacccgaagtagatgttatgtgcacagcttttcacgacaacgaggagacgttccttaagaaatatctgtatgaaatagcccgtcggcacccctatttttatgctcccgaactattgttcttcgccaaacgatacaaggctgcgttcactgagtgctgtcaggctgcagacaaagcagcctgcttgcttcccaaattggacgaactacgggacgaaggtaaggcgagctccgcaaaacagcggttgaaatgcgcgtcactacagaagtttggggaaagagcgttcaaagcttgggctgtggcacgattgagccaacgcttccccaaagcagagtttgcggaagtttcgaaactcgtgacagacttaacaaaggttcacaccgagtgctgtcacggcgatctgctcgaatgcgctgatgaccgcgctgacttggctaaatacatttgtgagaaccaggactctatatcgagcaaactcaaggaatgctgcgaaaagcccctgcttgagaagtcccactgcatcgctgaggtagagaatgatgagatgcctgcggatcttccgagtttagcagctgatttcgtcgagagcaaagatgtgtgcaaaaattatgccgaagcaaaagacgtatttcttgggatgttcttatacgagtatgcacggcgccacccagattactccgtagtgctactgttaagattggcgaagacctatgaaaccacattggaaaagtgctgcgccgccgccgacccccacgagtgttacgcgaaggtgttcgatgaatttaaaccgcttgttgaggagccgcaaaacctaataaagcaaaactgtgagctctttgagcaactaggtgaatacaagtttcagaatgcactcctagttcggtacaccaaaaaagtacctcaggtatctacgccaacgttagtcgaggtctcgcggaatttgggtaaagtaggttccaagtgttgcaaacacccggaagctaaacgtatgccgtgtgctgaggactatctcagcgttgtgttaaaccaactttgcgtactccacgagaaaacacctgtctcagatcgggtaaccaaatgctgcacggagtcgctagtaaatcgtcgcccatgcttttctgcgttagaggtggacgaaacttatgtaccgaaagaatttaacgcggaaacctttacattccatgcagatatctgtacactgtccgagaaagagagacagattaagaaacagacggcgctggtggagcttgtgaagcacaagcctaaagctacgaaggagcaactgaaggcagtcatggatgactttgcggcgtttgtggagaagtgctgtaaagcggacgataaggaaacatgcttcgcagaagaaggaaagaagctggtcgccgctagccaagcggctctgggcctgtag

SEQ ID NO: 150 - DNA, 1758 bp, Artificial Sequence
  Albumin of M1
gatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctctag

SEQ ID NO: 151 - DNA, 1758 bp, Artificial Sequence
  Albumin of M2
gacgcccacaagtctgaagtggctcaccggtttaaggaccttggcgaggagaactttaaagccctggtgctgattgcctttgcccagtatttacaacaatgccctttcgaagaccacgtgaagctcgtcaatgaggtcaccgagttcgctaagacctgcgtagccgacgaaagtgccgagaactgcgacaagagcctgcacaccctgttcggggacaaactctgtaccgtggccaccctacgggagacatatggggagatggccgactgctgcgcaaaacaggagcccgagagaaatgagtgcttcctgcagcacaaggatgacaaccccaatctgcccagactggtgcgccccgaggtagacgttatgtgcaccgccttccatgacaatgaggagacgttcctgaagaaatacctgtacgagatcgcaagacgtcacccctatttctatgcacctgagctgcttttcttcgccaagagatataaggccgccttcaccgaatgctgccaggcagccgataaggcagcttgcctcctgccaaagctggacgagctgagagatgagggcaaggcctccagcgcgaagcagagactcaaatgcgcaagccttcagaagttcggagaacgcgcctttaaagcctgggccgtcgccagactgagccagcgcttccctaaagccgaattcgcagaagtgagcaagctggtaacggacctgacaaaggtgcatactgagtgctgccatggcgatctgctggagtgcgctgatgacagagcagatttggcgaaatatatttgcgaaaatcaggatagcatcagctctaagctcaaggagtgttgtgagaagcccctgctggaaaaaagccactgcattgcagaggttgagaacgatgaaatgccagccgaccttccatcattggccgccgatttcgtggagtcgaaggatgtgtgtaagaactacgccgaggccaaggacgtgttcctgggcatgttcctgtacgagtatgctagaagacatcccgattacagtgtggtgctgctattgagactggccaagacctacgaaaccaccctggagaaatgttgcgccgcggcagatcctcacgaatgttacgccaaagtgtttgacgaattcaagccactggtagaggagccccagaacttaataaagcagaattgcgagctattcgagcagttgggcgagtacaaattccagaacgcccttctggtgaggtataccaaaaaggtgccccaggtgtctacccctaccctggtggaggtcagccgaaatctgggaaaggtcggatccaagtgctgcaagcacccggaggccaagaggatgccttgcgctgaggactatctcagtgtcgtcctgaatcagctatgcgtgttgcacgagaaaaccccagtgagtgaccgcgtgactaaatgctgcaccgaaagcttggtgaatcggaggccctgtttctctgcactggaagttgacgagacttacgtcccgaaggaattcaacgccgagacattcaccttccatgctgacatatgtactctgtcagaaaaggagcgtcagatcaagaagcagacagccctggtggaactggttaagcataagcctaaagcgaccaaagagcagctgaaagccgtgatggacgattttgccgccttcgtggagaaatgttgtaaggcagacgacaaagagacatgtttcgccgaagaggggaagaaactggtggccgcaagccaggccgctctgggtctgtag

SEQ ID NO: 152 - DNA, 1758 bp, Artificial Sequence
  Albumin of M3
gatgcccacaaaagcgaagtcgcacacagattcaaggacttgggtgaggagaactttaaagccctggtgctgatcgccttcgcgcagtatctccagcagtgccccttcgaagatcatgtgaaactggtgaacgaggtaaccgagttcgcgaagacatgcgttgctgatgagagcgccgaaaattgcgacaaaagcctgcatactctgttcggggacaagctgtgcacggtcgcaaccctgagagaaacctacggcgagatggcagactgctgcgccaagcaggagcctgagaggaacgagtgttttctgcagcacaaggacgataatcctaaccttcctcgtctagtgagacccgaagtggacgttatgtgtaccgcctttcacgacaatgaggaaacattcctgaaaaagtacctgtacgagatcgccagacggcacccatatttctacgcccccgagctgctcttcttcgcaaagaggtacaaggctgccttcaccgagtgctgccaggcggccgacaaggcggcgtgtttgctgcctaagctggacgaactacgtgacgaaggaaaagctagcagcgccaagcagagacttaagtgcgcgtccttacagaagtttggcgaaagagcgtttaaggcctgggccgtggcaaggctgtctcaaagattccccaaggcggagttcgccgaggtgtcaaaactggtgaccgacttaaccaaggtgcacaccgaatgctgccacggcgatctgctcgagtgcgccgacgacagagccgatctggcaaaatacatctgcgaaaaccaggatagcatcagctccaaactgaaggagtgctgtgaaaaaccactgcttgaaaaatcgcattgtatagcggaggtggagaatgacgagatgcccgccgacctgccaagcctggccgccgatttcgttgaatccaaggacgtttgcaagaactatgcagaagcgaaggacgtgttcttaggaatgttcctatacgagtacgcgagaagacatcccgactacagcgtggttctgctgttgagattagccaagacgtatgagacaaccctcgaaaagtgctgcgccgccgccgacccccacgagtgttacgcaaaggtgttcgatgagtttaaaccgctggttgaggaaccgcaaaacctgatcaagcagaactgcgagctgttcgagcagctgggtgaatacaagtttcagaatgcactgttggtgcgatataccaagaaggtgcctcaggtgagcacccctacccttgttgaggtgtcccgcaatctgggtaaggttgggagcaagtgttgcaaacaccccgaggccaagagaatgccctgcgcggaagattatctcagtgtcgtgcttaatcagttatgtgtcctgcatgagaagacccccgtgagcgacagagtgaccaagtgctgtaccgaatctctcgtgaacagacgcccgtgcttcagcgccttggaggtagacgagacctacgtgcccaaggagttcaacgcagagaccttcacctttcacgccgatatctgcaccctgtccgagaaggagagacaaatcaagaaacaaacggccctcgtggagctggtcaagcacaaacccaaggccacaaaagagcagctgaaggccgtgatggacgacttcgcagcctttgtggagaaatgctgcaaggctgacgacaaggagacatgcttcgccgaggaaggcaagaagttggtggccgccagccaggcggccctgggcctgtag

SEQ ID NO: 153 - DNA, 1758 bp, Artificial Sequence
  Albumin of H1
gacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgagtgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaagagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctttag

SEQ ID NO: 154 - DNA, 1758 bp, Artificial Sequence
  Albumin of H2
gacgcccacaagagcgaagtggcccatagattcaaggacctgggcgaggagaacttcaaggccctcgtgctgatcgccttcgcccagtacctgcagcagtgccctttcgaggaccacgttaaactggtgaatgaagtgaccgagttcgccaaaacctgcgtggccgacgagtctgccgaaaattgcgacaaaagcttacacaccctgttcggcgacaagctgtgcaccgtggccaccttaagagaaacctacggcgagatggccgactgctgcgctaagcaggagcccgagagaaacgaatgcttcctgcagcacaaggacgataacccaaatctgcctagactggtgagacccgaggtggacgtgatgtgcacagccttccacgataacgaagagacattcctgaaaaagtacctgtacgagatcgccagaagacatccttacttctatgcccccgagcttctgttcttcgccaagagatataaggccgccttcaccgagtgctgccaagccgccgacaaggcagcctgcctgctgccaaagcttgacgagctgagagacgagggcaaagccagcagcgccaagcagagactgaagtgcgcctccctgcagaagttcggcgagagagcctttaaggcctgggccgtggccagattgagtcagagattccccaaggccgagttcgccgaggtgagcaaactggtgaccgacctcactaaagtgcacacagaatgttgccatggcgatctcctggaatgcgccgatgacagggccgacctggccaagtacatctgtgagaaccaggacagcatttcgagcaagctgaaggagtgctgcgagaaacccctgctggagaagtcccactgcatcgccgaggtggagaacgacgagatgcccgccgacctgcccagcctggccgcagatttcgtggagagcaaggacgtatgcaagaactacgcagaggccaaggatgtgttcctgggcatgttcctgtacgaatacgccagaaggcaccccgactacagcgtcgtgctgttactgagactggccaagacctacgagacaaccttagagaagtgctgcgcggccgcagacccgcacgagtgctacgccaaggtgttcgacgagttcaagcccctggtggaggaacctcagaatctgattaagcagaattgtgagctgttcgagcagctgggagagtacaagttccagaacgcgctgctggtgagatataccaagaaggtgccccaggtgagcacccccaccctggtggaggtgagtcgcaacctgggcaaggtggggagcaagtgttgcaagcatcccgaggccaaaagaatgccctgcgcagaggactatctgagcgttgtgcttaaccagctgtgcgtgctgcacgagaagacccccgtgagcgacagagtgaccaagtgttgcaccgagagcctggtaaacagaagaccctgcttcagcgccctggaggtggacgagacctacgtgcccaaggagttcaacgccgagacctttaccttccacgcagacatttgcaccctgagcgagaaagagaggcagatcaagaaacagaccgctctggtcgagcttgtgaagcacaagcccaaagctaccaaggagcagctgaaggccgtgatggatgatttcgccgccttcgtagagaaatgctgcaaggccgacgataaagagacctgctttgccgaggagggcaagaaactggtggccgccagccaggcggccctgggcctgtag

SEQ ID NO: 155 - DNA, 1758 bp, Artificial Sequence
  Albumin of H3
gacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctcttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggagaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctgtag

SEQ ID NO: 156 - DNA, 1758 bp, Artificial Sequence
  Albumin of CO
gacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 157 - DNA, 1755 bp, Artificial Sequence
  Albumin of CP
gatgcccacaagtctgaggtggcccaccgcttcaaggacctgggggaggagaacttcaaggccctggtgctgattgcctttgcccagtacctgcagcagtgcccctttgaggaccacgtgaagctggtgaatgaggtgacagaatttgccaagacctgtgtggctgatgaatctgctgagaactgtgacaagagcctgcacaccctgtttggagacaagctgtgcaccgtggccaccctgcgggagacctatggagagatggctgactgctgtgccaagcaggagcctgagagaaatgaatgcttcctgcagcacaaggatgacaaccccaacctgccccggctggtgcggcctgaggtggatgtgatgtgcacagccttccatgacaatgaggagaccttcctgaagaagtacctgtatgaaattgcccggcggcacccctacttctacgcccctgagctgctgttctttgccaagcgctacaaggccgccttcacagagtgctgccaggccgctgacaaggccgcctgcctgctgcccaagctggatgagctgagagatgagggcaaggccagcagcgccaagcagaggctgaagtgtgccagcctgcagaagtttggagagcgggccttcaaggcctgggccgtggcccggctgagccagcgcttccccaaggccgagtttgctgaggtgtccaagctggtgacagacctgaccaaggtgcacacagagtgctgccacggggacctgctggagtgtgctgatgacagagctgacctggccaagtacatctgtgagaaccaggacagcatcagcagcaagctgaaggagtgctgtgagaagcccctgctggaaaagagccactgcatcgccgaggtggagaatgatgagatgcctgctgacctgcccagcctggccgctgactttgtggagagcaaggatgtgtgcaagaactatgcagaggccaaggatgtgttcctgggcatgttcctgtatgaatatgcccggcggcacccagactacagcgtggtgctgctgctgcggctggccaagacctatgagaccaccctggagaagtgctgtgccgctgctgacccccatgaatgttatgccaaggtgtttgatgagttcaagcccctggtggaggagccccagaacctgatcaagcagaactgtgagctgtttgagcagctgggggagtacaagttccagaatgccctgctggtgcgctacaccaagaaggtgccccaggtgtccacccccaccctggtggaggtgtccaggaacctgggcaaggtgggcagcaagtgctgcaagcaccctgaggccaagaggatgccctgtgccgaggactacctgtctgtggtgctgaaccagctgtgtgtgctgcacgagaagacccctgtgtctgacagagtgaccaagtgctgcacagagagcctggtgaacagaagaccctgcttcagcgccctggaggtggatgagacctacgtgcccaaggagttcaatgctgagaccttcaccttccacgccgacatctgcaccctgtctgagaaggagcggcagatcaagaagcagacagccctggtggagctggtgaagcacaagcccaaggccaccaaggagcagctgaaggctgtgatggatgactttgctgcctttgtggagaagtgctgcaaggcagatgacaaggagacctgctttgctgaggagggcaagaagctggtggccgccagccaggccgccctgggcctg

SEQ ID NO: 158 - DNA, 1758 bp, Artificial Sequence
  Albumin of vH1
gacgcccacaagagcgaggtggcccataggttcaaggacctgggcgaggagaacttcaaggccctggtactcatcgccttcgcccaatacctgcaacagtgccccttcgaggaccatgttaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgaacgtaacgagtgcttcctgcagcacaaggacgacaaccccaacctgccccgactggtcagacccgaggtggacgtgatgtgcacagccttccacgacaacgaggagaccttcctgaagaagtacctctacgagatcgccagaagacatccatacttctacgcccccgagctgctgttcttcgccaagaggtacaaggccgccttcacagagtgctgccaggccgccgacaaggccgcttgcctgctgcctaagttggacgagctgagagacgagggcaaggccagcagcgccaagcagagactgaagtgcgcaagcctgcagaaattcggcgagagagcctttaaggcctgggccgtggccagactgagccagcgcttccccaaggccgagttcgccgaggtgagcaagctggtgaccgacctgaccaaagtgcacaccgagtgctgccacggcgacctgctggagtgcgccgacgacagagccgacctggccaagtacatctgcgagaaccaggacagcatcagcagcaagttgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggagacaccccgactacagcgtggtgctgctgctgagactggccaagacctacgagaccaccctggagaagtgctgtgccgccgccgacccccacgagtgctacgccaaggtgttcgacgagttcaagcccctggtggaggagccccagaacctgatcaagcagaactgcgagctgttcgagcagctgggcgagtataagttccagaacgccctgctggtgagatacaccaagaaggtgccccaggtcagcacccccaccctggtggaggtgagcagaaatctgggcaaggtgggcagcaaatgctgcaagcaccccgaggccaagagaatgccctgcgccgaggactacctgtcagtggtgctgaaccagctgtgtgtgctgcacgagaagacccccgtgagcgacagagtgaccaagtgctgcaccgagagtctcgtgaacagacggccctgcttcagcgccctggaggtggacgaaacatacgtgcccaaggagttcaacgcagagaccttcaccttccacgcagacatctgcaccctgagcgagaaggagagacagatcaagaagcagaccgccctggttgagcttgtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 159 - DNA, 1758 bp, Artificial Sequence
  Albumin of vH2
gacgctcacaagagcgaggtggcccacagattcaaggacctgggcgaggagaacttcaaggccctggtcctgatcgccttcgcccagtacctgcagcagtgccccttcgaggaccacgtgaagctggtgaacgaggtgaccgagttcgccaagacctgcgtggccgacgagtcggccgagaactgcgacaagagcctgcacaccctgttcggcgacaagctgtgcaccgtggccaccctgagagaaacctacggggagatggccgactgctgcgcaaagcaggagcccgagcgaaacgagtgcttcctgcagcacaaggacgacaaccccaacttgcccagactggtaagacccgaggtggacgtcatgtgcaccgctttccacgacaacgaggagaccttcctgaagaagtacctgtacgagatcgccagaagacacccctatttctatgcccctgagctgctgttcttcgccaagcgctacaaggccgccttcaccgagtgctgccaggccgccgacaaggccgcctgcctgctccccaagctggacgagctgagagacgaaggcaaggccagcagcgccaagcagagactgaagtgcgccagcctgcagaagttcggcgagagagccttcaaggcctgggccgtggccagactgtcgcagagattccccaaggccgagttcgccgaggtgagcaagctggttaccgacctgactaaggtgcacaccgagtgctgccacggcgacctgctggagtgcgccgacgacagagccgacctggccaagtacatctgcgagaaccaggatagcatcagcagcaagctgaaggagtgctgcgagaagccccttctggagaagtcccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagactacccttgagaagtgctgcgccgccgccgacccacatgagtgctacgccaaggtgttcgacgagttcaagcccctggtggaagagccccagaacctgatcaagcagaactgcgagctgttcgagcagctgggcgagtacaagttccagaacgcccttctggtgagatacaccaagaaggtgcctcaggtgagcacccccacccttgtggaggtgagcagaaacctcggcaaggtgggcagcaagtgctgcaagcatccagaggccaagagaatgccctgcgccgaggactacctgagcgtggtgctgaaccagctgtgcgtgctgcacgagaaaactcccgtgagcgacagagtgaccaagtgctgcaccgagagtctggtgaacagaagaccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctcaaggccgtgatggacgacttcgccgcgttcgtggagaagtgctgcaaggccgacgacaaagagacctgcttcgccgaggagggcaagaagcttgtggccgccagccaggccgccctgggcctgtag

SEQ ID NO: 160 - DNA, 1758 bp, Artificial Sequence
  Albumin of vH3
gacgcccacaagagcgaggtggcccacagattcaaggacctcggcgaggagaacttcaaggccctggtgctgatcgctttcgcccagtacctgcagcagtgccccttcgaggaccacgtgaaactggtgaacgaggtgaccgagttcgccaagacctgcgtggccgacgagagcgccgagaactgcgacaagagcctgcacacgttgttcggcgacaagctgtgcaccgtggccaccctgagagagacctacggcgagatggccgactgctgcgccaagcaggagcccgagagaaacgaatgcttcctgcagcacaaggacgacaaccccaacctgccccgcctggtgagacccgaggtggacgtgatgtgcaccgccttccacgacaacgaggagaccttcctgaagaagtacttgtacgagatcgcaagaagacacccgtacttctacgcccccgagctgctgttcttcgccaagagatacaaggccgccttcaccgagtgctgccaggccgccgacaaggccgcctgcctgctgcccaagctggacgagctcagagacgagggcaaggccagcagcgccaagcagagactgaagtgcgccagcctgcagaagttcggcgagagagcctttaaggcctgggccgtggccagactgagccagagattccccaaggccgagttcgccgaagtgagcaagctggtgaccgacctgacgaaggtgcacaccgagtgctgccacggcgacctgctggagtgcgccgacgacagagcagacctggccaagtacatctgcgagaaccaggacagcatcagcagcaagctgaaggagtgctgcgagaagcccctgctggagaagagccactgcatcgccgaggtggagaacgacgagatgcccgccgacctgcccagtctggccgcagacttcgtggagagcaaggatgtgtgcaagaactacgccgaggccaaggacgtgttcctgggcatgttcctgtacgagtacgcaagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgaactgttcgagcagcttggcgagtacaagtttcagaacgccctgctggtcagatacaccaagaaggtgccccaggtgagcacccccaccctggtggaggtgtccagaaacctgggcaaggtgggcagcaagtgctgcaagcaccccgaggcaaagagaatgccctgcgccgaggactatctgagcgtggtgctgaaccagctgtgcgtgctgcacgagaagacccccgtgagcgacagagtgaccaagtgctgcaccgagagcctggtgaatagaagaccctgcttcagcgcactggaggtggacgagacctacgtgcccaaggagttcaacgccgagacattcaccttccacgccgatatctgcaccctgagcgagaaggagagacagatcaagaagcagaccgccctggtggagctggtgaagcacaagcccaaggccaccaaggagcagttgaaggccgtgatggacgacttcgccgccttcgtggagaaatgctgcaaggccgacgacaaggagacctgcttcgccgaggagggcaagaagctggtggccgccagccaggccgccctgggcctgtag

SEQ ID NO: 161 - DNA, 1755 bp, Artificial Sequence
  Albumin of B1
gacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgaatgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaaaagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctt

SEQ ID NO: 162 - DNA, 1755 bp, Artificial Sequence
  Albumin of B2
gacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctgttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggaaaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctg

SEQ ID NO: 163 - DNA, 1755 bp, Artificial Sequence
  Albumin of B3
gacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaaaagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctg

SEQ ID NO: 164 - DNA, 1755 bp, Artificial Sequence; identical to SEQ ID NO: 157
  Albumin of B4

SEQ ID NO: 165 - DNA, 1755 bp, Artificial Sequence
  Albumin of C1
gatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctc

SEQ ID NO: 166 - DNA, 1755 bp, Artificial Sequence; identical to SEQ ID NO: 161
  Albumin of C2

SEQ ID NO: 167 - DNA, 1755 bp, Artificial Sequence; identical to SEQ ID NO: 162
  Albumin of C3

SEQ ID NO: 168 - DNA, 24 bp, Artificial Sequence
  GS Linker of C1
ggtggcgggtctggtggcggttct

SEQ ID NO: 169 - DNA, 24 bp, Artificial Sequence
  GS Linker of C2
ggtggcgggtctggtggcggttct

SEQ ID NO: 170 - DNA, 24 bp, Artificial Sequence
  GS Linker of C3
ggtggcgggtctggtggcggttct

SEQ ID NO: 171 - DNA, 960 bp, Artificial Sequence
  Lumican of C1
cagtactacgattacgacttccccctgagtatctacggtcagtcctcacctaactgcgccccagagtgtaactgccccgaaagctacccgagcgccatgtactgcgacgagctgaagttgaagtccgtgcccatggtgcccccaggcatcaagtacttatacttgaggaacaaccaaatcgatcatatcgacgagaaggccttcgaaaacgtaaccgacctgcagtggctgatactggatcacaatctgctagagaattccaagatcaagggcagagtgttctcgaagctgaaacaactgaagaagctgcacatcaaccataacaatctcaccgagagcgtgggtcccctgcccaagtcgctggaggacctgcagctgacccacaacaaaataaccaaactaggcagcttcgaggggttggtgaatctgaccttcatccatttgcagcataacagactaaaggaggatgccgtgagcgccgccttcaaaggcctcaagagccttgagtacctggacctgagcttcaaccagatcgcccggctgcccagtgggctgcccgtgagcctgctgacgctgtatctggacaataacaaaatcagcaacatccccgacgaatacttcaaaagattcaacgccttacagtacctgcgactcagccacaatgagctcgctgacagcggcatccctggcaacagcttcaacgtgtcatccctggtggagctggacctgagttacaataagctgaagaacatcccaactgtcaatgagaatttggaaaactactacctggaggtgaaccagctggagaagttcgacattaagagcttttgcaaaatcctgggcccactgtcatatagcaagatcaagcacctgcgactggacggcaaccgaatcagtgaaacttccctaccccctgacatgtacgagtgcctgagagtagcaaatgaggtgaccctgaac

SEQ ID NO: 172 - DNA, 960 bp, Artificial Sequence
  Lumican of C2
cagtattacgactacgatttccccctgagcatctatggccagagcagccctaactgcgccccggagtgcaactgccccgaaagctacccaagcgccatgtactgcgatgagctgaagctgaagtctgtgcctatggtgcctcccggcatcaagtacctgtacctgagaaacaaccagatagaccacatcgatgagaaagccttcgagaacgtcaccgacctgcagtggctgattctggaccacaatttactggagaactccaagatcaagggcagagtgttctccaagttaaagcagctgaagaaactgcacatcaatcacaacaacctgaccgagagcgtgggcccactgcccaagagcctagaggatctgcagctcacccacaacaagatcactaagttgggcagcttcgagggcctcgtaaacttgacattcatacatctgcagcacaacagacttaaggaagacgccgtgagtgcggcctttaagggtctgaaaagcctggagtacttagacctgagcttcaaccagatcgcaaggctgcccagcggccttccggtcagtctgctgaccctgtatctggacaacaacaagatcagcaacatccccgacgagtacttcaagcggtttaacgccctccagtacctgagactgagccacaacgagttagctgactcgggcatacccggtaacagcttcaatgttagcagcctagttgagcttgacttgagctacaacaagcttaagaacatcccaaccgtgaacgagaacctcgagaattactacctggaagtcaaccagctggagaagttcgacattaagagcttctgcaaaatcctgggcccactgtcctatagcaagatcaagcacctgcgccttgacggaaacagaattagcgagaccagccttccaccagacatgtacgagtgcctgagggtggccaacgaggtgaccctgaac

SEQ ID NO: 173 - DNA, 960 bp, Artificial Sequence
  Lumican of C3
cagtactacgactacgatttccccctatccatctacgggcagagctcgcctaactgcgcccccgagtgtaactgccccgagtcgtaccccagcgccatgtactgtgacgagctgaagctgaaaagcgtgcccatggtgccccccggcatcaagtacctgtacttgagaaacaaccagatcgaccacattgacgaaaaggccttcgagaacgtaaccgacctgcagtggctgatcctggaccacaacctgcttgagaacagcaagatcaagggccgcgtgttcagcaagctgaagcagctgaagaagctgcacatcaaccacaacaacttgactgagtctgttggccccctaccaaagagcctggaggacctgcagctgacccacaataagataaccaagctgggctcattcgagggcctggtgaacttgacctttattcacctgcagcataacagactgaaggaggacgccgtgagcgccgcctttaaggggctgaaaagcctggagtacctggacctgagttttaaccagatcgccagactgccctcaggcctgcccgtgagtttgctgactctgtacctggacaacaataagatcagcaacattcctgacgagtatttcaaaagattcaatgctctgcagtacctgagactaagccacaacgagctggccgacagcggaatccccggcaacagcttcaacgtgagcagcttggtggagttggacctgagctacaacaaactgaagaacatccccaccgtcaatgagaacttggagaattactacctcgaggttaaccagcttgagaagttcgacatcaagagcttctgcaagatcctgggccccctcagctacagcaagatcaagcacttgagactggacgggaacagaatcagcgaaaccagccttcctcccgacatgtacgagtgccttagagtggcaaatgaggtgaccctgaac

SEQ ID NO: 174 - DNA, 13 bp, Artificial Sequence
  Kozak Sequence
gccgccrccaugg

SEQ ID NO: 175-179 - skipped (5 entries)

SEQ ID NO: 180 - protein, 21 aa, Artificial Sequence
  Leader Sequence
MRVPAQLLGLLLLWLPGARCA

SEQ ID NO: 181 - DNA, 34 bp, Artificial Sequence
  Forward Primer
taatacgactcactataatggactacgacatagt

SEQ ID NO: 182 - protein, 547 aa, Artificial Sequence
  Wild-type IL-12
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 183 - protein, 219 aa, Artificial Sequence
  Wild-type IL12a
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 184 - protein, 328 aa, Artificial Sequence
  Wild-type IL12b
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 185 - DNA, 35 bp, Artificial Sequence
  Reverse Primer
gaaatattaaaaacaaaatccgattcggaaaagaa

(19) United States
(12) Patent Application Publication
(10) Pub. No.: US 2024/0166707 A1
(43) Pub. Date: May 23, 2024

(54) EXPRESSION CONSTRUCTS AND USES THEREOF

(71) Applicant: STRAND THERAPEUTICS INC., Boston, MA (US)

(72) Inventors: Tasuku KITADA, Boston, MA (US); Jacob BECRAFT, Boston, MA (US); Ryan SOWELL, Boston, MA (US); Jaspreet KHURANA, Boston, MA (US); Anna SIMON, Cambridge, MA (US); Joseph BARBERIO, Boston, MA (US); Weiyu ZHAO, Boston, MA (US); Alexander LEMAIRE, Boston, MA (US); Aalok SHAH, Shrewsbury, MA (US)

(73) Assignee: STRAND THERAPEUTICS INC., Boston, MA (US)

(21) Appl. No.: 18/260,831

(22) PCT Filed: Jan. 10, 2022
(86) PCT No.: PCT/US2022/011841

§ 371 Date: Jul. 10, 2023

Related U.S. Application Data
(60) Provisional application No. 63/135,501, filed on Jan. 8, 2021.

(51) Int. Cl. C07K 14/54; A61P 35/00

(52) U.S. Cl. CPC C07K 14/5434; A61P 35/00

(57) ABSTRACT

The present disclosure relates to an isolated polynucleotide comprising a sequence that encodes an interleukin (IL)-12 molecule, and a delivery system that can carry the polynucleotides. The disclosure also includes methods of making the polynucleotides and methods of treatment using the same.

### CROSS REFERENCE TO RELATED APPLICATIONS

This application claims the priority benefit of U.S. Provisional Application No. 63/135,501, filed Jan. 8, 2021, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4597_005PC01_SequenceListing_ST25.txt; Size: 246,918 bytes; and Date of Creation: Jan. 9, 2022) filed with the application is herein incorporated by reference in its entirety.

### BACKGROUND OF THE DISCLOSURE

Due to its ability to activate both NK cells and cytotoxic T cells, IL-12 protein has been studied as a promising anti-cancer therapeutic since 1994. See Nastala, C. L. et al., J Immunol 153: 1697-1706 (1994). But despite high expectations, early clinical studies did not yield satisfactory results. Lasek W. et al., Cancer Immunol Immunother 63: 419-435, 424 (2014). Repeated administration of IL12, in most patients, led to adaptive response and a progressive decline of IL-12-induced interferon gamma (IFN-γ) levels in blood. Id. Moreover, while it was recognized that IL-12-induced anti-cancer activity is largely mediated by the secondary secretion of IFN-γ, the concomitant induction of IFN-γ along with other cytokines (e.g., TNF-α) or chemokines (IP-10 or MIG) by IL-12 caused severe toxicity. Id.

In addition to the negative feedback and toxicity, the marginal efficacy of the IL-12 therapy in clinical settings can be caused by the strong immunosuppressive environment in humans. Id. To minimize IFN-γ toxicity and improve IL-12 efficacy, scientists tried different approaches, such as different dose and time protocols for IL-12 therapy. See Sacco, S. et al., Blood 90: 4473-4479 (1997); Leonard, J. P. et al., Blood 90: 2541-2548 (1997); Coughlin, C. M. et al., Cancer Res. 57: 2460-2467 (1997); Asselin-Paturel, C. et al., Cancer 91: 113-122 (2001); and Saudemont, A. et al., Leukemia 16: 1637-1644 (2002). Nonetheless, these approaches have not significantly impacted patient survival. Kang, W. K., et al., Human Gene Therapy 12: 671-684 (2001). Thus, there is a need in the art for an improved therapeutic approach for using IL12 to treat tumors.

### BRIEF SUMMARY OF THE DISCLOSURE

Provided herein is an isolated polynucleotide comprising a nucleic acid molecule encoding a beta subunit of an IL-12 protein (“IL-12β”). In some aspects, the nucleic acid molecule comprises a nucleotide sequence that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75.

In some aspects, the nucleic acid molecule encoding the IL-12β comprises a nucleotide sequence that is at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in 51. In some aspects, the nucleic acid molecule encoding the IL-12β comprises a nucleotide sequence that is at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 52. In some aspects, the nucleic acid molecule encoding the IL-12β comprises a nucleotide sequence that is at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 53. In some aspects, the nucleic acid molecule encoding the IL-12β comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 54. In some aspects, the nucleic acid molecule encoding the IL-12β comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 55. In some aspects, the nucleic acid molecule encoding the IL-12β comprises a nucleotide sequence that is at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 56. In some aspects, the nucleic acid molecule encoding the IL-12β comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 57. In some aspects, the nucleic acid molecule encoding the IL-12β comprises a nucleotide sequence that is at least 92%, at least 93%, at least 94%, at least 95%, at least 96%, at least 97%, at least 98%, at least 99%, or 100% identical to the sequence set forth in SEQ ID NO: 58. In some aspects, the nucleic acid molecule encoding the IL-12β comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 59. In some aspects, the nucleic acid molecule encoding the IL-12β comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 65, 69, or 74. In some aspects, the nucleic acid molecule encoding the IL-12β comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 66, 70, or 75. In some aspects, the nucleic acid molecule encoding the IL-12β comprises a nucleotide sequence that is at least 97%, at least 98%, at least 99%, or 100% identical to the sequence set forth in SEQ ID NO: 62. In some aspects, the nucleic acid molecule encoding the IL-12β comprises a nucleotide sequence that is at least 99% or 100% identical to the sequence set forth in SEQ ID NO: 63. In some aspects, the nucleic acid molecule encoding the IL-12β comprises a nucleotide sequence that is at least 98%, at least 99%, or 100% identical to the sequence set forth in SEQ ID NO: 64.

Also provided herein is an isolated polynucleotide comprising a nucleic acid molecule encoding an alpha subunit of an IL-12 protein (“IL-12α”). In some aspects, the nucleic acid molecule comprises a nucleotide sequence that is at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

In some aspects, the nucleic acid molecule encoding the IL-12α comprises a nucleotide sequence that is at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 101. In some aspects, the nucleic acid molecule encoding the IL-12α comprises a nucleotide sequence that is at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 102. In some aspects, the nucleic acid molecule encoding the IL-12α comprises a nucleotide sequence that is at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 103. In some aspects, the nucleic acid molecule encoding the IL-12α comprises a nucleotide sequence that is at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 104. In some aspects, the nucleic acid molecule encoding the IL-12α comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 105. In some aspects, the nucleic acid molecule encoding the IL-12α comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 106. In some aspects, the nucleic acid molecule encoding the IL-12α comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 107. In some aspects, the nucleic acid molecule encoding the IL-12α comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 108. In some aspects, the nucleic acid molecule encoding the IL-12α comprises a nucleotide sequence that is at least 92%, at least 93%, at least 94%, at least 95%, at least 96%, at least 97%, at least 98%, at least 99%, or 100% identical to the sequence set forth in SEQ ID NO: 109. In some aspects, the nucleic acid molecule encoding the IL-12α comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 115, 119, or 124. In some aspects, the nucleic acid molecule encoding the IL-12α comprises a nucleotide sequence that is at least 92%, at least 93%, at least 94%, at least 95%, at least 96%, at least 97%, at least 98%, at least 99%, or 100% identical to the sequence set forth in SEQ ID NO: 116, 120, or 125. In some aspects, the nucleic acid molecule encoding the IL-12α comprises a nucleotide sequence that is at least 98%, at least 99%, or 100% identical to the sequence set forth in SEQ ID NO: 112. In some aspects, the nucleic acid molecule encoding the IL-12α comprises a nucleotide sequence that is at least 98%, at least 99%, or 100% identical to the sequence set forth in SEQ ID NO: 113. In some aspects, the nucleic acid molecule encoding the IL-12α comprises a nucleotide sequence that is at least 98%, at least 99%, or 100% identical to the sequence set forth in SEQ ID NO: 114.

The present disclosure further provides an isolated polynucleotide comprising a first nucleic acid molecule and a second nucleic acid molecule, wherein the first nucleic acid molecule encodes a beta subunit of an IL-12 protein (“IL-12β”) and comprises a nucleotide sequence that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75; and the second nucleic acid molecule encodes an alpha subunit of an IL-12 protein (“IL-12α”) and comprises a nucleotide sequence that is at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

In some aspects, the first nucleic acid molecule comprises a nucleotide sequence that is at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 51 and/or the second nucleic acid molecule comprises a nucleotide sequence that is at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 101. In some aspects, the first nucleic acid molecule comprises a nucleotide sequence that is at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 52 and/or the second nucleic acid molecule comprises a nucleotide sequence that is at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 102. In some aspects, the first nucleic acid molecule comprises a nucleotide sequence that is at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 53 and/or the second nucleic acid molecule comprises a nucleotide sequence that is at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 103. In some aspects, the first nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 54 and/or the second nucleic acid molecule comprises a nucleotide sequence that is at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 104. In some aspects, the first nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 55 and/or the second nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 105. In some aspects, the first nucleic acid molecule comprises a nucleotide sequence that is at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 56 and/or the second nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 106. In some aspects, the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 57 and/or the second nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 107. In some aspects, the first nucleic acid molecule comprises a nucleotide sequence that is at least 92%, at least 93%, at least 94%, at least 95%, at least 96%, at least 97%, at least 98%, at least 99%, or 100% identical to the sequence set forth in SEQ ID NO: 58 and/or the second nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 108. In some aspects, the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 59 and/or the second nucleic acid molecule comprises a nucleotide sequence that is at least 92%, at least 93%, at least 94%, at least 95%, at least 96%, at least 97%, at least 98%, at least 99%, or 100% identical to the sequence set forth in SEQ ID NO: 109. In some aspects, the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 65, 69, or 74 and/or the second nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 115, 119, or 124. In some aspects, the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 66, 70, or 75 and/or the second nucleic acid molecule comprises a nucleotide sequence that is at least 92%, at least 93%, at least 94%, at least 95%, at least 96%, at least 97%, at least 98%, at least 99%, or 100% identical to the sequence set forth in SEQ ID NO: 116, 120, or 125. In some aspects, the first nucleic acid molecule comprises a nucleotide sequence that is at least 97%, at least 98%, at least 99%, or 100% identical to the sequence set forth in SEQ ID NO: 62 and/or the second nucleic acid molecule comprises a nucleotide sequence that is at least 98%, at least 99%, or 100% identical to the sequence set forth in SEQ ID NO: 112. In some aspects, the first nucleic acid molecule comprises a nucleotide sequence that is at least 99% or 100% identical to the sequence set forth in SEQ ID NO: 63 and/or the second nucleic acid molecule comprises a nucleotide sequence that is at least 98%, at least 99%, or 100% identical to the sequence set forth in SEQ ID NO: 113. In some aspects, the first nucleic acid molecule comprises a nucleotide sequence that is at least 98%, at least 99%, or 100% identical to the sequence set forth in SEQ ID NO: 64 and/or the second nucleic acid molecule comprises a nucleotide sequence that is at least 98%, at least 99%, or 100% identical to the sequence set forth in SEQ ID NO: 114.

In some aspects, an isolated polynucleotide disclosed herein further comprises a third nucleic acid molecule encoding a linker that joins the first nucleic acid molecule and the second nucleic acid molecule. In certain aspects, the linker comprises an amino acid linker of at least about 2, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 amino acids. In some aspects, the linker comprises a (GS) linker. In some aspects, the (GS) linker has a formula of (Gly3 Ser)n or S(Gly3 Ser)n, wherein n is a positive integer selected from the group consisting of 1, 2, 3, 4, 5, 6, 7, 8, 9, 10, 11, 12, 13, 14, 15, 16, 17, 18, 19, 20, 30, 40, 50, 60, 70, 80, or 100. In some aspects, the (Gly3Ser)n linker is (Gly3Ser)3 or (Gly3Ser)4. In certain aspects, the third nucleic acid molecule encoding the linker comprises the sequence set forth in any one of SEQ ID NOs: 168 to 170.

In some aspects, an isolated polynucleotide of the present disclosure further comprises an additional nucleic acid molecule encoding a half-life extending moiety. In certain aspects, the half-life extending moiety comprises a Fc, an albumin or a fragment thereof, an albumin binding moiety, a PAS, a HAP, a transferrin or a fragment thereof, a XTEN, or any combinations thereof.

In some aspects, an isolated polynucleotide described herein further comprises an additional nucleic acid molecule encoding a leader sequence. In certain aspects, the additional nucleic acid molecule encoding a leader sequence comprises any one of the sequence set forth in SEQ ID NO: 26 to 50.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 26; (b) the second nucleic acid molecule comprises the sequence set forth in SEQ ID NO: 51; (c) the third nucleic acid molecule comprises the sequence set forth in SEQ ID NO: 76; (d) the fourth nucleic acid molecule comprises the sequence set forth in SEQ ID NO: 101; (e) the fifth nucleic acid molecule comprises the sequence set forth in SEQ ID NO: 126; and (f) the sixth nucleic acid molecule comprise the sequence set forth in SEQ ID NO: 147.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 27; (b) the second nucleic acid molecule comprises the sequence set forth in SEQ ID NO: 52; (c) the third nucleic acid molecule comprises the sequence set forth in SEQ ID NO: 77; (d) the fourth nucleic acid molecule comprises the sequence set forth in SEQ ID NO: 102; (e) the fifth nucleic acid molecule comprises the sequence set forth in SEQ ID NO: 127; and (f) the sixth nucleic acid molecule comprise the sequence set forth in SEQ ID NO: 148.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 28; (b) the second nucleic acid molecule comprises the sequence set forth in SEQ ID NO: 53; (c) the third nucleic acid molecule comprises the sequence set forth in SEQ ID NO: 78; (d) the fourth nucleic acid molecule comprises the sequence set forth in SEQ ID NO: 103; (e) the fifth nucleic acid molecule comprises the sequence set forth in SEQ ID NO: 128; and (f) the sixth nucleic acid molecule comprise the sequence set forth in SEQ ID NO: 149.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 29; (b) the second nucleic acid molecule comprises the sequence set forth in SEQ ID NO: 54; (c) the third nucleic acid molecule comprises the sequence set forth in SEQ ID NO: 79; (d) the fourth nucleic acid molecule comprises the sequence set forth in SEQ ID NO: 104; (e) the fifth nucleic acid molecule comprises the sequence set forth in SEQ ID NO: 129; and (f) the sixth nucleic acid molecule comprise the sequence set forth in SEQ ID NO: 150.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 30; (b) the second nucleic acid molecule comprises the sequence set forth in SEQ ID NO: 55; (c) the third nucleic acid molecule comprises the sequence set forth in SEQ ID NO: 80; (d) the fourth nucleic acid molecule comprises the sequence set forth in SEQ ID NO: 105; (e) the fifth nucleic acid molecule comprises the sequence set forth in SEQ ID NO: 130; and (f) the sixth nucleic acid molecule comprise the sequence set forth in SEQ ID NO: 151.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 31; (b) the second nucleic acid molecule comprises the sequence set forth in SEQ ID NO: 56; (c) the third nucleic acid molecule comprises the sequence set forth in SEQ ID NO: 81; (d) the fourth nucleic acid molecule comprises the sequence set forth in SEQ ID NO: 106; (e) the fifth nucleic acid molecule comprises the sequence set forth in SEQ ID NO: 131; and (f) the sixth nucleic acid molecule comprise the sequence set forth in SEQ ID NO: 152.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 32; (b) the second nucleic acid molecule comprises the sequence set forth in SEQ ID NO: 57; (c) the third nucleic acid molecule comprises the sequence set forth in SEQ ID NO: 82; (d) the fourth nucleic acid molecule comprises the sequence set forth in SEQ ID NO: 107; (e) the fifth nucleic acid molecule comprises the sequence set forth in SEQ ID NO: 132; and (f) the sixth nucleic acid molecule comprise the sequence set forth in SEQ ID NO: 153.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 33; (b) the second nucleic acid molecule comprises the sequence set forth in SEQ ID NO: 58; (c) the third nucleic acid molecule comprises the sequence set forth in SEQ ID NO: 83; (d) the fourth nucleic acid molecule comprises the sequence set forth in SEQ ID NO: 108; (e) the fifth nucleic acid molecule comprises the sequence set forth in SEQ ID NO: 133; and (f) the sixth nucleic acid molecule comprise the sequence set forth in SEQ ID NO: 154.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 34; (b) the second nucleic acid molecule comprises the sequence set forth in SEQ ID NO: 59; (c) the third nucleic acid molecule comprises the sequence set forth in SEQ ID NO: 84; (d) the fourth nucleic acid molecule comprises the sequence set forth in SEQ ID NO: 109; (e) the fifth nucleic acid molecule comprises the sequence set forth in SEQ ID NO: 134; and (f) the sixth nucleic acid molecule comprise the sequence set forth in SEQ ID NO: 155.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 37; (b) the second nucleic acid molecule comprises the sequence set forth in SEQ ID NO: 62; (c) the third nucleic acid molecule comprises the sequence set forth in SEQ ID NO: 87; (d) the fourth nucleic acid molecule comprises the sequence set forth in SEQ ID NO: 112; (e) the fifth nucleic acid molecule comprises the sequence set forth in SEQ ID NO: 137; and (f) the sixth nucleic acid molecule comprise the sequence set forth in SEQ ID NO: 158.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 38; (b) the second nucleic acid molecule comprises the sequence set forth in SEQ ID NO: 63; (c) the third nucleic acid molecule comprises the sequence set forth in SEQ ID NO: 88; (d) the fourth nucleic acid molecule comprises the sequence set forth in SEQ ID NO: 113; (e) the fifth nucleic acid molecule comprises the sequence set forth in SEQ ID NO: 138; and (f) the sixth nucleic acid molecule comprise the sequence set forth in SEQ ID NO: 159.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 39; (b) the second nucleic acid molecule comprises the sequence set forth in SEQ ID NO: 64; (c) the third nucleic acid molecule comprises the sequence set forth in SEQ ID NO: 89; (d) the fourth nucleic acid molecule comprises the sequence set forth in SEQ ID NO: 114; (e) the fifth nucleic acid molecule comprises the sequence set forth in SEQ ID NO: 139; and (f) the sixth nucleic acid molecule comprise the sequence set forth in SEQ ID NO: 160.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 44; (b) the second nucleic acid molecule comprises the sequence set forth in SEQ ID NO: 69; (c) the third nucleic acid molecule comprises the sequence set forth in SEQ ID NO: 94; (d) the fourth nucleic acid molecule comprises the sequence set forth in SEQ ID NO: 119; (e) the fifth nucleic acid molecule comprises the sequence set forth in SEQ ID NO: 140; and (f) the sixth nucleic acid molecule comprise the sequence set forth in SEQ ID NO: 161.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 45; (b) the second nucleic acid molecule comprises the sequence set forth in SEQ ID NO: 70; (c) the third nucleic acid molecule comprises the sequence set forth in SEQ ID NO: 95; (d) the fourth nucleic acid molecule comprises the sequence set forth in SEQ ID NO: 120; (e) the fifth nucleic acid molecule comprises the sequence set forth in SEQ ID NO: 141; and (f) the sixth nucleic acid molecule comprise the sequence set forth in SEQ ID NO: 162.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 46; (b) the second nucleic acid molecule comprises the sequence set forth in SEQ ID NO: 71; (c) the third nucleic acid molecule comprises the sequence set forth in SEQ ID NO: 96; (d) the fourth nucleic acid molecule comprises the sequence set forth in SEQ ID NO: 121; (e) the fifth nucleic acid molecule comprises the sequence set forth in SEQ ID NO: 142; and (f) the sixth nucleic acid molecule comprise the sequence set forth in SEQ ID NO: 163.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 47; (b) the second nucleic acid molecule comprises the sequence set forth in SEQ ID NO: 72; (c) the third nucleic acid molecule comprises the sequence set forth in SEQ ID NO: 97; (d) the fourth nucleic acid molecule comprises the sequence set forth in SEQ ID NO: 122; (e) the fifth nucleic acid molecule comprises the sequence set forth in SEQ ID NO: 143; and (f) the sixth nucleic acid molecule comprise the sequence set forth in SEQ ID NO: 164.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, and (vi) a sixth nucleic acid molecule encoding a human serum albumin, wherein: (a) the first nucleic acid molecule comprises the sequence set forth in SEQ ID NO: 36; (b) the second nucleic acid molecule comprises the sequence set forth in SEQ ID NO: 61; (c) the third nucleic acid molecule comprises the sequence set forth in SEQ ID NO: 86; (d) the fourth nucleic acid molecule comprises the sequence set forth in SEQ ID NO: 111; (e) the fifth nucleic acid molecule comprises the sequence set forth in SEQ ID NO: 136; and (f) the sixth nucleic acid molecule comprise the sequence set forth in SEQ ID NO: 157.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, (vi) a sixth nucleic acid molecule encoding a human serum albumin, (vii) a seventh nucleic acid molecule encoding a third linker; and (viii) an eighth nucleic acid molecule encoding a lumican protein, wherein: (a) the first nucleic acid molecule comprises the sequence set forth in SEQ ID NO: 48; (b) the second nucleic acid molecule comprises the sequence set forth in SEQ ID NO: 73; (c) the third nucleic acid molecule comprises the sequence set forth in SEQ ID NO: 98; (d) the fourth nucleic acid molecule comprises the sequence set forth in SEQ ID NO: 123; (e) the fifth nucleic acid molecule comprises the sequence set forth in SEQ ID NO: 144; (f) the sixth nucleic acid molecule comprises the sequence set forth in SEQ ID NO: 165; (g) the seventh nucleic acid molecule comprises the sequence set forth in SEQ ID NO: 168; and (h) the eighth nucleic acid molecule comprises the sequence set forth in SEQ ID NO: 171.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, (vi) a sixth nucleic acid molecule encoding a human serum albumin, (vii) a seventh nucleic acid molecule encoding a third linker; and (viii) an eighth nucleic acid molecule encoding a lumican protein, wherein: (a) the first nucleic acid molecule comprises the sequence set forth in SEQ ID NO: 49; (b) the second nucleic acid molecule comprises the sequence set forth in SEQ ID NO: 74; (c) the third nucleic acid molecule comprises the sequence set forth in SEQ ID NO: 99; (d) the fourth nucleic acid molecule comprises the sequence set forth in SEQ ID NO: 124; (e) the fifth nucleic acid molecule comprises the sequence set forth in SEQ ID NO: 145; (f) the sixth nucleic acid molecule comprises the sequence set forth in SEQ ID NO: 166; (g) the seventh nucleic acid molecule comprises the sequence set forth in SEQ ID NO: 169; and (h) the eighth nucleic acid molecule comprises the sequence set forth in SEQ ID NO: 172.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, (iv) a fourth nucleic acid molecule encoding an alpha subunit of an IL-12 protein (“IL-12α”), (v) a fifth nucleic acid molecule encoding a second linker, (vi) a sixth nucleic acid molecule encoding a human serum albumin, (vii) a seventh nucleic acid molecule encoding a third linker; and (viii) an eighth nucleic acid molecule encoding a lumican protein, wherein: (a) the first nucleic acid molecule comprises the sequence set forth in SEQ ID NO: 50; (b) the second nucleic acid molecule comprises the sequence set forth in SEQ ID NO: 75; (c) the third nucleic acid molecule comprises the sequence set forth in SEQ ID NO: 100; (d) the fourth nucleic acid molecule comprises the sequence set forth in SEQ ID NO: 125; (e) the fifth nucleic acid molecule comprises the sequence set forth in SEQ ID NO: 146; (f) the sixth nucleic acid molecule comprises the sequence set forth in SEQ ID NO: 167; (g) the seventh nucleic acid molecule comprises the sequence set forth in SEQ ID NO: 170; and (h) the eighth nucleic acid molecule comprises the sequence set forth in SEQ ID NO: 173.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, and (iv) a fourth nucleic acid molecule encoding an alpha subunit of an IL-12 protein (“IL-12α”), wherein: (a) the first nucleic acid molecule comprises the sequence set forth in SEQ ID NO: 40; (b) the second nucleic acid molecule comprises the sequence set forth in SEQ ID NO: 65; (c) the third nucleic acid molecule comprises the sequence set forth in SEQ ID NO: 90; and (d) the fourth nucleic acid molecule comprises the sequence set forth in SEQ ID NO: 115.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, and (iv) a fourth nucleic acid molecule encoding an alpha subunit of an IL-12 protein (“IL-12α”), wherein: (a) the first nucleic acid molecule comprises the sequence set forth in SEQ ID NO: 41; (b) the second nucleic acid molecule comprises the sequence set forth in SEQ ID NO: 66; (c) the third nucleic acid molecule comprises the sequence set forth in SEQ ID NO: 91; and (d) the fourth nucleic acid molecule comprises the sequence set forth in SEQ ID NO: 116.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, and (iv) a fourth nucleic acid molecule encoding an alpha subunit of an IL-12 protein (“IL-12α”), wherein: (a) the first nucleic acid molecule comprises the sequence set forth in SEQ ID NO: 42; (b) the second nucleic acid molecule comprises the sequence set forth in SEQ ID NO: 67; (c) the third nucleic acid molecule comprises the sequence set forth in SEQ ID NO: 92; and (d) the fourth nucleic acid molecule comprises the sequence set forth in SEQ ID NO: 117.

Provided herein is an isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, and (iv) a fourth nucleic acid molecule encoding an alpha subunit of an IL-12 protein (“IL-12α”), wherein: (a) the first nucleic acid molecule comprises the sequence set forth in SEQ ID NO: 43; (b) the second nucleic acid molecule comprises the sequence set forth in SEQ ID NO: 68; (c) the third nucleic acid molecule comprises the sequence set forth in SEQ ID NO: 93; and (d) the fourth nucleic acid molecule comprises the sequence set forth in SEQ ID NO: 118.

In some aspects, an isolated polynucleotide described herein further comprises a 5′-cap. In certain aspects, the 5′-cap is selected from the group consisting of m₂^{7,2′-O}GppₛpGRNA, m⁷GpppG, m⁷Gppppm⁷G, m₂^{(7,3′-O)}GpppG, m₂^{(7,2′-O)}GppspG(D1), m₂^{(7,2′-O)}GppspG(D2), m₂^{7,3′-O}Gppp (m₁^{2′-O)}ApG, (m⁷G-3′ mppp-G; which can equivalently be designated 3′ O-Me-m7G(5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, and combinations thereof.

In some aspects, an isolated polynucleotide of the present disclosure further comprises a regulatory element. In certain aspects, the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, and combinations thereof.

In some aspects, an isolated polynucleotide described herein further comprises a 3′ tailing region of linked nucleosides. In certain aspects, the 3′ tailing region of linked nucleosides comprises a poly-A tail, a polyA-G quartet, or a stem loop sequence.

In some aspects, an isolated polynucleotide of the present disclosure comprises at least one modified nucleoside. In certain aspects, the at least one modified nucleoside is selected from the group consisting of 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, an isolated polynucleotide described herein is capable of self-replicating. In certain aspects, the polynucleotide is a self-amplifying replicon RNA. In some aspects, the self-amplifying replicon RNA is derived from an alphavirus. In certain aspects, the alphavirus comprises a Venezuela Equine Encephalitis virus, Semliki Forest virus, Sindbis virus, or combinations thereof.

Present disclosure further provides a vector comprising any of the isolated polynucleotides described herein.

Present disclosure also provides a lipid nanoparticle (LNP) comprising (i) any of the isolated polynucleotides described herein, and (ii) one or more types of lipids. In certain aspects, the one or more types of lipid comprises a cationic lipid. In some aspects, the lipid is an ionizable lipid. In some aspects, the lipid is a lipidoid, e.g., N1,N3,N5-tris(3-(didodecylamino)propyl)benzene-1,3,5-tricarboxamide (TT3). In some aspects, a LNP comprises 1,2-Dioleoyl-sn-glycero-3-phosphoethanolamine (DOPE), cholesterol, C14-PEG2000, or any combination thereof.

In some aspects, a LNP described herein has a diameter of about 30-500 nm. In certain aspects, the LNP has a diameter of about 50-400 nm. In some aspects, the LNP has a diameter of about 70-300 nm. In some aspects, the LNP has a diameter of about 100-200 nm. In some aspects, the LNP has a diameter of about 100-175 nm. In some aspects, the LNP has a diameter of about 100-160 nm.

In some aspects, the lipid and the isolated polynucleotide (e.g., modified RNA) have a mass ratio of about 1:2 to about 2:1. In some aspects, the lipid and the isolated polynucleotide (e.g., modified RNA) have a mass ratio of 1:2, 1:1.5, 1:1.2, 1:1.1, 1:1, 1.1:1, 1.2:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, or 15:1. In some aspects, the lipid and the isolated polynucleotide (e.g., modified RNA) have a mass ratio of about 10:1.

Provided herein is a pharmaceutical composition comprising any of the isolated polynucleotides, vectors, or LNPs described herein, and a pharmaceutically acceptable carrier. In certain aspects, the pharmaceutical composition is formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

Provided herein is a cell comprising any of the isolated polynucleotides, vectors, or LNPs described herein. In some aspects, the cell is an in vitro cell, an ex vivo cell, or an in vivo cell.

Provided herein is a method of making a polynucleotide comprising enzymatically or chemically synthesizing any of the isolated polynucleotides described herein. Provided herein is a method of producing an IL-12 protein, comprising contacting a cell with any of the isolated polynucleotides, cells, or LNPs described herein. In certain aspects, the contacting occurs in vivo or ex vivo.

Present disclosure further provides a method of treating a disease or disorder in a subject in need thereof, comprising administering to the subject any of the isolated polynucleotides, vectors, LNPs, or pharmaceutical compositions described herein. In certain aspects, the disease or disorder comprises a cancer. In some aspects, the cancer comprises a melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, head and neck cancer, or combinations thereof.

In some aspects, a method of treating a disease or disorder disclosed herein further comprises administering at least one additional therapeutic agent to the subject. In certain aspects, the at least one additional therapeutic agent comprises a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof. In some aspects, the immune checkpoint inhibitor comprises an anti-PD-1 antibody, anti-PD-L1 antibody, anti-LAG-3 antibody, anti-CTLA-4 antibody, anti-GITR antibody, anti-TIM3 antibody, or any combination thereof.

### BRIEF DESCRIPTION OF THE DRAWINGS/FIGURES

FIGS. 1A and 1B provide comparison of tumor volume in tumor-bearing mice that were treated with a single intratumoral administration of one of the following: (i) PBS (control; open circle); (ii) repRNA co-transcriptionally capped with a 5′ cap analog (closed circle in FIG. 1A; solid line in FIG. 1B); and (iii) repRNA post-transcriptionally capped by enzymatic addition of 5′ cap (closed square in FIG. 1A; dashed line in FIG. 1B). The tumor volumes were measured at various time points post-administration (x-axis). FIG. 1A shows the average tumor volume. FIG. 1B shows the tumor volume of the individual animals.

FIG. 2 provides a comparison of the IL-12 protein expression in the tumor of mice treated with one of the following: (i) PBS (control; 1^{st} column from the left); (ii) repRNA made using the A3G +E1 vector and Cap Analog co-transcriptional capping (2^{nd} column); (iii) modified mRNA (non-self-replicating) made using Cap Analog co-transcriptional capping (3^{rd} column); (iv) repRNA made using the Alternative Evolved vector and Cap Analog co-transcriptional capping (4^{th} column); and (v) repRNA made using the Alternative Evolved vector and enzymatic post-translational capping (last column).

FIG. 3 provides a comparison of the Kaplan-Meyer survival analysis of tumor-bearing mice that were treated with a single intratumor administration of one of the following: (i) PBS (control; open circle); (ii) repRNA made using the A3G +E1 vector and Cap Analog co-transcriptional capping (open triangle); (iii) modified mRNA (non-self-replicating) made using Cap Analog co-transcriptional capping (open square); (iv) repRNA made using the Alternative Evolved vector and Cap Analog co-transcriptional capping (closed circle); and (v) repRNA made using the Alternative Evolved vector and enzymatic post-translational capping (closed square).

FIG. 4A provides a comparison of the in vitro transfection efficiency of the different RNA constructs described herein, as measured using FACS analysis. FIG. 4B provides a comparison of IL-12 protein concentration detected in the supernatant of cells transfected with the different RNA constructs described herein. In both FIGS. 4A and 4B, the RNA constructs tested included the following: (i) repRNA made using the A3G +E1 vector and Cap Analog co-transcriptional capping (closed circle); (ii) repRNA made using the A3G +E1 vector and enzymatic post-translational capping (closed square); (iii) repRNA made using the Alternative Evolved +E1 vector and Cap Analog co-transcriptional capping (closed triangle); (iv) repRNA made using the Alternative Evolved +E1 vector and enzymatic post-translational capping (closed inverted triangle); (v) repRNA made using the Alternative +E1 vector and Cap Analog co-transcriptional capping (diamond); (vi) repRNA made using the A3G +E1 Evolved vector and Cap Analog co-transcriptional capping (open circle); (vii) repRNA made using the A3G −E1 vector and Cap Analog co-transcriptional capping (open square); and (viii) repRNA made using the Alternative Evolved −E1 SGP scar end vector (open triangle). The transfection efficiency is shown as the frequency of IL-12+ cells observed in the different groups. The x-axis represents the hours post-transfection when the transfection efficiency was assessed.

FIG. 5 provides a comparison of the in vitro transfection efficiency of the following RNA constructs, as measured using FACS analysis: (i) repRNA made using the Cap Analog co-transcriptional capping method and A3G +E1 vector with a 3′-end terminating with a restriction enzyme scar (circle); (ii) repRNA made using the Cap Analog (alternate source) method and A3G +E1 vector with a 3′-end terminating with a restriction enzyme scar (square); (iii) repRNA made using the Cap Analog co-transcriptional capping method and A3G +E1 vector with a 3′-end terminating with a clean polyA sequence (triangle); (iv) repRNA made using the Cap Analog (alternate source) method and A3G +E1 vector with a 3′-end terminating with a clean polyA sequence (inverted triangle). PBS treated cells were used as control (diamond). The transfection efficiency is shown as the frequency of IL-12+ cells observed in the different groups. The x-axis represents the hours post-transfection when the transfection efficiency was assessed.

FIGS. 6A and 6B provide comparison of IL-12 protein concentration observed in tumor and serum of tumor-bearing mice treated with a single intratumoral administration of an RNA construct encoding one of the following mouse IL-12 proteins: (i) mIL-12 alone (“IL-12”); (ii) mIL-12 conjugated to albumin (“IL-12-alb”); and (iii) mIL-12 conjugated to albumin and lumican (“IL-12-alb-lum”). The different RNA constructs were administered to the animals at one of the two doses shown along the x-axis. FIG. 6A shows the IL-12 protein concentration in tumor (top graph) and serum (bottom graph) at 24 hours post-administration. FIG. 6B shows the IL-12 protein concentration in tumor (top graph) and serum (bottom graph) at 96 hours post-administration.

FIGS. 7A, 7B, and 7C provide comparison of IL-12 protein concentration observed in spleen, draining lymph nodes, and non-draining lymph nodes, respectively, of tumor-bearing mice at 24 hours after a single intratumoral administration of an RNA construct encoding one of the following mouse IL-12 proteins: (i) mIL-12 alone (“IL-12”); (ii) mIL-12 conjugated to albumin (“IL-12-alb”); and (iii) mIL-12 conjugated to albumin and lumican (“IL-12-alb-lum”). The different RNA constructs were administered to the animals at one of the two doses shown along the x-axis.

FIGS. 8A, 8B, and 8C provide comparison of IL-12 protein concentration observed in spleen, draining lymph nodes, and non-draining lymph nodes, respectively, of tumor-bearing mice at 96 hours after a single intratumoral administration of an RNA construct encoding one of the following mouse IL-12 proteins: (i) mIL-12 alone (“IL-12”); (ii) mIL-12 conjugated to albumin (“IL-12-alb”); and (iii) mIL-12 conjugated to albumin and lumican (“IL-12-alb-lum”). The different RNA constructs were administered to the animals at one of the two doses shown along the x-axis.

FIGS. 9A and 9B provide comparison of IL-12 protein and IFN-γ protein concentrations, respectively, measured in the serum of tumor-bearing mice treated with a single intratumoral administration of an RNA construct encoding one of the following mouse IL-12 proteins: (i) mIL-12 alone (triangle); (ii) mIL-12 conjugated to albumin (square); and (iii) mIL-12 conjugated to albumin and lumican (circle). The RNA constructs were administered the animals at the following doses: 0.25 μg (closed symbols) or 2.5 μg (open symbols). The x-axis provides the time points (post-administration) at which the IL-12 and IFN-γ concentrations were measured.

FIG. 10 presents a graphical representation of data related to optimality (avg_codon_score) vs minimum folding free energy in kcal/mol (MFE) for 1137 distinct sequences encoding the fusion protein human light chain leader—hIL12p40—GGS(GGGS)3 linker—hIL12p35—GSGGGS linker—Human serum albumin in accordance with Example 5. The L1, L2, L3, M1, M2, M3, H1, H2, and H3 codon-optimized constructs are indicated. As described in Example 5, the diamond symbol represents sequence with very high codon optimality and MFE. The triangle represents the codon optimal sequence containing the most frequently used triplet at each amino acid position.

FIG. 11 provides a comparison of the IL-12 protein secretion observed in the supernatant of cells transfected with different RNA constructs comprising a codon-optimized IL-12 sequence (x-axis). As indicated, the IL-12 of constructs A1-A4 were not conjugated to any other moieties. IL-12 of constructs B1-B4 were conjugated to albumin. IL-12 of constructs C1-C3 were conjugated to albumin and lumican. Individual triplicate measurements are shown as triangular, square, octagonal, or circular points, and horizontal bars show the average of triplicate measurements. The X-axis represents the variant used and the Y-axis represents the concentration of IL-12 in ng/ml.

FIGS. 12A, 12B, and 12C provide comparison of IL-12 protein concentration observed in the tumor (FIG. 12A), serum (FIG. 12B), and spleen (FIG. 12C) of tumor-bearing mice that received a single intratumor administration of a RNA construct comprising (i) codon-optimized IL-12 sequence conjugated to albumin and lumican (1^{st} set of circles from the left in each of PDX1, PDX2, and PDX3); (ii) codon-optimized IL-12 sequence conjugated to albumin (2^{nd} set of circles in each of PDX1, PDX2, and PDX3); or (iii) codon-optimized IL-12 sequence alone (3^{rd} set of circles in each of PDX1, PDX2, and PDX3). Non-treated animals were used as control (last set of circles in each of PDX1, PDX2, and PDX3). “PDX1,” “PDX2,” and “PDX3” represent xenografts derived from tumor resections from three individual patients with triple-negative breast cancer (TNBC). PDX1 and PDX3 were established from primary resected ER,PR,HER2 negative lesions, and PDX2 was established from a metastatic ER,PR,HER2-negative lesion in the lung of a TNBC patient.

FIG. 13 provides a comparison of tumor volume in TNBC mice that were treated (weekly for a total of four doses) with the vehicle control (open square) or repRNA encoding IL-12 (repRNA). The repRNA was administered to the animals at one of the following doses: (i) 5 μg (closed circle), (ii) 0.5 μg (closed square), or (iii) 0.05 μg (closed triangle).

FIG. 14 provides a comparison of tumor volume in TNBC mice that were treated with rIL-12-encoding repRNA modified to comprise different amounts of modified nucleoside triphosphates (modNTPs): (i) 0% modNTP (i.e., non-modified repRNA) (closed circle); (ii) 25% modNTP (closed square); (iii) 37.5% modNTP (closed triangle); or (iv) 50% modNTP (closed inversed triangle). Animals treated with the vehicle control were used as control (open square).

FIGS. 15A and 15B show the effects of the repRNA constructs described herein on both treated and non-treated tumors, respectively, in a B16-F10 mouse syngeneic cancer model. As further described in Example 8, the left and right flanks of the animals were subcutaneously injected with B16-F10 tumor cells. Once optimal tumor size was reached, the vehicle control or repRNA (2.5 μg) was intratumorally administered in the left flank (i.e., treated tumor). Then, tumor volume was assessed in both the left flank and the right flank (i.e., non-treated tumor).

FIG. 16 provides a comparison of luciferase expression (shown as bioluminescence signal) in TNBC mouse model after re-dosing. As further described in Example 9, the mice were injected with a first dose of one of two firefly luciferase-encoding repRNA constructs (mRNA1 and mRNA2) (5 μg) (“Dose 1”) and then 1 week later, injected with a second dose with the same repRNA construct (5 μg) (“Dose 2”). In some of the animals, they additionally received weekly (two total doses) administration of an anti-IFNAR1 antibody (10 mg/kg).

FIG. 17 provides a comparison of IFN-γ concentration in the supernatant collected from activated human PBMCs treated with recombinant human IL-12 protein (rhIL12) or conditioned media as described in Example 10 (i.e., supernatant collected from BT20 cells transfected with IL-12 encoding repRNA). The PBMCs were treated with the rhIL12 at one of the following concentrations: 100 ng/mL (“3”), 10 ng/mL (“4”), 1 ng/mL (“5”), 0.1 ng/mL (“6”), or 0.01 ng/mL (“7”). The conditioned media contained one of the following amounts of IL-12: 1 ng/mL (“1”) and 10 ng/mL (“2”). Non-treated cells were used as control (“8”).

### DETAILED DESCRIPTION OF THE DISCLOSURE

### Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures.

“Polynucleotide” or “nucleic acid” as used herein means a sequence of nucleotides connected by phosphodiester linkages. Polynucleotides are presented herein in the direction from the 5′ to the 3′ direction. A polynucleotide of the present disclosure can be a deoxyribonucleic acid (DNA) molecule or ribonucleic acid (RNA) molecule. Nucleotide bases are indicated herein by a single letter code: adenine (A), guanine (G), thymine (T), cytosine (C), inosine (I) and uracil (U).

As used herein, the term “polypeptide” encompasses both peptides and proteins, unless indicated otherwise.

The term “coding sequence” or sequence “encoding” is used herein to mean a DNA or RNA region (the transcribed region) which “encodes” a particular protein, e.g., such as an IL-12. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

A Kozak consensus sequence, Kozak consensus or Kozak sequence, is known as a sequence which occurs on eukaryotic mRNA and has the consensus (gcc)gccRccAUGG (SEQ ID NO: 174), where R is a purine (adenine or guanine) three bases upstream of the start codon (AUG), which is followed by another “G.” In some aspects, the polynucleotide comprises a nucleic acid sequence having at least about 95% or more, e.g., at least 99% sequence identity, to the Kozak consensus sequence. In some aspects, the polynucleotide comprises a Kozak consensus sequence.

The term “RNA” is used herein to mean a molecule which comprises at least one ribonucleotide residue. “Ribonucleotide” relates to a nucleotide with a hydroxyl group at the 2′-position of a β-D-ribofuranosyl group. The term comprises double-stranded RNA, single-stranded RNA, isolated RNA such as partially or completely purified RNA, essentially pure RNA, synthetic RNA, recombinantly generated RNA such as modified RNA which differs from naturally occurring RNA by addition, deletion, substitution and/or alteration of one or more nucleotides. The term “mRNA” means “messenger-RNA” and relates to a “transcript” which is generated by using a DNA template and encodes a peptide or protein. Typically, an mRNA comprises a 5′-UTR, a protein coding region and a 3′-UTR. mRNA only possesses limited half-life in cells and in vitro. In the context of the present disclosure, mRNA can be generated by in vitro transcription from a DNA template. The in vitro transcription methodology is known to the skilled person. For example, there is a variety of in vitro transcription kits commercially available. In some aspects of the disclosure, the RNA, preferably the mRNA, is modified with a 5′-cap structure.

The term “sequence identity” is used herein to mean a relationship between two or more amino acid (polypeptide or protein) sequences or two or more nucleic acid (polynucleotide) sequences, as determined by comparing the sequences. In certain aspects, sequence identity is calculated based on the full length of two given SEQ ID NO or on part thereof. Part thereof can mean at least 10%, 20%, 30%, 40%, 50%, 60%, 70%, 80%, 90%, or 100% of both SEQ ID NO, or any other specified percentage. The term “identity” can also mean the degree of sequence relatedness between amino acid or nucleic acid sequences, as the case can be, as determined by the match between strings of such sequences.

In certain aspects, methods to determine identity are designed to give the largest match between the sequences tested. Methods to determine identity and similarity are codified in publicly available computer programs.

“Substantial homology” or “substantial similarity,” means, when referring to a nucleic acid or fragment thereof, indicates that, when optimally aligned with appropriate nucleotide insertions or deletions with another nucleic acid (or its complementary strand), there is nucleotide sequence identity in at least about 95 to 99% of the sequence.

As used herein, the terms “effective amount,” “therapeutically effective amount,” and a “sufficient amount” of, e.g., a composition comprising a polynucleotide disclosed herein, refer to a quantity sufficient to, when administered to the subject, including a human, effect beneficial or desired results, including clinical results, and, as such, an “effective amount” or synonym thereto depends on the context in which it is being applied.

The amount of a given therapeutic agent or composition will correspond to such an amount will vary depending upon various factors, such as the given agent, the pharmaceutical formulation, the route of administration, the type of disease or disorder, the identity of the subject (e.g., age, sex, and/or weight) or host being treated, and the like.

The term “half-life” relates to the period of time which is needed to eliminate half of the activity, amount, or number of molecules. In the context of the present disclosure, the half-life of an RNA is indicative for the stability of said RNA. “Development” or “progression” of a disease means initial manifestations and/or ensuing progression of the disease. Development of the disease can be detectable and assessed using standard clinical techniques as well known in the art. However, development also refers to progression that can be unpredictable. As used herein, development or progression refers to the biological course of symptoms. Development includes occurrence, recurrence, and onset. As used herein, onset or occurrence of a target disease or disorder includes initial onset and/or recurrence.

### IL-12 Expressing Nucleotides

The present disclosure is directed to a polynucleotide (e.g., isolated polynucleotide) comprising a nucleic acid molecule encoding an IL-12 protein. As described herein, the polynucleotides disclosed herein comprise one or more features, such that they are distinct (e.g., structurally and/or functionally) from a reference polynucleotide that exists in nature. For instance, as further described elsewhere in the present disclosure, the nucleic acid molecules, e.g., encoding an IL-12 protein (e.g., IL-12α subunit and/or IL-12β subunit), have been codon-optimized (i.e., synthetic).

In some aspects, the nucleotide sequence encoding IL-12 comprises a translatable region and one, two, or more than two modifications. In some aspects, the nucleotide sequence encoding IL-12 exhibits reduced degradation in a cell into which the nucleic acid is introduced, relative to a corresponding unmodified nucleic acid.

In some aspects, the modification can be located on the sugar moiety of the nucleotide. In some aspects, the modification can be located on the phosphate backbone of the nucleotide.

In some aspects, it is desirable to intracellularly degrade a modified nucleic acid introduced into the cell, for example if precise timing of protein production is desired. Thus, in some aspects, the nucleotide sequence encoding IL-12 comprises a degradation domain, which is capable of being acted on in a directed manner within a cell.

In some aspects, the nucleotide sequence encoding IL-12 comprises at least one of a modified 5′-cap, a half-life extending moiety, or a regulatory element.

In some aspects, the modified 5′-cap increases the stability of the RNA, increases translation efficiency of the RNA, prolongs translation of the RNA, increases total protein expression of the RNA when compared to the same RNA without the 5′-cap structure.

In some aspects, the nucleotide sequence encoding IL-12 is cyclized, or concatemerized, to generate a translation competent molecule to assist interactions between poly-A binding proteins and 5′-end binding proteins. The mechanism of cyclization or concatemerization can occur through at least 3 different routes: 1) chemical, 2) enzymatic, and 3) ribozyme catalyzed. The newly formed 5′-/3′-linkage can be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end can contain an NETS-ester reactive group and the 3′-end can contain a 3′-amino-terminated nucleotide such that in an organic solvent the 3′-amino-terminated nucleotide on the 3′-end of a synthetic mRNA molecule will undergo a nucleophilic attack on the 5′—NHS-ester moiety forming a new 5′-/3′-amide bond.

In the second route, T4 RNA ligase can be used to enzymatically link a 5′-phosphorylated nucleic acid molecule to the 3′-hydroxyl group of a nucleic acid forming a new phosphorodiester linkage. In an example reaction, 1 μg of a nucleic acid molecule is incubated at 37° C. for 1 hour with 1-10 units of T4 RNA ligase (New England Biolabs, Ipswich, Mass.) according to the manufacturer's protocol. The ligation reaction can occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme can be derived from the Group I Intron, Group I Intron, Hepatitis Delta Virus, Hairpin ribozyme or can be selected by SELEX (systematic evolution of ligands by exponential enrichment). The ribozyme ligase reaction can take 1 to 24 hours at temperatures between 0 and 37° C.

In some aspects, multiple distinct nucleic acids, nucleotide sequence encoding IL-12 or primary constructs can be linked together through the 3′-end using nucleotides which are modified at the 3′-terminus. Chemical conjugation can be used to control the stoichiometry of delivery into cells. For example, the glyoxylate cycle enzymes, isocitrate lyase and malate synthase, can be supplied into HepG2 cells at a 1:1 ratio to alter cellular fatty acid metabolism. This ratio can be controlled by chemically linking nucleic acids or modified RNA using a 3′-azido terminated nucleotide on one nucleic acids or modified RNA species and a C5-ethynyl or alkynyl-containing nucleotide on the opposite nucleic acids or nucleotide sequence species encoding IL-12. The nucleotide sequence encoding IL-12 is added post-transcriptionally using terminal transferase (New England Biolabs, Ipswich, Mass.) according to the manufacturer's protocol. After the addition of the 3′-modified nucleotide, the two nucleic acids or nucleotide sequence encoding IL-12 can be combined in an aqueous solution, in the presence or absence of copper, to form a new covalent linkage via a click chemistry mechanism as described in the literature.

In some aspects, more than two polynucleotides can be linked together using a functionalized linker molecule. For example, a functionalized saccharide molecule can be chemically modified to contain multiple chemical reactive groups (SH—, NH2-, N3, etc. . . . ) to react with the cognate moiety on a 3′-functionalized mRNA molecule (i.e., a 3′-maleimide ester, 3′-NHS-ester, alkynyl). The number of reactive groups on the modified saccharide can be controlled in a stoichiometric fashion to directly control the stoichiometric ratio of conjugated nucleic acid or mRNA.

In some aspects, to further enhance protein production, nucleotide sequence encoding IL-12, polynucleotides or primary constructs of the present disclosure can be designed to be conjugated to other polynucleotides, dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug.

Conjugation can result in increased stability and/or half-life and can be particularly useful in targeting the nucleotide sequence encoding IL-12 or the primary constructs to specific sites in the cell, tissue or organism.

In some aspects, the primary construct is designed to encode one or more polypeptides of interest or fragments thereof. A polypeptide of interest can include, but is not limited to, whole polypeptides, a plurality of polypeptides or fragments of polypeptides, which independently can be encoded by one or more nucleic acids, a plurality of nucleic acids, fragments of nucleic acids or variants of any of the aforementioned. As used herein, the term “polypeptides of interest” refers to any polypeptide which is selected to be encoded in the primary construct of the present disclosure. As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide can be a single molecule or can be a multi-molecular complex such as a dimer, trimer or tetramer. They can also comprise single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants can possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

As such, polynucleotides encoding polypeptides of interest containing substitutions, insertions and/or additions, deletions and covalent modifications with respect to reference sequences are included within the scope of this disclosure. For example, sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein can optionally be deleted providing for truncated sequences. Certain amino acids (e.g., C-terminal or N-terminal residues) can alternatively be deleted depending on the use of the sequence, as for example, expression of the sequence as part of a larger sequence which is soluble, or linked to a solid support.

As recognized by those skilled in the art, protein fragments, functional protein domains, and homologous proteins are also considered to be within the scope of polypeptides of interest of this disclosure. For example, provided herein is any protein fragment (meaning an polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the disclosure. In certain aspects, a polypeptide to be utilized in accordance with the disclosure includes 2, 3, 4, 5, 6, 7, 8, 9, 10, or more mutations as shown in any of the sequences provided or referenced herein.

In some aspects, the nucleotide sequence encoding IL-12 comprises a modified 5′-cap, a half-life extending moiety, a regulatory element, or combinations thereof.

In some aspects, the modified 5′-cap is selected from the group consisting of m₂^{7,2′-O}GppₛpGRNA, m⁷GpppG, m⁷Gppppm⁷G, m₂^{(7,3′-O)}GpppG, m₂^{(7,2′-O)}GppspG(D1), m₂^{(7,2′-O)}GppspG(D2), m₂^{7,3′-O}Gppp(m₁^{2′-O)}ApG, (m⁷G-3′ mppp-G; which can equivalently be designated 3′ O-Me-m7G(5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, and combinations thereof.

The disclosure also includes a polynucleotide that comprises both a 5′ Cap and a nucleotide sequence encoding IL-12 of the disclosure (e.g., a polynucleotide comprising a nucleotide sequence encoding an IL12B polypeptide, an IL12A polypeptide, and/or IL12B and IL12A fusion polypeptides).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

In some aspects, the 5′ terminal cap structure is a CapO, Cap1, ARC A, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

Non-limiting additional Caps include the 5′ Caps disclosed in WO/2017/201350, published Nov. 23, 2017, which is incorporated herein by reference.

In some aspects, the half-life extending moiety comprises an Fc, an albumin or a fragment thereof, an albumin binding moiety, a PAS sequence, a HAP sequence, transferrin or a fragment thereof, an XTEN, or any combinations thereof.

In some aspects, the half-life extending moiety comprises an Fc. In some aspects, the half-life extending moiety comprises an albumin or a fragment thereof.

In some aspects, the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, and combinations thereof.

In some aspects, the regulatory element further comprises a polyA region. In some aspects, the nucleotide sequence encoding IL-12 of the present disclosure (e.g., a polynucleotide comprising a nucleotide sequence encoding an IL12B polypeptide, an IL12A polypeptide, and/or IL12B and IL12A fusion polypeptides) further comprise a poly-A tail. In some aspects, terminal groups on the poly-A tail can be incorporated for stabilization. In other aspects, a poly-A tail comprises des-3′ hydroxyl tails. During RNA processing, a long chain of adenine nucleotides (poly-A tail) can be added to a polynucleotide such as an mRNA molecule in order to increase stability.

Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

PolyA tails can also be added after the construct is exported from the nucleus.

According to the present disclosure, terminal groups on the poly A tail can be incorporated for stabilization. Polynucleotides of the present disclosure can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety). See also WO/2017/201350, published Nov. 23, 2017, which is incorporated herein by reference, for additional poly-A tails.

In some aspects, the nucleotide sequence encoding IL-12 comprises any modification or combination of modifications described herein.

### Terminal Architecture Modifications: Untranslated Regions (UTRs)

Untranslated regions (UTRs) of a gene are transcribed but not translated. The 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the RNA (e.g., modified RNA) of the present disclosure to enhance the stability of the molecule. The specific features can also be incorporated to ensure controlled down-regulation of the transcript in case they are misdirected to undesired organs sites.

### 5′ UTR and Translation Initiation

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

5′UTR secondary structures involved in elongation factor binding can interact with other RNA binding molecules in the 5′UTR or 3′UTR to regulate gene expression. For example, the elongation factor EIF4A2 binding to a secondarily structured element in the 5′UTR is necessary for microRNA mediated repression (Meijer H A et al., Science, 2013, 340, 82-85, herein incorporated by reference in its entirety). The different secondary structures in the 5′UTR can be incorporated into the flanking region to either stabilize or selectively destalized mRNAs in specific tissues or cells.

By engineering the features typically found in abundantly expressed genes of specific target organs, one can enhance the stability and protein production of the nucleic acids or mRNA of the disclosure. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a mmRNA, in hepatic cell lines or liver. Likewise, use of 5′ UTR from other tissue-specific mRNA to improve expression in that tissue is possible—for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences can be incorporated into the 5′ (or 3′ UTR) UTRs. For example, introns or portions of introns sequences can be incorporated into the flanking regions of the nucleic acids or mRNA of the disclosure. Incorporation of intronic sequences can increase protein production as well as mRNA levels.

In some aspects of the disclosure, at least one fragment of IRES sequences from a GTX gene can be included in the 5′UTR. As a non-limiting example, the fragment can be an 18 nucleotide sequence from the IRES of the GTX gene. As another non-limiting example, an 18 nucleotide sequence fragment from the IRES sequence of a GTX gene can be tandemly repeated in the 5′UTR of a polynucleotide described herein. The 18 nucleotide sequence can be repeated in the 5′UTR at least one, at least twice, at least three times, at least four times, at least five times, at least six times, at least seven times, at least eight times, at least nine times or more than ten times.

Nucleotides can be mutated, replaced and/or removed from the 5′ (or 3′) UTRs. For example, one or more nucleotides upstream of the start codon can be replaced with another nucleotide. The nucleotide or nucleotides to be replaced can be 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more than 60 nucleotides upstream of the start codon. As another example, one or more nucleotides upstream of the start codon can be removed from the UTR.

### 3′UTR and Translation Enhancer Elements (TEEs)

In some aspects, the 5′UTR of the nucleotide sequence encoding IL-12 comprises at least one translational enhancer polynucleotide, translation enhancer element, translational enhancer elements (collectively referred to as “TEE”s). In some aspects, the TEE is located between the transcription promoter and the start codon. In some aspects, the RNA (e.g., modified RNA) with at least one TEE in the 5′UTR comprises a cap at the 5′UTR. In some aspects, the at least one TEE can be located in the 5′UTR of nucleotide sequence encoding IL-12 undergoing cap-dependent or cap-independent translation.

The term “translational enhancer element” or “translation enhancer element” (herein collectively referred to as “TEE”) refers to sequences that increase the amount of polypeptide or protein produced from an mRNA.

In one aspect, TEEs are conserved elements in the UTR which can promote translational activity of a nucleic acid such as, but not limited to, cap-dependent or cap-independent translation. The conservation of these sequences has been previously shown by Panek et al (Nucleic Acids Research, 2013, 1-10; herein incorporated by reference in its entirety) across 14 species including humans.

In some aspects, the nucleotide sequence encoding IL-12 has at least one TEE that has at least about 50%, at least about 55%, at least about 60%, at least about 65%, at least about 70%, at least about 75%, at least about 80%, at least about 85%, at least about 90%, at least about 95% or at least about 99% identity with the disclosed in U.S. Application Number 2014/0147454, which is hereby incorporated by reference in its entirety. In some aspects, the RNA (e.g., modified RNA) includes at least one TEE that has at least about 50%, at least about 55%, at least about 60%, at least about 65%, at least about 70%, at least about 75%, at least about 80%, at least about 85%, at least about 90%, at least about 95% or at least about 99% identity with the TEEs described in US Patent Publication Nos. US20090226470, US20070048776, US20130177581 and US20110124100, International Patent Publication No. WO1999024595, WO2012009644, WO2009075886 and WO2007025008, European Patent Publication No. EP2610341A1 and EP2610340A1, U.S. Pat. Nos. 6,310,197, 6,849,405, 7,456,273, 7,183,395, each of which is herein incorporated by reference in its entirety.

In some aspects, the 5′UTR of the nucleotide sequence encoding IL-12 can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some aspects, the TEE sequences in the 5′UTR of the RNA (e.g., modified RNA) are the same or different TEE sequences. In some aspects, the TEE sequences are in a pattern such as ABABAB or AABBAABBAABB or ABCABCABC or variants thereof repeated once, twice, or more than three times. In these patterns, each letter, A, B, or C represent a different TEE sequence at the nucleotide level.

In some aspects, the spacer separating two TEE sequences includes other sequences known in the art which regulate the translation of the RNA (e.g., modified RNA) such as, but not limited to, miR sequences described herein (e.g., miR binding sites and miR seeds). In some aspects, each spacer used to separate two TEE sequences includes a different miR sequence or component of a miR sequence (e.g., miR seed sequence).

In some aspects, the TEE used in the 5′UTR of the nucleotide sequence encoding IL-12 of the present disclosure is an IRES sequence such as, but not limited to, those described in U.S. Pat. No. 7,468,275 and International Patent Publication No. WO2001055369, each of which is herein incorporated by reference in its entirety.

In some aspects, the TEEs described herein are located in the 5′UTR and/or the 3′UTR of the nucleotide sequence encoding IL-12. In some aspects, the TEEs located in the 3′UTR are the same and/or different than the TEEs located in and/or described for incorporation in the 5′UTR.

In some aspects, the 3′UTR of the nucleotide sequence encoding IL-12 can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some aspects, the TEE sequences in the 3′UTR of the nucleotide sequence encoding IL-12 of the present disclosure is the same or different TEE sequences. The TEE sequences is in a pattern such as ABABAB or AABBAABBAABB or ABCABCABC or variants thereof repeated once, twice, or more than three times. In these patterns, each letter, A, B, or C represent a different TEE sequence at the nucleotide level.

In some aspects, the 3′UTR includes a spacer to separate two TEE sequences. In some aspects, the spacer is a 15 nucleotide spacer and/or other spacers known in the art. In some aspects, the 3′UTR can include a TEE sequence-spacer module repeated at least once, at least twice, at least 3 times, at least 4 times, at least 5 times, at least 6 times, at least 7 times, at least 8 times and at least 9 times or more than 9 times in the 3′UTR.

In some aspects, the spacer separating two TEE sequences includes other sequences known in the art which regulate the translation of the nucleotide sequence encoding IL-12, such as, but not limited to, miR sequences described herein (e.g., miR binding sites and miR seeds). In some aspects, each spacer used to separate two TEE sequences includes a different miR sequence or component of a miR sequence (e.g., miR seed sequence).

Incorporating microRNA Binding Sites

In some aspects, the nucleotide sequence encoding IL-12 further comprises a sensor sequence. Sensor sequences include, for example, microRNA binding sites, transcription factor binding sites, structured mRNA sequences and/or motifs, artificial binding sites engineered to act as pseudo-receptors for endogenous nucleic acid binding molecules. Non-limiting examples, of polynucleotides comprising at least one sensor sequence are described U.S. Application No. 2014/0147454, which is hereby incorporated by reference in its entirety.

In some aspects, microRNA (miRNA) profiling of the target cells or tissues is conducted to determine the presence or absence of miRNA in the cells or tissues.

MicroRNAs (or miRNA) are 19-25 nucleotide long noncoding RNAs that bind to the 3′UTR of nucleic acid molecules and down-regulate gene expression either by reducing nucleic acid molecule stability or by inhibiting translation. In some aspects, the RNA (e.g., modified RNA), comprises one or more microRNA target sequences, microRNA sequences, or microRNA seeds. Such sequences can correspond to any known microRNA such as those taught in US Publication US2005/0261218 and US Publication US2005/0059005, the contents of which are incorporated herein by reference in their entirety. As a non-limiting example, known microRNAs, their sequences and seed sequences in human genome are described in U.S. Application No. 2014/0147454, which is herein incorporated by reference in its entirety.

A microRNA sequence comprises a “seed” region, i.e., a sequence in the region of positions 2-8 of the mature microRNA, which sequence has perfect Watson-Crick complementarity to the miRNA target sequence. A microRNA seed comprises positions 2-8 or 2-7 of the mature microRNA. In some aspects, a microRNA seed comprises 7 nucleotides (e.g., nucleotides 2-8 of the mature microRNA), wherein the seed-complementary site in the corresponding miRNA target is flanked by an adenine (A) opposed to microRNA position 1. In some aspects, a microRNA seed comprises 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K, Johnston W K, Garrett-Engele P, Lim L P, Bartel D P; Mol Cell. 2007 Jul. 6; 27(1):91-105. The bases of the microRNA seed have complete complementarity with the target sequence. By engineering microRNA target sequences into the 3′UTR of nucleic acids or mRNA of the disclosure one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/1eu.2011.356); Bartel Cell 2009 136:215-233; Landgraf et al, Cell, 2007 129:1401-1414; Gentner and Naldini, Tissue Antigens. 2012 80:393-403 and all references therein; each of which is herein incorporated by reference in its entirety).

For example, if the mRNA is not intended to be delivered to the liver but ends up there, then miR-122, a microRNA abundant in liver, can inhibit the expression of the gene of interest if one or multiple target sites of miR-122 are engineered into the 3′UTR of the modified nucleic acids, enhanced modified RNA or ribonucleic acids. Introduction of one or multiple binding sites for different microRNA can be engineered to further decrease the longevity, stability, and protein translation of a modified nucleic acids, enhanced modified RNA or ribonucleic acids. As used herein, the term “microRNA site” refers to a microRNA target site or a microRNA recognition site, or any nucleotide sequence to which a microRNA binds or associates. It should be understood that “binding” can follow traditional Watson-Crick hybridization rules or can reflect any stable association of the microRNA with the target sequence at or adjacent to the microRNA site.

Conversely, for the purposes of the nucleotide sequence encoding IL-12 of the present disclosure, microRNA binding sites can be engineered out of (i.e. removed from) sequences in which they naturally occur in order to increase protein expression in specific tissues. For example, miR-122 binding sites can be removed to improve protein expression in the liver.

In some aspects, the nucleotide sequence encoding IL-12 includes at least one miRNA-binding site in the 3′UTR in order to direct cytotoxic or cytoprotective mRNA therapeutics to specific cells such as, but not limited to, normal and/or cancerous cells (e.g., HEP3B or SNU449).

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, microRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, particularly abundant in myeloid dendritic cells. It was demonstrated in the art that the immune response to exogenous nucleic acid molecules was shut-off by adding miR-142 binding sites to the 3′UTR of the delivered gene construct, enabling more stable gene transfer in tissues and cells. miR-142 efficiently degrades the exogenous mRNA in antigen presenting cells and suppresses cytotoxic elimination of transduced cells (Annoni A et al., blood, 2009, 114, 5152-5161; Brown B D, et al., Nat med. 2006, 12(5), 585-591; Brown B D, et al., blood, 2007, 110(13): 4144-4152, each of which is herein incorporated by reference in its entirety).

Many microRNA expression studies are conducted in the art to profile the differential expression of microRNAs in various cancer cells/tissues and other diseases. Some microRNAs are abnormally over-expressed in certain cancer cells and others are under-expressed. For example, microRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lympho nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in their entirety.)

At least one microRNA site can be engineered into the 3′ UTR of the nucleotide sequence encoding IL-12. In some aspects, at least two, at least three, at least four, at least five, at least six, at least seven, at least eight, at least nine, at least ten or more microRNA sites can be engineered into the 3′ UTR of the nucleotide sequence encoding IL-12. In some aspects, the microRNA sites incorporated into the nucleotide sequence encoding IL-12 are the same or different microRNA sites. In some aspects, the microRNA sites incorporated into the nucleotide sequence encoding IL-12 targets the same or different tissues in the body. As a non-limiting example, through the introduction of tissue-, cell-type-, or disease-specific microRNA binding sites in the 3′ UTR of a modified nucleic acid mRNA, the degree of expression in specific cell types (e.g., hepatocytes, myeloid cells, endothelial cells, cancer cells, etc.) can be reduced.

In some aspects, a microRNA site is engineered near the 5′ terminus of the 3′UTR, about halfway between the 5′ terminus and 3′ terminus of the 3′UTR and/or near the 3′ terminus of the 3′UTR. In some aspects, a microRNA site is engineered near the 5′ terminus of the 3′UTR and about halfway between the 5′ terminus and 3′ terminus of the 3′UTR. In some aspects, a microRNA site is engineered near the 3′ terminus of the 3′UTR and about halfway between the 5′ terminus and 3′ terminus of the 3′UTR. In some aspects, a microRNA site is engineered near the 5′ terminus of the 3′UTR and near the 3′ terminus of the 3′UTR.

In some aspects, a modified messenger RNA comprises microRNA binding region sites that either have 100% identity to known seed sequences or have less than 100% identity to seed sequences. The seed sequence can be partially mutated to decrease microRNA binding affinity and as such result in reduced downmodulation of that mRNA transcript. In essence, the degree of match or mis-match between the target mRNA and the microRNA seed can act as a rheostat to more finely tune the ability of the microRNA to modulate protein expression. In addition, mutation in the non-seed region of a microRNA binding site can also impact the ability of a microRNA to modulate protein expression.

### RNA Motifs for RNA Binding Proteins (RBPs)

RNA binding proteins (RBPs) can regulate numerous aspects of co- and post-transcription gene expression such as, but not limited to, RNA splicing, localization, translation, turnover, polyadenylation, capping, modification, export and localization. RNA-binding domains (RBDs), such as, but not limited to, RNA recognition motif (RR) and hnRNP K-homology (KH) domains, typically regulate the sequence association between RBPs and their RNA targets (Ray et al. Nature 2013. 499:172-177; herein incorporated by reference in its entirety). In some aspects, the canonical RBDs bind short RNA sequences. In some aspects, the canonical RBDs recognize RNA structure.

Non limiting examples of RNA binding proteins and related nucleic acid and protein sequences are described in U.S. Application No. 2014/0147454, which is herein incorporated by reference in its entirety.

In some aspects, to increase the stability of the mRNA of interest, an mRNA encoding HuR is co-transfected or co-injected along with the mRNA of interest into the cells or into the tissue. These proteins can also be tethered to the mRNA of interest in vitro and then administered to the cells together. Poly A tail binding protein, PABP interacts with eukaryotic translation initiation factor eIF4G to stimulate translational initiation. Co-administration of mRNAs encoding these RBPs along with the mRNA drug and/or tethering these proteins to the mRNA drug in vitro and administering the protein-bound mRNA into the cells can increase the translational efficiency of the mRNA. The same concept can be extended to co-administration of mRNA along with mRNAs encoding various translation factors and facilitators as well as with the proteins themselves to influence RNA stability and/or translational efficiency.

In some aspects, the nucleotide sequence encoding IL-12 comprises at least one RNA-binding motif such as, but not limited to a RNA-binding domain (RBD).

In some aspects, the first region of linked nucleosides and/or at least one flanking region comprises at least on RBD. In some aspects, the first region of linked nucleosides comprises a RBD related to splicing factors and at least one flanking region comprises a RBD for stability and/or translation factors.

### Other Regulatory Elements in 3′UTR

In addition to microRNA binding sites, other regulatory sequences in the 3′-UTR of natural mRNA, which regulate mRNA stability and translation in different tissues and cells, can be removed or introduced into RNA (e.g., modified messenger RNA). Such cis-regulatory elements can include, but are not limited to, Cis-RNP (Ribonucleoprotein)/RBP (RNA binding protein) regulatory elements, AU-rich element (AUE), structured stem-loop, constitutive decay elements (CDEs), GC-richness and other structured mRNA motifs (Parker B J et al., Genome Research, 2011, 21, 1929-1943, which is herein incorporated by reference in its entirety). For example, CDEs are a class of regulatory motifs that mediate mRNA degradation through their interaction with Roquin proteins. In particular, CDEs are found in many mRNAs that encode regulators of development and inflammation to limit cytokine production in macrophage (Leppek K et al., 2013, Cell, 153, 869-881, which is herein incorporated by reference in its entirety).

In some aspects, the RNA (e.g., modified mRNA) is auxotrophic. As used herein, the term “auxotrophic” refers to mRNA that comprises at least one feature that triggers, facilitates or induces the degradation or inactivation of the mRNA in response to spatial or temporal cues such that protein expression is substantially prevented or reduced. Such spatial or temporal cues include the location of the mRNA to be translated such as a particular tissue or organ or cellular environment. Also contemplated are cues involving temperature, pH, ionic strength, moisture content and the like.

### 3′ UTR and the AU Rich Elements

3′UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids or mRNA of the disclosure. When engineering specific nucleic acids or mRNA, one or more copies of an ARE can be introduced to make nucleic acids or mRNA of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids or mRNA of the disclosure and protein production can be assayed at various time points post-transfection. For example, cells can be transfected with different ARE-engineering molecules and by using an ELISA kit to the relevant protein and assaying protein produced at about 6 hr, about 12 hr, about 24 hr, about 48 hr, and/or about 7 days post-transfection.

### 3′ UTR and Triple Helices

In some aspects, the nucleotide sequence encoding IL-12 comprises a triple helix on the 3′ end of the modified nucleic acid, enhanced nucleotide sequence encoding IL-12 or ribonucleic acid. In some aspects, the 3′ end of the nucleotide sequence encoding IL-12 include a triple helix alone or in combination with a Poly-A tail.

In some aspects, the nucleotide sequence encoding IL-12 comprises at least a first and a second U-rich region, a conserved stem loop region between the first and second region and an A-rich region. In some aspects, the first and second U-rich region and the A-rich region associate to form a triple helix on the 3′ end of the nucleic acid. This triple helix can stabilize the nucleic acid, enhance the translational efficiency of the nucleic acid and/or protect the 3′ end from degradation. Exemplary triple helices include, but are not limited to, the triple helix sequence of metastasis-associated lung adenocarcinoma transcript 1 (MALAT1), MEN-β and polyadenylated nuclear (PAN) RNA (See Wilusz et al., Genes & Development 2012 26:2392-2407; herein incorporated by reference in its entirety).

### Stem Loop

In some aspects, the nucleotide sequence encoding IL-12 includes a stem loop such as, but not limited to, a histone stem loop. In some aspects, the stem loop is a nucleotide sequence that is about 25 or about 26 nucleotides in length such as, but not limited to, SEQ ID NOs: 7-17 as described in International Patent Publication No. WO2013103659, herein incorporated by reference in its entirety. The histone stem loop can be located 3′ relative to the coding region (e.g., at the 3′ terminus of the coding region). As a non-limiting example, the stem loop can be located at the 3′ end of a nucleic acid described herein.

In some aspects, the nucleotide sequence encoding IL-12, which comprises the histone stem loop can be stabilized by the addition of at least one chain terminating nucleoside. Not wishing to be bound by theory, the addition of at least one chain terminating nucleoside can slow the degradation of a nucleic acid and thus can increase the half-life of the nucleic acid.

In some aspects, the chain terminating nucleoside is one described in International Patent Publication No. WO2013103659, herein incorporated by reference in its entirety. In some aspects, the chain terminating nucleosides are 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O-methylnucleoside.

In some aspects, the nucleotide sequence encoding IL-12 includes a histone stem loop, a polyA tail sequence and/or a 5′ cap structure. In some aspects, the histone stem loop is before and/or after the polyA tail sequence. The nucleic acids comprising the histone stem loop and a polyA tail sequence can include a chain terminating nucleoside described herein.

In some aspects, the nucleotide sequence encoding IL-12 comprises a histone stem loop and a 5′ cap structure. The 5′ cap structure can include, but is not limited to, those described herein and/or known in the art.

### 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

Modifications to the RNA of the present disclosure can generate a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′ mppp-G; which can equivalently be designated 3′ O-Me-m7G(5′)ppp(5′)G). The 3′-O atom of the other, unmodified, guanine becomes linked to the 5′-terminal nucleotide of the capped nucleic acid molecule (e.g., an mRNA or mmRNA). The N7- and 3′-O-methylated guanine provides the terminal moiety of the capped nucleic acid molecule (e.g., mRNA or mmRNA).

Another exemplary cap is mCAP, which is similar to ARCA but has a 2′-β-methyl group on guanosine (i.e., N7,2′-O-dimethyl-guanosine-5′-triphosphate-5′-guanosine, m7Gm-ppp-G).

In some aspects, the cap is a dinucleotide cap analog. In some aspects, the dinucleotide cap analog is modified at different phosphate positions with a boranophosphate group or a phosphoroselenoate group such as the dinucleotide cap analogs described in U.S. Pat. No. 8,519,110, the contents of which are herein incorporated by reference in its entirety.

In some aspects, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In some aspects, a cap analog of the present disclosure is a 4-chloro/bromophenoxyethyl analog.

While cap analogs allow for the concomitant capping of a nucleic acid molecule in an in vitro transcription reaction, up to about 20% of transcripts remain uncapped. This, as well as the structural differences of a cap analog from an endogenous 5′-cap structures of nucleic acids produced by the endogenous, cellular transcription machinery, can lead to reduced translational competency and reduced cellular stability. Accordingly, in some aspects, the methods provided herein (see, e.g., Examples 1-3) are capable of increasing the capping efficiency of the produced IL-12 expressing nucleotides described herein. In some aspects, with the methods provided herein, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100% of the polynucleotides are capped. In some aspects, with the methods provided herein, at least about 50% of the polynucleotides are capped. In some aspects, at least about 60% of the polynucleotides are capped. In some aspects, at least about 70% of the polynucleotides are capped. In some aspects, at least about 80% of the polynucleotides are capped. In some aspects, at least about 85% of the polynucleotides are capped. In some aspects, at least about 90% of the polynucleotides are capped. In some aspects, at least about 95% of the polynucleotides are capped. In some aspects, about 100% of the polynucleotides are capped. In some aspects, at least about 80% to about 100% of the polynucleotides are capped.

In some aspects, providing an RNA with a 5′-cap or 5′-cap analog is achieved by in vitro transcription of a DNA template in the presence of said 5′-cap or 5′-cap analog, wherein said 5′-cap is co-transcriptionally incorporated into the generated RNA strand,

In some aspects, RNA can be generated, for example, by in vitro transcription, and the 5′-cap can be attached to the RNA post-transcriptionally using capping enzymes, for example, capping enzymes of vaccinia virus. In some aspects, the nucleotide sequence encoding IL-12 is capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present disclosure are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. This cap results in a higher translational-competency and cellular stability and a reduced activation of cellular pro-inflammatory cytokines, as compared, e.g., to other 5′ cap analog structures known in the art. Cap structures include 7mG(5′)ppp(5′)N,pN2p, 7mG(5′)ppp(5′)NlmpNp, 7mG(5′)-ppp(5′)NlmpN2 mp and m(7)Gpppm(3)(6,6,2′)Apm(2′)Apm(2′)Cpm(2)(3,2′)Up.

In some aspects, 5′ terminal caps include endogenous caps or cap analogs. In some aspects, a 5′ terminal cap comprises a guanine analog. Useful guanine analogs include inosine, N1-methyl-guanosine, 2′ fluoro-guanosine, 7-deaza-guanosine, 8-oxo-guanosine, 2-amino-guanosine, LNA-guanosine, and 2-azido-guanosine.

In some aspects, the 5′ cap comprises a 5′ to 5′ triphosphate linkage. In some aspects, the 5′ cap comprises a 5′ to 5′ triphosphate linkage including thiophosphate modification. In some aspects, the 5′ cap comprises a 2′-O or 3′-O-ribose-methylated nucleotide. In some aspects, the 5′ cap comprises a modified guanosine nucleotide or modified adenosine nucleotide. In some aspects, the 5′ cap comprises 7-methylguanylate. Exemplary cap structures include m7G(5′)ppp(5′)G, m7,2′ O-mG(5′)ppSp(5′)G, m7G(5′)ppp(5′)2′O-mG, and m7,3′O-mG(5′)ppp(5′)2′O-mA.

In some aspects, the nucleotide sequence encoding IL-12 comprises a modified 5′ cap. A modification on the 5′ cap can increase the stability of mRNA, increase the half-life of the mRNA, and could increase the mRNA translational efficiency. In some aspects, the modified 5′ cap comprises one or more of the following modifications: modification at the 2′ and/or 3′ position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

The 5′ cap structure that can be modified includes, but is not limited to, the caps described in U.S. Application No. 2014/0147454 and WO2018/160540 which is incorporated herein by reference in its entirety.

### IRES Sequences

In some aspects, the nucleotide sequence encoding IL-12 comprises an internal ribosome entry site (IRES). First identified as a feature Picorna virus RNA, IRES plays an important role in initiating protein synthesis in absence of the 5′ cap structure. An IRES can act as the sole ribosome binding site, or can serve as one of multiple ribosome binding sites of an mRNA. Nucleic acids or mRNA containing more than one functional ribosome binding site can encode several peptides or polypeptides that are translated independently by the ribosomes (“multicistronic nucleic acid molecules”). When nucleic acids or mRNA are provided with an IRES, further optionally provided is a second translatable region. Examples of IRES sequences that can be used according to the disclosure include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Terminal Architecture Modifications: Poly-A Tails

During RNA processing, a long chain of adenine nucleotides (poly-A tail) is normally added to a messenger RNA (mRNA) molecules to increase the stability of the molecule. Immediately after transcription, the 3′ end of the transcript is cleaved to free a 3′ hydroxyl. Then poly-A polymerase adds a chain of adenine nucleotides to the RNA. The process, called polyadenylation, adds a poly-A tail that is between 100 and 250 residues long.

In some aspects, the length of the 3′ tail is greater than about 30 nucleotides in length. In some aspects, the poly-A tail is greater than about 35 nucleotides in length. In some aspects, the length is at least about 40 nucleotides. In some aspects, the length is at least about 45 nucleotides. In some aspects, the length is at least about 55 nucleotides. In some aspects, the length is at least about 60 nucleotides. In some aspects, the length is at least 70 nucleotides. In some aspects, the length is at least about 80 nucleotides. In some aspects, the length is at least about 90 nucleotides. In some aspects, the length is at least about 100 nucleotides. In some aspects, the length is at least about 120 nucleotides. In some aspects, the length is at least about 140 nucleotides. In some aspects, the length is at least about 160 nucleotides. In some aspects, the length is at least about 180 nucleotides. In some aspects, the length is at least about 200 nucleotides. In some aspects, the length is at least about 250 nucleotides. In some aspects, the length is at least about 300 nucleotides. In some aspects, the length is at least about 350 nucleotides. In some aspects, the length is at least about 400 nucleotides. In some aspects, the length is at least about 450 nucleotides. In some aspects, the length is at least about 500 nucleotides. In some aspects, the length is at least about 600 nucleotides. In some aspects, the length is at least about 700 nucleotides. In some aspects, the length is at least about 800 nucleotides. In some aspects, the length is at least about 900 nucleotides. In some aspects, the length is at least about 1000 nucleotides. In some aspects, the length is at least about 1100 nucleotides. In some aspects, the length is at least about 1200 nucleotides. In some aspects, the length is at least about 1300 nucleotides. In some aspects, the length is at least about 1400 nucleotides. In some aspects, the length is at least about 1500 nucleotides. In some aspects, the length is at least about 1600 nucleotides. In some aspects, the length is at least about 1700 nucleotides. In some aspects, the length is at least about 1800 nucleotides. In some aspects, the length is at least about 1900 nucleotides. In some aspects, the length is at least about 2000 nucleotides. In some aspects, the length is at least about 2500 nucleotides. In some aspects, the length is at least about 3000 nucleotides.

In some aspects, the nucleotide sequence encoding IL-12 is designed to include a polyA-G Quartet. The G-quartet is a cyclic hydrogen bonded array of four guanine nucleotides that can be formed by G-rich sequences in both DNA and RNA. In this aspect, the G-quartet is incorporated at the end of the poly-A tail. The resultant nucleic acid or mRNA can be assayed for stability, protein production and other parameters including half-life at various time points. It has been discovered that the polyA-G quartet results in protein production equivalent to at least 75% of that seen using a poly-A tail of 120 nucleotides alone.

In some aspects, the nucleotide sequence encoding IL-12 comprises a polyA tail and is stabilized by the addition of a chain terminating nucleoside. In some aspects, the nucleotide sequence encoding IL-12 with a polyA tail further comprise a 5′ cap structure.

In some aspects, the nucleotide sequence encoding IL-12 comprises a polyA-G Quartet. In some aspects, the nucleotide sequence encoding IL-12 with a polyA-G Quartet further comprises a 5′ cap structure.

In some aspects, the nucleotide sequence encoding IL-12, which comprise a polyA tail or a polyA-G Quartet is stabilized by the addition of an oligonucleotide that terminates in a 3′-deoxynucleoside, 2′,3′-dideoxynucleoside 3′-0-methylnucleosides, 3′-0-ethylnucleosides, 3′-arabinosides, and other modified nucleosides known in the art and/or described herein.

### Modified Nucleosides

In some aspects, the nucleotide sequence encoding IL-12 comprises one or more modified nucleosides. In some aspects, the one or more modified nucleosides comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, one or more uridine in the nucleotide sequence encoding IL-12 is replaced by a modified nucleoside. In some aspects, the modified nucleoside replacing uridine is pseudouridine (ψ), N1-methyl-pseudouridine (m1ψ) or 5-methyl-uridine (m5U).

In some aspects, the nucleotide sequence encoding IL-12 comprises a nucleotide sequence encoding IL-12 as described in U.S. Application Number 2014/0147454, International Application WO2018160540, International Application WO2015/196118, or International Application WO2015/089511, which are incorporated herein by reference in their entirety.

### Cytotoxic Nucleosides

In some aspects, the nucleotide sequence encoding IL-12 comprises one or more cytotoxic nucleosides. For example, cytotoxic nucleosides can be incorporated into polynucleotides such as bifunctional nucleotide sequence encoding IL-12s or mRNAs. Cytotoxic nucleoside anti-cancer agents include, but are not limited to, adenosine arabinoside, cytarabine, cytosine arabinoside, 5-fluorouracil, fludarabine, floxuridine, FTORAFUR® (a combination of tegafur and uracil), tegafur ((RS)-5-fluoro-1-(tetrahydrofuran-2-yl)pyrimidine-2,4(1H,3H)-dione), and 6-mercaptopurine.

A number of cytotoxic nucleoside analogues are in clinical use, or have been the subject of clinical trials, as anticancer agents. Examples of such analogues include, but are not limited to, cytarabine, gemcitabine, troxacitabine, decitabine, tezacitabine, 2′-deoxy-2′-methylidenecytidine (DMDC), cladribine, clofarabine, 5-azacytidine, 4′-thio-aracytidine, cyclopentenylcytosine and 1-(2-C-cyano-2-deoxy-beta-D-arabino-pentofuranosyl)-cytosine. Another example of such a compound is fludarabine phosphate. These compounds can be administered systemically and can have side effects which are typical of cytotoxic agents such as, but not limited to, little or no specificity for tumor cells over proliferating normal cells.

A number of prodrugs of cytotoxic nucleoside analogues are also reported in the art. Examples include, but are not limited to, N4-behenoyl-1-beta-D-arabinofuranosylcytosine, N4-octadecyl-1-b eta-D-arab inofuranosyl cytosine, N4-palmitoyl-1-(2-C-cyano-2-deoxy-beta-D-arabino-pentofuranosyl) cytosine, and P-4055 (cytarabine 5′-elaidic acid ester). In general, these prodrugs can be converted into the active drugs mainly in the liver and systemic circulation and display little or no selective release of active drug in the tumor tissue. For example, capecitabine, a prodrug of 5′-deoxy-5-fluorocytidine (and eventually of 5-fluorouracil), is metabolized both in the liver and in the tumor tissue. A series of capecitabine analogues containing “an easily hydrolysable radical under physiological conditions” has been claimed by Fujiu et al. (U.S. Pat. No. 4,966,891) and is herein incorporated by reference. The series described by Fujiu includes N4 alkyl and aralkyl carbamates of 5′-deoxy-5-fluorocytidine and the implication that these compounds will be activated by hydrolysis under normal physiological conditions to provide 5′-deoxy-5-fluorocytidine.

A series of cytarabine N4-carbamates has been by reported by Fadl et al (Pharmazie. 1995, 50, 382-7, herein incorporated by reference in its entirety) in which compounds were designed to convert into cytarabine in the liver and plasma. WO 2004/041203, herein incorporated by reference in its entirety, discloses prodrugs of gemcitabine, where some of the prodrugs are N4-carbamates. These compounds were designed to overcome the gastrointestinal toxicity of gemcitabine and were intended to provide gemcitabine by hydrolytic release in the liver and plasma after absorption of the intact prodrug from the gastrointestinal tract. Nomura et al (Bioorg Med. Chem. 2003, 11, 2453-61, herein incorporated by reference in its entirety) have described acetal derivatives of 1-(3-C-ethynyl-β-D-ribo-pentofuranosyl) cytosine which, on bioreduction, produced an intermediate that required further hydrolysis under acidic conditions to produce a cytotoxic nucleoside compound.

Cytotoxic nucleotides which can be chemotherapeutic also include, but are not limited to, pyrazolo[3,4-D]-pyrimidines, allopurinol, azathioprine, capecitabine, cytosine arabinoside, fluorouracil, mercaptopurine, 6-thioguanine, acyclovir, ara-adenosine, ribavirin, 7-deaza-adenosine, 7-deaza-guanosine, 6-aza-uracil, 6-aza-cytidine, thymidine ribonucleotide, 5-bromodeoxyuridine, 2-chloro-purine, and inosine, or combinations thereof.

### Coding Sequences

In some aspects of the disclosure, the nucleotide sequence encoding IL-12 comprises a sequence that encodes an interleukin (IL)-12 molecule. In some aspects, the IL-12 molecule comprises is IL-12, an IL-12 subunit (e.g., IL-12 beta subunit or IL-12 alpha subunit), or a mutant IL-12 molecule that retains immunomodulatory function.

IL-12 is a heterodimeric cytokine with multiple biological effects on the immune system. It is composed of two subunits, p35 (also known as the alpha subunit) and p40 (also known as the beta subunit), which interact to produce the active heterodimer (also referred to as “p′70”). The IL-12 p35 subunit is also known in the art as IL-12α; IL-12A; Natural Killer Cell Stimulatory Factor 1; Cytotoxic Lymphocyte Maturation Factor 1, p35; CLMF P35, NKSF1; CLMF; or NF SK. The IL-12 p40 subunit is also known in the art as IL-12β; IL-12B; Natural Killer Cell Stimulatory Factor 2; Cytotoxic Lymphocyte Maturation Factor 2, P40; CLMF P40; NKSF2; CLMF2; IMD28; or IMD29. Unless indicated otherwise, the term “IL-12” (or a grammatical variant thereof) can refer to IL-12 p35 subunit, IL-12 p40 subunit, or the heterodimeric IL-12 p70.

The wild-type human IL-12 p35 protein is 219 amino acids in length. The wild-type human IL-12 p40 protein is 328 amino acids in length. The amino acid of the wild-type human IL-12 proteins are provided in Table 1 further below.

In some aspects, the IL-12 protein (e.g., encoded by a nucleic acid molecule described herein) comprises an amino acid sequence that is at least about 70%, at least about 75%, at least about 80%, at least about 85%, at least about 90%, at least about 91%, at least about 92%, at least about 93%, at least about 94% at least about 95%, at least about 96%, at least about 97%, at least about 98%, at least about 99%, or about 100% identical to SEQ ID NO: 182.

In some aspects, the IL-12 molecule comprises IL-12α and/or IL-12β subunits. In some aspects, the IL-12α subunit comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 183.

In some aspects, the IL-12β subunit comprises an amino acid sequence that is at least about 70%, at least about 75%, at least about 80%, at least about 85%, at least about 90%, at least about 91%, at least about 92%, at least about 93%, at least about 94% at least about 95%, at least about 96%, at least about 97%, at least about 98%, at least about 99%, or about 100% identical to SEQ ID NO: 184.

As described herein, the nucleic acid molecules of the present disclosure have been codon optimized. Accordingly, in some aspects, the nucleotide sequence encoding an IL-12 protein (e.g., IL-12 p35 subunit, IL-12 p40 subunit, or the heterodimeric IL-12 p′70) disclosed herein differs from that of the wild-type nucleotide sequence (e.g., SEQ ID NO: 185 or SEQ ID NO: 186).

In some aspects, a nucleic acid molecule described herein encodes an IL-12β subunit and comprises a nucleotide sequence that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in any one of SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75. In certain aspects, the nucleotide sequence is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the IL-12β subunit sequence of any of the constructs provided in Table 1.

In some aspects, the nucleic acid molecule encoding the IL-12 p40 subunit comprises a nucleotide sequence that is (i) at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 51; (ii) at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 52; (iii) at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 53; (iv) at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 54; (v) at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 55; (vi) at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 56; (vii) at least 91%, at least 92%, at least 93%, at least 94%, at least 95%, at least 96%, at least 97%, at least 98%, at least 99%, or 100% identical to the sequence set forth in SEQ ID NO: 57; (viii) at least 92%, at least 93%, at least 94%, at least 95%, at least 96%, at least 97%, at least 98%, at least 99%, or 100% identical to the sequence set forth in SEQ ID NO: 58; (ix) at least 91%, at least 92%, at least 93%, at least 94%, at least 95%, at least 96%, at least 97%, at least 98%, at least 99%, or 100% identical to the sequence set forth in SEQ ID NO: 59; (x) at least 91%, at least 92%, at least 93%, at least 94%, at least 95%, at least 96%, at least 97%, at least 98%, at least 99%, or 100% identical to the sequence set forth in SEQ ID NO: 65, 69, or 74; (xi) at least 91%, at least 92%, at least 93%, at least 94%, at least 95%, at least 96%, at least 97%, at least 98%, at least 99%, or 100% identical to the sequence set forth in SEQ ID NO: 66, 70, or 75; (xii) at least 97%, at least 98%, at least 99%, or 100% identical to the sequence set forth in SEQ ID NO: 62; (xiii) at least 99% or 100% identical to the sequence set forth in SEQ ID NO: 63; or (xiv) at least 98%, at least 99%, or 100% identical to the sequence set forth in SEQ ID NO: 64.

In some aspects, the nucleic acid molecule encoding the IL-12β subunit comprises the sequence set forth in SEQ ID NO: 51. In some aspects, the nucleic acid molecule encoding the IL-12β subunit comprises the sequence set forth in SEQ ID NO: 52. In some aspects, the nucleic acid molecule encoding the IL-12β subunit comprises the sequence set forth in SEQ ID NO: 53. In some aspects, the nucleic acid molecule encoding the IL-12β subunit comprises the sequence set forth in SEQ ID NO: 54. In some aspects, the nucleic acid molecule encoding the IL-12β subunit comprises the sequence set forth in SEQ ID NO: 55. In some aspects, the nucleic acid molecule encoding the IL-12β subunit comprises the sequence set forth in SEQ ID NO: 56 In some aspects, the nucleic acid molecule encoding the IL-12β subunit comprises the sequence set forth in SEQ ID NO: 57. In some aspects, the nucleic acid molecule encoding the IL-12β subunit comprises the sequence set forth in SEQ ID NO: 58. In some aspects, the nucleic acid molecule encoding the IL-120 subunit comprises the sequence set forth in SEQ ID NO: 59. In some aspects, the nucleic acid molecule encoding the IL-12β subunit comprises the sequence set forth in SEQ ID NO: 65. In some aspects, the nucleic acid molecule encoding the IL-12β subunit comprises the sequence set forth in SEQ ID NO: 66. In some aspects, the nucleic acid molecule encoding the IL-12β subunit comprises the sequence set forth in SEQ ID NO: 67. In some aspects, the nucleic acid molecule encoding the IL-12β subunit comprises the sequence set forth in SEQ ID NO: 68. In some aspects, the nucleic acid molecule encoding the IL-12β subunit comprises the sequence set forth in SEQ ID NO: 69. In some aspects, the nucleic acid molecule encoding the IL-12β subunit comprises the sequence set forth in SEQ ID NO: 70. In some aspects, the nucleic acid molecule encoding the IL-12β subunit comprises the sequence set forth in SEQ ID NO: 71. In some aspects, the nucleic acid molecule encoding the IL-12β subunit comprises the sequence set forth in SEQ ID NO: 72. In some aspects, the nucleic acid molecule encoding the IL-12β subunit comprises the sequence set forth in SEQ ID NO: 73. In some aspects, the nucleic acid molecule encoding the IL-12β subunit comprises the sequence set forth in SEQ ID NO: 74. In some aspects, the nucleic acid molecule encoding the IL-120 subunit comprises the sequence set forth in SEQ ID NO: 75. In some aspects, the nucleic acid molecule encoding the IL-12β subunit comprises the sequence set forth in SEQ ID NO: 62. In some aspects, the nucleic acid molecule encoding the IL-12β subunit comprises the sequence set forth in SEQ ID NO: 63. In some aspects, the nucleic acid molecule encoding the IL-12β subunit comprises the sequence set forth in or SEQ ID NO: 64. In some aspects, the nucleic acid molecule encoding the IL-12β subunit comprises the sequence set forth in SEQ ID NO: 60. In some aspects, the nucleic acid molecule encoding the IL-12β subunit comprises the sequence set forth in SEQ ID NO: 61.

In some aspects, a nucleic acid molecule described herein encodes an IL-12 p35 subunit and comprises a nucleotide sequence that is at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in any one of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125. In certain aspects, the nucleotide sequence is at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the IL-12α subunit sequence of any of the constructs provided in Table 1.

In some aspects, the nucleic acid molecule encoding the IL-12 p35 subunit comprises a nucleotide sequence that is (i) at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 101; (ii) at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 102; (iii) at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 103; (iv) at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 104; (v) at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 105; (vi) at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 106; (vii) at least 91%, at least 92%, at least 93%, at least 94%, at least 95%, at least 96%, at least 97%, at least 98%, at least 99%, or 100% identical to the sequence set forth in SEQ ID NO: 107; (viii) at least 91%, at least 92%, at least 93%, at least 94%, at least 95%, at least 96%, at least 97%, at least 98%, at least 99%, or 100% identical to the sequence set forth in SEQ ID NO: 108; (ix) at least 92%, at least 93%, at least 94%, at least 95%, at least 96%, at least 97%, at least 98%, at least 99%, or 100% identical to the sequence set forth in SEQ ID NO: 109; (x) at least 91%, at least 92%, at least 93%, at least 94%, at least 95%, at least 96%, at least 97%, at least 98%, at least 99%, or 100% identical to the sequence set forth in SEQ ID NO: 115, 119, or 124; (xi) at least 92%, at least 93%, at least 94%, at least 95%, at least 96%, at least 97%, at least 98%, at least 99%, or 100% identical to the sequence set forth in SEQ ID NO: 116, 120, or 125; (xii) at least 98%, at least 99%, or 100% identical to the sequence set forth in SEQ ID NO: 112; (xiii) at least 98%, at least 99%, or 100% identical to the sequence set forth in SEQ ID NO: 113; or (xiv) at least 98%, at least 99%, or 100% identical to the sequence set forth in SEQ ID NO: 114.

In some aspects, the nucleic acid molecule encoding the IL-12α subunit comprises the sequence set forth in SEQ ID NO: 101. In some aspects, the nucleic acid molecule encoding the IL-12α subunit comprises the sequence set forth in SEQ ID NO: 102. In some aspects, the nucleic acid molecule encoding the IL-12α subunit comprises the sequence set forth in SEQ ID NO: 103. In some aspects, the nucleic acid molecule encoding the IL-12α subunit comprises the sequence set forth in SEQ ID NO: 104. In some aspects, the nucleic acid molecule encoding the IL-12α subunit comprises the sequence set forth in SEQ ID NO: 105. In some aspects, the nucleic acid molecule encoding the IL-12α subunit comprises the sequence set forth in SEQ ID NO: 106. In some aspects, the nucleic acid molecule encoding the IL-12α subunit comprises the sequence set forth in SEQ ID NO: 107. In some aspects, the nucleic acid molecule encoding the IL-12α subunit comprises the sequence set forth in SEQ ID NO: 108. In some aspects, the nucleic acid molecule encoding the IL-12α subunit comprises the sequence set forth in SEQ ID NO: 109. In some aspects, the nucleic acid molecule encoding the IL-12α subunit comprises the sequence set forth in SEQ ID NO: 115. In some aspects, the nucleic acid molecule encoding the IL-12α subunit comprises the sequence set forth in SEQ ID NO: 116. In some aspects, the nucleic acid molecule encoding the IL-12β subunit comprises the sequence set forth in SEQ ID NO: 117. In some aspects, the nucleic acid molecule encoding the IL-12β subunit comprises the sequence set forth in SEQ ID NO: 118. In some aspects, the nucleic acid molecule encoding the IL-12β subunit comprises the sequence set forth in SEQ ID NO: 119]. In some aspects, the nucleic acid molecule encoding the IL-12β subunit comprises the sequence set forth in SEQ ID NO: 120. In some aspects, the nucleic acid molecule encoding the IL-12β subunit comprises the sequence set forth in SEQ ID NO: 121. In some aspects, the nucleic acid molecule encoding the IL-12β subunit comprises the sequence set forth in SEQ ID NO: 122. In some aspects, the nucleic acid molecule encoding the IL-12β subunit comprises the sequence set forth in SEQ ID NO: 123. In some aspects, the nucleic acid molecule encoding the IL-12β subunit comprises the sequence set forth in SEQ ID NO: 124. In some aspects, the nucleic acid molecule encoding the IL-12β subunit comprises the sequence set forth in SEQ ID NO: 125. In some aspects, the nucleic acid molecule encoding the IL-12α subunit comprises the sequence set forth in SEQ ID NO: 112. In some aspects, the nucleic acid molecule encoding the IL-12α subunit comprises the sequence set forth in SEQ ID NO: 113. In some aspects, the nucleic acid molecule encoding the IL-12α subunit comprises the sequence set forth in SEQ ID NO: 114. In some aspects, the nucleic acid molecule encoding the IL-12β subunit comprises the sequence set forth in SEQ ID NO: 110. In some aspects, the nucleic acid molecule encoding the IL-12β subunit comprises the sequence set forth in SEQ ID NO: 111.

In some aspects, the nucleic acid molecule encoding the IL-12 p40 subunit and the nucleic acid molecule encoding the IL-12 p35 subunit can be conjugated to each other. For instance, in some aspects, the present disclosure provides an isolated polynucleotide comprising a first nucleic acid and a second nucleic acid, wherein the first nucleic acid encodes the IL-12 p40 subunit and the second nucleic acid encodes the IL-12 p35 subunit. In some aspects, the IL-12α subunit and the IL-12β subunit are linked by a linker. In some aspects, the linker comprises an amino acid linker of at least about 2, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 amino acids. In some aspects, the linker comprises a (GS) linker. In some aspects, the GS linker has a formula of (Gly3 Ser)n or S(Gly3 Ser)n, wherein n is a positive integer selected from the group consisting of 1, 2, 3, 4, 5, 6, 7, 8, 9, 10, 11, 12, 13, 14, 15, 16, 17, 18, 19, 20, 30, 40, 50, 60, 70, 80, or 100. In some aspects, the (Gly3Ser)n linker is (Gly3Ser)3 or (Gly3 Ser)4.

In some aspects, the first nucleic acid molecule encoding the IL-12β subunit comprises a nucleotide sequence that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75; and the second nucleic acid molecule encoding the IL-12α subunit comprises a nucleotide sequence that is at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

In some aspects, the first nucleic acid molecule encoding the IL-12β subunit comprises a nucleotide sequence that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the IL-12β subunit sequence of any of the constructs provided in Table 1; and the second nucleic acid molecule encoding the IL-12a subunit comprises a nucleotide sequence that is at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the IL-12α subunit sequence of any of the constructs provided in Table 1.

In some aspects, (i) the first nucleic acid molecule comprises a nucleotide sequence that is at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 51 and/or the second nucleic acid molecule comprises a nucleotide sequence that is at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 101; (ii) the first nucleic acid molecule comprises a nucleotide sequence that is at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 52 and/or the second nucleic acid molecule comprises a nucleotide sequence that is at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 102; (iii) the first nucleic acid molecule comprises a nucleotide sequence that is at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 53 and/or the second nucleic acid molecule comprises a nucleotide sequence that is at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 103; (iv) the first nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 54 and/or the second nucleic acid molecule comprises a nucleotide sequence that is at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 104; (v) the first nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 55 and/or the second nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 105; (vi) the first nucleic acid molecule comprises a nucleotide sequence that is at least 87%, at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 56 and/or the second nucleic acid molecule comprises a nucleotide sequence that is at least 88%, at least 89%, at least 90%, at least 91%, at least 92%, at least 93%, at least 94%, at least 95%, at least 96%, at least 97%, at least 98%, at least 99%, or 100% identical to the sequence set forth in SEQ ID NO: 106; (vii) the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 57 and/or the second nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 107; (viii) the first nucleic acid molecule comprises a nucleotide sequence that is at least 92%, at least 93%, at least 94%, at least 95%, at least 96%, at least 97%, at least 98%, at least 99%, or 100% identical to the sequence set forth in SEQ ID NO: 58 and/or the second nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 108; (ix) the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 59 and/or the second nucleic acid molecule comprises a nucleotide sequence that is at least 92%, at least 93%, at least 94%, at least 95%, at least 96%, at least 97%, at least 98%, at least 99%, or 100% identical to the sequence set forth in SEQ ID NO: 109; (x) the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 65, 69, or 74 and/or the second nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 115, 119, or 124; (xi) the first nucleic acid molecule comprises a nucleotide sequence that is at least 91%, at least 92%, at least 93%, at least 94%, at least 95%, at least 96%, at least 97%, at least 98%, at least 99%, or 100% identical to the sequence set forth in SEQ ID NO: 66, 70, or 75 and/or the second nucleic acid molecule comprises a nucleotide sequence that is at least 92%, at least 93%, at least 94%, at least 95%, at least 96%, at least 97%, at least 98%, at least 99%, or 100% identical to the sequence set forth in SEQ ID NO: 116, 120, or 125; (xii) the first nucleic acid molecule comprises a nucleotide sequence that is at least 97%, at least 98%, at least 99%, or 100% identical to the sequence set forth in SEQ ID NO: 62 and/or the second nucleic acid molecule comprises a nucleotide sequence that is at least 98%, at least 99%, or 100% identical to the sequence set forth in SEQ ID NO: 112; (xiii) the first nucleic acid molecule comprises a nucleotide sequence that is at least 99% or 100% identical to the sequence set forth in SEQ ID NO: 63 and/or the second nucleic acid molecule comprises a nucleotide sequence that is at least 98%, at least 99%, or 100% identical to the sequence set forth in SEQ ID NO: 113; or (xiv) the first nucleic acid molecule comprises a nucleotide sequence that is at least 98%, at least 99%, or 100% identical to the sequence set forth in SEQ ID NO: 65 and/or the second nucleic acid molecule comprises a nucleotide sequence that is at least 98%, at least 99%, or 100% identical to the sequence set forth in SEQ ID NO: 115.

In some aspects, the first nucleic acid molecule comprises the sequence set forth in SEQ ID NO: 51 and the second nucleic acid molecule comprises the sequence set forth in SEQ ID NO: 101. In some aspects, the first nucleic acid molecule comprises the sequence set forth in SEQ ID NO: 52 and the second nucleic acid molecule comprises the sequence set forth in SEQ ID NO: 102. In some aspects, In some aspects, the first nucleic acid molecule comprises the sequence set forth in SEQ ID NO: 53 and the second nucleic acid molecule comprises the sequence set forth in SEQ ID NO: 103. In some aspects, In some aspects, the first nucleic acid molecule comprises the sequence set forth in SEQ ID NO: 54 and the second nucleic acid molecule comprises the sequence set forth in SEQ ID NO: 104 In some aspects, the first nucleic acid molecule comprises the sequence set forth in SEQ ID NO: 55 and the second nucleic acid molecule comprises the sequence set forth in SEQ ID NO: 105. In some aspects, the first nucleic acid molecule comprises the sequence set forth in SEQ ID NO: 56 and the second nucleic acid molecule comprises the sequence set forth in SEQ ID NO: 106. In some aspects, the first nucleic acid molecule comprises the sequence set forth in SEQ ID NO: 57 and the second nucleic acid molecule comprises the sequence set forth in SEQ ID NO: 107. In some aspects, the first nucleic acid molecule comprises the sequence set forth in SEQ ID NO: 58 and the second nucleic acid molecule comprises the sequence set forth in SEQ ID NO: 118. In some aspects, the first nucleic acid molecule comprises the sequence set forth in SEQ ID NO: 59 and the second nucleic acid molecule comprises the sequence set forth in SEQ ID NO: 119. In some aspects, the first nucleic acid molecule comprises the sequence set forth in SEQ ID NO: 65 and the second nucleic acid molecule comprises the sequence set forth in SEQ ID NO: 115. In some aspects, the first nucleic acid molecule comprises the sequence set forth in SEQ ID NO: 66 and the second nucleic acid molecule comprises the sequence set forth in SEQ ID NO: 116. In some aspects, the first nucleic acid molecule comprises the sequence set forth in SEQ ID NO: 67 and the second nucleic acid molecule comprises the sequence set forth in SEQ ID NO: 117. In some aspects, the first nucleic acid molecule comprises the sequence set forth in SEQ ID NO: 68 and the second nucleic acid molecule comprises the sequence set forth in SEQ ID NO: 118. In some aspects, the first nucleic acid molecule comprises the sequence set forth in SEQ ID NO: 69 and the second nucleic acid molecule comprises the sequence set forth in SEQ ID NO: 119. In some aspects, the first nucleic acid molecule comprises the sequence set forth in SEQ ID NO: 70 and the second nucleic acid molecule comprises the sequence set forth in SEQ ID NO: 120. In some aspects, the first nucleic acid molecule comprises the sequence set forth in SEQ ID NO: 71 and the second nucleic acid molecule comprises the sequence set forth in SEQ ID NO: 121. In some aspects, the first nucleic acid molecule comprises the sequence set forth in SEQ ID NO: 72 and the second nucleic acid molecule comprises the sequence set forth in SEQ ID NO: 122. In some aspects, the first nucleic acid molecule comprises the sequence set forth in SEQ ID NO: 73 and the second nucleic acid molecule comprises the sequence set forth in SEQ ID NO: 123. In some aspects, the first nucleic acid molecule comprises the sequence set forth in SEQ ID NO: 74 and the second nucleic acid molecule comprises the sequence set forth in SEQ ID NO: 124. In some aspects, the first nucleic acid molecule comprises the sequence set forth in SEQ ID NO: 75 and the second nucleic acid molecule comprises the sequence set forth in SEQ ID NO: 125. In some aspects, the first nucleic acid molecule comprises the sequence set forth in SEQ ID NO: 62 and the second nucleic acid molecule comprises the sequence set forth in SEQ ID NO: 112. In some aspects, the first nucleic acid molecule comprises the sequence set forth in SEQ ID NO: 63 and the second nucleic acid molecule comprises the sequence set forth in SEQ ID NO: 113. In some aspects, the first nucleic acid molecule comprises the sequence set forth in SEQ ID NO: 64 and the second nucleic acid molecule comprises the sequence set forth in SEQ ID NO: 114. In some aspects, the first nucleic acid molecule comprises the sequence set forth in SEQ ID NO: 60 and the second nucleic acid molecule comprises the sequence set forth in SEQ ID NO: 110. In some aspects, the first nucleic acid molecule comprises the sequence set forth in SEQ ID NO: 61 and the second nucleic acid molecule comprises the sequence set forth in SEQ ID NO: 111. In some aspects, the first nucleic acid molecule comprises the IL-120 subunit sequence of any of the constructs provided in Table 1, and the second nucleic acid molecule comprises the IL-12α subunit sequence of any of the constructs provided in Table 1.

TABLE 1

| Description | Sequence |
|---|---|
| Wild-type human | mcparslllvatlvlldhlslaRNLPVATPDPGMFPCLHHSONLLRAVSNMLQKAROTLEF |
| IL-12 (i.e., | YPCTSEEIDHEDITKDKTSTVEACLPLELTKNESCLNSRETSFITNGSCLASRKTSFMMAL |
| combination of the | CLSSIYEDLKMYQVEFKTMNAKLLMDPKRQIFLDQNMLAVIDELMQALNFNSETVPQKSSL |
| IL-12α and IL-12β | EEPDFYKTKIKLCILLHAFRIRAVTIDRVMSYLNASmchqqlviswfslvflasplvaIWE |
| subunits) (amino | LKKDVYVVELDWYPDAPGEMVVLTCDTPEEDGITWTLDQSSEVLGSGKTLTIQVKEFGDAG |
| acid sequence) (SEQ | QYTCHKGGEVLSHSLLLLHKKEDGIWSTDILKDQKEPKNKTFLRCEAKNYSGRFTCWWLTT |
| ID NO: 182) (signal | ISTDLTFSVKSSRGSSDPQGVTCGAATLSAERVRGDNKEYEYSVECQEDSACPAAEESLPI |
| peptides for the | EVMVDAVHKLKYENYTSSFFIRDIIKPDPPKNLQLKPLKNSRQVEVSWEYPDTWSTPHSYF |
| subunits are in lower | SLTFCVQVQGKSKREKKDRVFTDKTSATVICRKNASISVRAQDRYYSSSWSEWASVPCS |
| case) | |
| Wild-type human | mcparslllvatlvlldhlslaRNLPVATPDPGMFPCLHHSONLLRAVSNMLQKAROTLEF |
| IL-12α subunit | YPCTSEEIDHEDITKDKTSTVEACLPLELTKNESCLNSRETSFITNGSCLASRKTSFMMAL |
| (signal peptide is in | CLSSIYEDLKMYQVEFKTMNAKLLMDPKRQIFLDQNMLAVIDELMQALNFNSETVPQKSSL |
| lower case) (SEQ ID | EEPDFYKTKIKLCILLHAFRIRAVTIDRVMSYLNAS |
| NO: 183) | |
| Wild-type human | mchqqlviswfslvflasplvaIWELKKDVYVVELDWYPDAPGEMVVLTCDTPEEDGITWT |
| IL-12β subunit | LDQSSEVLGSGKTLTIQVKEFGDAGQYTCHKGGEVLSHSLLLLHKKEDGIWSTDILKDQKE |
| (signal peptide is in | PKNKTFLRCEAKNYSGRFTCWWLTTISTDLTFSVKSSRGSSDPQGVTCGAATLSAERVRGD |
| lower case) (SEQ ID | NKEYEYSVECQEDSACPAAEESLPIEVMVDAVHKLKYENYTSSFFIRDIIKPDPPKNLQLK |
| NO: 184) | PLKNSRQVEVSWEYPDTWSTPHSYFSLTFCVQVQGKSKREKKDRVFTDKTSATVICRKNAS |
| | ISVRAQDRYYSSSWSEWASVPCS |
| L1 construct (SEQ | atgcgagttcctgctcagctgcttggtctgttactgctgtggttgccgggcgcacgatgtg |
| ID NO: 1)- | ctATCTGGGAATTAAAGAAAGATGTGTACGTGGTGGAATTGGATTGGTACCCAGATGCTCC |
| includes (1) leader | GGGCGAAATGGTTGTACTCACATGCGATACTCCGGAGGAAGACGGTATCACTTGGACATTG |
| sequence (lower | GATCAGTCGAGTGAGGTTCTCGGTAGTGGTAAAACACTAACGATCCAAGTCAAAGAATTCG |
| case; SEQ ID NO: | GTGATGCGGGGCAATATACCTGTCATAAAGGCGGCGAAGTACTATCTCATAGCCTCCTGCT |
| 26); (2) IL-12β | GTTACACAAGAAGGAAGATGGCATATGGTCCACCGACATCCTTAAGGATCAGAAAGAACCC |
| (bolded and | AAGAACAAAACTTTCTTGCGTTGCGAAGCTAAGAACTACTCCGGCCGCTTCACATGCTGGT |
| underlined; SEQ ID | GGTTGACAACGATCAGTACGGATCTAACCTTCTCTGTTAAGTCCAGTCGGGGGAGTTCGGA |
| NO: 51); (3) first GS | CCCCCAAGGCGTCACGTGTGGAGCTGCCACACTTTCCGCTGAGCGCGTACGTGGAGATAAT |
| linker (italicized; | AAAGAGTATGAATACTCCGTTGAGTGCCAGGAGGACTCCGCGTGCCCCGCTGCCGAGGAGA |
| SEQ ID NO: 76); | GTCTCCCCATAGAGGTGATGGTCGACGCTGTTCACAAACTGAAATATGAGAACTATACCTC |
| (4) IL-12α (bolded; | ATCCTTCTTTATACGTGACATAATTAAGCCAGATCCCCCGAAGAACTTACAATTGAAACCA |
| SEQ ID NO: 101); | TTGAAGAATTCACGTCAAGTCGAGGTATCCTGGGAGTATCCCGACACCTGGTCCACGCCAC |
| (5) second GS linker | ACTCATATTTCTCTCTGACCTTCTGTGTGCAGGTACAAGGCAAGAGCAAACGAGAAAAAAA |
| (italicized and | GGACAGAGTTTTCACGGATAAGACTAGCGCCACAGTGATATGTAGGAAAAACGCATCGATC |
| underlined; SEQ ID | TCAGTCCGCGCGCAAGATCGGTATTACTCAAGCAGTTGGTCAGAGTGGGCATCGGTGCCCT |
| NO: 126); and (6) | GCTCGGGGGGTTCTGGTGGGGGCAGTGGAGGAGGATCAGGTGGCGGCAGTCGCAATCTACC |
| albumin (lower case | CGTGGCAACACCAGACCCGGGAATGTTCCCATGTCTGCACCACAGTCAAAACCTCTTAAGG |
| and underlined; SEQ | GCCGTGTCAAATATGCTACAGAAGGCGAGACAGACTTTAGAATTCTACCCTTGTACAAGCG |
| ID NO: 147) | AGGAGATTGACCACGAGGACATCACCAAAGATAAGACGAGCACCGTCGAGGCTTGCCTGCC |
| | TCTAGAACTAACAAAAAATGAATCATGCTTGAACTCGAGGGAGACCAGTTTCATTACTAAC |
| | GGTTCATGTCTTGCATCGAGGAAGACCTCATTCATGATGGCCCTGTGCCTCTCGTCCATTT |
| | ATGAAGACCTAAAGATGTACCAGGTAGAGTTTAAGACCATGAACGCCAAGCTCCTCATGGA |
| | TCCAAAACGGCAAATATTCTTAGATCAGAATATGCTCGCTGTTATCGACGAACTCATGCAG |
| | GCGCTTAACTTCAACTCAGAAACCGTTCCCCAAAAGTCGAGTCTAGAAGAACCGGACTTTT |
| | ATAAGACCAAAATTAAACTGTGTATACTACTTCACGCCTTCAGGATAAGAGCAGTGACGAT |
| | TGACAGGGTGATGTCCTACTTGAATGCATCAGGATCAGGGGGAGGCTCGgacgcccataag |
| | agcgaagtcgcccaccgcttcaaggatttgggtgaggaaaacttcaaagccctggtcctga |
| | tagcgtttgcccaatatttgcagcagtgtccattcgaagatcacgtgaaattggtgaacga |
| | ggtaacagaatttgctaagacttgtgtggctgacgagtcggccgaaaactgtgataagagt |
| | cttcatacactgtttggcgataagctatgtactgtcgctacacttagggagacttacggtg |
| | agatggccgactgctgcgccaagcaagagccagaacgaaacgagtgttttctgcaacataa |
| | ggacgacaatcccaacctgcccagattggttcgccctgaagttgatgttatgtgcaccgca |
| | tttcacgacaacgaagaaacctttcttaaaaagtatctgtacgagatagctcgacgtcacc |
| | cttacttctacgcgcccgaacttctgtttttcgccaagcgatacaaagccgctttcacaga |
| | gtgttgccaagctgccgacaaagccgcttgccttctaccaaagcttgacgagctcagagat |
| | gaagggaaagctagttcggcaaagcaacgattaaagtgtgcatcactgcaaaaattcggcg |
| | aacgagcctttaaagcatgggcagttgccaggttatcccaaaggttcccgaaagctgaatt |
| | cgctgaggtgagcaagttagtcacggaccttacgaaggtacataccgaatgctgccacggg |
| | gacctcttggagtgcgctgacgacagggcggacttagctaaatacatttgcgagaatcagg |
| | actcaatcagttctaaacttaaagaatgctgcgagaaaccgctcctggaaaaatcacattg |
| | catcgccgaggtggaaaacgatgagatgccagcagatttaccatctctagccgccgacttc |
| | gtggaaagtaaggatgtgtgtaaaaactatgcggaagcaaaagacgtgttcctcggaatgt |
| | ttctatacgaatacgctagaaggcatcctgactattctgtcgttttactgcttagactagc |
| | gaagacatatgaaacgacgttagagaaatgctgcgcggccgctgacccccacgaatgttac |
| | gcgaaggtctttgatgagttcaagcccctggttgaggagccgcaaaaccttattaaacaga |
| | attgtgagctatttgagcagttaggcgaatataaattccagaatgcacttctagtacgata |
| | caccaaaaaggtccctcaagtgagcacccccactcttgtggaggtatccagaaatctagga |
| | aaggtaggctctaaatgctgcaagcatcccgaagccaagagaatgccatgcgctgaagact |
| | accttagcgttgttctgaatcagttgtgtgtccttcacgaaaagacgccggtgagtgatcg |
| | tgtcacgaagtgctgtacagagagcctcgtcaaccgtaggccatgtttctccgctctcgag |
| | gtggatgaaacatatgtacctaaggaatttaatgcggaaactttcacctttcacgcggaca |
| | tctgtaccctgagcgagaaggagaggcagataaaaaagcagacggctcttgtagagttggt |
| | caaacataagcctaaggccactaaagagcagctaaaggcagtaatggacgactttgcggct |
| | ttcgttgagaagtgctgcaaggccgacgataaagagacctgtttcgcggaagaaggtaaaa |
| | agttagtggccgcctcccaggcggccctgggcctgtag |
| L2 Construct (SEQ | atgcgcgtgcccgcacaattgctcggactgctgctactgtggctgcccggggctcgctgcg |
| ID NO: 2)- | caATTTGGGAACTTAAGAAGGACGTATACGTTGTGGAACTTGACTGGTACCCTGATGCTCC |
| includes (1) leader | AGGGGAGATGGTGGTTTTGACGTGTGACACCCCGGAAGAAGATGGAATTACATGGACCTTA |
| sequence (lower | GACCAATCCTCCGAGGTTCTTGGCTCGGGCAAAACCTTGACCATTCAGGTCAAGGAATTTG |
| case; SEQ ID NO: | GCGATGCTGGCCAATACACCTGCCATAAAGGTGGTGAAGTTTTATCTCACTCCCTACTGTT |
| 27); (2) IL-12ß | GCTCCATAAGAAAGAAGACGGCATTTGGTCGACAGACATATTGAAGGATCAGAAGGAACCT |
| (bolded and | AAGAATAAGACCTTCTTACGATGTGAGGCCAAGAATTATTCCGGACGTTTTACGTGTTGGT |
| underlined; SEQ ID | GGTTGACCACGATCTCCACTGACTTAACCTTCTCAGTGAAATCCTCACGAGGTAGTTCCGA |
| NO: | TCCCCAGGGTGTGACGTGCGGTGCGGCCACGTTAAGTGCTGAGAGAGTACGGGGCGACAAT |
| 52); (3) first GS | AAGGAATACGAGTACTCAGTTGAATGCCAAGAGGACTCGGCCTGTCCCGCGGCAGAGGAGA |
| linker (italicized; | GTCTCCCTATCGGAGTGATGGTGGATGCGGTGCACAAGCTCAAGTATGAAAATTACACATC |
| SEQ ID NO: 77); | ATCTTTTTTCATCAGAGACATTATAAAGCCCGACCCACCAAAGAACCTCCAGTTAAAGCCC |
| (4) IL-12α (bolded; | TTAAAGAACAGTAGACAGGTTGAAGTATCATGGGAATACCCAGACACCTGGTCCACACCCC |
| SEQ ID NO: 102); | ATTCGTATTTTTCCTTGACGTTTTGCGTACAAGTTCAGGGAAAGTCCAAACGGGAAAAGAA |
| (5) second GS linker | AGACCGCGTTTTTACAGACAAAACTTCTGCCACTGTCATCTGTAGAAAGAACGCATCAATT |
| (italicized and | AGCGTGCGAGCGCAAGACAGATACTATTCAAGTAGCTGGAGCGAGTGGGCCAGTGTTCCAT |
| underlined; SEQ ID | GTTCTGGCGGTTCGGGCGGAGGGTCCGGCGGTGGTAGCGGAGGCGGGAGCAGGAACTTGCC |
| NO: 127); and (6) | CGTGGCTACTCCAGACCCTGGCATGTTCCCCTGTTTACACCACTCCCAGAACTTATTACGT |
| albumin (lower case | GCTGTTAGCAACATGTTGCAAAAGGCCCGTCAAACCCTCGAGTTTTACCCCTGTACTAGTG |
| and underlined; SEQ | AAGAAATCGACCACGAAGACATAACAAAAGACAAGACAAGCACAGTTGAGGCATGCTTACC |
| ID NO: 148) | CCTGGAGTTAACAAAGAACGAGAGCTGTCTGAACTCTCGAGAGACGAGCTTCATCACCAAT |
| | GGGAGTTGCCTTGCTTCTCGAAAAACGTCGTTCATGATGGCCCTGTGCCTGTCGTCTATCT |
| | ATGAGGATCTGAAAATGTATCAGGTTGAATTCAAGACAATGAATGCCAAACTACTCATGGA |
| | TCCAAAACGGCAGATATTCCTCGATCAGAATATGCTCGCAGTTATTGACGAACTAATGCAG |
| | GCTCTGAATTTTAACAGCGAGACCGTTCCTCAGAAGTCAAGTTTGGAAGAACCTGACTTCT |
| | ACAAAACTAAAATAAAATTATGCATCTTACTGCATGCTTTCAGAATTAGAGCTGTCACTAT |
| | TGATCGAGTGATGTCATACTTGAATGCTTCCGGATCAGGAGGTGGGAGCgacgcgcacaaa |
| | agcgaggtagcgcatcgctttaaagacttaggagaggaaaactttaaggcgctggtgctca |
| | tcgcatttgcccaatacttacagcagtgtccttttgaggaccacgtaaagcttgtaaacga |
| | agtcactgaattcgccaagacatgtgttgctgacgaaagcgcagagaactgtgacaaaagc |
| | cttcataccctctttggtgacaaactctgcaccgtggcaactctaagagaaacctacgggg |
| | aaatggcagactgctgcgcaaagcaggaacccgaacgcaatgagtgtttcctgcagcacaa |
| | ggatgataatcccaatctgccacgacttgtacggccggaggtagatgttatgtgcactgct |
| | tttcatgacaacgaggaaactttccttaagaaatatctgtatgagatcgcaaggcggcacc |
| | cttacttctacgcacccgaactgttgtttttcgcaaagaggtataaggccgcatttaccga |
| | gtgttgtcaggccgctgataaagccgcctgtcttttgccaaaattagatgaactaagggac |
| | gaaggcaaagcgagtagcgccaaacaaagattaaaatgtgcaagcctccaaaaattcggtg |
| | aaagagcatttaaggcgtgggctgtcgcccgactttcacaacgcttccccaaagctgaatt |
| | cgctgaggtttcgaagctggttaccgacctaactaaagtgcatacagagtgctgtcatggg |
| | gatctcttagagtgcgcggatgaccgggcagacctggctaagtacatatgtgagaaccagg |
| | acagtatatcatcaaagctgaaagagtgttgtgagaagccactactcgagaagagtcactg |
| | tattgccgaggtggaaaatgatgagatgccagccgatcttccttctttggccgctgacttt |
| | gtagagagcaaggatgtctgtaagaactacgctgaggccaaggatgtctttttggggatgt |
| | tcctctatgagtacgcccgacgacaccctgactatagtgtagtacttttgcttagactggc |
| | taaaacatatgagacgactctcgaaaagtgctgtgccgctgccgatccacacgagtgctac |
| | gctaaggtgtttgatgagtttaagccgctggtggaggaaccccagaacctgatcaagcaga |
| | actgtgaactattcgagcaactaggggagtacaaattccagaacgcacttttagtgcggta |
| | caccaaaaaagtgccacaggtcagtacaccaacattagtggaagtatccaggaacctgggc |
| | aaagtgggcagcaaatgctgcaaacatccggaggctaagcggatgccctgtgcagaggact |
| | acctgtccgtggtgcttaaccagctgtgtgtgcttcacgagaaaacgcctgtgtccgaccg |
| | ggtgaccaagtgctgtacggagtcactggtaaatcgacgaccgtgtttttcagcactagaa |
| | gttgatgaaacttatgtaccgaaagagtttaacgcagagacctttacattccacgccgaca |
| | tctgcacgctgtccgagaaggaaagacagattaaaaagcagactgccctagtcgagcttgt |
| | caaacacaaaccgaaggcaaccaaggaacagttaaaagcagtgatggatgattttgctgcg |
| | ttcgtcgaaaaatgttgcaaagcggacgacaaggagacttgcttcgcagaggaagggaaga |
| | aattggttgcggcgtcccaagcggccttagggctatag |
| L3 Construct (SEQ | atgagagttcctgctcaactactagggctgctgttgttgtggttgcccggcgcgcgatgcg |
| ID NO: 3)- | caATATGGGAGCTCAAGAAGGACGTTTATGTCGTTGAGCTGGATTGGTACCCTGATGCCCC |
| includes (1) leader | GGGCGAAATGGTTGTGCTTACGTGCGATACCCCCGAGGAGGATGGCATAACATGGACGTTA |
| sequence (lower | GATCAGTCTTCCGAGGTCCTTGGTTCCGGTAAGACTCTTACTATCCAGGTGAAGGAGTTCG |
| case; SEQ ID NO: | GCGATGCCGGCCAGTACACTTGCCATAAAGGCGGTGAAGTTCTAAGCCACTCTCTACTGCT |
| 28); (2) IL-12ß | TTTGCACAAGAAGGAAGATGGAATATGGTCCACCGACATCTTGAAGGACCAGAAAGAACCA |
| (bolded and | AAAAATAAGACATTTTTGAGGTGTGAGGCAAAAAATTATTCGGGACGCTTCACCTGCTGGT |
| underlined; SEQ ID | GGTTGACGACGATTTCAACCGACCTCACCTTCTCAGTAAAGAGTTCGAGAGGTAGTTCCGA |
| NO: 53); (3) first GS | TCCCCAAGGTGTGACATGTGGCGCTGCGACTCTAAGCGCTGAACGCGTAAGAGGTGATAAC |
| linker (italicized; | AAAGAGTACGAATACAGTGTGGAATGCCAAGAAGATAGCGCGTGTCCAGCCGCAGAAGAAT |
| SEQ ID NO: 78); | CTTTACCAATAGAGGTTATGGTTGATGCCGTTCACAAATTGAAATATGAGAATTACACCTC |
| (4) IL-12α (bolded; | AAGCTTTTTCATTCGAGACATAATAAAGCCCGACCCTCCTAAGAATCTTCAGTTAAAACCG |
| SEQ ID NO: 103); | CTGAAGAACAGTAGACAAGTTGAGGTTAGCTGGGAATATCCTGATACCTGGTCAACGCCGC |
| (5) second GS linker | ACTCGTATTTCTCCCTGACTTTCTGTGTTCAGGTTCAAGGAAAATCTAAAAGGGAGAAGAA |
| (italicized and | AGACCGTGTTTTCACCGACAAGACATCTGCCACAGTCATATGTAGGAAGAACGCTTCAATC |
| underlined; SEQ ID | AGCGTGCGAGCGCAGGACCGATACTACAGCTCTAGTTGGTCCGAATGGGCCAGCGTACCAT |
| NO: 128); and (6) | GCTCTGGCGGTTCCGGCGGGGGCTCGGGGGGCGGGAGCGGTGGAGGCAGCAGGAATCTTCC |
| albumin (lower case | TGTCGCGACCCCTGATCCCGGCATGTTTCCTTGCCTGCACCACAGTCAGAACTTATTACGC |
| and underlined; SEQ | GCCGTTTCCAATATGTTACAGAAGGCCAGACAGACATTAGAGTTTTATCCTTGCACATCGG |
| ID NO: 149) | AGGAGATCGACCATGAAGATATCACAAAAGATAAAACATCCACCGTTGAGGCCTGCCTGCC |
| | ACTTGAACTTACTAAAAACGAGAGCTGCCTGAATAGCCGGGAAACTTCTTTCATCACTAAT |
| | GGATCGTGTCTAGCAAGCCGAAAAACCAGCTTCATGATGGCTTTGTGCCTCTCGTCCATCT |
| | ATGAAGACCTGAAAATGTATCAAGTAGAATTTAAAACGATGAATGCCAAACTGCTTATGGA |
| | TCCCAAACGCCAGATATTTCTAGACCAGAATATGCTGGCCGTCATTGATGAGCTAATGCAG |
| | GCTCTCAATTTTAATAGCGAAACAGTGCCCCAAAAAAGCTCTTTGGAAGAGCCGGATTTTT |
| | ACAAAACCAAGATTAAGCTATGTATCCTGCTGCATGCTTTCAGAATTCGAGCTGTTACAAT |
| | TGATCGGGTTATGAGCTACTTAAACGCCTCGGGTAGTGGCGGGGGGAGCgacgcccacaag |
| | tctgaagtggcacaccggttcaaggacctcggggaggagaattttaaagccctcgtgctga |
| | tcgctttcgcgcagtacttgcagcagtgcccttttgaggaccatgtcaaattagtaaacga |
| | agtgacggaattcgcaaagacttgcgtagcggatgagtcagcagagaattgcgacaagtcg |
| | ctacacactctgttcggggataagttgtgcacagttgctaccttacgagagacctatggag |
| | agatggctgactgctgcgccaaacaagagcctgaaagaaacgagtgcttcttacaacacaa |
| | agacgataacccgaatttgccaaggcttgtaagacccgaagtagatgttatgtgcacagct |
| | tttcacgacaacgaggagacgttccttaagaaatatctgtatgaaatagcccgtcggcacc |
| | cctatttttatgctcccgaactattgttcttcgccaaacgatacaaggctgcgttcactga |
| | gtgctgtcaggctgcagacaaagcagcctgcttgcttcccaaattggacgaactacgggac |
| | gaaggtaaggcgagctccgcaaaacagcggttgaaatgcgcgtcactacagaagtttgggg |
| | aaagagcgttcaaagcttgggctgtggcacgattgagccaacgcttccccaaagcagagtt |
| | tgcggaagtttcgaaactcgtgacagacttaacaaaggttcacaccgagtgctgtcacggc |
| | gatctgctcgaatgcgctgatgaccgcgctgacttggctaaatacatttgtgagaaccagg |
| | actctatatcgagcaaactcaaggaatgctgcgaaaagcccctgcttgagaagtcccactg |
| | catcgctgaggtagagaatgatgagatgcctgcggatcttccgagtttagcagctgatttc |
| | gtcgagagcaaagatgtgtgcaaaaattatgccgaagcaaaagacgtatttcttgggatgt |
| | tcttatacgagtatgcacggcgccacccagattactccgtagtgctactgttaagattggc |
| | gaagacctatgaaaccacattggaaaagtgctgcgccgccgccgacccccacgagtgttac |
| | gcgaaggtgttcgatgaatttaaaccgcttgttgaggagccgcaaaacctaataaagcaaa |
| | actgtgagctctttgagcaactaggtgaatacaagtttcagaatgcactcctagttcggta |
| | caccaaaaaagtacctcaggtatctacgccaacgttagtcgaggtctcgcggaatttgggt |
| | aaagtaggttccaagtgttgcaaacacccggaagctaaacgtatgccgtgtgctgaggact |
| | atctcagcgttgtgttaaaccaactttgcgtactccacgagaaaacacctgtctcagatcg |
| | ggtaaccaaatgctgcacggagtcgctagtaaatcgtcgcccatgcttttctgcgttagag |
| | gtggacgaaacttatgtaccgaaagaatttaacgcggaaacctttacattccatgcagata |
| | tctgtacactgtccgagaaagagagacagattaagaaacagacggcgctggtggagcttgt |
| | gaagcacaagcctaaagctacgaaggagcaactgaaggcagtcatggatgactttgcggcg |
| | tttgtggagaagtgctgtaaagcggacgataaggaaacatgcttcgcagaagaaggaaaga |
| | agctggtcgccgctagccaagcggctctgggcctgtag |
| M1 Construct (SEQ | atgagagtccccgcccagctgctggggcttcttttgctttggcttcctggcgcaagatgcg |
| ID NO: 4)- | caATATGGGAGCTCAAGAAGGACGTTTATGTCGTTGAGCTGGATTGGTACCCTGATGCCCC |
| includes (1) leader | GGGCGAAATGGTTGTGCTTACGTGCGATACCCCCGAGGAGGATGGCATAACATGGACGTTA |
| sequence (lower | GATCAGTCTTCCGAGGTCCTTGGTTCCGGTAAGACTCTTACTATCCAGGTGAAGGAGTTCG |
| case; SEQ ID NO: | GCGATGCCGGCCAGTACACTTGCCATAAAGGCGGTGAAGTTCTAAGCCACTCTCTACTGCT |
| 29); (2) IL-12ß | TTTGCACAAGAAGGAAGATGGAATATGGTCCACCGACATCTTGAAGGACCAGAAAGAACCA |
| (bolded and | AAAAATAAGACATTTTTGAGGTGTGAGGCAAAAAATTATTCGGGACGCTTCACCTGCTGGT |
| underlined; SEQ ID | GGTTGACGACGATTTCAACCGACCTCACCTTCTCAGTAAAGAGTTCGAGAGGTAGTTCCGA |
| NO: 54); (3) first GS | TCCCCAAGGTGTGACATGTGGCGCTGCGACTCTAAGCGCTGAACGCGTAAGAGGTGATAAC |
| linker (italicized; | AAAGAGTACGAATACAGTGTGGAATGCCAAGAAGATAGCGCGTGTCCAGCCGCAGAAGAAT |
| SEQ ID NO: 79); | CTTTACCAATAGAGGTTATGGTTGATGCCGTTCACAAATTGAAATATGAGAATTACACCTC |
| (4) IL-1α (bolded; | AAGCTTTTTCATTCGAGACATAATAAAGCCCGACCCTCCTAAGAATCTTCAGTTAAAACCG |
| SEQ ID NO: 104); | CTGAAGAACAGTAGACAAGTTGAGGTTAGCTGGGAATATCCTGATACCTGGTCAACGCCGC |
| (5) second GS linker | ACTCGTATTTCTCCCTGACTTTCTGTGTTCAGGTTCAAGGAAAATCTAAAAGGGAGAAGAA |
| (italicized and | AGACCGTGTTTTCACCGACAAGACATCTGCCACAGTCATATGTAGGAAGAACGCTTCAATC |
| underlined; SEQ ID | AGCGTGCGAGCGCAGGACCGATACTACAGCTCTAGTTGGTCCGAATGGGCCAGCGTACCAT |
| NO: 129); and (6) | GCTCTGGCGGCAGCGGCGGCGGGAGCGGCGGAGGCTCCGGGGGAGGTAGCCGGAATCTGCC |
| albumin (lower case | TGTCGCCACTCCAGACCCCGGCATGTTCCCATGTCTGCATCATTCTCAGAACCTGCTGAGG |
| and underlined; SEQ | GCCGTATCCAATATGCTGCAGAAAGCCAGACAGACCTTAGAGTTCTATCCCTGTACAAGCG |
| ID NO: 150) | AGGAGATAGATCACGAGGATATTACGAAGGACAAAACTTCTACTGTTGAGGCGTGTCTTCC |
| | ATTAGAGCTGACCAAGAACGAAAGCTGTCTGAATAGCAGAGAGACTTCATTTATCACCAAT |
| | GGGAGTTGCTTGGCTAGCAGAAAGACCAGCTTCATGATGGCCCTTTGCTTGTCTTCGATAT |
| | ACGAAGATCTTAAGATGTATCAAGTGGAATTTAAGACGATGAACGCCAAGCTGCTTATGGA |
| | TCCCAAGCGCCAAATCTTCCTGGATCAGAACATGTTGGCCGTGATTGACGAGCTGATGCAA |
| | GCCCTGAATTTCAACTCCGAGACCGTGCCTCAGAAGAGCAGCCTCGAGGAGCCCGACTTCT |
| | ACAAAACAAAGATCAAACTCTGCATCCTTCTGCACGCCTTCAGAATTAGAGCCGTGACCAT |
| | CGACAGAGTTATGAGCTACCTGAATGCCAGCGGCAGCGGCGGCGGATCCgatgcccataaa |
| | tctgaggtggcccatagattcaaggatctgggcgaagaaaacttcaaagccttggtcttga |
| | tcgcctttgcccagtacctgcagcagtgcccctttgaggaccacgtgaagctggtgaatga |
| | agtgaccgagtttgccaagacgtgcgtggctgatgagagcgccgaaaactgcgacaaaagc |
| | ctgcacaccctgtttggcgacaagctgtgcaccgtagccaccctgagagaaacttacggcg |
| | agatggctgactgctgcgccaagcaggagcccgagagaaacgagtgctttctgcagcacaa |
| | ggacgacaatcccaacctgcccagactggtgagacccgaagtggatgttatgtgcaccgct |
| | ttccacgacaatgaagagacatttctcaagaagtacttgtacgagattgcaagaagacacc |
| | cttacttttacgcccccgaattactgttcttcgctaagaggtataaggcagccttcactga |
| | atgctgccaggctgccgacaaagcagcttgcctgctgccaaagctggatgaactgcgagac |
| | gaaggaaaggcgtcctccgccaagcagcgtttgaagtgcgccagccttcagaagtttggcg |
| | agcgggccttcaaggcatgggccgtggctcgacttagccagcgttttcccaaggctgaatt |
| | tgcagaggtgagtaaactggttaccgatctgacaaaggtgcacaccgagtgctgtcacggt |
| | gacctcttagagtgcgccgacgacagagccgacctcgccaagtacatttgtgaaaaccaag |
| | actcaatctcttcaaagttaaaggagtgctgcgaaaagcccctgcttgaaaagagccactg |
| | cattgccgaagtcgagaatgatgagatgcctgcagacttgcccagcttggcagccgacttc |
| | gttgagtctaaggacgtgtgcaagaattacgccgaggcaaaagacgtgttcctgggcatgt |
| | tcctttatgagtacgctagaagacatcccgactacagcgtggtccttctccttaggctcgc |
| | taagacttacgagacgacgttggagaagtgttgtgccgctgcggacccccacgagtgctat |
| | gccaaagtgttcgatgagtttaaacccctggtggaggaacctcagaaccttatcaagcaga |
| | attgtgagttgttcgaacagctaggcgagtacaagttccagaatgccctgctggtgagata |
| | cacaaaaaaggtgccccaggtgtcaaccccgaccttagtggaagtgtccagaaacctgggc |
| | aaggtgggcagcaagtgctgcaagcaccccgaagctaagagaatgccgtgcgcggaggatt |
| | acctgagcgtggtgctcaaccagctgtgtgtgcttcacgagaaaacacccgtgagcgacag |
| | ggtgacaaaatgttgcacagaaagccttgtgaaccggagaccttgtttcagcgccctggag |
| | gttgacgagacctatgttcctaaggagttcaacgctgagactttcacatttcacgctgata |
| | tatgtaccctgagcgagaaagaaagacagatcaagaagcagaccgccctggtcgagctggt |
| | gaaacacaagcctaaggccacgaaggagcagctgaaggccgtcatggacgacttcgcagcc |
| | ttcgtcgagaaatgctgcaaagccgacgacaaggaaacctgcttcgccgaagagggaaaga |
| | agctggtggccgcctcccaggccgcccttgggctctag |
| M2 Construct (SEQ | atgagagtccccgcccagctgctagggctgctgctgctgtggttacccggcgcccggtgtg |
| ID NO: 5)- | caATTTGGGAGTTGAAGAAGGACGTGTACGTGGTGGAGCTGGACTGGTACCCGGATGCTCC |
| includes (1) leader | CGGCGAGATGGTGGTACTCACCTGCGACACACCTGAGGAAGACGGCATCACCTGGACCCTC |
| sequence (lower | GATCAGAGCAGCGAGGTTCTGGGAAGCGGCAAAACCCTGACCATCCAAGTGAAAGAGTTTG |
| case; SEQ ID NO: | GCGACGCCGGTCAGTACACCTGCCACAAGGGAGGCGAGGTCCTGTCTCACTCTCTGCTGCT |
| 30); (2) IL-12ß | GCTCCATAAGAAGGAGGACGGTATTTGGAGCACTGACATCTTGAAGGATCAAAAAGAGCCA |
| (bolded and | AAGAATAAAACGTTCCTGAGGTGCGAAGCTAAGAATTACTCCGGGCGTTTTACGTGCTGGT |
| underlined; SEQ ID | GGCTGACCACGATCAGCACCGATCTGACCTTCAGCGTGAAGAGCAGCCGGGGCAGCAGCGA |
| NO: 55); (3) first GS | CCCCCAAGGCGTGACTTGCGGCGCTGCGACCCTGAGCGCTGAGCGTGTGCGCGGCGACAAC |
| linker (italicized; | AAGGAGTATGAGTATTCAGTGGAGTGTCAGGAGGACTCCGCCTGTCCCGCGGCCGAAGAGA |
| SEQ ID NO: 80); | GTCTGCCTATTGAGGTGATGGTGGACGCCGTGCACAAGCTGAAGTACGAGAACTACACATC |
| (4) IL-12α (bolded; | GTCATTCTTTATCCGCGACATCATAAAGCCCGACCCCCCCAAGAACCTGCAGCTGAAGCCT |
| SEQ ID NO: 105); | CTCAAGAATTCCCGGCAAGTGGAGGTGAGCTGGGAGTACCCTGATACCTGGTCTACCCCTC |
| (5) second GS linker | ACAGCTACTTTAGCCTGACCTTCTGCGTCCAGGTGCAAGGAAAGTCGAAGCGCGAGAAGAA |
| (italicized and | AGATAGAGTCTTCACCGATAAAACCAGTGCCACCGTGATTTGCCGCAAAAACGCCTCCATC |
| underlined; SEQ ID | AGCGTGCGGGCTCAGGATAGATACTACTCTAGCAGCTGGAGCGAATGGGCCTCAGTTCCTT |
| NO: 130); and (6) | GCAGCGGCGGCAGCGGTGGAGGAAGCGGCGGTGGCAGTGGGGGGGGGAGCAGAAATCTGCC |
| albumin (lower case | CGTCGCCACTCCAGATCCTGGCATGTTCCCGTGCCTGCATCACAGCCAAAACCTGCTGCGG |
| and underlined; SEQ | GCGGTGTCTAACATGCTGCAGAAGGCTAGGCAGACCTTGGAATTCTATCCCTGCACAAGCG |
| ID NO: 151) | AGGAAATAGACCATGAGGACATCACCAAGGATAAGACCAGCACGGTCGAAGCTTGCCTGCC |
| | ACTGGAACTGACAAAAAACGAGAGTTGCCTGAACTCCCGCGAGACATCCTTCATCACAAAC |
| | GGCAGCTGCCTGGCTAGCAGGAAGACCAGCTTCATGATGGCCCTGTGCCTGTCTTCCATCT |
| | ACGAGGACCTGAAAATGTACCAAGTGGAGTTCAAGACTATGAACGCCAAGCTGCTAATGGA |
| | TCCCAAGCGACAGATCTTTCTAGACCAGAACATGCTGGCCGTCATTGACGAGCTGATGCAG |
| | GCACTCAATTTTAACTCAGAGACCGTGCCACAGAAGTCCAGCCTGGAGGAGCCTGACTTCT |
| | ATAAGACCAAGATTAAGCTGTGCATCCTGCTGCATGCCTTCCGAATAAGAGCCGTGACCAT |
| | TGACCGAGTGATGTCATACTTGAACGCAAGCGGCTCAGGCGGAGGGAGTgacgcccacaag |
| | tctgaagtggctcaccggtttaaggaccttggcgaggagaactttaaagccctggtgctga |
| | ttgcctttgcccagtatttacaacaatgccctttcgaagaccacgtgaagctcgtcaatga |
| | ggtcaccgagttcgctaagacctgcgtagccgacgaaagtgccgagaactgcgacaagagc |
| | ctgcacaccctgttcggggacaaactctgtaccgtggccaccctacgggagacatatgggg |
| | agatggccgactgctgcgcaaaacaggagcccgagagaaatgagtgcttcctgcagcacaa |
| | ggatgacaaccccaatctgcccagactggtgcgccccgaggtagacgttatgtgcaccgcc |
| | ttccatgacaatgaggagacgttcctgaagaaatacctgtacgagatcgcaagacgtcacc |
| | cctatttctatgcacctgagctgcttttcttcgccaagagatataaggccgccttcaccga |
| | atgctgccaggcagccgataaggcagcttgcctcctgccaaagctggacgagctgagagat |
| | gagggcaaggcctccagcgcgaagcagagactcaaatgcgcaagccttcagaagttcggag |
| | aacgcgcctttaaagcctgggccgtcgccagactgagccagcgcttccctaaagccgaatt |
| | cgcagaagtgagcaagctggtaacggacctgacaaaggtgcatactgagtgctgccatggc |
| | gatctgctggagtgcgctgatgacagagcagatttggcgaaatatatttgcgaaaatcagg |
| | atagcatcagctctaagctcaaggagtgttgtgagaagcccctgctggaaaaaagccactg |
| | cattgcagaggttgagaacgatgaaatgccagccgaccttccatcattggccgccgatttc |
| | gtggagtcgaaggatgtgtgtaagaactacgccgaggccaaggacgtgttcctgggcatgt |
| | tcctgtacgagtatgctagaagacatcccgattacagtgtggtgctgctattgagactggc |
| | caagacctacgaaaccaccctggagaaatgttgcgccgcggcagatcctcacgaatgttac |
| | gccaaagtgtttgacgaattcaagccactggtagaggagccccagaacttaataaagcaga |
| | attgcgagctattcgagcagttgggcgagtacaaattccagaacgcccttctggtgaggta |
| | taccaaaaaggtgccccaggtgtctacccctaccctggtggaggtcagccgaaatctggga |
| | aaggtcggatccaagtgctgcaagcacccggaggccaagaggatgccttgcgctgaggact |
| | atctcagtgtcgtcctgaatcagctatgcgtgttgcacgagaaaaccccagtgagtgaccg |
| | cgtgactaaatgctgcaccgaaagcttggtgaatcggaggccctgtttctctgcactggaa |
| | gttgacgagacttacgtcccgaaggaattcaacgccgagacattcaccttccatgctgaca |
| | tatgtactctgtcagaaaaggagcgtcagatcaagaagcagacagccctggtggaactggt |
| | taagcataagcctaaagcgaccaaagagcagctgaaagccgtgatggacgattttgccgcc |
| | ttcgtggagaaatgttgtaaggcagacgacaaagagacatgtttcgccgaagaggggaaga |
| | aactggtggccgcaagccaggccgctctgggtctgtag |
| M3 Construct (SEQ | atgagagtcccagctcagctgctgggcctactgctgctatggctccccggcgcgcggtgcg |
| ID NO: 6)- | CCATTTGGGAGCTCAAGAAGGACGTGTACGTGGTGGAACTTGACTGGTACCCGGACGCGCC |
| includes (1) leader | CGGGGAAATGGTGGTGCTAACCTGTGACACCCCCGAAGAGGACGGCATCACCTGGACCCTG |
| sequence (lower | GACCAGAGCAGTGAGGTGCTAGGTAGTGGCAAAACGTTAACCATCCAGGTCAAGGAGTTCG |
| case; SEQ ID NO: | GCGACGCCGGGCAATACACCTGTCACAAGGGGGGGGAGGTACTATCCCACTCCCTGCTGCT |
| 31); (2) IL-12 | CCTGCACAAGAAAGAGGACGGGATCTGGAGCACCGACATTCTGAAAGACCAAAAGGAGCCC |
| (bolded and | AAAAACAAAACCTTCCTTAGATGTGAAGCCAAGAACTACAGCGGCCGTTTCACCTGCTGGT |
| underlined; SEQ ID | GGCTGACCACCATATCTACGGACCTTACCTTTTCGGTGAAGAGCAGCAGGGGGAGTTCCGA |
| NO: 56); (3) first GS | CCCGCAAGGCGTAACTTGCGGAGCCGCAACCCTGAGCGCCGAGAGAGTGCGCGGCGACAAC |
| linker (italicized; | AAGGAGTACGAGTATAGCGTGGAGTGCCAAGAGGACAGCGCATGCCCAGCCGCCGAAGAGA |
| SEQ ID NO: 81); | GCCTGCCAATAGAGGTCATGGTAGACGCCGTGCACAAGCTAAAATATGAAAACTACACCAG |
| (4) IL-12α (bolded; | CAGCTTTTTCATCAGGGATATCATCAAACCCGACCCACCAAAAAACTTACAGCTTAAGCCT |
| SEQ ID NO: 106); | CTGAAAAACAGCAGACAAGTTGAGGTCAGCTGGGAGTACCCCGACACTTGGAGCACACCCC |
| (5) second GS linker | ACTCCTATTTCAGTTTGACATTCTGCGTGCAGGTGCAGGGTAAAAGCAAGAGAGAAAAGAA |
| (italicized and | GGACAGAGTGTTCACAGATAAGACCTCAGCCACAGTGATCTGCCGTAAGAATGCCAGCATC |
| underlined; SEQ ID | AGCGTCCGGGCTCAGGACAGGTACTACTCTTCCTCATGGAGCGAGTGGGCCTCTGTCCCCT |
| NO: 131); and (6) | GCAGCGGCGGCAGCGGCGGTGGCAGCGGCGGCGGTTCTGGCGGCGGTTCAAGAAACCTCCC |
| albumin (lower case | AGTCGCTACCCCCGATCCCGGAATGTTCCCCTGCCTGCACCACTCCCAGAATCTGCTCCGA |
| and underlined; SEQ | GCCGTTAGCAACATGCTGCAAAAGGCCCGGCAGACCCTGGAGTTCTACCCATGCACCTCGG |
| ID NO: 152) | AAGAAATCGATCACGAGGACATCACCAAGGACAAGACTAGCACCGTCGAGGCCTGCCTGCC |
| | GCTGGAACTAACCAAGAATGAAAGCTGCCTCAACTCGCGGGAGACCTCTTTCATAACCAAC |
| | GGCTCATGCCTGGCCAGCCGGAAAACTAGCTTTATGATGGCTCTGTGCTTAAGCAGCATCT |
| | ACGAGGATCTGAAGATGTACCAGGTAGAGTTCAAGACCATGAATGCCAAGCTGCTGATGGA |
| | CCCCAAGAGACAAATCTTCCTGGACCAGAACATGCTGGCCGTAATTGATGAACTGATGCAG |
| | GCCCTGAATTTCAACAGCGAGACCGTACCCCAGAAAAGCTCACTGGAGGAGCCCGACTTTT |
| | ATAAGACGAAAATAAAGTTGTGCATCCTTCTTCACGCTTTCCGGATTAGAGCCGTGACCAT |
| | CGATAGAGTGATGTCATACCTGAACGCATCGGGGAGTGGCGGTGGCAGCgatgcccacaaa |
| | agcgaagtcgcacacagattcaaggacttgggtgaggagaactttaaagccctggtgctga |
| | tcgccttcgcgcagtatctccagcagtgccccttcgaagatcatgtgaaactggtgaacga |
| | ggtaaccgagttcgcgaagacatgcgttgctgatgagagcgccgaaaattgcgacaaaagc |
| | ctgcatactctgttcggggacaagctgtgcacggtcgcaaccctgagagaaacctacggcg |
| | agatggcagactgctgcgccaagcaggagcctgagaggaacgagtgttttctgcagcacaa |
| | ggacgataatcctaaccttcctcgtctagtgagacccgaagtggacgttatgtgtaccgcc |
| | tttcacgacaatgaggaaacattcctgaaaaagtacctgtacgagatcgccagacggcacc |
| | catatttctacgcccccgagctgctcttcttcgcaaagaggtacaaggctgccttcaccga |
| | gtgctgccaggcggccgacaaggcggcgtgtttgctgcctaagctggacgaactacgtgac |
| | gaaggaaaagctagcagcgccaagcagagacttaagtgcgcgtccttacagaagtttggcg |
| | aaagagcgtttaaggcctgggccgtggcaaggctgtctcaaagattccccaaggcggagtt |
| | cgccgaggtgtcaaaactggtgaccgacttaaccaaggtgcacaccgaatgctgccacggc |
| | gatctgctcgagtgcgccgacgacagagccgatctggcaaaatacatctgcgaaaaccagg |
| | atagcatcagctccaaactgaaggagtgctgtgaaaaaccactgcttgaaaaatcgcattg |
| | tatagcggaggtggagaatgacgagatgcccgccgacctgccaagcctggccgccgatttc |
| | gttgaatccaaggacgtttgcaagaactatgcagaagcgaaggacgtgttcttaggaatgt |
| | tcctatacgagtacgcgagaagacatcccgactacagcgtggttctgctgttgagattagc |
| | caagacgtatgagacaaccctcgaaaagtgctgcgccgccgccgacccccacgagtgttac |
| | gcaaaggtgttcgatgagtttaaaccgctggttgaggaaccgcaaaacctgatcaagcaga |
| | actgcgagctgttcgagcagctgggtgaatacaagtttcagaatgcactgttggtgcgata |
| | taccaagaaggtgcctcaggtgagcacccctacccttgttgaggtgtcccgcaatctgggt |
| | aaggttgggagcaagtgttgcaaacaccccgaggccaagagaatgccctgcgcggaagatt |
| | atctcagtgtcgtgcttaatcagttatgtgtcctgcatgagaagacccccgtgagcgacag |
| | agtgaccaagtgctgtaccgaatctctcgtgaacagacgcccgtgcttcagcgccttggag |
| | gtagacgagacctacgtgcccaaggagttcaacgcagagaccttcacctttcacgccgata |
| | tctgcaccctgtccgagaaggagagacaaatcaagaaacaaacggccctcgtggagctggt |
| | caagcacaaacccaaggccacaaaagagcagctgaaggccgtgatggacgacttcgcagcc |
| | tttgtggagaaatgctgcaaggctgacgacaaggagacatgcttcgccgaggaaggcaaga |
| | agttggtggccgccagccaggcggccctgggcctgtag |
| H1 Construct (SEQ | atgagagtgcccgcccagttgcttggcctgctgctcctatggcttcccggcgccagatgcg |
| ID NO: 7)- | ccATCTGGGAGCTGAAGAAAGACGTGTACGTGGTGGAGCTGGACTGGTACCCCGACGCCCC |
| includes (1) leader | CGGTGAAATGGTGGTTCTGACCTGCGACACACCAGAGGAGGACGGCATCACCTGGACCCTG |
| sequence (lower | GACCAGTCCAGCGAGGTCCTGGGCTCTGGCAAGACCCTGACCATCCAGGTTAAGGAATTTG |
| case; SEQ ID NO: | GCGACGCCGGCCAGTACACCTGCCACAAAGGCGGCGAGGTCCTTTCGCACAGTCTGCTGCT |
| 32); (2) IL-12ß | GCTGCATAAAAAGGAGGACGGCATTTGGAGCACCGACATTCTGAAGGATCAGAAAGAGCCC |
| (bolded and | AAGAACAAGACCTTTCTGAGATGTGAGGCCAAAAACTACTCTGGACGCTTCACCTGTTGGT |
| underlined; SEQ ID | GGCTGACCACCATCAGCACAGACCTGACCTTCTCGGTGAAGTCTAGTAGGGGCAGCAGTGA |
| NO: 57); (3) first GS | CCCCCAGGGCGTAACATGCGGCGCCGCTACCCTGAGCGCCGAGAGAGTGAGAGGCGATAAC |
| linker (italicized; | AAGGAGTACGAGTACTCCGTGGAGTGCCAAGAGGACTCAGCCTGCCCCGCCGCCGAGGAGT |
| SEQ ID NO: 82); | CGCTGCCCATCGAGGTGATGGTGGATGCAGTGCACAAGCTGAAGTACGAGAACTACACCAG |
| (4) IL-12α (bolded; | TAGCTTTTTCATCAGAGATATCATTAAACCCGACCCTCCCAAGAACCTGCAGCTGAAGCCC |
| SEQ ID NO: 107); | TTAAAGAACAGCCGGCAGGTGGAAGTGTCATGGGAGTACCCAGACACCTGGAGCACTCCGC |
| (5) second GS linker | ACAGCTACTTCAGCCTGACCTTCTGCGTTCAGGTGCAGGGAAAAAGCAAGAGAGAGAAGAA |
| (italicized and | AGACAGAGTGTTCACCGACAAGACCAGCGCAACCGTGATCTGTAGAAAGAACGCCTCGATC |
| underlined; SEQ ID | AGCGTGAGAGCCCAGGACAGATACTACAGCAGCAGCTGGAGCGAGTGGGCCAGTGTACCTT |
| NO: 132); and (6) | GCAGCGGTGGCAGCGGCGGCGGCTCCGGCGGCGGGAGTGGCGGCGGCAGCAGAAATCTGCC |
| albumin (lower case | CGTAGCCACCCCCGACCCCGGCATGTTTCCCTGTCTGCACCATTCTCAAAACCTGTTACGG |
| and underlined; SEQ | GCCGTGAGCAACATGCTGCAGAAGGCCAGACAGACACTGGAGTTTTACCCCTGTACCTCAG |
| ID NO: 153) | AGGAGATCGATCATGAGGACATTACTAAGGACAAGACCAGCACCGTGGAAGCCTGCCTGCC |
| | CCTAGAGCTAACCAAGAACGAGAGCTGCCTGAACTCTAGAGAGACAAGCTTCATCACGAAC |
| | GGCTCATGCCTGGCCAGTAGGAAAACCAGCTTCATGATGGCTCTGTGCCTGAGCTCCATAT |
| | ATGAGGACCTTAAGATGTACCAGGTGGAGTTCAAAACCATGAACGCCAAGCTGCTGATGGA |
| | CCCAAAGAGACAGATCTTCCTTGACCAGAACATGCTGGCCGTTATCGATGAGCTGATGCAG |
| | GCCCTGAACTTCAACAGCGAGACCGTGCCCCAGAAGAGCAGCCTGGAGGAACCCGACTTCT |
| | ACAAAACCAAGATCAAGCTGTGCATTCTGCTGCATGCCTTCCGCATTAGAGCCGTGACCAT |
| | CGATAGGGTGATGAGCTACCTGAACGCCAGCGGCTCTGGCGGCGGCAGTgacgcccacaag |
| | tccgaggtcgcccacagattcaaggatttgggcgaggagaacttcaaggccctggtgctga |
| | tcgccttcgcccagtacttgcagcagtgtcccttcgaggaccatgtgaagctggtgaacga |
| | ggtgaccgagttcgccaagacctgtgtggccgacgagagcgccgagaactgcgataagtct |
| | ctgcacaccctttttggcgacaaactgtgcaccgtggccaccctgagagagacctacggcg |
| | agatggccgactgctgtgcgaagcaggagcccgagcgcaatgagtgtttcctgcagcataa |
| | ggacgacaaccccaacctgcccagactggtgagacccgaggtggacgtgatgtgcaccgcc |
| | ttccacgacaacgaggagacctttctgaaaaaatacctgtacgagatcgcaagacgccacc |
| | cctacttctacgcccccgagctgctgttcttcgccaagcgctacaaggctgccttcaccga |
| | gtgctgccaggccgccgataaggccgcgtgcttactgccaaagctggacgagctgagagac |
| | gaggggaaagcctctagcgccaaacagagattgaagtgtgccagcctgcagaaattcggtg |
| | agagagccttcaaggcctgggccgtggccagattatcacagcggttccccaaggctgaatt |
| | cgccgaggtgagcaaacttgtcaccgatctgacaaaagtgcacaccgagtgctgccatggc |
| | gacctgctggagtgcgccgacgaccgggccgacctggccaagtacatctgcgagaaccagg |
| | acagcatctccagcaagctgaaggagtgctgcgagaagcccctgctggagaagagccactg |
| | catcgccgaggtggagaatgacgaaatgcccgccgacctgcccagcctggccgccgacttc |
| | gtggaaagcaaggacgtgtgcaaaaattacgccgaagccaaggatgtgttcttgggcatgt |
| | tcttgtacgagtacgccagacgccaccccgactacagcgtggtgctgctgctgcggctggc |
| | caagacctacgagaccaccctggagaagtgctgtgctgccgccgacccccacgagtgctac |
| | gccaaggtatttgacgagttcaagcccctggtggaggagcctcagaacctgattaagcaga |
| | actgtgagctgttcgagcagctgggcgagtacaagttccagaacgccctcctggtgagata |
| | caccaaaaaggtgcctcaggtaagcactcccaccctggtggaggtgagcaggaacctcggc |
| | aaggtgggcagcaaatgctgcaagcacccagaggccaaaagaatgccctgcgcagaagact |
| | acctcagcgtggtcctgaaccagctgtgcgtgctgcacgaaaagacccctgtgagcgatag |
| | agtgacaaagtgctgcaccgagagcctggtgaacagaagaccctgttttagcgccctggag |
| | gtggacgagacctacgtgcccaaggagttcaacgccgagacgttcactttccacgcggaca |
| | tctgcaccctgagcgagaaggagagacaaatcaagaagcagaccgccctagtcgagctggt |
| | aaaacacaagcccaaggccaccaaggagcagctgaaggccgtgatggacgactttgcagcc |
| | ttcgtggagaagtgctgcaaggctgacgacaaggagacctgcttcgccgaggagggcaaga |
| | agctcgtagccgccagcaggccgctctcggcctttag |
| H2 Construct (SEQ | atgagagtgcccgcccagctgctgggcctgctgctcttatggctgcccggcgcccgctgtg |
| ID NO: 8)- | ctATTTGGGAGCTGAAGAAGGACGTGTACGTGGTGGAGCTGGATTGGTATCCAGACGCCCC |
| includes (1) leader | CGGCGAGATGGTCGTGTTGACCTGCGATACTCCCGAGGAGGACGGAATCACCTGGACACTT |
| sequence (lower | GACCAGAGCAGCGAGGTGCTGGGCAGCGGGAAGACCCTGACTATCCAGGTGAAGGAGTTTG |
| case; SEQ ID NO: | GCGATGCCGGACAGTACACCTGCCACAAGGGCGGCGAGGTGTTATCTCATAGCCTGCTGCT |
| 33); (2) IL-12ß | GCTGCACAAAAAGGAGGACGGGATCTGGAGCACTGACATCCTGAAGGACCAGAAGGAGCCC |
| (bolded and | AAAAACAAGACCTTCCTGCGTTGCGAGGCCAAGAACTACAGCGGGAGATTCACCTGTTGGT |
| underlined; SEQ ID | GGCTGACCACTATCAGCACTGACCTAACCTTCAGCGTGAAGAGCAGCCGGGGAAGCTCTGA |
| NO: 58); (3) first GS | CCCCCAGGGGGTGACATGCGGCGCCGCCACACTGAGCGCCGAGAGGGTGAGAGGGGACAAT |
| linker (italicized; | AAGGAATACGAGTATAGCGTGGAGTGTCAGGAAGATTCCGCCTGCCCCGCCGCCGAGGAGA |
| SEQ ID NO: 83); | GCCTGCCTATCGAGGTCATGGTGGACGCCGTCCATAAACTGAAGTACGAAAACTATACTTC |
| (4) IL-12α (bolded; | AAGCTTCTTCATCAGAGACATCATAAAGCCCGACCCCCCCAAGAACCTGCAGCTAAAGCCC |
| SEQ ID NO: 108); | CTGAAGAACAGCAGACAGGTCGAAGTGAGCTGGGAGTACCCCGATACCTGGAGCACCCCGC |
| (5) second GS linker | ACAGCTACTTCAGCCTGACCTTTTGCGTCCAGGTGCAGGGCAAGAGCAAGAGAGAGAAGAA |
| (italicized and | GGACAGGGTGTTCACTGACAAGACAAGCGCCACTGTTATCTGCAGAAAGAACGCCAGTATC |
| underlined; SEQ ID | AGCGTGCGCGCCCAAGACAGGTATTACTCCAGCAGCTGGTCTGAATGGGCCAGCGTGCCTT |
| NO: 133); and (6) | GCAGCGGGGGGAGCGGCGGTGGCAGCGGGGGGGGCAGCGGCGGGGGCAGCAGAAACCTGCC |
| albumin (lower case | CGTGGCCACACCCGATCCCGGCATGTTCCCCTGTCTGCACCACAGCCAAAACCTGCTGCGT |
| and underlined; SEQ | GCCGTGAGCAACATGCTGCAGAAGGCGCGGCAGACCCTGGAGTTCTATCCCTGCACCAGTG |
| ID NO: 154) | AGGAGATTGACCACGAGGATATCACCAAAGACAAGACCAGCACCGTGGAGGCCTGCCTCCC |
| | CCTGGAGCTGACCAAGAACGAGTCCTGCTTGAATTCAAGAGAGACCAGCTTCATCACCAAC |
| | GGCTCCTGCTTAGCCAGCAGAAAGACTAGCTTCATGATGGCCTTGTGCTTGTCTAGCATCT |
| | ATGAGGATCTGAAGATGTACCAGGTCGAGTTCAAGACTATGAACGCCAAGCTGCTGATGGA |
| | CCCCAAAAGACAGATCTTCCTGGACCAGAACATGCTGGCCGTCATCGACGAGCTGATGCAG |
| | GCCCTTAATTTCAATAGCGAGACAGTGCCCCAGAAATCTTCTCTGGAGGAGCCCGATTTTT |
| | ACAAAACCAAGATCAAACTATGCATCCTGTTGCACGCCTTCCGGATCCGCGCCGTGACCAT |
| | CGACAGAGTAATGTCCTACCTGAACGCCAGCGGCAGCGGCGGCGGTAGCgacgcccacaag |
| | agcgaagtggcccatagattcaaggacctgggcgaggagaacttcaaggccctcgtgctga |
| | tcgccttcgcccagtacctgcagcagtgccctttcgaggaccacgttaaactggtgaatga |
| | agtgaccgagttcgccaaaacctgcgtggccgacgagtctgccgaaaattgcgacaaaagc |
| | ttacacaccctgttcggcgacaagctgtgcaccgtggccaccttaagagaaacctacggcg |
| | agatggccgactgctgcgctaagcaggagcccgagagaaacgaatgcttcctgcagcacaa |
| | ggacgataacccaaatctgcctagactggtgagacccgaggtggacgtgatgtgcacagcc |
| | ttccacgataacgaagagacattcctgaaaaagtacctgtacgagatcgccagaagacatc |
| | cttacttctatgcccccgagcttctgttcttcgccaagagatataaggccgccttcaccga |
| | gtgctgccaagccgccgacaaggcagcctgcctgctgccaaagcttgacgagctgagagac |
| | gagggcaaagccagcagcgccaagcagagactgaagtgcgcctccctgcagaagttcggcg |
| | agagagcctttaaggcctgggccgtggccagattgagtcagagattccccaaggccgagtt |
| | cgccgaggtgagcaaactggtgaccgacctcactaaagtgcacacagaatgttgccatggc |
| | gatctcctggaatgcgccgatgacagggccgacctggccaagtacatctgtgagaaccagg |
| | acagcatttcgagcaagctgaaggagtgctgcgagaaacccctgctggagaagtcccactg |
| | catcgccgaggtggagaacgacgagatgcccgccgacctgcccagcctggccgcagatttc |
| | gtggagagcaaggacgtatgcaagaactacgcagaggccaaggatgtgttcctgggcatgt |
| | tcctgtacgaatacgccagaaggcaccccgactacagcgtcgtgctgttactgagactggc |
| | caagacctacgagacaaccttagagaagtgctgcgcggccgcagacccgcacgagtgctac |
| | gccaaggtgttcgacgagttcaagcccctggtggaggaacctcagaatctgattaagcaga |
| | attgtgagctgttcgagcagctgggagagtacaagttccagaacgcgctgctggtgagata |
| | taccaagaaggtgccccaggtgagcacccccaccctggtggaggtgagtcgcaacctgggc |
| | aaggtggggagcaagtgttgcaagcatcccgaggccaaaagaatgccctgcgcagaggact |
| | atctgagcgttgtgcttaaccagctgtgcgtgctgcacgagaagacccccgtgagcgacag |
| | agtgaccaagtgttgcaccgagagcctggtaaacagaagaccctgcttcagcgccctggag |
| | gtggacgagacctacgtgcccaaggagttcaacgccgagacctttaccttccacgcagaca |
| | tttgcaccctgagcgagaaagagaggcagatcaagaaacagaccgctctggtcgagcttgt |
| | gaagcacaagcccaaagctaccaaggagcagctgaaggccgtgatggatgatttcgccgcc |
| | ttcgtagagaaatgctgcaaggccgacgataaagagacctgctttgccgaggagggcaaga |
| | aactggtggccgccagccaggcggccctgggcctgtag |
| H3 Construct (SEQ | atgagagtgcccgcccagctgctgggcctgctgttgctgtggctgcccggcgccagatgcg |
| ID NO: 9)- | ccATCTGGGAACTGAAGAAGGACGTCTACGTGGTGGAGCTGGATTGGTACCCCGACGCCCC |
| includes (1) leader | CGGCGAGATGGTCGTGCTGACCTGTGACACCCCTGAGGAGGACGGAATCACATGGACCCTG |
| sequence (lower | GACCAGAGCAGCGAAGTGTTGGGCAGCGGCAAGACCCTGACCATCCAAGTGAAGGAATTCG |
| case; SEQ ID NO: | GCGACGCGGGCCAGTATACTTGCCACCAGGGCGGGGAGGTTCTGAGCCACAGCCTGCTGCT |
| 34); (2) IL-12ß | GCTCCACAAGAAAGAGGATGGCATCTGGTCTACCGACATCCTGAAGGACCAAAAGGAGCCA |
| (bolded and | AAGAACAAGACCTTCCTAAGATGCGAGGCCAAGAACTACTCAGGTCGTTTCACCTGCTGGT |
| underlined; SEQ ID | GGCTGACCACAATCTCCACCGACCTGACCTTCAGCGTGAAGAGCAGCCGCGGCAGTAGCGA |
| NO: 59); (3) first GS | CCCACAGGGCGTGACCTGCGGGGCCGCCACTCTGAGCGCCGAGAGAGTGCGCGGCGATAAT |
| linker (italicized; | AAGGAATACGAGTACAGCGTGGAGTGCCAGGAAGACTCAGCCTGCCCCGCCGCAGAAGAAA |
| SEQ ID NO: 84); | GTCTGCCAATAGAGGTGATGGTTGACGCCGTGCACAAGCTAAAGTACGAGAACTACACCAG |
| (4) IL-12α (bolded; | CAGCTTCTTTATCCGGGACATCATCAAGCCAGATCCCCCCAAGAACCTGCAGCTTAAGCCC |
| SEQ ID NO: 109); | CTGAAGAACAGCAGACAGGTGGAGGTGTCATGGGAATACCCCGACACCTGGAGCACCCCCC |
| (5) second GS linker | ATAGCTACTTCTCACTGACCTTCTGCGTGCAGGTGCAGGGCAAGAGCAAGAGAGAGAAGAA |
| (italicized and | GGATAGGGTATTCACGGACAAGACCTCAGCCACCGTGATCTGCCGAAAGAACGCCAGCATC |
| underlined; SEQ ID | AGCGTGAGAGCCCAGGACAGATACTATAGCTCCTCGTGGAGCGAGTGGGCCAGCGTACCTT |
| NO: 134); and (6) | GCAGCGGCGGGAGCGGCGGCGGCAGCGGCGGTGGCAGTGGCGGGGGCAGCAGAAACCTGCC |
| albumin (lower case | CGTGGCCACCCCCGACCCCGGGATGTTTCCTTGCCTGCACCACTCCCAGAACCTCCTGAGA |
| and underlined; SEQ | GCCGTGTCCAACATGCTGCAGAAAGCCAGACAGACCCTCGAGTTCTATCCCTGCACAAGCG |
| ID NO: 155) | AGGAGATCGACCACGAGGACATCACTAAGGACAAGACCAGCACAGTGGAAGCCTGCCTCCC |
| | GCTGGAGCTGACTAAGAACGAGAGCTGTCTGAACAGCAGGGAGACCAGCTTCATCACCAAC |
| | GGCAGCTGCCTGGCGAGCAGAAAAACCTCCTTTATGATGGCGCTCTGCCTGAGCTCAATCT |
| | ATGAGGACCTGAAGATGTACCAGGTGGAGTTTAAAACCATGAATGCCAAGCTGCTTATGGA |
| | CCCTAAGAGACAGATTTTCCTGGATCAGAACATGCTGGCCGTGATTGATGAATTAATGCAG |
| | GCGCTGAACTTTAACAGCGAGACCGTGCCCCAGAAGAGCAGCCTGGAGGAGCCCGACTTTT |
| | ACAAGACCAAGATCAAGTTGTGCATCCTCCTGCACGCCTTCAGAATCAGAGCGGTGACGAT |
| | CGACAGAGTCATGAGCTACCTCAACGCTAGCGGCAGCGGTGGCGGCAGCgacgcccacaag |
| | agcgaggtggcccacagattcaaggatctcggggaggagaacttcaaggccctggtgctga |
| | tcgccttcgcacagtacctgcagcagtgccccttcgaggaccacgtaaaactggtgaacga |
| | ggtgacggagttcgccaagacctgtgttgccgacgagtcggccgagaattgcgacaagagc |
| | ctgcataccctgttcggcgacaagctgtgcaccgtggccaccctgagagagacctacggcg |
| | agatggccgactgctgcgccaagcaagagcccgagagaaacgagtgcttcctgcagcacaa |
| | ggacgacaaccccaacctgccccggctggtgagacccgaggtggacgtgatgtgcaccgcc |
| | ttccacgacaacgaggagaccttcctgaagaagtatctgtacgagatcgccagaagacacc |
| | cttatttttacgcccccgagctgctcttcttcgctaagagatataaggcagccttcaccga |
| | gtgttgtcaggccgccgataaggccgcttgcctgctgcccaagttggacgagctcagagac |
| | gagggcaaggcgagcagcgccaagcagagactgaagtgcgccagcctgcagaagttcggcg |
| | agagggccttcaaggcctgggcggtggccagactgtcccagagatttcccaaggccgagtt |
| | cgccgaggtaagcaagctggtcaccgacctgaccaaggtgcacaccgagtgttgccacggc |
| | gacctgctggaatgcgccgatgaccgtgccgacctggccaagtacatctgcgagaatcagg |
| | actccatcagcagcaaactgaaggagtgttgcgagaagcccctgctggagaagagccattg |
| | catcgctgaggtggaaaacgacgagatgcccgcagacctgcccagcctggccgcagacttt |
| | gtggaaagtaaggacgtgtgcaagaactacgcggaggccaaagacgtgtttctgggcatgt |
| | tcctatacgagtatgccagaagacaccccgactacagcgttgtgttattgctgagactggc |
| | caagacctacgagactaccttggagaagtgctgcgccgccgccgacccccacgagtgctat |
| | gccaaggtgttcgacgagttcaagcccctggtggaggagccccagaatctgattaagcaga |
| | attgcgagctttttgagcagctgggcgagtataagttccagaacgccctgctggtgagata |
| | caccaaaaaggtacctcaggtgagcacccccaccctggtggaggtgagcagaaatctgggc |
| | aaggtgggcagcaagtgctgcaagcacccggaggccaagagaatgccctgtgccgaggatt |
| | acctgtcagtggtgctgaaccagctgtgcgttctgcacgaaaagacgcccgtgtcggacag |
| | agtgaccaagtgctgcacggagagcctggtgaacagaagaccgtgcttcagcgccctagag |
| | gtggacgagacctacgtgcccaaggagttcaacgccgagaccttcaccttccacgccgaca |
| | tctgtaccctgtcagagaaggagagacagatcaagaagcagaccgccttagtggagctggt |
| | gaagcacaagcccaaggccaccaaggagcagctgaaagccgtgatggacgatttcgcagcc |
| | ttcgtcgagaagtgctgcaaggccgacgacaaggaaacttgcttcgccgaggagggcaaga |
| | agctggtggctgcctcgcaggccgccctcggcctgtag |
| CO Construct (SEQ | atgagagtgcccgcccagctgctgggcctgctgctgctgtggctgcccggcgccagatgcg |
| ID NO: 10)- | ccATCTGGGAGCTGAAGAAGGACGTGTACGTGGTGGAGCTGGACTGGTACCCCGACGCCCC |
| includes (1) leader | CGGCGAGATGGTGGTGCTGACCTGCGACACCCCCGAGGAGGACGGCATCACCTGGACCCTG |
| sequence (lower | GACCAGAGCAGCGAGGTGCTGGGCAGCGGCAAGACCCTGACCATCCAGGTGAAGGAGTTCG |
| case; SEQ ID NO: | GCGACGCCGGCCAGTACACCTGCCACAAGGGCGGCGAGGTGCTGAGCCACAGCCTGCTGCT |
| 35); (2) IL-12ß | GCTGCACAAGAAGGAGGACGGCATCTGGAGCACCGACATCCTGAAGGACCAGAAGGAGCCC |
| (bolded and | AAGAACAAGACCTTCCTGAGATGCGAGGCCAAGAACTACAGCGGCAGATTCACCTGCTGGT |
| underlined; SEQ ID | GGCTGACCACCATCAGCACCGACCTGACCTTCAGCGTGAAGAGCAGCAGAGGCAGCAGCGA |
| NO: 60); (3) first GS | CCCCCAGGGCGTGACCTGCGGCGCCGCCACCCTGAGCGCCGAGAGAGTGAGAGGCGACAAC |
| linker (italicized; | AAGGAGTACGAGTACAGCGTGGAGTGCCAGGAGGACAGCGCCTGCCCCGCCGCCGAGGAGA |
| SEQ ID NO: 85); | GCCTGCCCATCGAGGTGATGGTGGACGCCGTGCACAAGCTGAAGTACGAGAACTACACCAG |
| (4) IL-12α (bolded; | CAGCTTCTTCATCAGAGACATCATCAAGCCCGACCCCCCCAAGAACCTGCAGCTGAAGCCC |
| SEQ ID NO: 110); | CTGAAGAACAGCAGACAGGTGGAGGTGAGCTGGGAGTACCCCGACACCTGGAGCACCCCCC |
| (5) second GS linker | ACAGCTACTTCAGCCTGACCTTCTGCGTGCAGGTGCAGGGCAAGAGCAAGAGAGAGAAGAA |
| (italicized and | GGACAGAGTGTTCACCGACAAGACCAGCGCCACCGTGATCTGCAGAAAGAACGCCAGCATC |
| underlined; SEQ ID | AGCGTGAGAGCCCAGGACAGATACTACAGCAGCAGCTGGAGCGAGTGGGCCAGCGTGCCCT |
| NO: 135); and (6) | GCAGCGGCGGCAGCGGCGGCGGCAGCGGCGGCGGCAGCGGCGGCGGCAGCAGAAACCTGCC |
| albumin (lower case | CGTGGCCACCCCCGACCCCGGCATGTTCCCCTGCCTGCACCACAGCCAGAACCTGCTGAGA |
| and underlined; SEQ | GCCGTGAGCAACATGCTGCAGAAGGCCAGACAGACCCTGGAGTTCTACCCCTGCACCAGCG |
| ID NO: 156) | AGGAGATCGACCACGAGGACATCACCAAGGACAAGACCAGCACCGTGGAGGCCTGCCTGCC |
| | CCTGGAGCTGACCAAGAACGAGAGCTGCCTGAACAGCAGAGAGACCAGCTTCATCACCAAC |
| | GGCAGCTGCCTGGCCAGCAGAAAGACCAGCTTCATGATGGCCCTGTGCCTGAGCAGCATCT |
| | ACGAGGACCTGAAGATGTACCAGGTGGAGTTCAAGACCATGAACGCCAAGCTGCTGATGGA |
| | CCCCAAGAGACAGATCTTCCTGGACCAGAACATGCTGGCCGTGATCGACGAGCTGATGCAG |
| | GCCCTGAACTTCAACAGCGAGACCGTGCCCCAGAAGAGCAGCCTGGAGGAGCCCGACTTCT |
| | ACAAGACCAAGATCAAGCTGTGCATCCTGCTGCACGCCTTCAGAATCAGAGCCGTGACCAT |
| | CGACAGAGTGATGAGCTACCTGAACGCCAGCGGCAGCGGCGGCGGCAGCgacgcccacaag |
| | agcgaggtggcccacagattcaaggacctgggcgaggagaacttcaaggccctggtgctga |
| | tcgccttcgcccagtacctgcagcagtgccccttcgaggaccacgtgaagctggtgaacga |
| | ggtgaccgagttcgccaagacctgcgtggccgacgagagcgccgagaactgcgacaagagc |
| | ctgcacaccctgttcggcgacaagctgtgcaccgtggccaccctgagagagacctacggcg |
| | agatggccgactgctgcgccaagcaggagcccgagagaaacgagtgcttcctgcagcacaa |
| | ggacgacaaccccaacctgcccagactggtgagacccgaggtggacgtgatgtgcaccgcc |
| | ttccacgacaacgaggagaccttcctgaagaagtacctgtacgagatcgccagaagacacc |
| | cctacttctacgcccccgagctgctgttcttcgccaagagatacaaggccgccttcaccga |
| | gtgctgccaggccgccgacaaggccgcctgcctgctgcccaagctggacgagctgagagac |
| | gagggcaaggccagcagcgccaagcagagactgaagtgcgccagcctgcagaagttcggcg |
| | agagagccttcaaggcctgggccgtggccagactgagccagagattccccaaggccgagtt |
| | cgccgaggtgagcaagctggtgaccgacctgaccaaggtgcacaccgagtgctgccacggc |
| | gacctgctggagtgcgccga gacagagccgacctggccaagtacatctgcgagaaccagg |
| | acagcatcagcagcaagctgaaggagtgctgcgagaagcccctgctggagaagagccactg |
| | catcgccgaggtggagaacgacgagatgcccgccgacctgcccagcctggccgccgacttc |
| | gtggagagcaaggacgtgtgcaagaactacgccgaggccaaggacgtgttcctgggcatgt |
| | tcctgtacgagtacgccagaagacaccccgactacagcgtggtgctgctgctgagactggc |
| | caagacctacgagaccaccctggagaagtgctgcgccgccgccgacccccacgagtgctac |
| | gccaaggtgttcgacgagttcaagcccctggtggaggagccccagaacctgatcaagcaga |
| | actgcgagctgttcgagcagctgggcgagtacaagttccagaacgccctgctggtgagata |
| | caccaagaaggtgccccaggtgagcacccccaccctggtggaggtgagcagaaacctgggc |
| | aaggtgggcagcaagtgctgcaagcaccccgaggccaagagaatgccctgcgccgaggact |
| | acctgagcgtggtgctgaaccagctgtgcgtgctgcacgagaagacccccgtgagcgacag |
| | agtgaccaagtgctgcaccgagagcctggtgaacagaagaccctgcttcagcgccctggag |
| | gtggacgagacctacgtgcccaaggagttcaacgccgagaccttcaccttccacgccgaca |
| | tctgcaccctgagcgagaaggagagacagatcaagaagcagaccgccctggtggagctggt |
| | gaagcacaagcccaaggccaccaaggagcagctgaaggccgtgatggacgacttcgccgcc |
| | ttcgtggagaagtgctgcaaggccgacgacaaggagacctgcttcgccgaggagggcaaga |
| | agctggtggccgccagccaggccgccctgggcctgtag |
| CP Construct (SEQ | atgagggtccccgctcagctcctggggctcctgctgctctggctcccaggtgcacgatgtg |
| ID NO: 11)- | ccATCTGGGAGCTGAAGAAGGATGTGTATGTGGTGGAGCTGGACTGGTACCCAGATGCCCC |
| includes (1) leader | TGGAGAGATGGTGGTGCTGACCTGTGACACCCCAGAGGAGGATGGCATCACCTGGACCCTG |
| sequence (lower | GACCAGAGCAGCGAGGTGCTGGGCAGCGGCAAGACCCTGACCATCCAGGTGAAGGAGTTTG |
| case; SEQ ID NO: | GCGACGCCGGCCAGTACACCTGCCACAAGGGCGGCGAGGTGCTGAGCCACAGCCTGCTGCT |
| 36); (2) IL-12ß | GCTGCACAAGAAGGAGGATGGCATCTGGAGCACAGACATCCTGAAGGACCAGAAGGAGCCC |
| (bolded and | AAGAACAAGACCTTCCTGAGATGCGAGGCCAAGAACTACAGCGGCAGATTCACCTGCTGGT |
| underlined; SEQ ID | GGCTGACCACCATCAGCACCGACCTGACCTTCAGCGTGAAGAGCAGCAGAGGCAGCAGCGA |
| NO: 61); (3) first GS | CCCCCAGGGCGTGACCTGCGGCGCCGCCACCCTGAGCGCCGAGAGAGTGAGAGGCGACAAC |
| linker (italicized; | AAGGAGTACGAGTACAGCGTGGAGTGCCAGGAGGACAGCGCCTGCCCCGCCGCCGAGGAGA |
| SEQ ID NO: 86); | GCCTGCCCATCGAGGTGATGGTGGACGCCGTGCACAAGCTGAAGTACGAGAACTACACCAG |
| (4) IL-12α (bolded; | CAGCTTCTTCATCAGAGACATCATCAAGCCCGACCCCCCCAAGAACCTGCAGCTGAAGCCC |
| SEQ ID NO: 111); | CTGAAGAACAGCAGACAGGTGGAGGTGAGCTGGGAGTACCCCGACACCTGGAGCACCCCCC |
| (5) second GS linker | ACAGCTACTTCAGCCTGACCTTCTGCGTGCAGGTGCAGGGCAAGAGCAAGAGAGAGAAGAA |
| (italicized and | GGACAGAGTGTTCACCGACAAGACCAGCGCCACCGTGATCTGCAGAAAGAACGCCAGCATC |
| underlined; SEQ ID | AGCGTGAGAGCCCAGGACAGATACTACAGCAGCAGCTGGAGCGAGTGGGCCAGCGTGCCCT |
| NO: 136); and (6) | GCAGCGGCGGCAGCGGCGGCGGCAGCGGCGGCGGCAGCGGCGGCGGCAGCAGAAACCTGCC |
| albumin (lower case | CGTGGCCACCCCCGACCCCGGCATGTTCCCCTGCCTGCACCACAGCCAGAACCTGCTGCGG |
| and underlined; SEQ | GCTGTGAGCAACATGCTGCAGAAGGCCCGGCAGACCCTGGAGTTCTACCCCTGCACCAGCG |
| ID NO: 157) | AGGAGATTGACCACGAGGACATCACCAAGGACAAGACCAGCACAGTGGAGGCCTGCCTGCC |
| | CCTGGAGCTGACCAAGAATGAAAGCTGCCTGAACAGCCGGGAGACCAGCTTCATCACCAAC |
| | GGCAGCTGCCTGGCCAGCAGGAAGACCAGCTTCATGATGGCCCTGTGCCTGAGCAGCATCT |
| | ATGAGGACCTGAAGATGTACCAGGTGGAGTTCAAGACCATGAATGCCAAGCTGCTGATGGA |
| | CCCCAAGAGGCAGATCTTCCTGGACCAGAACATGCTGGCCGTGATTGATGAGCTGATGCAG |
| | GCCCTGAACTTCAACAGCGAGACCGTGCCCCAGAAAAGCAGCCTGGAGGAGCCAGACTTCT |
| | ACAAGACCAAGATCAAGCTGTGCATCCTGCTGCACGCCTTCCGCATCCGGGCTGTGACCAT |
| | CGACAGAGTGATGAGCTACCTGAATGCCAGCGGCAGCGGCGGCGGCAGTgatgcccacaag |
| | tctgaggtggcccaccgcttcaaggacctgggggaggagaacttcaaggccctggtgctga |
| | ttgcctttgcccagtacctgcagcagtgcccctttgaggaccacgtgaagctggtgaatga |
| | ggtgacagaatttgccaagacctgtgtggctgatgaatctgctgagaactgtgacaagagc |
| | ctgcacaccctgtttggagacaagctgtgcaccgtggccaccctgcgggagacctatggag |
| | agatggctgactgctgtgccaagcaggagcctgagagaaatgaatgcttcctgcagcacaa |
| | ggatgacaaccccaacctgccccggctggtgcggcctgaggtggatgtgatgtgcacagcc |
| | ttccatgacaatgaggagaccttcctgaagaagtacctgtatgaaattgcccggcggcacc |
| | cctacttctacgcccctgagctgctgttctttgccaagcgctacaaggccgccttcacaga |
| | gtgctgccaggccgctgacaaggccgcctgcctgctgcccaagctggatgagctgagagat |
| | gagggcaaggccagcagcgccaagcagaggctgaagtgtgccagcctgcagaagtttggag |
| | agcgggccttcaaggcctgggccgtggcccggctgagccagcgcttccccaaggccgagtt |
| | tgctgaggtgtccaagctggtgacagacctgaccaaggtgcacacagagtgctgccacggg |
| | gacctgctggagtgtgctgatgacagagctgacctggccaagtacatctgtgagaaccagg |
| | acagcatcagcagcaagctgaaggagtgctgtgagaagcccctgctggaaaagagccactg |
| | catcgccgaggtggagaatgatgagatgcctgctgacctgcccagcctggccgctgacttt |
| | gtggagagcaaggatgtgtgcaagaactacgccagggccaaggatgtgttcctgggcatgt |
| | tcctgtacgagtacgccagaagacaccccgactacagcgtggtgctgctgctgagactggc |
| | caagacctatgagaccaccctggagaagtgctgtgccgctgctgacccccatgaatgttat |
| | gccaaggtgtttgatgagttcaagcccctggtggaggagccccagaacctgatcaagcaga |
| | actgtgagctgtttgagcagctgggggagtacaagttccagaatgccctgctggtgcgcta |
| | caccaagaaggtgccccaggtgtccacccccaccctggtggaggtgtccaggaacctgggc |
| | aaggtgggcagcaagtgctgcaagcaccctgaggccaagaggatgccctgtgccgaggact |
| | acctgtctgtggtgctgaaccagctgtgtgtgctgcacgagaagacccctgtgtctgacag |
| | agtgaccaagtgctgcacagagagcctggtgaacagaagaccctgcttcagcgccctggag |
| | gtggatgagacctacgtgcccaaggagttcaatgctgagaccttcaccttccacgccgaca |
| | tctgcaccctgtctgagaaggagcggcagatcaagaagcagacagccctggtggagctggt |
| | gaagcacaagcccaaggccaccaaggagcagctgaaggctgtgatggatgactttgctgcc |
| | tttgtggagaagtgctgcaaggcagatgacaaggagacctgctttgctgaggagggcaaga |
| | agctggtggccgccagccaggccgccctgggcctg |
| vH1 Construct (SEQ | atgagagtgcccgcccagctgctgggcctgctgctgctgtggctgcccggcgccagatgcg |
| ID NO: 12)- | ccATCTGGGAGCTGAAGAAGGACGTGTACGTGGTGGAGCTGGACTGGTACCCCGACGCCCC |
| includes (1) leader | CGGCGAGATGGTGGTGCTGACCTGCGACACCCCCGAGGAGGACGGGATCACCTGGACCCTG |
| case; SEQ ID NO: | GATCAGAGCAGCGAGGTGCTGGGCAGCGGCAAGACCCTGACCATCCAGGTGAAGGAGTTTG |
| 37); (2) IL-12ß | GTGACGCCGGCCAGTACACCTGCCACAAGGGCGGCGAGGTGCTGAGCCACTCACTGCTGCT |
| (bolded and | GCTGCACAAGAAGGAGGACGGCATCTGGAGCACCGACATTCTGAAGGATCAGAAGGAGCCC |
| underlined; SEQ ID | AAGAACAAGACCTTCCTGAGATGCGAGGCCAAGAACTACAGCGGCAGATTCACCTGTTGGT |
| NO: 62); (3) first GS | GGCTGACTACTATCAGCACTGATCTGACCTTCAGCGTGAAGAGCTCAAGAGGCAGCAGCGA |
| linker (italicized; | TCCCCAGGGCGTGACCTGCGGCGCCGCCACCCTGAGCGCCGAGAGAGTGAGAGGCGACAAC |
| SEQ ID NO: 87); | AAGGAGTACGAGTACAGCGTGGAGTGCCAGGAAGACAGCGCCTGCCCCGCTGCAGAGGAGT |
| (4) IL-12α (bolded; | CTCTGCCCATCGAGGTGATGGTGGACGCCGTGCACAAGCTTAAGTACGAGAACTACACCAG |
| SEQ ID NO: 112); | CTCCTTCTTCATTAGAGACATCATCAAGCCCGACCCGCCCAAAAACCTGCAGCTGAAGCCC |
| (5) second GS linker | CTGAAGAACAGCAGACAGGTGGAGGTCAGCTGGGAGTACCCCGACACCTGGAGCACCCCCC |
| (italicized and | ACAGCTACTTCAGCCTGACCTTCTGCGTGCAGGTGCAGGGCAAGAGCAAGAGAGAGAAGAA |
| underlined; SEQ ID | GGACAGAGTGTTCACCGACAAAACCAGCGCCACCGTGATCTGCAGAAAAAACGCCAGCATT |
| NO: 137); and (6) | AGCGTGAGAGCTCAGGATAGATACTACAGCAGCAGCTGGAGTGAGTGGGCCAGCGTGCCCT |
| albumin (lower case | GCAGCGGCGGCTCAGGCGGCGGCTCAGGCGGCGGCAGCGGCGGCGGCAGCAGAAACCTGCC |
| and underlined; SEQ | CGTGGCCACGCCCGACCCCGGCATGTTTCCCTGCCTGCACCATAGCCAGAATCTGCTGAGA |
| ID NO: 158) | GCCGTGAGCAACATGCTGCAGAAGGCCAGACAGACGCTCGAGTTCTACCCCTGCACCAGCG |
| | AGGAGATTGACCACGAGGACATCACCAAGGACAAGACCAGCACCGTGGAGGCCTGCCTGCC |
| | CCTCGAGCTGACCAAGAACGAGAGCTGCCTGAACAGCAGAGAGACCAGCTTCATCACCAAC |
| | GGCAGCTGCCTGGCCAGCAGAAAGACCAGCTTCATGATGGCCCTGTGCCTGAGCAGCATCT |
| | ACGAGGACCTGAAGATGTACCAGGTGGAGTTCAAGACAATGAACGCCAAGCTGCTGATGGA |
| | CCCCAAGAGGCAGATTTTTCTGGACCAGAACATGCTGGCCGTTATCGACGAGCTGATGCAG |
| | GCCCTGAATTTCAATAGCGAAACCGTGCCCCAGAAGAGCAGCCTGGAGGAGCCTGACTTCT |
| | ACAAAACCAAGATCAAGCTTTGCATCCTGCTGCACGCCTTCAGAATCAGAGCCGTGACCAT |
| | CGACAGAGTGATGAGCTACCTGAACGCCAGCGGCAGCGGCGGCGGCAGCgacgcccacaag |
| | agcgaggtggcccataggttcaaggacctgggcgaggagaacttcaaggccctggtactca |
| | tcgccttcgcccaatacctgcaacagtgccccttcgaggaccatgttaagctggtgaacga |
| | ggtgaccgagttcgccaagacctgcgtggccgacgagagcgccgagaactgcgacaagagc |
| | ctgcacaccctgttcggcgacaagctgtgcaccgtggccaccctgagagagacctacggcg |
| | agatggccgactgctgcgccaagcaggagcccgaacgtaacgagtgcttcctgcagcacaa |
| | ggacgacaaccccaacctgccccgactggtcagacccgaggtggacgtgatgtgcacagcc |
| | ttccacgacaacgaggagaccttcctgaagaagtacctctacgagatcgccagaagacatc |
| | catacttctacgcccccgagctgctgttcttcgccaagaggtacaaggccgccttcacaga |
| | gtgctgccaggccgccgacaaggccgcttgcctgctgcctaagttggacgagctgagagac |
| | gagggcaaggccagcagcgccaagcagagactgaagtgcgcaagcctgcagaaattcggcg |
| | agagagcctttaaggcctgggccgtggccagactgagccagcgcttccccaaggccgagtt |
| | cgccgaggtgagcaagctggtgaccgacctgaccaaagtgcacaccgagtgctgccacggc |
| | gacctgctggagtgcgccgacgacagagccgacctggccaagtacatctgcgagaaccagg |
| | acagcatcagcagcaagttgaaggagtgctgcgagaagcccctgctggagaagagccactg |
| | catcgccgaggtggagaacgacgagatgcccgccgacctgcccagcctggccgccgacttc |
| | gtggagagcaaggacgtgtgcaagaactacgccgaggccaaggacgtgttcctgggcatgt |
| | tcctgtacgagtacgcccggagacaccccgactacagcgtggtgctgctgctgagactggc |
| | caagacctacgagaccaccctggagaagtgctgtgccgccgccgacccccacgagtgctac |
| | gccaaggtgttcgacgagttcaagcccctggtggaggagccccagaacctgatcaagcaga |
| | actgcgagctgttcgagcagctgggcgagtataagttccagaacgccctgctggtgagata |
| | caccaagaaggtgccccaggtcagcacccccaccctggtggaggtgagcagaaatctgggc |
| | aaggtgggcagcaaatgctgcaagcaccccgaggccaagagaatgccctgcgccgaggact |
| | acctgtcagtggtgctgaaccagctgtgtgtgctgcacgagaagacccccgtgagcgacag |
| | agtgaccaagtgctgcaccgagagtctcgtgaacagacggccctgcttcagcgccctggag |
| | gtggacgaaacatacgtgcccaaggagttcaacgcagagaccttcaccttccacgcagaca |
| | tctgcaccctgagcgagaaggagagacagatcaagaagcagaccgccctggttgagcttgt |
| | gaagcacaagcccaaggccaccaaggagcagctgaaggccgtgatggacgacttcgccgcc |
| | ttcgtggagaagtgctgcaaggccgacgacaaggagacctgcttcgccgaggagggcaaga |
| | agctggtggccgccagccaggccgccctgggcctgtag |
| vH2 Construct (SEQ | atgagagtgcccgcccagctgctgggcctgctgctgctgtggctgcccggcgccagatgtg |
| ID NO: 13)- | ccATCTGGGAGCTGAAGAAGGACGTGTACGTGGTGGAGCTGGACTGGTACCCCGACGCCCC |
| includes (1) leader | CGGAGAGATGGTGGTGCTGACCTGCGAGACCCCCGAAGAGGACGGCATCACCTGGACCCTG |
| sequence (lower | GACCAGAGCAGCGAGGTGCTGGGCAGCGGGAAGACCCTGACCATCCAGGTGAAGGAGTTCT |
| case; SEQ ID NO: | GCGACGCCGGCCAGTACACCTGTCACAAGGGCGGCGAGGTGCTGAGCCACAGCCTGCTGCT |
| 38); (2) IL-12ß | GCTGCACAAGAAGGAGGACGGCATATGGAGCACCGACATCCTGAAGGACCAGAAGGAGCCC |
| (bolded and | AAGAACAAGACCTTTCTGAGATGCGAGGCTAAGAATTACAGCGGCAGATTCACCTGCTGGT |
| underlined; SEQ ID | GGCTGACCACCATCAGCACCGACCTGACCTTCAGCGTGAAGAGCAGCAGAGGCAGCAGCGA |
| NO: 63); (3) first GS | CCCCCAGGGCGTGACATGCGGCGCCGCCACCCTGAGCGCCGAGAGAGTGAGAGGCGACAAC |
| linker (italicized; | AAGGAGTACGAGTACAGCGTGGAGTGCCAGGAGGACTCCGCTTGCCCCGCCGCCGAGGAGA |
| SEQ ID NO: 88); | GCTTGCCCATCGAGGTGATGGTGGACGCCGTGCACAAGCTCAAGTACGAAAACTACACCAG |
| (4) IL-12α (bolded; | CAGCTTCTTCATCAGAGACATCATCAAGCCCGACCCCCCCAAGAACCTGCAGCTGAAGCCC |
| SEQ ID NO: 113); | CTGAAAAACAGCAGACAGGTGGAGGTGAGCTGGGAGTACCCCGACACCTGGAGCACCCCCC |
| (5) second GS linker | ACAGCTACTTCAGCCTGACCTTCTGCGTGCAGGTGCAGGGAAAAAGCAAGAGAGAGAAGAA |
| (italicized and | GGACAGAGTGTTCACCGACAAGACCAGCGCCACCGTGATCTGCAGAAAGAACGCCAGCATC |
| underlined; SEQ ID | TCCGTGAGAGCCCAGGACAGATACTACAGCAGCAGCTGGAGCGAGTGGGCCAGCGTGCCCT |
| NO: 138); and (6) | GTAGCGGCGGCAGCGGCGGCGGCAGCGGCGGCGGCAGCGGCGGCGGCAGCAGAAACCTGCC |
| albumin (lower case | CGTGGCCACCCCCGACCCCGGCATGTTCCCCTGCCTGCACCACAGCCAGAACCTGCTGAGA |
| and underlined; SEQ | GCCGTGAGCAACATGCTGCAGAAGGCCAGACAGACCCTGGAGTTCTACCCCTGCACCAGCG |
| ID NO: 159) | AGGAGATCGACCATGAGGACATCACCAAGGACAAGACCAGCACCGTGGAGGCCTGCCTGCC |
| | CCTGGAGCTGACCAAGAACGAAAGCTGCCTGAACAGCAGAGAGACCAGCTTCATCACCAAC |
| | GGCAGCTGTCTGGCCTCAAGAAAGACCAGCTTTATGATGGCCCTGTGCCTGTCTAGCATCT |
| | ACGAGGATCTGAAGATGTACCAGGTGGAGTTCAAGACCATGAACGCCAAGCTGCTGATGGA |
| | CCCCAAGAGACAGATCTTCCTGGACCAGAACATGTTAGCCGTGATTGACGAGCTGATGCAG |
| | GCCCTGAACTTCAATAGCGAGACCGTGCCCCAGAAAAGCAGCCTGGAGGAGCCCGACTTCT |
| | ACAAGACCAAGATCAAGCTGTGCATCCTGCTGCACGCCTTCAGAATCAGAGCCGTAACCAT |
| | CGACAGAGTGATGAGCTACCTGAACGCCAGTGGCAGCGGCGGTGGCAGCgacgctcacaag |
| | agcgaggtggcccacagattcaaggacctgggcgaggagaacttcaaggccctggtcctga |
| | tcgccttcgcccagtacctgcagcagtgccccttcgaggaccacgtgaagctggtgaacga |
| | ggtgaccgagttcgccaagacctgcgtggccgacgagtcggccgagaactgcgacaagagc |
| | ctgcacaccctgttcggcgacaagctgtgcaccgtggccaccctgagagaaacctacgggg |
| | agatggccgactgctgcgcaaagcaggagcccgagcgaaacgagtgcttcctgcagcacaa |
| | ggacgacaaccccaacttgcccagactggtaagacccgaggtggacgtcatgtgcaccgct |
| | ttccacgacaacgaggagaccttcctgaagaagtacctgtacgagatcgccagaagacacc |
| | cctatttctatgcccctgagctgctgttcttcgccaagcgctacaaggccgccttcaccga |
| | gtgctgccaggccgccgacaaggccgcctgcctgctccccaagctggacgagctgagagac |
| | gaaggcaaggccagcagcgccaagcagagactgaagtgcgccagcctgcagaagttcggcg |
| | agagagccttcaaggcctgggccgtggccagactgtcgcagagattccccaaggccgagtt |
| | cgccgaggtgagcaagctggttaccgacctgactaaggtgcacaccgagtgctgccacggc |
| | gacctgctggagtgcgccgacgacagagccgacctggccaagtacatctgcgagaaccagg |
| | atagcatcagcagcaagctgaaggagtgctgcgagaagccccttctggagaagtcccactg |
| | catcgccgaggtggagaacgacgagatgcccgccgacctgcctagcctggccgccgacttc |
| | gtggagagcaaggacgtgtgcaagaactacgccgaggccaaggacgtgttcctgggcatgt |
| | tcctgtacgagtacgccagaagacaccccgactacagcgtggtgctgctgctgagactggc |
| | caagacctacgagactacccttgagaagtgctgcgccgccgccgacccacatgagtgctac |
| | gccaaggtgttcgacgagttcaagcccctggtggaagagccccagaacctgatcaagcaga |
| | actgcgagctgttcgagcagctgggcgagtacaagttccagaacgcccttctggtgagata |
| | caccaagaaggtgcctcaggtgagcacccccacccttgtggaggtgagcagaaacctcggc |
| | aaggtgggcagcaagtgctgcaagcatccagaggccaagagaatgccctgcgccgaggact |
| | acctgagcgtggtgctgaaccagctgtgcgtgctgcacgagaaaactcccgtgagcgacag |
| | agtgaccaagtgctgcaccgagagtctggtgaacagaagaccctgcttcagcgccctggag |
| | gtggacgagacctacgtgcccaaggagttcaacgccgagaccttcaccttccacgccgaca |
| | tctgcaccctgagcgagaaggagcggcagatcaagaagcagaccgccctggtggagctggt |
| | gaagcacaagcccaaggccaccaaggagcagctcaaggccgtgatggacgacttcgccgcg |
| | ttcgtggagaagtgctgcaaggccgacgacaaagagacctgcttcgccgaggagggcaaga |
| | agcttgtggccgccagccaggccgccctgggcctgtag |
| vH3 Construct (SEQ | atgagagtgcccgcccagcttctgggcctgctgctgctgtggctgcccggcgccagatgcg |
| ID NO: 14)- | ccATCTGGGAGCTGAAGAAGGACGTGTACGTGGTGGAGCTGGACTGGTACCCGGACGCCCC |
| includes (1) leader | GGGCGAGATGGTCGTGCTGACCTGCGACACCCCCGAGGAGGACGGCATCACCTGGACCCTG |
| sequence (lower | GACCAGTCTAGCGAGGTGCTGGGCAGCGGCAAGACCCTGACCATCCAGGTGAAGGAGTTCG |
| case; SEQ ID NO: | GCGACGCCGGCCAGTACACCTGCCACAAGGGCGGCGAGGTGCTGAGCCACAGCCTGCTGCT |
| 39); (2) IL-12ß | GCTGCACAAGAAGGAGGACGGCATCTGGAGCACCGACATCCTGAAGGACCAGAAGGAGCCT |
| (bolded and | AAGAACAAGACCTTCCTGAGATGTGAGGCCAAGAACTACAGCGGCAGATTCACCTGCTGGT |
| underlined; SEQ ID | GGCTGACCACCATCAGCACCGATCTGACCTTCAGCGTGAAGAGCAGCAGAGGCAGCTCAGA |
| NO: 64); (3) first GS | CCCCCAGGGCGTGACCTGCGGCGCCGCGACCCTGAGCGCCGAGAGAGTGAGAGGCGACAAC |
| linker (italicized; | AAGGAGTACGAGTACAGCGTGGAGTGCCAGGAGGACAGCGCCTGCCCCGCGGCCGAGGAGA |
| SEQ ID NO: 89); | GCCTGCCCATCGAGGTGATGGTGGACGCCGTGCATAAGCTGAAGTACGAGAACTACACCAG |
| (4) IL-12α (bolded; | CAGCTTCTTCATCAGAGACATCATCAAGCCCGATCCCCCCAAGAACCTGCAGCTGAAGCCA |
| SEQ ID NO: 114); | CTGAAGAACAGCAGACAGGTGGAGGTGAGCTGGGAGTACCCCGACACCTGGAGCACCCCCC |
| (5) second GS linker | ACAGCTACTTCAGCCTGACCTTCTGCGTGCAGGTGCAGGGCAAGAGCAAGAGAGAGAAGAA |
| (italicized and | GGACCGGGTGTTCACCGACAAGACCAGCGCCACTGTGATCTGCAGAAAGAACGCCAGCATC |
| underlined; SEQ ID | AGCGTGAGAGCCCAGGACAGATACTACAGCTCCAGCTGGTCAGAGTGGGCCAGCGTGCCCT |
| NO: 139); and (6) | GCAGCGGCGGCAGCGGTGGCGGGAGCGGCGGCGGGAGCGGCGGCGGAAGCAGAAACCTGCC |
| albumin (lower case | CGTGGCCACCCCTGACCCCGGCATGTTCCCCTGCCTGCACCACAGCCAGAACCTGCTGAGA |
| and underlined; SEQ | GCCGTAAGCAACATGCTGCAGAAGGCCAGACAGACTCTGGAGTTCTACCCCTGCACCAGCG |
| ID NO: 160) | AGGAGATCGATCACGAGGACATCACCAAGGACAAGACCAGTACCGTGGAGGCCTGCCTGCC |
| | CCTGGAGCTGACCAAGAACGAGAGCTGCCTGAACAGCAGAGAGACCAGCTTTATAACCAAC |
| | GGCAGCTGTCTGGCTAGCAGAAAGACCAGCTTCATGATGGCCCTGTGCCTGAGCAGCATCT |
| | ACGAGGACTTGAAGATGTACCAGGTGGAGTTCAAGACCATGAACGCCAAGCTTCTGATGGA |
| | CCCCAAGAGACAGATCTTTCTGGACCAGAACATGCTGGCCGTGATCGATGAACTGATGCAG |
| | GCCCTGAACTTCAACAGCGAGACCGTGCCCCAGAAGAGCAGTCTGGAGGAGCCCGACTTCT |
| | ACAAGACTAAGATCAAGCTGTGCATCCTGCTGCACGCCTTCAGAATCAGAGCCGTGACCAT |
| | CGACAGAGTGATGAGCTACCTGAACGCCTCAGGCAGCGGCGGCGGGAGCgacgcccacaag |
| | agcgaggtggcccacagattcaaggacctcggcgaggagaacttcaaggccctggtgctga |
| | tcgctttcgcccagtacctgcagcagtgccccttcgaggaccacgtgaaactggtgaacga |
| | ggtgaccgagttcgccaagacctgcgtggccgacgagagcgccgagaactgcgacaagagc |
| | ctgcacacgttgttcggcgacaagctgtgcaccgtggccaccctgagagagacctacggcg |
| | agatggccgactgctgcgccaagcaggagcccgagagaaacgaatgcttcctgcagcacaa |
| | ggacgacaaccccaacctgccccgcctggtgagacccgaggtggacgtgatgtgcaccgcc |
| | ttccacgacaacgaggagaccttcctgaagaagtacttgtacgagatcgcaagaagacacc |
| | cgtacttctacgcccccgagctgctgttcttcgccaagagatacaaggccgccttcaccga |
| | gtgctgccaggccgccgacaaggccgcctgcctgctgcccaagctggacgagctcagagac |
| | gagggcaaggccagcagcgccaagcagagactgaagtgcgccagcctgcagaagttcggcg |
| | agagagcctttaaggcctgggccgtggccagactgagccagagattccccaaggccgagtt |
| | cgccgaagtgagcaagctggtgaccgacctgacgaaggtgcacaccgagtgctgccacggc |
| | gacctgctggagtgcgccgacgacagagcagacctggccaagtacatctgcgagaaccagg |
| | acagcatcagcagcaagctgaaggagtgctgcgagaagcccctgctggagaagagccactg |
| | catcgccgaggtggagaacgacgagatgcccgccgacctgcccagtctggccgcagacttc |
| | gtggagagcaaggatgtgtgcaagaactacgccgaggccaaggacgtgttcctgggcatgt |
| | tcctgtacgagtacgcaagaagacaccccgactacagcgtggtgctgctgctgagactggc |
| | caagacctacgagaccaccctggagaagtgctgcgccgccgccgacccccacgagtgctac |
| | gccaaggtgttcgacgagttcaagcccctggtggaggagccccagaacctgatcaagcaga |
| | actgcgaactgttcgagcagcttggcgagtacaagtttcagaacgccctgctggtcagata |
| | caccaagaaggtgccccaggtgagcacccccaccctggtggaggtgtccagaaacctgggc |
| | aaggtgggcagcaagtgctgcaagcaccccgaggcaaagagaatgccctgcgccgaggact |
| | atctgagcgtggtgctgaaccagctgtgcgtgctgcacgagaagacccccgtgagcgacag |
| | agtgaccaagtgctgcaccgagagcctggtgaatagaagaccctgcttcagcgcactggag |
| | gtggacgagacctacgtgcccaaggagttcaacgccgagacattcaccttccacgccgata |
| | tctgcaccctgagcgagaaggagagacagatcaagaagcagaccgccctggtggagctggt |
| | gaagcacaagcccaaggccaccaaggagcagttgaaggccgtgatggacgacttcgccgcc |
| | ttcgtggagaaatgctgcaaggccgacgacaaggagacctgcttcgccgaggagggcaaga |
| | agctggtggccgccagccaggccgccctgggcctgtag |
| A1 Construct (SEQ | atgagggtccccgctcagctcctggggctcctgctgctctggctcccaggtgcacgatgtg |
| ID NO: 15)- | ccATCTGGGAGCTGAAGAAAGACGTGTACGTGGTGGAGCTTGACTGGTACCCCGACGCCCC |
| includes (1) leader | CGGTGAAATGGTGGTTCTGACCTGCGACACACCAGAGGAGGACGGCATCACCTGGACCCTG |
| sequence (lower | GACCAGTCCAGCGAGGTCCTGGGATCTGGCAAGACCCTGACCATCCAGGTTAAGGAATTTG |
| case; SEQ ID NO: | GCGACGCCGGCCAGTACACCTGCCACAAAGGCGGCGAGGTCCTTTCGCACAGTCTGCTGCT |
| 40); (2) IL-12ß | GCTGCATAAAAAGGAGGACGGCATTTGGAGCACCGACATTCTGAAGGATCAGAAAGAGCCC |
| (bolded and | AAGAACAAGACCTTTCTGAGATGTGAGGCCAAAAACTACTCTGGACGCTTCACCTGTTGGT |
| underlined; SEQ ID | GGCTGACCACCATCAGCACAGACCTGACCTTCTCGGTGAAGTCTAGTAGGGGCAGCAGTGA |
| NO: 65); (3) GS | CCCCCAGGGCGTAACATGCGGCGCCGCTACCCTGAGCGCCGAGAGAGTGAGAGGCGATAAC |
| linker (italicized; | AAGGAGTACGAGTACTCCGTGGAGTGCCAAGAGGACTCAGCCTGCCCCGCCGCCGAGGAGT |
| SEQ ID NO: 90); | CGCTGCCCATCGAGGTGATGGTGGATGCAGTGCACAAGCTGAAGTACGAGAACTACACCAG |
| and (4) IL-12α | TAGCTTTTTCATCAGAGATATCATTAAACCCGACCCTCCCAAGAACCTGCAGCTGAAGCCC |
| (bolded; SEQ ID | TTAAAGAACAGCCGGCAGGTGGAAGTGTCATGGGAGTACCCAGACACCTGGAGCACTCCGC |
| NO: 115) | ACAGCTACTTCAGCCTGACCTTCTGCGTTCAGGTGCAGGGAAAAAGCAAGAGAGAGAAGAA |
| | AGACAGAGTGTTCACCGACAAGACCAGCGCAACCGTGATCTGTAGAAAGAACGCCTCGATC |
| | AGCGTGAGAGCCCAGGACAGATACTACAGCAGCAGCTGGAGCGAGTGGGCCAGTGTACCTT |
| | GCAGCGGTGGAAGCGGCGGAGGCTCTGGCGGAGGGAGTGGCGGAGGCAGCAGAAATCTTCC |
| | AGTAGCTACCCCCGACCCCGGCATGTTTCCCTGTCTGCACCATTCTCAAAACCTGTTACGG |
| | GCCGTGAGCAACATGCTGCAGAAGGCCAGACAGACACTGGAGTTTTACCCCTGTACCTCAG |
| | AGGAGATCGATCATGAGGACATTACTAAGGACAAGACCAGCACCGTGGAAGCCTGCCTGCC |
| | CCTAGAGCTAACCAAGAACGAGAGCTGCCTGAACTCTAGAGAGACAAGCTTCATCACGAAC |
| | GGCTCATGCCTGGCCAGTAGGAAAACCAGCTTCATGATGGCTCTGTGCCTGAGCTCCATAT |
| | ATGAGGACCTTAAGATGTACCAGGTGGAGTTCAAAACCATGAACGCCAAGCTGCTGATGGA |
| | CCCAAAGAGACAGATCTTCCTTGACCAGAACATGCTGGCCGTTATCGATGAGCTGATGCAG |
| | GCCCTGAACTTCAACAGCGAGACCGTGCCCCAGAAAAGCAGCCTGGAGGAACCCGACTTCT |
| | ACAAAACCAAGATCAAGCTGTGCATTCTGCTGCATGCCTTCCGCATTAGAGCCGTGACCAT |
| | CGATAGGGTGATGAGCTACCTGAACGCCAGC |
| A2 Construct (SEQ | atgagggtccccgctcagctcctggggctcctgctgctctggctcccaggtgcacgatgtg |
| ID NO: 16)- | ccATCTGGGAACTGAAGAAGGACGTCTACGTGGTGGAGCTGGATTGGTACCCCGACGCCCC |
| includes (1) leader | CGGCGAGATGGTCGTGCTGACCTGTGACACCCCTGAGGAGGACGGAATCACATGGACCCTG |
| sequence (lower | GACCAGAGCAGCGAAGTGTTGGGCAGCGGCAAGACCCTGACCATCCAAGTGAAGGAATTCG |
| case; SEQ ID NO: | GCGACGCGGGCCAGTATACTTGCCACAAGGGCGGGGAGGTTCTGAGCCACAGCCTGCTGCT |
| 41); (2) IL-12ß | GCTCCACAAGAAAGAGGATGGCATCTGGTCTACCGACATCCTGAAGGACCAAAAGGAGCCA |
| (bolded and | AAGAACAAGACCTTCCTAAGATGCGAGGCCAAGAACTACTCAGGTCGTTTCACCTGCTGGT |
| underlined; SEQ ID | GGCTGACCACAATCTCCACCGACCTGACCTTCAGCGTGAAAAGCAGCCGCGGCAGTAGCGA |
| NO: 66); (3) GS | CCCACAGGGCGTGACCTGCGGGGCCGCCACTCTGAGCGCCGAGAGAGTGCGCGGCGATAAT |
| linker (italicized; | AAGGAATACGAGTACAGCGTGGAGTGCCAGGAAGACTCAGCCTGCCCCGCCGCAGAAGAAA |
| SEQ ID NO: 91); | GTCTGCCAATAGAGGTGATGGTTGACGCCGTGCACAAGCTAAAGTACGAGAACTACACCAG |
| and (4) IL-12α | CAGCTTCTTTATCCGGGACATCATCAAGCCAGATCCCCCCAAGAACCTGCAGCTTAAGCCC |
| (bolded; SEQ ID | CTGAAGAACAGCAGACAGGTGGAGGTGTCATGGGAATACCCCGACACCTGGAGCACCCCCC |
| NO: 116) | ATAGCTACTTCTCACTGACCTTCTGCGTGCAGGTGCAGGGCAAGAGCAAGAGAGAGAAGAA |
| | GGATAGGGTATTCACGGACAAGACCTCAGCCACCGTGATCTGCCGAAAGAACGCCAGCATC |
| | AGCGTGAGAGCCCAGGACAGATACTATAGCTCCTCGTGGAGCGAGTGGGCCAGCGTACCTT |
| | GCAGCGGCGGGAGCGGCGGCGGCAGCGGAGGTGGCAGTGGCGGAGGCAGCAGAAACCTCCC |
| | CGTGGCAACCCCTGACCCCGGGATGTTTCCTTGCCTGCACCACTCCCAGAACCTCCTGAGA |
| | GCCGTGTCCAACATGCTGCAGAAAGCCAGACAGACCCTCGAGTTCTATCCCTGCACAAGCG |
| | AGGAGATCGACCACGAGGACATCACTAAGGACAAGACCAGCACAGTGGAAGCCTGCCTCCC |
| | GCTGGAGCTGACTAAGAACGAGAGCTGTCTGAACAGCAGGGAGACCAGCTTCATCACCAAC |
| | GGCAGCTGCCTGGCGAGCAGAAAAACCTCCTTTATGATGGCGCTCTGCCTGAGCTCAATCT |
| | ATGAGGACCTGAAGATGTACCAGGTGGAGTTTAAAACCATGAATGCCAAGCTGCTTATGGA |
| | CCCTAAGAGACAGATTTTCCTGGATCAGAACATGCTGGCCGTGATTGATGAATTAATGCAG |
| | GCGCTGAACTTTAACAGCGAGACCGTGCCCCAGAAAAGCAGCCTGGAGGAGCCCGACTTTT |
| | ACAAGACCAAGATCAAGTTGTGCATCCTCCTGCACGCCTTCAGAATCAGAGCGGTGACGAT |
| | CGACAGAGTCATGAGCTACCTCAACGCT |
| A3 Construct (SEQ | atgagggtccccgctcagctcctggggctcctgctgctctggctcccaggtgcacgatgtg |
| ID NO: 17)- | ccATCTGGGAGCTGAAGAAGGACGTGTACGTGGTGGAGCTGGACTGGTACCCCGACGCCCC |
| includes (1) leader | CGGCGAGATGGTGGTGCTGACCTGCGACACCCCCGAGGAGGACGGCATCACCTGGACCCTG |
| sequence (lower | GACCAGAGCAGCGAGGTGCTGGGCAGCGGCAAGACCCTGACCATCCAGGTGAAGGAGTTCG |
| case; SEQ ID NO: | GCGACGCCGGCCAGTACACCTGCCACAAGGGCGGCGAGGTGCTGAGCCACAGCCTGCTGCT |
| 42); (2) IL-12ß | GCTGCACAAGAAGGAGGACGGCATCTGGAGCACCGACATCCTGAAGGACCAGAAGGAGCCC |
| (bolded and | AAGAACAAGACCTTCCTGAGATGCGAGGCCAAGAACTACAGCGGCAGATTCACCTGCTGGT |
| underlined; SEQ ID | GGCTGACCACCATCAGCACCGACCTGACCTTCAGCGTGAAAAGCAGCAGAGGCAGCAGCGA |
| NO: 67); (3) GS | CCCCCAGGGCGTGACCTGCGGCGCCGCCACCCTGAGCGCCGAGAGAGTGAGAGGCGACAAC |
| linker (italicized; | AAGGAGTACGAGTACAGCGTGGAGTGCCAGGAGGACAGCGCCTGCCCCGCCGCCGAGGAGA |
| SEQ ID NO: 92); | GCCTGCCCATCGAGGTGATGGTGGACGCCGTGCACAAGCTGAAGTACGAGAACTACACCAG |
| and (4) IL-12α | CAGCTTCTTCATCAGAGACATCATCAAGCCCGACCCCCCCAAGAACCTGCAGCTGAAGCCC |
| (bolded; SEQ ID | CTGAAGAACAGCAGACAGGTGGAGGTGAGCTGGGAGTACCCCGACACCTGGAGCACCCCCC |
| NO: 117) | ACAGCTACTTCAGCCTGACCTTCTGCGTGCAGGTGCAGGGCAAGAGCAAGAGAGAGAAGAA |
| | GGACAGAGTGTTCACCGACAAGACCAGCGCCACCGTGATCTGCAGAAAGAACGCCAGCATC |
| | AGCGTGAGAGCCCAGGACAGATACTACAGCAGCAGCTGGAGCGAGTGGGCCAGCGTGCCCT |
| | GCAGCGGCGGCAGCGGCGGCGGCAGCGGCGGCGGCAGCGGCGGCGGCAGCAGAAACCTGCC |
| | CGTGGCCACCCCCGACCCCGGCATGTTCCCCTGCCTGCACCACAGCCAGAACCTGCTGAGA |
| | GCCGTGAGCAACATGCTGCAGAAGGCCAGACAGACCCTGGAGTTCTACCCCTGCACCAGCG |
| | AGGAGATCGACCACGAGGACATCACCAAGGACAAGACCAGCACCGTGGAGGCCTGCCTGCC |
| | CCTGGAGCTGACCAAGAACGAGAGCTGCCTGAACAGCAGAGAGACCAGCTTCATCACCAAC |
| | GGCAGCTGCCTGGCCAGCAGAAAGACCAGCTTCATGATGGCCCTGTGCCTGAGCAGCATCT |
| | ACGAGGACCTGAAGATGTACCAGGTGGAGTTCAAGACCATGAACGCCAAGCTGCTGATGGA |
| | CCCCAAGAGACAGATCTTCCTGGACCAGAACATGCTGGCCGTGATCGACGAGCTGATGCAG |
| | GCCCTGAACTTCAACAGCGAGACCGTGCCCCAGAAAAGCAGCCTGGAGGAGCCCGACTTCT |
| | ACAAGACCAAGATCAAGCTGTGCATCCTGCTGCACGCCTTCAGAATCAGAGCCGTGACCAT |
| | CGACAGAGTGATGAGCTACCTGAACGCCAGC |
| A4 Construct (SEQ | atgagggtccccgctcagctcctggggctcctgctgctctggctcccaggtgcacgatgtg |
| ID NO: 18)- | ccATCTGGGAGCTGAAGAAGGATGTGTATGTGGTGGAGCTGGACTGGTACCCAGATGCCCC |
| includes (1) leader | TGGAGAGATGGTGGTGCTGACCTGTGACACCCCAGAGGAGGATGGCATCACCTGGACCCTG |
| sequence (lower | GACCAGAGCAGCGAGGTGCTGGGCAGCGGCAAGACCCTGACCATCCAGGTGAAGGAGTTTG |
| case; SEQ ID NO: | GAGATGCTGGCCAGTACACCTGCCACAAGGGCGGGGAGGTGCTGAGCCACAGCCTGCTGCT |
| 43); (2) IL-12ß | GCTGCACAAGAAGGAGGATGGCATCTGGAGCACAGACATCCTGAAGGACCAGAAGGAGCCC |
| (bolded and | AAGAACAAGACCTTCCTGCGCTGTGAGGCCAAGAACTACAGCGGCCGCTTCACCTGCTGGT |
| underlined; SEQ ID | GGCTGACCACCATCAGCACAGACCTGACCTTCTCTGTGAAAAGCAGCCGGGGCAGCAGTGA |
| NO: 68); (3) GS | CCCCCAGGGCGTGACCTGTGGGGCCGCCACCCTGTCTGCTGAGCGGGTGCGGGGGGACAAC |
| linker (italicized; | AAGGAGTATGAGTACAGCGTGGAGTGCCAGGAGGACAGCGCCTGCCCAGCTGCTGAGGAGA |
| SEQ ID NO: 93); | GCCTGCCCATCGAGGTGATGGTGGATGCTGTGCACAAGCTGAAGTATGAGAACTACACCAG |
| and (4) IL-12α | CAGCTTCTTCATCCGGGACATCATCAAGCCAGACCCCCCCAAGAACCTGCAGCTGAAGCCC |
| (bolded; SEQ ID | CTGAAGAACAGCCGGCAGGTGGAGGTGTCCTGGGAGTACCCAGACACCTGGAGCACCCCCC |
| NO: 118) | ACAGCTACTTCAGCCTGACCTTCTGTGTGCAGGTGCAGGGCAAGAGCAAGCGGGAGAAGAA |
| | GGACAGAGTCTTCACAGACAAGACCAGCGCCACCGTCATCTGCAGGAAGAATGCCAGCATC |
| | TCTGTGCGGGCCCAGGACCGCTACTACAGCAGCTCCTGGAGCGAGTGGGCCTCTGTGCCCT |
| | GCAGCGGCGGCAGCGGCGGCGGCAGCGGCGGCGGCAGCGGCGGCGGCAGCAGGAACCTGCC |
| | TGTGGCCACCCCAGACCCCGGCATGTTCCCCTGCCTGCACCACAGCCAGAACCTGCTGCGG |
| | GCTGTGAGCAACATGCTGCAGAAGGCCCGGCAGACCCTGGAGTTCTACCCCTGCACCAGCG |
| | AGGAGATTGACCACGAGGACATCACCAAGGACAAGACCAGCACAGTGGAGGCCTGCCTGCC |
| | CCTGGAGCTGACCAAGAATGAAAGCTGCCTGAACAGCCGGGAGACCAGCTTCATCACCAAC |
| | GGCAGCTGCCTGGCCAGCAGGAAGACCAGCTTCATGATGGCCCTGTGCCTGAGCAGCATCT |
| | ATGAGGACCTGAAGATGTACCAGGTGGAGTTCAAGACCATGAATGCCAAGCTGCTGATGGA |
| | CCCCAAGAGGCAGATCTTCCTGGACCAGAACATGCTGGCCGTGATTGATGAGCTGATGCAG |
| | GCCCTGAACTTCAACAGCGAGACCGTGCCCCAGAAAAGCAGCCTGGAGGAGCCAGACTTCT |
| | ACAAGACCAAGATCAAGCTGTGCATCCTGCTGCACGCCTTCCGCATCCGGGCTGTGACCAT |
| | CGACAGAGTGATGAGCTACCTGAATGCCAGC |
| B1 Construct (SEQ | atgagggtccccgctcagctcctggggctcctgctgctctggctcccaggtgcacgatgtg |
| ID NO: 19)- | ccATCTGGGAGCTGAAGAAAGACGTGTACGTGGTGGAGCTTGACTGGTACCCCGACGCCCC |
| includes (1) leader | CGGTGAAATGGTGGTTCTGACCTGCGACACACCAGAGGAGGACGGCATCACCTGGACCCTG |
| sequence (lower | GACCAGTCCAGCGAGGTCCTGGGATCTGGCAAGACCCTGACCATCCAGGTTAAGGAATTTG |
| case; SEQ ID NO: | GCGACGCCGGCCAGTACACCTGCCACAAAGGCGGCGAGGTCCTTTCGCACAGTCTGCTGCT |
| 44); (2) IL-12ß | GCTGCATAAAAAGGAGGACGGCATTTGGAGCACCGACATTCTGAAGGATCAGAAAGAGCCC |
| (bolded and | AAGAACAAGACCTTTCTGAGATGTGAGGCCAAAAACTACTCTGGACGCTTCACCTGTTGGT |
| underlined; SEQ ID | GGCTGACCACCATCAGCACAGACCTGACCTTCTCGGTGAAGTCTAGTAGGGGCAGCAGTGA |
| NO: 69); (3) first GS | CCCCCAGGGCGTAACATGCGGCGCCGCTACCCTGAGCGCCGAGAGAGTGAGAGGCGATAAC |
| linker (italicized; | AAGGAGTACGAGTACTCCGTGGAGTGCCAAGAGGACTCAGCCTGGCCCGCCGCCGAGGAGT |
| SEQ ID NO: 94); | CGCTGCCCATCGAGGTGATGGTGGATGCAGTGCACAAGCTGAAGTACGAGAACTACACCAG |
| (4) IL-12α (bolded; | TAGCTTTTTCATCAGAGATATCATTAAACCCGACCCTCCCAAGAACCTGCAGCTGAAGCCC |
| SEQ ID NO: 119); | TTAAAGAACAGCCGGCAGGTGGAAGTGTCATGGGAGTACCCAGACACCTGGAGCACTCCGC |
| (5) second GS linker | ACAGCTACTTCAGCCTGACCTTCTGCGTTCAGGTGCAGGGAAAAAGCAAGAGAGAGAAGAA |
| (italicized and | AGACAGAGTGTTCACCGACAAGACCAGCGCAACCGTGATCTGTAGAAAGAACGCCTCGATC |
| underlined; SEQ ID | AGCGTGAGAGCCCAGGACAGATACTACAGCAGCAGCTGGAGCGAGTGGGCCAGTGTACCTT |
| NO: 140); and (6) | GCAGCGGTGGAAGCGGCGGAGGCTCTGGCGGAGGGAGTGGCGGAGGCAGCAGAAATCTTCC |
| albumin (lower case | AGTAGCTACCCCCGACCCCGGCATGTTTCCCTGTCTGCACCATTCTCAAAACCTGTTACGG |
| and underlined; SEQ | GCCGTGAGCAACATGCTGCAGAAGGCCAGACAGACACTGGAGTTTTACCCCTGTACCTCAG |
| ID NO: 161) | AGGAGATCGATCATGAGGACATTACTAAGGACAAGACCAGCACCGTGGAAGCCTGCCTGCC |
| | CCTAGAGCTAACCAAGAACGAGAGCTGCCTGAACTCTAGAGAGACAAGCTTCATCACGAAC |
| | GGCTCATGCCTGGCCAGTAGGAAAACCAGCTTCATGATGGCTCTGTGCCTGAGCTCCATAT |
| | ATGAGGACCTTAAGATGTACCAGGTGGAGTTCAAAACCATGAACGCCAAGCTGCTGATGGA |
| | CCCAAAGAGACAGATCTTCCTTGACCAGAACATGCTGGCCGTTATCGATGAGCTGATGCAG |
| | GCCCTGAACTTCAACAGCGAGACCGTGCCCCAGAAAAGCAGCCTGGAGGAACCCGACTTCT |
| | ACAAAACCAAGATCAAGCTGTGCATTCTGCTGCATGCCTTCCGCATTAGAGCCGTGACCAT |
| | CGATAGGGTGATGAGCTACCTGAACGCCAGCGGCTCTGGCGGCGGCAGTgacgcccacaag |
| | tccgaggtcgcccacagattcaaggatttgggcgaggagaacttcaaggccctggtgctga |
| | tcgccttcgcccagtacttgcagcagtgtcccttcgaggaccatgtgaagctggtgaacga |
| | ggtgaccgagttcgccaagacctgtgtggccgacgagagcgccgagaactgcgataagtct |
| | ctgcacaccctttttggcgacaaactgtgcaccgtggccaccctgagagagacctacggcg |
| | agatggccgactgctgtgcgaagcaggagcccgagcgcaatgagtgtttcctgcagcataa |
| | ggacgacaaccccaacctgcccagactggtgagacccgaggtggacgtgatgtgcaccgcc |
| | ttccacgacaacgaggagacctttctgaaaaaatacctgtacgagatcgcaagacgccacc |
| | cctacttctacgcccccgagctgctgttcttcgccaagcgctacaaggctgccttcaccga |
| | atgctgccaggccgccgataaggccgcgtgcttactgccaaagctggacgagctgagagac |
| | gaggggaaagcctctagcgccaaacagagattgaagtgtgccagcctgcagaaattcggtg |
| | agagagccttcaaggcctgggccgtggccagattatcacagcggttccccaaggctgaatt |
| | cgccgaggtgagcaaacttgtcaccgatctgacaaaagtgcacaccgagtgctgccatggc |
| | gacctgctggagtgcgccgacgaccgggccgacctggccaagtacatctgcgagaaccagg |
| | acagcatctccagcaagctgaaggagtgctgcgagaagcccctgctggagaaaagccactg |
| | catcgccgaggtggagaatgacgaaatgcccgccgacctgcccagcctggccgccgacttc |
| | gtggaaagcaaggacgtgtgcaaaaattacgccgaagccaaggatgtgttcttgggcatgt |
| | tcttgtacgagtacgccagacgccaccccgactacagcgtggtgctgctgctgcggctggc |
| | caagacctacgagaccaccctggagaagtgctgtgctgccgccgacccccacgagtgctac |
| | gccaaggtatttgacgagttcaagcccctggtggaggagcctcagaacctgattaagcaga |
| | actgtgagctgttcgagcagctgggcgagtacaagttccagaacgccctcctggtgagata |
| | caccaaaaaggtgcctcaggtaagcactcccaccctggtggaggtgagcaggaacctcggc |
| | aaggtgggcagcaaatgctgcaagcacccagaggccaaaagaatgccctgcgcagaagact |
| | acctcagcgtggtcctgaaccagctgtgcgtgctgcacgaaaagacccctgtgagcgatag |
| | agtgacaaagtgctgcaccgagagcctggtgaacagaagaccctgttttagcgccctggag |
| | gtggacgagacctacgtgcccaaggagttcaacgccgagacgttcactttccacgcggaca |
| | tctgcaccctgagcgagaaggagagacaaatcaagaagcagaccgccctagtcgagctggt |
| | aaaacacaagcccaaggccaccaaggagcagctgaaggccgtgatggacgactttgcagcc |
| | ttcgtggagaagtgctgcaaggctgacgacaaggagacctgcttcgccgaggagggcaaga |
| | agctcgtagccgccagccaggccgctctcggcctt |
| B2 Construct (SEQ | atgagggtccccgctcagctcctggggctcctgctgctctggctcccaggtgcacgatgtg |
| ID NO: 20)- | ccATCTGGGAACTGAAGAAGGACGTCTACGTGGTGGAGCTGGATTGGTACCCCGACGCCCC |
| includes (1) leader | CGGCGAGATGGTCGTGCTGACCTGTGACACCCCTGAGGAGGACGGAATCACATGGACCCTG |
| sequence (lower | GACCAGAGCAGCGAAGTGTTGGGCAGCGGCAAGACCCTGACCATCCAAGTGAAGGAATTCG |
| case; SEQ ID NO: | GCGACGCGGGCCAGTATACTTGCCACAAGGGCGGGGAGGTTCTGAGCCACAGCCTGCTGCT |
| 45); (2) IL-12ß | GCTCCACAAGAAAGAGGATGGCATCTGGTCTACCGACATCCTGAAGGACCAAAAGGAGCCA |
| (bolded and | AAGAACAAGACCTTCCTAAGATGCGAGGCCAAGAACTACTCAGGTCGTTTCACCTGCTGGT |
| underlined; SEQ ID | GGCTGACCACAATCTCCACCGACCTGACCTTCAGCGTGAAAAGCAGCCGCGGCAGTAGCGA |
| NO: 70); (3) first GS | CCCACAGGGCGTGACCTGCGGGGCCGCCACTCTGAGCGCCGAGAGAGTGCGCGGCGATAAT |
| linker (italicized; | AAGGAATACGAGTACAGCGTGGAGTGCCAGGAAGACTCAGCCTGCCCCGCCGCAGAAGAAA |
| SEQ ID NO: 95); | GTCTGCCAATAGAGGTGATGGTTGACGCCGTGCACAAGCTAAAGTACGAGAACTACACCAG |
| (4) IL-12α (bolded; | CAGCTTCTTTATCCGGGACATCATCAAGCCAGATCCCCCCAAGAACCTGCAGCTTAAGCCC |
| SEQ ID NO: 120); | CTGAAGAACAGCAGACAGGTGGAGGTGTCATGGGAATACCCCGACACCTGGAGCACCCCCC |
| (5) second GS linker | ATAGCTACTTCTCACTGACCTTCTGCGTGCAGGTGCAGGGCAAGAGCAAGAGAGAGAAGAA |
| (italicized and | GGATAGGGTATTCACGGACAAGACCTCAGCCACCGTGATCTGCCGAAAGAACGCCAGCATC |
| underlined; SEQ ID | AGCGTGAGAGCCCAGGACAGATACTATAGCTCCTCGTGGAGCGAGTGGGCCAGCGTACCTT |
| NO: 141); and (6) | GCAGCGGCGGGAGCGGCGGCGGCAGCGGAGGTGGCAGTGGCGGAGGCAGCAGAAACCTCCC |
| albumin (lower case | CGTGGCAACCCCTGACCCCGGGATGTTTCCTTGCCTGCACCACTCCCAGAACCTCCTGAGA |
| and underlined; SEQ | GCCGTGTCCAACATGCTGCAGAAAGCCAGACAGACCCTCGAGTTCTATCCCTGCACAAGCG |
| ID NO: 162) | AGGAGATCGACCACGAGGACATCACTAAGGACAAGACCAGCACAGTGGAAGCCTGCCTCCC |
| | GCTGGAGCTGACTAAGAACGAGAGCTGTCTGAACAGCAGGGAGACCAGCTTCATCACCAAC |
| | GGCAGCTGCCTGGCGAGCAGAAAAACCTCCTTTATGATGGCGCTCTGCCTGAGCTCAATCT |
| | ATGAGGACCTGAAGATGTACCAGGTGGAGTTTAAAACCATGAATGCCAAGCTGCTTATGGA |
| | CCCTAAGAGACAGATTTTCCTGGATCAGAACATGCTGGCCGTGATTGATGAATTAATGCAG |
| | GCGCTGAACTTTAACAGCGAGACCGTGCCCCAGAAAAGCAGCCTGGAGGAGCCCGACTTTT |
| | ACAAGACCAAGATCAAGTTGTGCATCCTCCTGCACGCCTTCAGAATCAGAGCGGTGACGAT |
| | CGACAGAGTCATGAGCTACCTCAACGCTAGCGGCAGCGGTGGCGGCAGCgacgcccacaag |
| | agcgaggtggcccacagattcaaggatctcggggaggagaacttcaaggccctggtgctga |
| | tcgccttcgcacagtacctgcagcagtgccccttcgaggaccacgtaaaactggtgaacga |
| | ggtgacggagttcgccaagacctgtgttgccgacgagtcggccgagaattgcgacaagagc |
| | ctgcataccctgttcggcgacaagctgtgcaccgtggccaccctgagagagacctacggcg |
| | agatggccgactgctgcgccaagcaagagcccgagagaaacgagtgcttcctgcagcacaa |
| | ggacgacaaccccaacctgccccggctggtgagacccgaggtggacgtgatgtgcaccgcc |
| | ttccacgacaacgaggagaccttcctgaagaagtatctgtacgagatcgccagaagacacc |
| | cttatttttacgcccccgagctgctgttcttcgctaagagatataaggcagccttcaccga |
| | gtgttgtcaggccgccgataaggccgcttgcctgctgcccaagttggacgagctcagagac |
| | gagggcaaggcgagcagcgccaagcagagactgaagtgcgccagcctgcagaagttcggcg |
| | agagggccttcaaggcctgggcggtggccagactgtcccagagatttcccaaggccgagtt |
| | cgccgaggtaagcaagctggtcaccgacctgaccaaggtgcacaccgagtgttgccacggc |
| | gacctgctggaatgcgccgatgaccgtgccgacctggccaagtacatctgcgagaatcagg |
| | actccatcagcagcaaactgaaggagtgttgcgagaagcccctgctggaaaagagccattg |
| | catcgctgaggtggaaaacgacgagatgcccgcagacctgcccagcctggccgcagacttt |
| | gtggaaagtaaggacgtgtgcaagaactacgcggaggccaaagacgtgtttctgggcatgt |
| | tcctatacgagtatgccagaagacaccccgactacagcgttgtgttattgctgagactggc |
| | caagacctacgagactaccttggagaagtgctgcgccgccgccgacccccacgagtgctat |
| | gccaaggtgttcgacgagttcaagcccctggtggaggagccccagaatctgattaagcaga |
| | attgcgagctttttgagcagctgggcgagtataagttccagaacgccctgctggtgagata |
| | caccaaaaaggtacctcaggtgagcacccccaccctggtggaggtgagcagaaatctgggc |
| | aaggtgggcagcaagtgctgcaagcacccggaggccaagagaatgccctgtgccgaggatt |
| | acctgtcagtggtgctgaaccagctgtgcgttctgcacgaaaagacgcccgtgtcggacag |
| | agtgaccaagtgctgcacggagagcctggtgaacagaagaccgtgcttcagcgccctagag |
| | gtggacgagacctacgtgcccaaggagttcaacgccgagaccttcaccttccacgccgaca |
| | tctgtaccctgtcagagaaggagagacagatcaagaagcagaccgccttagtggagctggt |
| | gaagcacaagcccaaggccaccaaggagcagctgaaagccgtgatggacgatttcgcagcc |
| | ttcgtcgagaagtgctgcaaggccgacgacaaggaaacttgcttcgccgaggagggcaaga |
| | agctggtggctgcctcgcaggccgccctcggcctg |
| B3 Construct (SEQ | atgagggtccccgctcagctcctggggctcctgctgctctggctcccaggtgcacgatgtg |
| ID NO: 21)- | ccATCTGGGAGCTGAAGAAGGACGTGTACGTGGTGGAGCTGGACTGGTACCCCGACGCCCC |
| includes (1) leader | CGGCGAGATGGTGGTGCTGACCTGCGACACCCCCGAGGAGGACGGCATCACCTGGACCCTG |
| sequence (lower | GACCAGAGCAGCGAGGTGCTGGGCAGCGGCAAGACCCTGACCATCCAGGTGAAGGAGTTCG |
| case; SEQ ID NO: | GCGACGCCGGCCAGTACACCTGCCACAAGGGCGGCGAGGTGCTGAGCCACAGCCTGCTGCT |
| 46); (2) IL-12ß | GCTGCACAAGAAGGAGGACGGCATCTGGAGCACCGACATCCTGAAGGACCAGAAGGAGCCC |
| (bolded and | AAGAACAAGACCTTCCTGAGATGCGAGGCCAAGAACTACAGCGGCAGATTCACCTGCTGGT |
| underlined; SEQ ID | GGCTGACCACCATCAGCACCGACCTGACCTTCAGCGTGAAAAGCAGCAGAGGCAGCAGCGA |
| NO: 71); (3) first GS | CCCCCAGGGCGTGACCTGCGGCGCCGCCACCCTGAGCGCCGAGAGAGTGAGAGGCGACAAC |
| linker (italicized; | AAGGAGTACGAGTACAGCGTGGAGTGCCAGGAGGACAGCGCCTGCCCCGCCGCCGAGGAGA |
| SEQ ID NO: 96); | GCCTGCCCATCGAGGTGATGGTGGACGCCGTGCACAAGCTGAAGTACGAGAACTACACCAG |
| (4) IL-12α (bolded; | CAGCTTCTTCATCAGAGACATCAATCAAGCCGACCCCCCCAAGAACCTGCAGCTGAAGCCC |
| SEQ ID NO: 121); | CTGAAGAACAGCAGACAGGTGGAGGTGAGCTGGGAGTACCCCGACACCTGGAGCACCCCCC |
| (5) second GS linker | ACAGCTACTTCAGCCTGACCTTCTGCGTGCAGGTGCAGGGCAAGAGCAAGAGAGAGAAGAA |
| (italicized and | GGACAGAGTGTTCACCGACAAGACCAGCGCCACCGTGATCTGCAGAAAGAACGCCAGCATC |
| underlined; SEQ ID | AGCGTGAGAGCCCAGGACAGATACTACAGCAGCAGCTGGAGCGAGTGGGCCAGCGTGCCCT |
| NO: 142); and (6) | GCAGCGGCGGCAGCGGCGGCGGCAGCGGCGGCGGCAGCGGCGGCGGCAGCAGAAACCTGCC |
| albumin (lower case | CGTGGCCACCCCCGACCCCGGCATGTTCCCCTGCCTGCACCACAGCCAGAACCTGCTGAGA |
| and underlined; SEQ | GCCGTGAGCAACATGCTGCAGAAGGCCAGACAGACCCTGGAGTTCTACCCCTGCACCAGCG |
| ID NO: 163) | AGGAGATCGACCACGAGGACATCACCAAGGACAAGACCAGCACCGTGGAGGCCTGCCTGCC |
| | CCTGGAGCTGACCAAGAACGAGAGCTGCCTGAACAGCAGAGAGACCAGCTTCATCACCAAC |
| | GGCAGCTGCCTGGCCAGCAGAAAGACCAGCTTCATGATGGCCCTGTGCCTGAGCAGCATCT |
| | ACGAGGACCTGAAGATGTACCAGGTGGAGTTCAAGACCATGAACGCCAAGCTGCTGATGGA |
| | CCCCAAGAGACAGATCTTCCTGGACCAGAACATGCTGGCCGTGATCGACGAGCTGATGCAG |
| | GCCCTGAACTTCAACAGCGAGACCGTGCCCCAGAAAAGCAGCCTGGAGGAGCCCGACTTCT |
| | ACAAGACCAAGATCAAGCTGTGCATCCTGCTGCACGCCTTCAGAATCAGAGCCGTGACCAT |
| | CGACAGAGTGATGAGCTACCTGAACGCCAGCGGCAGCGGCGGCGGCAGCgacgcccacaag |
| | agcgaggtggcccacagattcaaggacctgggcgaggagaacttcaaggccctggtgctga |
| | tcgccttcgcccagtacctgcagcagtgccccttcgaggaccacgtgaagctggtgaacga |
| | ggtgaccgagttcgccaagacctgcgtggccgacgagagcgccgagaactgcgacaagagc |
| | ctgcacaccctgttcggcgacaagctgtgcaccgtggccaccctgagagagacctacggcg |
| | agatggccgactgctgcgccaagcaggagcccgagagaaacgagtgcttcctgcagcacaa |
| | ggacgacaaccccaacctgcccagactggtgagacccgaggtggacgtgatgtgcaccgcc |
| | ttccacgacaacgaggagaccttcctgaagaagtacctgtacgagatcgccagaagacacc |
| | cctacttctacgcccccgagctgctgttcttcgccaagagatacaaggccgccttcaccga |
| | gtgctgccaggccgccgacaaggccgcctgcctgctgcccaagctggacgagctgagagac |
| | gagggcaaggccagcagcgccaagcagagactgaagtgcgccagcctgcagaagttcggcg |
| | agagagccttcaaggcctgggccgtggccagactgagccagagattccccaaggccgagtt |
| | cgccgaggtgagcaagctggtgaccgacctgaccaaggtgcacaccgagtgctgccacggc |
| | gacctgctggagtgcgccgacgacagagccgacctggccaagtacatctgcgagaaccagg |
| | acagcatcagcagcaagctgaaggagtgctgcgagaagcccctgctggagaaaagccactg |
| | catcgccgaggtggagaacgacgagatgcccgccgacctgcccagcctggccgccgacttc |
| | gtggagagcaaggacgtgtgcaagaactacgccgaggccaaggacgtgttcctgggcatgt |
| | tcctgtacgagtacgccagaagacaccccgactacagcgtggtgctgctgctgagactggc |
| | caagacctacgagaccaccctggagaagtgctgcgccgccgccgacccccacgagtgctac |
| | gccaaggtgttcgacgagttcaagcccctggtggaggagccccagaacctgatcaagcaga |
| | actgcgagctgttcgagcagctgggcgagtacaagttccagaacgccctgctggtgagata |
| | caccaagaaggtgccccaggtgagcacccccaccctggtggaggtgagcagaaacctgggc |
| | aaggtgggcagcaagtgctgcaagcaccccgaggccaagagaatgccctgcgccgaggact |
| | acctgagcgtggtgctgaaccagctgtgcgtgctgcacgagaagacccccgtgagcgacag |
| | agtgaccaagtgctgcaccgagagcctggtgaacagaagaccctgcttcagcgccctggag |
| | gtggacgagacctacgtgcccaaggagttcaacgccgagaccttcaccttccacgccgaca |
| | tctgcaccctgagcgagaaggagagacagatcaagaagcagaccgccctggtggagctggt |
| | gaagcacaagcccaaggccaccaaggagcagctgaaggccgtgatggacgacttcgccgcc |
| | ttcgtggagaagtgctgcaaggccgacgacaaggagacctgcttcgccgaggagggcaaga |
| | agctggtggccgccagccaggccgccctgggcctg |
| B4 Construct (SEQ | atgagggtccccgctcagctcctggggctcctgctgctctggctcccaggtgcacgatgtg |
| ID NO: 22)- | ccATCTGGGAGCTGAAGAAGGATGTGTATGTGGTGGAGCTGGACTGGTACCCAGATGCCCC |
| includes (1) leader | TGGAGAGATGGTGGTGCTGACCTGTGACACCCCAGAGGAGGATGGCATCACCTGGACCCTG |
| sequence (lower | GACCAGAGCAGCGAGGTGCTGGGCAGCGGCAAGACCCTGACCATCCAGGTGAAGGAGTTTG |
| case; SEQ ID NO: | GAGATGCTGGCCAGTACACCTGCCACAAGGGCGGGGAGGTGCTGAGCCACAGCCTGCTGCT |
| 47); (2) IL-12ß | GCTGCACAAGAAGGAGGATGGCATCTGGAGCACAGACATCCTGAAGGACCAGAAGGAGCCC |
| (bolded and | AAGAACAAGACCTTCCTGCGCTGTGAGGCCAAGAACTACAGCGGCCGCTTCACCTGCTGGT |
| underlined; SEQ ID | GGCTGACCACCATCAGCACAGACCTGACCTTCTCTGTGAAAAGCAGCCGGGGCAGCAGTGA |
| NO: 72); (3) first GS | CCCCCAGGGCGTGACCTGTGGGGCCGCCACCCTGTCTGCTGAGCGGGTGCGGGGGGACAAC |
| linker (italicized; | AAGGAGTATGAGTACAGCGTGGAGTGCCAGGAGGACAGCGCCTGCCCAGCTGCTGAGGAGA |
| SEQ ID NO: 97); | CAGCTTCTTCATCCGGGACATCATCAAGCCAGACCCCCCCAAGAACCTGCAGCTGAAGCCC |
| (4) IL-12α (bolded; | CTGAAGAACAGCCGGCAGGTGGAGGTGTCCTGGGAGTACCCAGACACCTGGAGCACCCCCC |
| SEQ ID NO: 122); | ACAGCTACTTCAGCCTGACCTTCTGTGTGCAGGTGCAGGGCAAGAGCAAGCGGGAGAAGAA |
| (5) second GS linker | GGACAGAGTCTTCACAGACAAGACCAGCGCCACCGTCATCTGCAGGAAGAATGCCAGCATC |
| (italicized and | TCTGTGCGGGCCCAGGACCGCTACTACAGCAGCTCCTGGAGCGAGTGGGCCTCTGTGCCCT |
| underlined; SEQ ID | GCAGCGGCGGCAGCGGCGGCGGCAGCGGCGGCGGCAGCGGCGGCGGCAGCAGGAACCTGCC |
| NO: 143); and (6) | GCAGCGGCGGCAGCGGCGGCGGCAGCGGCGGCGGCAGCGGCGGCGGCAGCAGGAACCTGCC |
| albumin (lower case | TGTGGCCACCCCAGACCCCGGCATGTTCCCCTGCCTGCACCACAGCCAGAACCTGCTGCGG |
| and underlined; SEQ | GCTGTGAGCAACATGCTGCAGAAGGCCCGGCAGACCCTGGAGTTCTACCCCTGCACCAGCG |
| ID NO: 164) | AGGAGATTGACCACGAGGACATCACCAAGGACAAGACCAGCACAGTGGAGGCCTGCCTGCC |
| | CCTGGAGCTGACCAAGAATGAAAGCTGCCTGAACAGCCGGGAGACCAGCTTCATCACCAAC |
| | GGCAGCTGCCTGGCCAGCAGGAAGACCAGCTTCATGATGGCCCTGTGCCTGAGCAGCATCT |
| | ATGAGGACCTGAAGATGTACCAGGTGGAGTTCAAGACCATGAATGCCAAGCTGCTGATGGA |
| | CCCCAAGAGGCAGATCTTCCTGGACCAGAACATGCTGGCCGTGATTGATGAGCTGATGCAG |
| | GCCCTGAACTTCAACAGCGAGACCGTGCCCCAGAAAAGCAGCCTGGAGGAGCCAGACTTCT |
| | ACAAGACCAAGATCAAGCTGTGCATCCTGCTGCACGCCTTCCGCATCCGGGCTGTGACCAT |
| | CGACAGAGTGATGAGCTACCTGAATGCCAGCGGCAGCGGCGGCGGCAGTgatgcccacaag |
| | tctgaggtggcccaccgcttcaaggacctgggggaggagaacttcaaggccctggtgctga |
| | ttgcctttgcccagtacctgcagcagtgcccctttgaggaccacgtgaagctggtgaatga |
| | ggtgacagaatttgccaagacctgtgtggctgatgaatctgctgagaactgtgacaagagc |
| | ctgcacaccctgtttggagacaagctgtgcaccgtggccaccctgcgggagacctatggag |
| | agatggctgactgctgtgccaagcaggagcctgagagaaatgaatgcttcctgcagcacaa |
| | ggatgacaaccccaacctgccccggctggtgcggcctgaggtggatgtgatgtgcacagcc |
| | ttccatgacaatgaggagaccttcctgaagaagtacctgtatgaaattgcccggcggcacc |
| | cctacttctacgcccctgagctgctgttctttgccaagcgctacaaggccgccttcacaga |
| | gtgctgccaggccgctgacaaggccgcctgcctgctgcccaagctggatgagctgagagat |
| | gagggcaaggccagcagcgccaagcagaggctgaagtgtgccagcctgcagaagtttggag |
| | agcgggccttcaaggcctgggccgtggcccggctgagccagcgcttccccaaggccgagtt |
| | tgctgaggtgtccaagctggtgacagacctgaccaaggtgcacacagagtgctgccacggg |
| | gacctgctggagtgtgctgatgacagagctgacctggccaagtacatctgtgagaaccagg |
| | acagcatcagcagcaagctgaaggagtgctgtgagaagcccctgctggaaaagagccactg |
| | catcgccgaggtggagaatgatgagatgcctgctgacctgcccagcctggccgctgacttt |
| | gtggagagcaaggatgtgtgcaagaactatgcagaggccaaggatgtgttcctgggcatgt |
| | tcctgtatgaatatgcccggcggcacccagactacagcgtggtgctgctgctgcggctggc |
| | caagacctatgagaccaccctggagaagtgctgtgccgctgctgacccccatgaatgttat |
| | gccaaggtgtttgatgagttcaagcccctggtggaggagccccagaacctgatcaagcaga |
| | actgtgagctgtttgagcagctgggggagtacaagttccagaatgccctgctggtgcgcta |
| | caccaagaaggtgccccaggtgtccacccccaccctggtggaggtgtccaggaacctgggc |
| | aaggtgggcagcaagtgctgcaagcaccctgaggccaagaggatgccctgtgccgaggact |
| | acctgtctgtggtgctgaaccagctgtgtgtgctgcacgagaagacccctgtgtctgacag |
| | agtgaccaagtgctgcacagagagcctggtgaacagaagaccctgcttcagcgccctggag |
| | gtggatgagacctacgtgcccaaggagttcaatgctgagaccttcaccttccacgccgaca |
| | tctgcaccctgtctgagaaggagcggcagatcaagaagcagacagccctggtggagctggt |
| | gaagcacaagcccaaggccaccaaggagcagctgaaggctgtgatggatgactttgctgcc |
| | tttgtggagaagtgctgcaaggcagatgacaaggagacctgctttgctgaggagggcaaga |
| | agctggtggccgccagccaggccgccctgggcctg |
| C1 Construct (SEQ | atgagggtccccgctcagctcctggggctcctgctgctctggctcccaggtgcacgatgtg |
| ID NO: 23)- | ccATCTGGGAGCTGAAAAAGGACGTGTACGTGGTGGAACTTGACTGGTACCCCGACGCCCC |
| includes (1) leader | CGGCGAGATGGTGGTACTGACCTGCGACACTCCCGAGGAAGACGGCATTACCTGGACCTTG |
| sequence (lower | GACCAGAGCAGCGAGGTTCTGGGCTCCGGAAAAACCTTGACAATCCAAGTGAAAGAATTCG |
| case; SEQ ID NO: | GCGACGCTGGCCAGTACACCTGCCACAAGGGCGGCGAGGTGCTGTCCCACAGCCTGCTGCT |
| 48); (2) IL-12ß | GCTGCATAAGAAAGAAGACGGGATTTGGAGCACCGATATACTGAAGGATCAGAAGGAGCCC |
| (bolded and | AAGAACAAGACCTTCCTGAGGTGCGAGGCCAAAAATTACAGCGGCAGATTCACCTGCTGGT |
| underlined; SEQ ID | GGCTGACCACCATTAGCACAGACCTGACTTTCAGCGTAAAGTCTTCAAGGGGCAGCTCAGA |
| NO: 73); (3) first GS | CCCCCAGGGAGTAACTTGCGGAGCGGCAACGTTGTCTGCCGAGCGGGTCAGAGGCGACAAT |
| linker (italicized; | AAGGAGTACGAGTATTCAGTAGAGTGTCAGGAAGATAGCGCCTGTCCCGCCGCGGAGGAGA |
| SEQ ID NO: 98); | GCCTCCCCATCGAGGTGATGGTGGACGCCGTGCACAAGTTAAAGTACGAGAATTACACCAG |
| (4) IL-12α (bolded; | CTCATTTTTTATCAGAGACATTATCAAGCCGGACCCCCCGAAGAACTTACAGCTTAAACCC |
| SEQ ID NO: 123); | CTAAAGAACAGCAGGCAGGTTGAGGTCAGCTGGGAATATCCTGACACCTGGTCAACCCCCC |
| (5) second GS linker | ACAGCTACTTCTCCCTTACTTTCTGTGTGCAAGTGCAGGGCAAGAGCAAGAGAGAAAAGAA |
| (italicized and | GGACCGGGTGTTTACCGACAAGACTAGCGCCACCGTGATTTGCAGAAAGAACGCCAGCATT |
| underlined; SEQ ID | AGTGTGAGAGCCCAGGACAGGTATTACTCCAGCTCATGGTCTGAGTGGGCTAGTGTGCCTT |
| NO: 144); (6) | GCTCTGGAGGCAGCGGTGGCGGGAGCGGCGGAGGCTCCGGGGGAGGTAGCCGGAATCTGCC |
| albumin (lower case | TGTCGCCACTCCAGACCCCGGCATGTTCCCATGTCTGCATCATTCTCAGAACCTGCTGAGG |
| and underlined; SEQ | GCCGTATCCAATATGCTGCAGAAAGCCAGACAGACCTTAGAGTTCTATCCCTGTACAAGCG |
| ID NO: 165); (7) | AGGAGATAGATCACGAGGATATTACGAAGGACAAAACTTCTACTGTTGAGGCGTGTCTTCC |
| third GS linker | ATTAGAGCTGACCAAGAACGAAAGCTGTCTGAATAGCAGAGAGACTTCATTTATCACCAAT |
| (lower case and | GGGAGTTGCTTGGCTAGCAGAAAGACCAGCTTCATGATGGCCCTTTGCTTGTCTTCGATAT |
| italicized; SEQ ID | ACGAAGATCTTAAGATGTATCAAGTGGAATTTAAGACGATGAACGCCAAGCTGCTTATGGA |
| NO: 168); and (8) | TCCCAAGCGCCAAATCTTCCTGGATCAGAACATGTTGGCCGTGATTGACGAGCTGATGCAA |
| lumican (lower case, | GCCCTGAATTTCAACTCCGAGACCGTGCCTCAGAAAAGCAGCCTCGAGGAGCCCGACTTCT |
| italicized, and | ACAAAACAAAGATCAAACTCTGCATCCTTCTGCACGCCTTCAGAATTAGAGCCGTGACCAT |
| underlined; SEQ ID | CGACAGAGTTATGAGCTACCTGAATGCCAGCGGCAGCGGCGGCGGATCCgatgcccataaa |
| NO: 171) | tctgaggtggcccatagattcaaggatctgggcgaagaaaacttcaaagccttggtcttga |
| | tcgcctttgcccagtacctgcagcagtgcccctttgaggaccacgtgaagctggtgaatga |
| | agtgaccgagtttgccaagacgtgcgtggctgatgagagcgccgaaaactgcgacaaaagc |
| | ctgcacaccctgtttggcgacaagctgtgcaccgtagccaccctgagagaaacttacggcg |
| | agatggctgactgctgcgccaagcaggagcccgagagaaacgagtgctttctgcagcacaa |
| | ggacgacaatcccaacctgcccagactggtgagacccgaagtggatgttatgtgcaccgct |
| | ttccacgacaatgaagagacatttctcaagaagtacttgtacgagattgcaagaagacacc |
| | cttacttttacgcccccgaattactgttcttcgctaagaggtataaggcagccttcactga |
| | atgctgccaggctgccgacaaagcagcttgcctgctgccaaagctggatgaactgcgagac |
| | gaaggaaaggcgtcctccgccaagcagcgtttgaagtgcgccagccttcagaagtttggcg |
| | agcgggccttcaaggcatgggccgtggctcgacttagccagcgttttcccaaggctgaatt |
| | tgcagaggtgagtaaactggttaccgatctgacaaaggtgcacaccgagtgctgtcacggt |
| | gacctcttagagtgcgccgacgacagagccgacctcgccaagtacatttgtgaaaaccaag |
| | actcaatctcttcaaagttaaaggagtgctgcgaaaagcccctgcttgaaaagagccactg |
| | cattgccgaagtcgagaatgatgagatgcctgcagacttgcccagcttggcagccgacttc |
| | gttgagtctaaggacgtgtgcaagaattacgccgaggcaaaagacgtgttcctgggcatgt |
| | tcctttatgagtacgctagaagacatcccgactacagcgtggtccttctccttaggctcgc |
| | taagacttacgagacgacgttggagaagtgttgtgccgctgcggacccccacgagtgctat |
| | gccaaagtgttcgatgagtttaaacccctggtggaggaacctcagaaccttatcaagcaga |
| | attgtgagttgttcgaacagctaggcgagtacaagttccagaatgccctgctggtgagata |
| | cacaaaaaaggtgccccaggtgtcaaccccgaccttagtggaagtgtccagaaacctgggc |
| | aaggtgggcagcaagtgctgcaagcaccccgaagctaagagaatgccgtgcgcggaggatt |
| | acctgagcgtggtgctcaaccagctgtgtgtgcttcacgagaaaacacccgtgagcgacag |
| | ggtgacaaaatgttgcacagaaagccttgtgaaccggagaccttgtttcagcgccctggag |
| | gttgacgagacctatgttcctaaggagttcaacgctgagactttcacatttcacgctgata |
| | tatgtaccctgagcgagaaagaaagacagatcaagaagcagaccgccctggtcgagctggt |
| | gaaacacaagcctaaggccacgaaggagcagctgaaggccgtcatggacgacttcgcagcc |
| | ttcgtcgagaaatgctgcaaagccgacgacaaggaaacctgcttcgccgaagagggaaaga |
| | agctggtggccgcctcccaggccgcccttgggctcggtggcgggtctggtggcggttctca |
| | gtactacgattacgacttccccctgagtatctacggtcagtcctcacctaactgcgcccca |
| | gagtgtaactgccccgaaagctacccgagcgccatgtactgcgacgagctgaagttgaagt |
| | ccgtgcccatggtgcccccaggcatcaagtacttatacttgaggaacaaccaaatcgatca |
| | tatcgacgagaaggccttcgaaaacgtaaccgacctgcagtggctgatactggatcacaat |
| | ctgctagagaattccaagatcaagggcagagtgttctcgaagctgaaacaactgaagaagc |
| | tgcacatcaaccataacaatctcaccgagagcgtgggtcccctgcccaagtcgctggagga |
| | cctgcagctgacccacaacaaaataaccaaactaggcagcttcgaggggttggtgaatctg |
| | accttcatccatttgcagcataacagactaaaggaggatgccgtgagcgccgccttcaaag |
| | gcctcaagagccttgagtacctggacctgagcttcaaccagatcgcccggctgcccagtgg |
| | gctgcccgtgagcctgctgacgctgtatctggacaataacaaaatcagcaacatccccgac |
| | gaatacttcaaaagattcaacgccttacagtacctgcgactcagccacaatgagctcgctg |
| | acagcggcatccctggcaacagcttcaacgtgtcatccctggtggagctggacctgagtta |
| | caataagctgaagaacatcccaactgtcaatgagaatttggaaaactactacctggaggtg |
| | aaccagctggagaagttcgacattaagagcttttgcaaaatcctgggcccactgtcatata |
| | gcaagatcaagcacctgcgactggacggcaaccgaatcagtgaaacttccctaccccctga |
| | catgtacgagtgcctgagagtagcaaatgaggtgaccctgaac |
| C2 Construct (SEQ | atgagggtccccgctcagctcctggggctcctgctgctctggctcccaggtgcacgatgtg |
| ID NO: 24)- | ccATCTGGGAGCTGAAGAAAGACGTGTACGTGGTGGAGCTTGACTGGTACCCCGACGCCCC |
| includes (1) leader | CGGTGAAATGGTGGTTCTGACCTGCGACACACCAGAGGAGGACGGCATCACCTGGACCCTG |
| sequence (lower | GACCAGTCCAGCGAGGTCCTGGGATCTGGCAAGACCCTGACCATCCAGGTTAAGGAATTTG |
| case; SEQ ID NO: | GCGACGCCGGCCAGTACACCTGCCACAAAGGCGGCGAGGTCCTTTCGCACAGTCTGCTGCT |
| 49); (2) IL-12ß | GCTGCATAAAAAGGAGGACGGCATTTGGAGCACCGACATTCTGAAGGATCAGAAAGAGCCC |
| (bolded and | AAGAACAAGACCTTTCTGAGATGTGAGGCCAAAAACTACTCTGGACGCTTCACCTGTTGGT |
| underlined; SEQ ID | GGCTGACCACCATCAGCACAGACCTGACCTTCTCGGTGAAGTCTAGTAGGGGCAGCAGTGA |
| NO: 74); (3) first GS | CCCCCAGGGCGTAACATGCGGCGCCGCTACCCTGAGCGCCGAGAGAGTGAGAGGCGATAAC |
| linker (italicized; | AAGGAGTACGAGTACTCCGTGGAGTGCCAAGAGGACTCAGCCTGCCCCGCCGCCGAGGAGT |
| SEQ ID NO: 99); | CGCTGCCCATCGAGGTGATGGTGGATGCAGTGCACAAGCTGAAGTACGAGAACTACACCAG |
| (4) IL-12α (bolded; | TAGCTTTTTCATCAGAGATATCATTAAACCCGACCCTCCCAAGAACCTGCAGCTGAAGCCC |
| SEQ ID NO: 124); | TTAAAGAACAGCCGGCAGGTGGAAGTGTCATGGGAGTACCCAGACACCTGGAGCACTCCGC |
| (5) second GS linker | ACAGCTACTTCAGCCTGACCTTCTGCGTTCAGGTGCAGGGAAAAAGCAAGAGAGAGAAGAA |
| (italicized and | AGACAGAGTGTTCACCGACAAGACCAGCGCAACCGTGATCTGTAGAAAGAACGCCTCGATC |
| underlined; SEQ ID | AGCGTGAGAGCCCAGGACAGATACTACAGCAGCAGCTGGAGCGAGTGGGCCAGTGTACCTT |
| NO: 145); (6) | GCAGCGGTGGAAGCGGCGGAGGCTCTGGCGGAGGGAGTGGCGGAGGCAGCAGAAATCTTCC |
| albumin (lower case | AGTAGCTACCCCCGACCCCGGCATGTTTCCCTGTCTGCACCATTCTCAAAACCTGTTACGG |
| and underlined; SEQ | GCCGTGAGCAACATGCTGCAGAAGGCCAGACAGACACTGGAGTTTTACCCCTGTACCTCAG |
| ID NO: 166); (7) | AGGAGATCGATCATGAGGACATTACTAAGGACAAGACCAGCACCGTGGAAGCCTGCCTGCC |
| third GS linker | CCTAGAGCTAACCAAGAACGAGAGCTGCCTGAACTCTAGAGAGACAAGCTTCATCACGAAC |
| (lower case and | GGCTCATGCCTGGCCAGTAGGAAAACCAGCTTCATGATGGCTCTGTGCCTGAGCTCCATAT |
| italicized; SEQ ID | ATGAGGACCTTAAGATGTACCAGGTGGAGTTCAAAACCATGAACGCCAAGCTGCTGATGGA |
| NO: 169); and (8) | CCCAAAGAGACAGATCTTCCTTGACCAGAACATGCTGGCCGTTATCGATGAGCTGATGCAG |
| lumican (lower case, | GCCCTGAACTTCAACAGCGAGACCGTGCCCCAGAAAAGCAGCCTGGAGGAACCCGACTTCT |
| italicized, and | ACAAAACCAAGATCAAGCTGTGCATTCTGCTGCATGCCTTCCGCATTAGAGCCGTGACCAT |
| underlined; SEQ ID | CGATAGGGTGATGAGCTACCTGAACGCCAGCGGCTCTGGCGGCGGCAGTgacgcccacaag |
| NO: 172) | tccgaggtcgcccacagattcaaggatttgggcgaggagaacttcaaggccctggtgctga |
| | tcgccttcgcccagtacttgcagcagtgtcccttcgaggaccatgtgaagctggtgaacga |
| | ggtgaccgagttcgccaagacctgtgtggccgacgagagcgccgagaactgcgataagtct |
| | ctgcacaccctttttggcgacaaactgtgcaccgtggccaccctgagagagacctacggcg |
| | agatggccgactgctgtgcgaagcaggagcccgagcgcaatgagtgtttcctgcagcataa |
| | ggacgacaaccccaacctgcccagactggtgagacccgaggtggacgtgatgtgcaccgcc |
| | ttccacgacaacgaggagacctttctgaaaaaatacctgtacgagatcgcaagacgccacc |
| | cctacttctacgcccccgagctgctgttcttcgccaagcgctacaaggctgccttcaccga |
| | atgctgccaggccgccgataaggccgcgtgcttactgccaaagctggacgagctgagagac |
| | gaggggaaagcctctagcgccaaacagagattgaagtgtgccagcctgcagaaattcggtg |
| | agagagccttcaaggcctgggccgtggccagattatcacagcggttccccaaggctgaatt |
| | cgccgaggtgagcaaacttgtcaccgatctgacaaaagtgcacaccgagtgctgccatggc |
| | gacctgctggagtgcgccgacgaccgggccgacctggccaagtacatctgcgagaaccagg |
| | acagcatctccagcaagctgaaggagtgctgcgagaagcccctgctggagaaaagccactg |
| | catcgccgaggtggagaatgacgaaatgcccgccgacctgcccagcctggccgccgacttc |
| | gtggaaagcaaggacgtgtgcaaaaattacgccgaagccaaggatgtgttcttgggcatgt |
| | tcttgtacgagtacgccagacgccaccccgactacagcgtggtgctgctgctgcggctggc |
| | caagacctacgagaccaccctggagaagtgctgtgctgccgccgacccccacgagtgctac |
| | gccaaggtatttgacgagttcaagcccctggtggaggagcctcagaacctgattaagcaga |
| | actgtgagctgttcgagcagctgggcgagtacaagttccagaacgccctcctggtgagata |
| | caccaaaaaggtgcctcaggtaagcactcccaccctggtggaggtgagcaggaacctcggc |
| | aaggtgggcagcaaatgctgcaagcacccagaggccaaaagaatgccctgcgcagaagact |
| | acctcagcgtggtcctgaaccagctgtgcgtgctgcacgaaaagacccctgtgagcgatag |
| | agtgacaaagtgctgcaccgagagcctggtgaacagaagaccctgttttagcgccctggag |
| | gtggacgagacctacgtgcccaaggagttcaacgccgagacgttcactttccacgcggaca |
| | tctgcaccctgagcgagaaggagagacaaatcaagaagcagaccgccctagtcgagctggt |
| | aaaacacaagcccaaggccaccaaggagcagctgaaggccgtgatggacgactttgcagcc |
| | ttcgtggagaagtgctgcaaggctgacgacaaggagacctgcttcgccgaggagggcaaga |
| | agctcgtagccgccagccaggccgctctcggccttggtggcgggtctggtggcggttctca |
| | gtattacgactacgatttccccctgagcatctatggccagagcagccctaactgcgccccg |
| | gagtgcaactgccccgaaagctacccaagcgccatgtactgcgatgagctgaagctgaagt |
| | ctgtgcctatggtgcctcccggcatcaagtacctgtacctgagaaacaaccagatagacca |
| | catcgatgagaaagccttcgagaacgtcaccgacctgcagtggctgattctggaccacaat |
| | ttactggagaactccaagatcaagggcagagtgttctccaagttaaagcagctgaagaaac |
| | tgcacatcaatcacaacaacctgaccgagagcgtgggcccactgcccaagagcctagagga |
| | tctgcagctcacccacaacaagatcactaagttgggcagcttcgagggcctcgtaaacttg |
| | acattcatacatctgcagcacaacagacttaaggaagacgccgtgagtgcggcctttaagg |
| | gtctgaaaagcctggagtacttagacctgagcttcaaccagatcgcaaggctgcccagcgg |
| | ccttccggtcagtctgctgaccctgtatctggacaacaacaagatcagcaacatccccgac |
| | gagtacttcaagcggtttaacgccctccagtacctgagactgagccacaacgagttagctg |
| | actcgggcatacccggtaacagcttcaatgttagcagcctagttgagcttgacttgagcta |
| | caacaagcttaagaacatcccaaccgtgaacgagaacctcgagaattactacctggaagtc |
| | aaccagctggagaagttcgacattaagagcttctgcaaaatcctgggcccactgtcctata |
| | gcaagatcaagcacctgcgccttgacggaaacagaattagcgagaccagccttccaccaga |
| | catgtacgagtgcctgagggtggccaacgaggtgaccctgaac |
| C3 Construct (SEQ | atgagggtccccgctcagctcctggggctcctgctgctctggctcccaggtgcacgatgtg |
| ID NO: 25)- | ccATCTGGGAACTGAAGAAGGACGTCTACGTGGTGGAGCTGGATTGGTACCCCGACGCCCC |
| includes (1) leader | CGGCGAGATGGTCGTGCTGACCTGTGACACCCCTGAGGAGGACGGAATCACATGGACCCTG |
| sequence (lower | GACCAGAGCAGCGAAGTGTTGGGCAGCGGCAAGACCCTGACCATCCAAGTGAAGGAATTCG |
| case; SEQ ID NO: | GCGACGCGGGCCAGTATACTTGCCACAAGGGGGGGGAGGTTCTGAGCCACAGCCTGCTGCT |
| 50); (2) IL-12ß | GCTCCACAAGAAAGAGGATGGCATCTGGTCTACCGACATCCTGAAGGACCAAAAGGAGCCA |
| (bolded and | AAGAACAAGACCTTCCTAAGATGCGAGGCCAAGAACTACTCAGGTCGTTTCACCTGCTGGT |
| underlined; SEQ ID | GGCTGACCACAATCTCCACCGACCTGACCTTCAGCGTGAAAAGCAGCCGCGGCAGTAGCGA |
| NO: 75); (3) first GS | CCCACAGGGCGTGACCTGCGGGGCCGCCACTCTGAGCGCCGAGAGAGTGCGCGGCGATAAT |
| linker (italicized; | AAGGAATACGAGTACAGCGTGGAGTGCCAGGAAGACTCAGCCTGCCCCGCCGCAGAAGAAA |
| SEQ ID NO: 100); | GTCTGCCAATAGAGGTGATGGTTGACGCCGTGCACAAGCTAAAGTACGAGAACTACACCAG |
| (4) IL-12α (bolded; | CAGCTTCTTTATCCGGGACATCATCAAGCCAGATCCCCCCAAGAACCTGCAGCTTAAGCCC |
| SEQ ID NO: 125); | CTGAAGAACAGCAGACAGGTGGAGGTGTCATGGGAATACCCCGACACCTGGAGCACCCCCC |
| (5) second GS linker | ATAGCTACTTCTCACTGACCTTCTGCGTGCAGGTGCAGGGCAAGAGCAAGAGAGAGAAGAA |
| (italicized and | GGATAGGGTATTCACGGACAAGACCTCAGCCACCGTGATCTGCCGAAAGAACGCCAGCATC |
| underlined; SEQ ID | AGCGTGAGAGCCCAGGACAGATACTATAGCTCCTCGTGGAGCGAGTGGGCCAGCGTACCTT |
| NO: 146); (6) | GCAGCGGCGGGAGCGGCGGCGGCAGCGGAGGTGGCAGTGGCGGAGGCAGCAGAAACCTCCC |
| albumin (lower case | CGTGGCAACCCCTGACCCCGGGATGTTTCCTTGCCTGCACCACTCCCAGAACCTCCTGAGA |
| and underlined; SEQ | GCCGTGTCCAACATGCTGCAGAAAGCCAGACAGACCCTCGAGTTCTATCCCTGCACAAGCG |
| ID NO: 167); (7) | AGGAGATCGACCACGAGGACATCACTAAGGACAAGACCAGCACAGTGGAAGCCTGCCTCCC |
| third GS linker | GCTGGAGCTGACTAAGAACGAGAGCTGTCTGAACAGCAGGGAGACCAGCTTCATCACCAAC |
| (lower case and | GGCAGCTGCCTGGCGAGCAGAAAAACCTCCTTTATGATGGCGCTCTGCCTGAGCTCAATCT |
| italicized; SEQ ID | ATGAGGACCTGAAGATGTACCAGGTGGAGTTTAAAACCATGAATGCCAAGCTGCTTATGGA |
| NO: 170); and (8) | CCCTAAGAGACAGATTTTCCTGGATCAGAACATGCTGGCCGTGATTGATGAATTAATGCAG |
| lumican (lower case, | GCGCTGAACTTTAACAGCGAGACCGTGCCCCAGAAAAGCAGCCTGGAGGAGCCCGACTTTT |
| italicized, and | ACAAGACCAAGATCAAGTTGTGCATCCTCCTGCACGCCTTCAGAATCAGAGCGGTGACGAT |
| underlined; SEQ ID | CGACAGAGTCATGAGCTACCTCAACGCTAGCGGCAGCGGTGGCGGCAGCgacgcccacaag |
| NO: 173) | agcgaggtggcccacagattcaaggatctcggggaggagaacttcaaggccctggtgctga |
| | tcgccttcgcacagtacctgcagcagtgccccttcgaggaccacgtaaaactggtgaacga |
| | ggtgacggagttcgccaagacctgtgttgccgacgagtcggccgagaattgcgacaagagc |
| | ctgcataccctgttcggcgacaagctgtgcaccgtggccaccctgagagagacctacggcg |
| | agatggccgactgctgcgccaagcaagagcccgagagaaacgagtgcttcctgcagcacaa |
| | ggacgacaaccccaacctgccccggctggtgagacccgaggtggacgtgatgtgcaccgcc |
| | ttccacgacaacgaggagaccttcctgaagaagtatctgtacgagatcgccagaagacacc |
| | cttatttttacgcccccgagctgctgttcttcgctaagagatataaggcagccttcaccga |
| | gtgttgtcaggccgccgataaggccgcttgcctgctgcccaagttggacgagctcagagac |
| | gagggcaaggcgagcagcgccaagcagagactgaagtgcgccagcctgcagaagttcggcg |
| | agagggccttcaaggcctgggcggtggccagactgtcccagagatttcccaaggccgagtt |
| | cgccgaggtaagcaagctggtcaccgacctgaccaaggtgcacaccgagtgttgccacggc |
| | gacctgctggaatgcgccgatgaccgtgccgacctggccaagtacatctgcgagaatcagg |
| | actccatcagcagcaaactgaaggagtgttgcgagaagcccctgctggaaaagagccattg |
| | catcgctgaggtggaaaacgacgagatgcccgcagacctgcccagcctggccgcagacttt |
| | gtggaaagtaaggacgtgtgcaagaactacgcggaggccaaagacgtgtttctgggcatgt |
| | tcctatacgagtatgccagaagacaccccgactacagcgttgtgttattgctgagactggc |
| | caagacctacgagactaccttggagaagtgctgcgccgccgccgacccccacgagtgctat |
| | gccaaggtgttcgacgagttcaagcccctggtggaggagccccagaatctgattaagcaga |
| | attgcgagctttttgagcagctgggcgagtataagttccagaacgccctgctggtgagata |
| | caccaaaaaggtacctcaggtgagcacccccaccctggtggaggtgagcagaaatctgggc |
| | aaggtgggcagcaagtgctgcaagcacccggaggccaagagaatgccctgtgccgaggatt |
| | acctgtcagtggtgctgaaccagctgtgcgttctgcacgaaaagacgcccgtgtcggacag |
| | agtgaccaagtgctgcacggagagcctggtgaacagaagaccgtgcttcagcgccctagag |
| | gtggacgagacctacgtgcccaaggagttcaacgccgagaccttcaccttccacgccgaca |
| | tctgtaccctgtcagagaaggagagacagatcaagaagcagaccgccttagtggagctggt |
| | gaagcacaagcccaaggccaccaaggagcagctgaaagccgtgatggacgatttcgcagcc |
| | ttcgtcgagaagtgctgcaaggccgacgacaaggaaacttgcttcgccgaggagggcaaga |
| | agctggtggctgcctcgcaggccgccctcggcctgggtggcgggtctggtggcggttctca |
| | gtactacgactacgatttccccctatccatctacgggcagagctcgcctaactgcgccccc |
| | gagtgtaactgccccgagtcgtaccccagcgccatgtactgtgacgagctgaagctgaaaa |
| | gcgtgcccatggtgccccccggcatcaagtacctgtacttgagaaacaaccagatcgacca |
| | cattgacgaaaaggccttcgagaacgtaaccgacctgcagtggctgatcctggaccacaac |
| | ctgcttgagaacagcaagatcaagggccgcgtgttcagcaagctgaagcagctgaagaagc |
| | tgcacatcaaccacaacaacttgactgagtctgttggccccctaccaaagagcctggagga |
| | cctgcagctgacccacaataagataaccaagctgggctcattcgagggcctggtgaacttg |
| | acctttattcacctgcagcataacagactgaaggaggacgccgtgagcgccgcctttaagg |
| | ggctgaaaagcctggagtacctggacctgagttttaaccagatcgccagactgccctcagg |
| | cctgcccgtgagtttgctgactctgtacctggacaacaataagatcagcaacattcctgac |
| | gagtatttcaaaagattcaatgctctgcagtacctgagactaagccacaacgagctggccg |
| | acagcggaatccccggcaacagcttcaacgtgagcagcttggtggagttggacctgagcta |
| | caacaaactgaagaacatccccaccgtcaatgagaacttggagaattactacctcgaggtt |
| | aaccagcttgagaagttcgacatcaagagcttctgcaagatcctgggccccctcagctaca |
| | gcaagatcaagcacttgagactggacgggaacagaatcagcgaaaccagccttcctcccga |
| | catgtacgagtgccttagagtggcaaatgaggtgaccctgaac |

In some aspects, an isolated polynucleotide described herein, i.e., comprising a nucleic acid molecule encoding IL-12 (e.g., IL-12α subunit and/or IL-12β subunit), comprises one or more heterologous moieties (e.g., gene(s) of experimental and/or therapeutic interest). As used herein, the term “heterologous moiety” refers to any molecule (chemical or biological) that is different from an IL-12 protein (e.g., IL-12α subunit and/or IL-12β subunit) encoded by a nucleic acid molecule disclosed herein. Such heterologous moieties can be genetically fused, conjugated, and/or otherwise associated to an IL-12 protein. For instance, in some aspects, a heterologous moiety can be fused to the 3′-end of an IL-12α subunit. In some aspects, a heterologous moiety can be conjugated to the 3′-end of an IL-12α subunit via a linker (e.g., GS linker). In some aspects, a heterologous moiety can be fused to the 3′-end of an IL-12β subunit. In some aspects, a heterologous moiety can be conjugated to the 3′-end of an IL-12β subunit via a linker (e.g., GS linker).

In some aspects, a heterologous moiety comprises a half-life extending moiety. The term “half-life extending moiety” refers to a pharmaceutically acceptable moiety, domain, or molecule covalently linked (“conjugated” or “fused”) to an IL-12 protein (e.g., IL-12α subunit and/or IL-12β subunit) encoded by a nucleic acid molecule the present disclosure, optionally via a non-naturally encoded amino acid, directly or via a linker, that prevents or mitigates in vivo proteolytic degradation or other activity-diminishing chemical modification of the IL-12 protein, increases half-life, and/or improves or alters other pharmacokinetic or biophysical properties including but not limited to increasing the rate of absorption, reducing toxicity, improving solubility, reducing protein aggregation, increasing biological activity and/or target selectivity of the IL-12 protein, increasing manufacturability, and/or reducing immunogenicity of the IL-12 protein, compared to a reference compound such as a non-conjugated or non-fused form of the IL-12 protein.

In the context of the present disclosure, the terms “fused” or “fusion” indicate that at least two polypeptide chains (e.g., encoded by the nucleic acid molecules described herein) have been operably linked and recombinantly expressed. In some aspects, two polypeptide chains can be “fused” as a result of chemical synthesis. In the context of the present disclosure, the terms “conjugate” or “conjugation” denote that two molecular entities (e.g., two polypeptides, or a polypeptide and a polymer such as PEG) have been chemically linked.

In some aspects, the half-life extending moiety comprises an Fc region, albumin, albumin binding polypeptide, a fatty acid, Pro/Ala/Ser (PAS), a glycine-rich homo-amino-acid polymer (HAP), the β subunit of the C-terminal peptide (CTP) of human chorionic gonadotropin, polyethylene glycol (PEG), hydroxyethyl starch (HES), long unstructured hydrophilic sequences of amino acids (XTEN), albumin-binding small molecules, or a combination thereof. See, e.g., WO 2013/041730 A1, which is incorporated herein by reference in its entirety. In certain aspects, the half-life extending moiety is albumin (e.g., human serum albumin).

In some aspects, a heterologous moiety comprises a lumican. Lumican binds to collagen, which is abundantly and ubiquitously expressed in tumors. In certain aspects, conjugating a lumican to an IL-12 protein (e.g., IL-12α subunit and/or IL-12β subunit) can improve the targeting of the IL-12 protein to the tumors (e.g., by preventing and/or reducing the IL-12 protein from entering systemic circulation), and thereby reduce toxicity of the IL-12 protein. Additional disclosure relating to lumican (e.g., sequences) that can be used are provided elsewhere in the present disclosure.

In some aspects, the heterologous moieties encode cytokines, chemokines, or growth factors other than IL-12. Cytokines are known in the art, and the term itself refers to a generalized grouping of small proteins that are secreted by certain cells within the immune system and have an effect on other cells. Cytokines are known to enhance the cellular immune response and, as used herein, can include, but are not limited to, TNFα, IFN-γ, IFN-α, TGFβ, IL-1, IL-2, Il-4, IL-10, IL-13, IL-17, IL-18, and chemokines. Chemokines are useful for studies investigating response to infection, immune responses, inflammation, trauma, sepsis, cancer, and reproduction, among other applications. Chemokines are known in the art, and are a type of cytokine that induce chemotaxis in nearby responsive cells, typically of white blood cells, to sites of infection. Non-limiting examples of chemokines include, CCL14, CCL19, CCL20, CCL21, CCL25, CCL27, CXCL12, CXCL13, CXCL-8, CCL2, CCL3, CCL4, CCL5, CCL11, and CXCL10. Growth factors are known in the art and the term itself is sometimes interchangeable with the term cytokines. As used herein, the term “growth factors” refers to a naturally occurring substance capable of signaling between cells and stimulating cellular growth. While cytokines can be growth factors, certain types of cytokines can also have an inhibitory effect on cell growth, thus differentiating the two terms. Non-limiting examples of growth factors include Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (m-CSF), Granulocyte colony-stimulating factor (G-CSF), Granulocyte macrophage colony-stimulating factor (GM-CSF), Epidermal growth factor (EFG), Ephrin A1, Ephrin A2, Ephrin A3, Ephrin A4, Ephrin A5, Ephrin B1, Ephrin B2, Ephrin B3, Erythropoietin (EPO), Fibroblast growth factor-1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Fetal Bovine Somatotrophin (FBS), Glial cell line-derived neurotrophic factor (GDNF), Neurturin, Peresphin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG 4), Brain-derived neurotrophic factor (BDNF), Nerve growth factor (NGF), Neurotrophin-3 (NT-3), Neurotrophin-4 (NT-4), Placental growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

In some aspects, a polynucleotide (e.g., isolated polynucleotide) of the present disclosure further comprises a nucleic acid molecule encoding a leader sequence. As used herein, the term “leader sequence” refers to a sequence located at the amino terminal end of the precursor form of a protein. Leader sequences are cleaved off during maturation. In certain aspects, a leader sequence comprises a signal peptide. The term “signal peptide” refers to a leader sequence ensuring entry of the protein into the secretory pathway. Additional description of leader sequences are provided in, e.g., US 2007/0141666 A1, which is incorporated herein by reference in its entirety. In some aspects, a leader sequence that can be used with the present disclosure comprises the amino acid sequence MRVPAQLLGLLLLWLPGARCA (SEQ ID NO: 180). In some aspects, a nucleic acid molecule encoding such a leader sequence comprises the sequence set forth in any one of SEQ ID NOs: 26 to 50. In some aspects, a nucleic acid molecule encoding the leader sequence comprises a sequence encoding the leader sequence of any of the constructs provided in Table 1.

As will be apparent from the above disclosure, in some aspects, a polynucleotide (e.g., isolated polynucleotide) of the present disclosure comprises multiple nucleic acid molecules. For instance, in certain aspects, a polynucleotide (e.g., isolated polynucleotide) comprises (from 5′ to 3′): (i) a first nucleic acid molecule encoding a leader sequence; (ii) a second nucleic acid molecule encoding an IL-12β subunit; (iii) a third nucleic acid molecule encoding a linker (e.g., GS linker); and (iv) a fourth nucleic acid molecule encoding an IL-12α subunit. As described herein, in some aspects, such a polynucleotide further comprises one or more additional features described herein. For example, in some aspects, a polynucleotide described herein comprises (from 5′ to 3′): (1) a 5′-cap, (2) a first nucleotide sequence encoding a leader sequence, (3) a second nucleotide sequence encoding an IL-12β subunit, (4) a third nucleotide sequence encoding a linker (e.g., GS linker), (5) a fourth nucleotide sequence encoding an IL-12α subunit, and (6) a poly(A) tail. In some aspects, a polynucleotide described herein comprises (from 5′ to 3′): (1) a 5′-cap, (2) a 5′-UTR, (3) a promoter, (4) a first nucleotide sequence encoding a leader sequence, (5) a second nucleotide sequence encoding an IL-12β subunit, (6) a third nucleotide sequence encoding a linker (e.g., GS linker), (7) a fourth nucleotide sequence encoding an IL-12α subunit, (8) a 3′-UTR, and (9) a poly(A) tail. Additional description of exemplary constructs are provided below.

In some aspects, (i) the first nucleic acid molecule (i.e., encoding the leader sequence) comprises the sequence set forth in SEQ ID NO: 40; (ii) the second nucleic acid molecule (i.e., encoding the IL-12β subunit) comprises the sequence set forth in SEQ ID NO: 65; (iii) the third nucleic acid molecule (i.e., encoding the linker) comprises the sequence set forth in SEQ ID NO: 90; and (iv) the fourth nucleic acid molecule (i.e., encoding the IL-12α subunit) comprises the sequence set forth in SEQ ID NO: 115. Accordingly, in certain aspects, a polynucleotide (e.g., isolated polynucleotide) described herein comprises the sequence set forth in SEQ ID NO: 15. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 40 (i.e., leader sequence), (3) a second nucleotide sequence comprising the sequence set forth in SEQ ID NO: 65 (i.e., IL-12β subunit), (4) a third nucleotide sequence comprising the sequence set forth in SEQ ID NO: 90 (i.e., GS linker), (5) a fourth nucleotide sequence comprising the sequence set forth in SEQ ID NO: 115 (i.e., IL-12α subunit), and (6) a poly(A) tail. An example of such a polynucleotide is described herein as the “A1 Construct.”

In some aspects, (i) the first nucleic acid molecule comprises the sequence set forth in SEQ ID NO: 41; (ii) the second nucleic acid molecule comprises the sequence set forth in SEQ ID NO: 66; (iii) the third nucleic acid molecule comprises the sequence set forth in SEQ ID NO: 91; and (iv) the fourth nucleic acid molecule comprises the sequence set forth in SEQ ID NO: 116. Accordingly, in certain aspects, a polynucleotide (e.g., isolated polynucleotide) described herein comprises the sequence set forth in SEQ ID NO: 16. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 41 (i.e., leader sequence), (3) a second nucleotide sequence comprising the sequence set forth in SEQ ID NO: 66 (i.e., IL-12β subunit), (4) a third nucleotide sequence comprising the sequence set forth in SEQ ID NO: 91 (i.e., GS linker), (5) a fourth nucleotide sequence comprising the sequence set forth in SEQ ID NO: 116 (i.e., IL-12α subunit), and (6) a poly(A) tail. An example of such a polynucleotide is described herein as the “A2 Construct.”

In some aspects, (i) the first nucleic acid molecule comprises the sequence set forth in SEQ ID NO: 42; (ii) the second nucleic acid molecule comprises the sequence set forth in SEQ ID NO: 67; (iii) the third nucleic acid molecule comprises the sequence set forth in SEQ ID NO: 92; and (iv) the fourth nucleic acid molecule comprises the sequence set forth in SEQ ID NO: 117. Accordingly, in certain aspects, a polynucleotide (e.g., isolated polynucleotide) described herein comprises the sequence set forth in SEQ ID NO: 17. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 42 (i.e., leader sequence), (3) a second nucleotide sequence comprising the sequence set forth in SEQ ID NO: 67 (i.e., IL-12β subunit), (4) a third nucleotide sequence comprising the sequence set forth in SEQ ID NO: 92 (i.e., GS linker), (5) a fourth nucleotide sequence comprising the sequence set forth in SEQ ID NO: 117 (i.e., IL-12α subunit), and (6) a poly(A) tail. An example of such a polynucleotide is described herein as the “A3 Construct.”

In some aspects, (i) the first nucleic acid molecule comprises the sequence set forth in SEQ ID NO: 43; (ii) the second nucleic acid molecule comprises the sequence set forth in SEQ ID NO: 68; (iii) the third nucleic acid molecule comprises the sequence set forth in SEQ ID NO: 93; and (iv) the fourth nucleic acid molecule comprises the sequence set forth in SEQ ID NO: 118. Accordingly, in certain aspects, a polynucleotide (e.g., isolated polynucleotide) described herein comprises the sequence set forth in SEQ ID NO: 18. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 43 (i.e., leader sequence), (3) a second nucleotide sequence comprising the sequence set forth in SEQ ID NO: 68 (i.e., IL-12β subunit), (4) a third nucleotide sequence comprising the sequence set forth in SEQ ID NO: 93 (i.e., GS linker), (5) a fourth nucleotide sequence comprising the sequence set forth in SEQ ID NO: 118 (i.e., IL-12α subunit), and (6) a poly(A) tail. An example of such a polynucleotide is described herein as the “A4 Construct.”

In some aspects, a polynucleotide (e.g., isolated polynucleotide) described herein comprises (from 5′ to 3′): (i) a first nucleic acid molecule encoding a leader sequence; (ii) a second nucleic acid molecule encoding an IL-12β subunit; (iii) a third nucleic acid molecule encoding a first linker (e.g., first GS linker); (iv) a fourth nucleic acid molecule encoding an IL-12α subunit; (v) a fifth nucleic acid molecule encoding a second linker (e.g., second GS linker); and (vi) a sixth nucleic acid molecule encoding a half-life extending moiety (e.g., human serum albumin). As described herein, in some aspects, such a polynucleotide further comprises one or more additional features described herein. For example, in some aspects, a polynucleotide described herein comprises (from 5′ to 3′): (1) a 5′-cap, (2) a first nucleotide sequence encoding a leader sequence, (3) a second nucleotide sequence encoding an IL-12β subunit, (4) a third nucleotide sequence encoding a first linker (e.g., GS linker), (5) a fourth nucleotide sequence encoding an IL-12α subunit, (6) a fifth nucleotide sequence encoding a second linker (e.g., GS linker), (7) a sixth nucleotide sequence encoding a half-life extending moiety (e.g., human serum albumin), and (8) a poly(A) tail. In some aspects, a polynucleotide described herein comprises (from 5′ to 3′): (1) a 5′-cap, (2) a 5′-UTR, (3) a promoter, (4) a first nucleotide sequence encoding a leader sequence, (5) a second nucleotide sequence encoding an IL-12β subunit, (6) a third nucleotide sequence encoding a first linker (e.g., GS linker), (7) a fourth nucleotide sequence encoding an IL-12α subunit, (8) a fifth nucleotide sequence encoding a second linker (e.g., GS linker), (9) a sixth nucleotide sequence encoding a heterologous moiety (e.g., albumin), (10) a 3′-UTR, and (11) a poly(A) tail. Additional description of such exemplary constructs are provided below.

In some aspects, (i) the first nucleic acid molecule (i.e., encoding the leader sequence) comprises the sequence set forth in SEQ ID NO: 26; (ii) the second nucleic acid molecule (i.e., encoding the IL-12β subunit) comprises the sequence set forth in SEQ ID NO: 51; (iii) the third nucleic acid molecule (i.e., encoding the first linker) comprises the sequence set forth in SEQ ID NO: 76; (iv) the fourth nucleic acid molecule (i.e., encoding the IL-12α subunit) comprises the sequence set forth in SEQ ID NO: 101; (v) the fifth nucleic acid molecule (i.e., encoding the second linker) comprises the sequence set forth in SEQ ID NO: 126; and (vi) the sixth nucleic acid molecule (i.e., encoding the half-life extending moiety) comprises the sequence set forth in SEQ ID NO: 147. Accordingly, in certain aspects, a polynucleotide (e.g., isolated polynucleotide) described herein comprises the sequence set forth in SEQ ID NO: 1. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 26 (i.e., leader sequence), (3) a second nucleotide sequence comprising the sequence set forth in SEQ ID NO: 51 (i.e., IL-12β subunit), (4) a third nucleotide sequence comprising the sequence set forth in SEQ ID NO: 76 (i.e., first GS linker), (5) a fourth nucleotide sequence comprising the sequence set forth in SEQ ID NO: 101 (i.e., IL-12α subunit), (6) a fifth nucleotide sequence comprising the sequence set forth in SEQ ID NO: 126 (i.e., second GS linker), (7) a sixth nucleotide sequence comprising the sequence set forth in SEQ ID NO: 147 (i.e., human serum albumin), and (8) a poly(A) tail. An example of such a polynucleotide is described herein as the “L1 Construct.”

In some aspects, (i) the first nucleic acid molecule comprises the sequence set forth in SEQ ID NO: 27; (ii) the second nucleic acid molecule comprises the sequence set forth in SEQ ID NO: 52; (iii) the third nucleic acid molecule comprises the sequence set forth in SEQ ID NO: 77 (iv) the fourth nucleic acid molecule comprises the sequence set forth in SEQ ID NO: 102; (v) the fifth nucleic acid molecule comprises the sequence set forth in SEQ ID NO: 127; and (vi) the sixth nucleic acid molecule comprises the sequence set forth in SEQ ID NO: 148. Accordingly, in certain aspects, a polynucleotide (e.g., isolated polynucleotide) described herein comprises the sequence set forth in SEQ ID NO: 2. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 27 (i.e., leader sequence), (3) a second nucleotide sequence comprising the sequence set forth in SEQ ID NO: 52 (i.e., IL-12β subunit), (4) a third nucleotide sequence comprising the sequence set forth in SEQ ID NO: 77 (i.e., GS linker), (5) a fourth nucleotide sequence comprising the sequence set forth in SEQ ID NO: 102 (i.e., IL-12α subunit), (6) a fifth nucleotide sequence comprising the sequence st forth in SEQ ID NO: 127 (i.e., second GS linker), (7) a sixth nucleotide sequence comprising the sequence set forth in SEQ ID NO: 148 (i.e., human serum albumin), and (8) a poly(A) tail. An example of such a polynucleotide is described herein as the “L2 Construct.”

In some aspects, (i) the first nucleic acid molecule comprises the sequence set forth in SEQ ID NO: 28; (ii) the second nucleic acid molecule comprises the sequence set forth in SEQ ID NO: 53; (iii) the third nucleic acid molecule comprises the sequence set forth in SEQ ID NO: 78; (iv) the fourth nucleic acid molecule comprises the sequence set forth in SEQ ID NO: 103; (v) the fifth nucleic acid molecule comprises the sequence set forth in SEQ ID NO: 128; and (vi) the sixth nucleic acid molecule comprises the sequence set forth in SEQ ID NO: 149. Accordingly, in certain aspects, a polynucleotide (e.g., isolated polynucleotide) described herein comprises the sequence set forth in SEQ ID NO: 3. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 28 (i.e., leader sequence), (3) a second nucleotide sequence comprising the sequence set forth in SEQ ID NO: 53 (i.e., IL-12β subunit), (4) a third nucleotide sequence comprising the sequence set forth in SEQ ID NO: 78 (i.e., GS linker), (5) a fourth nucleotide sequence comprising the sequence set forth in SEQ ID NO: 103 (i.e., IL-12α subunit), (6) a fifth nucleotide sequence comprising the sequence st forth in SEQ ID NO: 128 (i.e., second GS linker), (7) a sixth nucleotide sequence comprising the sequence set forth in SEQ ID NO: 149 (i.e., human serum albumin), and (8) a poly(A) tail. An example of such a polynucleotide is described herein as the “L3 Construct.”

In some aspects, (i) the first nucleic acid molecule comprises the sequence set forth in SEQ ID NO: 29; (ii) the second nucleic acid molecule comprises the sequence set forth in SEQ ID NO: 54; (iii) the third nucleic acid molecule comprises the sequence set forth in SEQ ID NO: 79; (iv) the fourth nucleic acid molecule comprises the sequence set forth in SEQ ID NO: 104; (v) the fifth nucleic acid molecule comprises the sequence set forth in SEQ ID NO: 129; and (vi) the sixth nucleic acid molecule comprises the sequence set forth in SEQ ID NO: 150. Accordingly, in certain aspects, a polynucleotide (e.g., isolated polynucleotide) described herein comprises the sequence set forth in SEQ ID NO: 4. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 29 (i.e., leader sequence), (3) a second nucleotide sequence comprising the sequence set forth in SEQ ID NO: 54 (i.e., IL-12β subunit), (4) a third nucleotide sequence comprising the sequence set forth in SEQ ID NO: 79 (i.e., GS linker), (5) a fourth nucleotide sequence comprising the sequence set forth in SEQ ID NO: 104 (i.e., IL-12α subunit), (6) a fifth nucleotide sequence comprising the sequence st forth in SEQ ID NO: 129 (i.e., second GS linker), (7) a sixth nucleotide sequence comprising the sequence set forth in SEQ ID NO: 150 (i.e., human serum albumin), and (8) a poly(A) tail. An example of such a polynucleotide is described herein as the “M1 Construct.”

In some aspects, (i) the first nucleic acid molecule comprises the sequence set forth in SEQ ID NO: 30; (ii) the second nucleic acid molecule comprises the sequence set forth in SEQ ID NO: 55; (iii) the third nucleic acid molecule comprises the sequence set forth in SEQ ID NO: 80; (iv) the fourth nucleic acid molecule comprises the sequence set forth in SEQ ID NO: 105; (v) the fifth nucleic acid molecule comprises the sequence set forth in SEQ ID NO: 130; and (vi) the sixth nucleic acid molecule comprises the sequence set forth in SEQ ID NO: 151. Accordingly, in certain aspects, a polynucleotide (e.g., isolated polynucleotide) described herein comprises the sequence set forth in SEQ ID NO: 5. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 30 (i.e., leader sequence), (3) a second nucleotide sequence comprising the sequence set forth in SEQ ID NO: 55 (i.e., IL-12β subunit), (4) a third nucleotide sequence comprising the sequence set forth in SEQ ID NO: 80 (i.e., GS linker), (5) a fourth nucleotide sequence comprising the sequence set forth in SEQ ID NO: 105 (i.e., IL-12α subunit), (6) a fifth nucleotide sequence comprising the sequence st forth in SEQ ID NO: 130 (i.e., second GS linker), (7) a sixth nucleotide sequence comprising the sequence set forth in SEQ ID NO: 151 (i.e., human serum albumin), and (8) a poly(A) tail. An example of such a polynucleotide is described herein as the “M2 Construct.”

In some aspects, (i) the first nucleic acid molecule comprises the sequence set forth in SEQ ID NO: 31; (ii) the second nucleic acid molecule comprises the sequence set forth in SEQ ID NO: 56; (iii) the third nucleic acid molecule comprises the sequence set forth in SEQ ID NO: 81; (iv) the fourth nucleic acid molecule comprises the sequence set forth in SEQ ID NO: 106; (v) the fifth nucleic acid molecule comprises the sequence set forth in SEQ ID NO: 131; and (vi) the sixth nucleic acid molecule comprises the sequence set forth in SEQ ID NO: 152. Accordingly, in certain aspects, a polynucleotide (e.g., isolated polynucleotide) described herein comprises the sequence set forth in SEQ ID NO: 6. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 31 (i.e., leader sequence), (3) a second nucleotide sequence comprising the sequence set forth in SEQ ID NO: 56 (i.e., IL-12β subunit), (4) a third nucleotide sequence comprising the sequence set forth in SEQ ID NO: 81 (i.e., GS linker), (5) a fourth nucleotide sequence comprising the sequence set forth in SEQ ID NO: 106 (i.e., IL-12α subunit), (6) a fifth nucleotide sequence comprising the sequence st forth in SEQ ID NO: 131 (i.e., second GS linker), (7) a sixth nucleotide sequence comprising the sequence set forth in SEQ ID NO: 152 (i.e., human serum albumin), and (8) a poly(A) tail. An example of such a polynucleotide is described herein as the “M3 Construct.”

In some aspects, (i) the first nucleic acid molecule comprises the sequence set forth in SEQ ID NO: 32; (ii) the second nucleic acid molecule comprises the sequence set forth in SEQ ID NO: 57; (iii) the third nucleic acid molecule comprises the sequence set forth in SEQ ID NO: 82; (iv) the fourth nucleic acid molecule comprises the sequence set forth in SEQ ID NO: 107; (v) the fifth nucleic acid molecule comprises the sequence set forth in SEQ ID NO: 132; and (vi) the sixth nucleic acid molecule comprises the sequence set forth in SEQ ID NO: 153. Accordingly, in certain aspects, a polynucleotide (e.g., isolated polynucleotide) described herein comprises the sequence set forth in SEQ ID NO: 7. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 32 (i.e., leader sequence), (3) a second nucleotide sequence comprising the sequence set forth in SEQ ID NO: 57 (i.e., IL-12β subunit), (4) a third nucleotide sequence comprising the sequence set forth in SEQ ID NO: 82 (i.e., GS linker), (5) a fourth nucleotide sequence comprising the sequence set forth in SEQ ID NO: 107 (i.e., IL-12α subunit), (6) a fifth nucleotide sequence comprising the sequence st forth in SEQ ID NO: 132 (i.e., second GS linker), (7) a sixth nucleotide sequence comprising the sequence set forth in SEQ ID NO: 153 (i.e., human serum albumin), and (8) a poly(A) tail. An example of such a polynucleotide is described herein as the “H1 Construct.”

In some aspects, (i) the first nucleic acid molecule comprises the sequence set forth in SEQ ID NO: 33; (ii) the second nucleic acid molecule comprises the sequence set forth in SEQ ID NO: 58; (iii) the third nucleic acid molecule comprises the sequence set forth in SEQ ID NO: 83; (iv) the fourth nucleic acid molecule comprises the sequence set forth in SEQ ID NO: 108; (v) the fifth nucleic acid molecule comprises the sequence set forth in SEQ ID NO: 133; and (vi) the sixth nucleic acid molecule comprises the sequence set forth in SEQ ID NO: 154. Accordingly, in certain aspects, a polynucleotide (e.g., isolated polynucleotide) described herein comprises the sequence set forth in SEQ ID NO: 8. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 33 (i.e., leader sequence), (3) a second nucleotide sequence comprising the sequence set forth in SEQ ID NO: 58 (i.e., IL-12β subunit), (4) a third nucleotide sequence comprising the sequence set forth in SEQ ID NO: 83 (i.e., GS linker), (5) a fourth nucleotide sequence comprising the sequence set forth in SEQ ID NO: 108 (i.e., IL-12α subunit), (6) a fifth nucleotide sequence comprising the sequence st forth in SEQ ID NO: 133 (i.e., second GS linker), (7) a sixth nucleotide sequence comprising the sequence set forth in SEQ ID NO: 154 (i.e., human serum albumin), and (8) a poly(A) tail. An example of such a polynucleotide is described herein as the “H2 Construct.”

In some aspects, (i) the first nucleic acid molecule comprises the sequence set forth in SEQ ID NO: 34; (ii) the second nucleic acid molecule comprises the sequence set forth in SEQ ID NO: 59; (iii) the third nucleic acid molecule comprises the sequence set forth in SEQ ID NO: 84; (iv) the fourth nucleic acid molecule comprises the sequence set forth in SEQ ID NO: 109; (v) the fifth nucleic acid molecule comprises the sequence set forth in SEQ ID NO: 134; and (vi) the sixth nucleic acid molecule comprises the sequence set forth in SEQ ID NO: 155. Accordingly, in certain aspects, a polynucleotide (e.g., isolated polynucleotide) described herein comprises the sequence set forth in SEQ ID NO: 9. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 34 (i.e., leader sequence), (3) a second nucleotide sequence comprising the sequence set forth in SEQ ID NO: 59 (i.e., IL-12β subunit), (4) a third nucleotide sequence comprising the sequence set forth in SEQ ID NO: 84 (i.e., GS linker), (5) a fourth nucleotide sequence comprising the sequence set forth in SEQ ID NO: 109 (i.e., IL-12α subunit), (6) a fifth nucleotide sequence comprising the sequence st forth in SEQ ID NO: 134 (i.e., second GS linker), (7) a sixth nucleotide sequence comprising the sequence set forth in SEQ ID NO: 155 (i.e., human serum albumin), and (8) a poly(A) tail. An example of such a polynucleotide is described herein as the “H3 Construct.”

In some aspects, (i) the first nucleic acid molecule comprises the sequence set forth in SEQ ID NO: 37; (ii) the second nucleic acid molecule comprises the sequence set forth in SEQ ID NO: 62; (iii) the third nucleic acid molecule comprises the sequence set forth in SEQ ID NO: 87; (iv) the fourth nucleic acid molecule comprises the sequence set forth in SEQ ID NO: 112; (v) the fifth nucleic acid molecule comprises the sequence set forth in SEQ ID NO: 137; and (vi) the sixth nucleic acid molecule comprises the sequence set forth in SEQ ID NO: 158. Accordingly, in certain aspects, a polynucleotide (e.g., isolated polynucleotide) described herein comprises the sequence set forth in SEQ ID NO: 12. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 37 (i.e., leader sequence), (3) a second nucleotide sequence comprising the sequence set forth in SEQ ID NO: 62 (i.e., IL-12β subunit), (4) a third nucleotide sequence comprising the sequence set forth in SEQ ID NO: 87 (i.e., GS linker), (5) a fourth nucleotide sequence comprising the sequence set forth in SEQ ID NO: 112 (i.e., IL-12α subunit), (6) a fifth nucleotide sequence comprising the sequence st forth in SEQ ID NO: 137 (i.e., second GS linker), (7) a sixth nucleotide sequence comprising the sequence set forth in SEQ ID NO: 158 (i.e., human serum albumin), and (8) a poly(A) tail. An example of such a polynucleotide is described herein as the “vH1 Construct.”

In some aspects, (i) the first nucleic acid molecule comprises the sequence set forth in SEQ ID NO: 38; (ii) the second nucleic acid molecule comprises the sequence set forth in SEQ ID NO: 63; (iii) the third nucleic acid molecule comprises the sequence set forth in SEQ ID NO: 88; (iv) the fourth nucleic acid molecule comprises the sequence set forth in SEQ ID NO: 113; (v) the fifth nucleic acid molecule comprises the sequence set forth in SEQ ID NO: 138; and (vi) the sixth nucleic acid molecule comprises the sequence set forth in SEQ ID NO: 159. Accordingly, in certain aspects, a polynucleotide (e.g., isolated polynucleotide) described herein comprises the sequence set forth in SEQ ID NO: 13. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 38 (i.e., leader sequence), (3) a second nucleotide sequence comprising the sequence set forth in SEQ ID NO: 63 (i.e., IL-12β subunit), (4) a third nucleotide sequence comprising the sequence set forth in SEQ ID NO: 88 (i.e., GS linker), (5) a fourth nucleotide sequence comprising the sequence set forth in SEQ ID NO: 113 (i.e., IL-12α subunit), (6) a fifth nucleotide sequence comprising the sequence st forth in SEQ ID NO: 138 (i.e., second GS linker), (7) a sixth nucleotide sequence comprising the sequence set forth in SEQ ID NO: 159 (i.e., human serum albumin), and (8) a poly(A) tail. An example of such a polynucleotide is described herein as the “vH2 Construct.”

In some aspects, (i) the first nucleic acid molecule comprises the sequence set forth in SEQ ID NO: 39; (ii) the second nucleic acid molecule comprises the sequence set forth in SEQ ID NO: 64; (iii) the third nucleic acid molecule comprises the sequence set forth in SEQ ID NO: 89; (iv) the fourth nucleic acid molecule comprises the sequence set forth in SEQ ID NO: 114; (v) the fifth nucleic acid molecule comprises the sequence set forth in SEQ ID NO: 139; and (vi) the sixth nucleic acid molecule comprises the sequence set forth in SEQ ID NO: 160. Accordingly, in certain aspects, a polynucleotide (e.g., isolated polynucleotide) described herein comprises the sequence set forth in SEQ ID NO: 14. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 39 (i.e., leader sequence), (3) a second nucleotide sequence comprising the sequence set forth in SEQ ID NO: 64 (i.e., IL-12β subunit), (4) a third nucleotide sequence comprising the sequence set forth in SEQ ID NO: 89 (i.e., GS linker), (5) a fourth nucleotide sequence comprising the sequence set forth in SEQ ID NO: 114 (i.e., IL-12α subunit), (6) a fifth nucleotide sequence comprising the sequence st forth in SEQ ID NO: 139 (i.e., second GS linker), (7) a sixth nucleotide sequence comprising the sequence set forth in SEQ ID NO: 160 (i.e., human serum albumin), and (8) a poly(A) tail. An example of such a polynucleotide is described herein as the “vH3 Construct.”

In some aspects, (i) the first nucleic acid molecule comprises the sequence set forth in SEQ ID NO: 44; (ii) the second nucleic acid molecule comprises the sequence set forth in SEQ ID NO: 69; (iii) the third nucleic acid molecule comprises the sequence set forth in SEQ ID NO: 94; (iv) the fourth nucleic acid molecule comprises the sequence set forth in SEQ ID NO: 119; (v) the fifth nucleic acid molecule comprises the sequence set forth in SEQ ID NO: 140; and (vi) the sixth nucleic acid molecule comprises the sequence set forth in SEQ ID NO: 161. Accordingly, in certain aspects, a polynucleotide (e.g., isolated polynucleotide) described herein comprises the sequence set forth in SEQ ID NO: 19. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 44 (i.e., leader sequence), (3) a second nucleotide sequence comprising the sequence set forth in SEQ ID NO: 69 (i.e., IL-12β subunit), (4) a third nucleotide sequence comprising the sequence set forth in SEQ ID NO: 94 (i.e., GS linker), (5) a fourth nucleotide sequence comprising the sequence set forth in SEQ ID NO: 119 (i.e., IL-12α subunit), (6) a fifth nucleotide sequence comprising the sequence st forth in SEQ ID NO: 140 (i.e., second GS linker), (7) a sixth nucleotide sequence comprising the sequence set forth in SEQ ID NO: 161 (i.e., human serum albumin), and (8) a poly(A) tail. An example of such a polynucleotide is described herein as the “B1 Construct.”

In some aspects, (i) the first nucleic acid molecule comprises the sequence set forth in SEQ ID NO: 45; (ii) the second nucleic acid molecule comprises the sequence set forth in SEQ ID NO: 70; (iii) the third nucleic acid molecule comprises the sequence set forth in SEQ ID NO: 95; (iv) the fourth nucleic acid molecule comprises the sequence set forth in SEQ ID NO: 120; (v) the fifth nucleic acid molecule comprises the sequence set forth in SEQ ID NO: 141; and (vi) the sixth nucleic acid molecule comprises the sequence set forth in SEQ ID NO: 162. Accordingly, in certain aspects, a polynucleotide (e.g., isolated polynucleotide) described herein comprises the sequence set forth in SEQ ID NO: 20. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 45 (i.e., leader sequence), (3) a second nucleotide sequence comprising the sequence set forth in SEQ ID NO: 70 (i.e., IL-12β subunit), (4) a third nucleotide sequence comprising the sequence set forth in SEQ ID NO: 95 (i.e., GS linker), (5) a fourth nucleotide sequence comprising the sequence set forth in SEQ ID NO: 120 (i.e., IL-12α subunit), (6) a fifth nucleotide sequence comprising the sequence st forth in SEQ ID NO: 141 (i.e., second GS linker), (7) a sixth nucleotide sequence comprising the sequence set forth in SEQ ID NO: 162 (i.e., human serum albumin), and (8) a poly(A) tail. An example of such a polynucleotide is described herein as the “B2 Construct.”

In some aspects, (i) the first nucleic acid molecule comprises the sequence set forth in SEQ ID NO: 46; (ii) the second nucleic acid molecule comprises the sequence set forth in SEQ ID NO: 71; (iii) the third nucleic acid molecule comprises the sequence set forth in SEQ ID NO: 96; (iv) the fourth nucleic acid molecule comprises the sequence set forth in SEQ ID NO: 121; (v) the fifth nucleic acid molecule comprises the sequence set forth in SEQ ID NO: 142; and (vi) the sixth nucleic acid molecule comprises the sequence set forth in SEQ ID NO: 163. Accordingly, in certain aspects, a polynucleotide (e.g., isolated polynucleotide) described herein comprises the sequence set forth in SEQ ID NO: 21. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 46 (i.e., leader sequence), (3) a second nucleotide sequence comprising the sequence set forth in SEQ ID NO: 71 (i.e., IL-12β subunit), (4) a third nucleotide sequence comprising the sequence set forth in SEQ ID NO: 96 (i.e., GS linker), (5) a fourth nucleotide sequence comprising the sequence set forth in SEQ ID NO: 121 (i.e., IL-12α subunit), (6) a fifth nucleotide sequence comprising the sequence st forth in SEQ ID NO: 142 (i.e., second GS linker), (7) a sixth nucleotide sequence comprising the sequence set forth in SEQ ID NO: 163 (i.e., human serum albumin), and (8) a poly(A) tail. An example of such a polynucleotide is described herein as the “B3 Construct.”

In some aspects, (i) the first nucleic acid molecule comprises the sequence set forth in SEQ ID NO: 47; (ii) the second nucleic acid molecule comprises the sequence set forth in SEQ ID NO: 72; (iii) the third nucleic acid molecule comprises the sequence set forth in SEQ ID NO: 97; (iv) the fourth nucleic acid molecule comprises the sequence set forth in SEQ ID NO: 122; (v) the fifth nucleic acid molecule comprises the sequence set forth in SEQ ID NO: 143; and (vi) the sixth nucleic acid molecule comprises the sequence set forth in SEQ ID NO: 164. Accordingly, in certain aspects, a polynucleotide (e.g., isolated polynucleotide) described herein comprises the sequence set forth in SEQ ID NO: 22. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 47 (i.e., leader sequence), (3) a second nucleotide sequence comprising the sequence set forth in SEQ ID NO: 72 (i.e., IL-12β subunit), (4) a third nucleotide sequence comprising the sequence set forth in SEQ ID NO: 97 (i.e., GS linker), (5) a fourth nucleotide sequence comprising the sequence set forth in SEQ ID NO: 122 (i.e., IL-12α subunit), (6) a fifth nucleotide sequence comprising the sequence st forth in SEQ ID NO: 143 (i.e., second GS linker), (7) a sixth nucleotide sequence comprising the sequence set forth in SEQ ID NO: 164 (i.e., human serum albumin), and (8) a poly(A) tail. An example of such a polynucleotide is described herein as the “B4 Construct.”

In some aspects, (i) the first nucleic acid molecule comprises the sequence set forth in SEQ ID NO: 35; (ii) the second nucleic acid molecule comprises the sequence set forth in SEQ ID NO: 60; (iii) the third nucleic acid molecule comprises the sequence set forth in SEQ ID NO: 85; (iv) the fourth nucleic acid molecule comprises the sequence set forth in SEQ ID NO: 110; (v) the fifth nucleic acid molecule comprises the sequence set forth in SEQ ID NO: 135; and (vi) the sixth nucleic acid molecule comprises the sequence set forth in SEQ ID NO: 156. Accordingly, in certain aspects, a polynucleotide (e.g., isolated polynucleotide) described herein comprises the sequence set forth in SEQ ID NO: 10. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 35 (i.e., leader sequence), (3) a second nucleotide sequence comprising the sequence set forth in SEQ ID NO: 60 (i.e., IL-12β subunit), (4) a third nucleotide sequence comprising the sequence set forth in SEQ ID NO: 85 (i.e., GS linker), (5) a fourth nucleotide sequence comprising the sequence set forth in SEQ ID NO: 110 (i.e., IL-12α subunit), (6) a fifth nucleotide sequence comprising the sequence st forth in SEQ ID NO: 135 (i.e., second GS linker), (7) a sixth nucleotide sequence comprising the sequence set forth in SEQ ID NO: 156 (i.e., human serum albumin), and (8) a poly(A) tail. An example of such a polynucleotide is described herein as the “CO Construct.”

In some aspects, (i) the first nucleic acid molecule comprises the sequence set forth in SEQ ID NO: 36; (ii) the second nucleic acid molecule comprises the sequence set forth in SEQ ID NO: 61; (iii) the third nucleic acid molecule comprises the sequence set forth in SEQ ID NO: 86; (iv) the fourth nucleic acid molecule comprises the sequence set forth in SEQ ID NO: 111; (v) the fifth nucleic acid molecule comprises the sequence set forth in SEQ ID NO: 136; and (vi) the sixth nucleic acid molecule comprises the sequence set forth in SEQ ID NO: 157. Accordingly, in certain aspects, a polynucleotide (e.g., isolated polynucleotide) described herein comprises the sequence set forth in SEQ ID NO: 11. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 36 (i.e., leader sequence), (3) a second nucleotide sequence comprising the sequence set forth in SEQ ID NO: 61 (i.e., IL-12β subunit), (4) a third nucleotide sequence comprising the sequence set forth in SEQ ID NO: 86 (i.e., GS linker), (5) a fourth nucleotide sequence comprising the sequence set forth in SEQ ID NO: 111 (i.e., IL-12α subunit), (6) a fifth nucleotide sequence comprising the sequence st forth in SEQ ID NO: 136 (i.e., second GS linker), (7) a sixth nucleotide sequence comprising the sequence set forth in SEQ ID NO: 157 (i.e., human serum albumin), and (8) a poly(A) tail. An example of such a polynucleotide is described herein as the “CP Construct.”

In some aspects, a polynucleotide (e.g., isolated polynucleotide) described herein comprises (from 5′ to 3′): (i) a first nucleic acid molecule encoding a leader sequence; (ii) a second nucleic acid molecule encoding an IL-12β subunit; (iii) a third nucleic acid molecule encoding a first linker (e.g., first GS linker); (iv) a fourth nucleic acid molecule encoding an IL-12α subunit; (v) a fifth nucleic acid molecule encoding a second linker (e.g., second GS linker); (vi) a sixth nucleic acid molecule encoding a half-life extending moiety (e.g., human serum albumin); (vii) a seventh nucleic acid molecule encoding a third linker (e.g., third GS linker); and (viii) an eighth nucleic acid molecule encoding a lumican. As described herein, in some aspects, such a polynucleotide further comprises one or more additional features described herein. In some aspects, a polynucleotide described herein comprises (from 5′ to 3′): (1) a 5′-cap, (2) a first nucleotide sequence encoding a leader sequence, (3) a second nucleotide sequence encoding an IL-12β subunit, (4) a third nucleotide sequence encoding a first linker (e.g., GS linker), (5) a fourth nucleotide sequence encoding an IL-12α subunit, (6) a fifth nucleotide sequence encoding a second linker (e.g., GS linker), (7) a sixth nucleotide sequence encoding a heterologous moiety (e.g., albumin), (8) a seventh nucleotide sequence encoding a third linker (e.g., GS linker), (9) an eighth nucleotide sequence encoding an additional moiety (e.g., lumican), and (12) a poly(A) tail. In some aspects, a polynucleotide described herein comprises (from 5′ to 3′): (1) a 5′-cap, (2) a 5′-UTR, (3) a promoter, (4) a first nucleotide sequence encoding a leader sequence, (5) a second nucleotide sequence encoding an IL-12β subunit, (6) a third nucleotide sequence encoding a first linker (e.g., GS linker), (7) a fourth nucleotide sequence encoding an IL-12α subunit, (8) a fifth nucleotide sequence encoding a second linker (e.g., GS linker), (9) a sixth nucleotide sequence encoding a heterologous moiety (e.g., albumin), (10) a seventh nucleotide sequence encoding a third linker (e.g., GS linker), (11) an eighth nucleotide sequence encoding an additional moiety (e.g., lumican), (12) a 3′-UTR, and (13) a poly(A) tail. Additional description of such exemplary constructs are provided below.

In some aspects, (i) the first nucleic acid molecule (i.e., encoding the leader sequence) comprises the sequence set forth in SEQ ID NO: 48; (ii) the second nucleic acid molecule (i.e., encoding the IL-12β subunit) comprises the sequence set forth in SEQ ID NO: 73; (iii) the third nucleic acid molecule (i.e., encoding the first linker) comprises the sequence set forth in SEQ ID NO: 98; (iv) the fourth nucleic acid molecule (i.e., encoding the IL-12α subunit) comprises the sequence set forth in SEQ ID NO: 123; (v) the fifth nucleic acid molecule (i.e., encoding the second linker) comprises the sequence set forth in SEQ ID NO: 144; (vi) the sixth nucleic acid molecule (i.e., encoding the half-life extending moiety) comprises the nucleic acid sequence set forth in SEQ ID NO: 165; (vii) the seventh nucleic acid molecule (i.e., encoding the third linker) comprises the sequence set forth in SEQ ID NO: 168; and (viii) the eighth nucleic acid molecule (i.e., encoding the lumican) comprises the sequence set forth in SEQ ID NO: 171. Accordingly, in certain aspects, a polynucleotide (e.g., isolated polynucleotide) described herein comprises the sequence set forth in SEQ ID NO: 23. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 48 (i.e., leader sequence), (3) a second nucleotide sequence comprising the sequence set forth in SEQ ID NO: 73 (i.e., IL-12β subunit), (4) a third nucleotide sequence comprising the sequence set forth in SEQ ID NO: 98 (i.e., first GS linker), (5) a fourth nucleotide sequence comprising the sequence set forth in SEQ ID NO: 123 (i.e., IL-12α subunit), (6) a fifth nucleotide sequence comprising the sequence set forth in SEQ ID NO: 144 (i.e., second GS linker), (7) a sixth nucleotide sequence comprising the sequence set forth in SEQ ID NO: 165 (i.e., human serum albumin), (8) a seventh nucleotide sequence comprising the sequence set forth in SEQ ID NO: 168 (i.e., third GS linker), (9) an eighth nucleotide sequence comprising the sequence set forth in SEQ ID NO: 171 (i.e., lumican), and (10) a poly(A) tail. An example of such a polynucleotide is described herein as the “C1 Construct.”

In some aspects, (i) the first nucleic acid molecule comprises the sequence set forth in SEQ ID NO: 49; (ii) the second nucleic acid molecule comprises the sequence set forth in SEQ ID NO: 74; (iii) the third nucleic acid molecule comprises the sequence set forth in SEQ ID NO: 99; (iv) the fourth nucleic acid molecule comprises the sequence set forth in SEQ ID NO: 124; (v) the fifth nucleic acid molecule comprises the sequence set forth in SEQ ID NO: 145; (vi) the sixth nucleic acid molecule comprises the sequence set forth in SEQ ID NO: 166; (vii) the seventh nucleic acid molecule comprises the sequence set forth in SEQ ID NO: 169; and (viii) the eighth nucleic acid molecule comprises the sequence set forth in SEQ ID NO: 172. Accordingly, in certain aspects, a polynucleotide (e.g., isolated polynucleotide) described herein comprises the sequence set forth in SEQ ID NO: 24. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 49 (i.e., leader sequence), (3) a second nucleotide sequence comprising the sequence set forth in SEQ ID NO: 74 (i.e., IL-12β subunit), (4) a third nucleotide sequence comprising the sequence set forth in SEQ ID NO: 99 (i.e., first GS linker), (5) a fourth nucleotide sequence comprising the sequence set forth in SEQ ID NO: 124 (i.e., IL-12α subunit), (6) a fifth nucleotide sequence comprising the sequence set forth in SEQ ID NO: 145 (i.e., second GS linker), (7) a sixth nucleotide sequence comprising the sequence set forth in SEQ ID NO: 166 (i.e., human serum albumin), (8) a seventh nucleotide sequence comprising the sequence set forth in SEQ ID NO: 169 (i.e., third GS linker), (9) an eighth nucleotide sequence comprising the sequence set forth in SEQ ID NO: 172 (i.e., lumican), and (10) a poly(A) tail. An example of such a polynucleotide is described herein as the “C2 Construct.”

In some aspects, (i) the first nucleic acid molecule comprises the sequence set forth in SEQ ID NO: 50; (ii) the second nucleic acid molecule comprises the sequence set forth in SEQ ID NO: 75; (iii) the third nucleic acid molecule comprises the sequence set forth in SEQ ID NO: 100; (iv) the fourth nucleic acid molecule comprises the sequence set forth in SEQ ID NO: 125; (v) the fifth nucleic acid molecule comprises the sequence set forth in SEQ ID NO: 146; (vi) the sixth nucleic acid molecule comprises the sequence set forth in SEQ ID NO: 167; (vii) the seventh nucleic acid molecule comprises the sequence set forth in SEQ ID NO: 170; and (viii) the eighth nucleic acid molecule comprises the sequence set forth in SEQ ID NO: 173. Accordingly, in certain aspects, a polynucleotide (e.g., isolated polynucleotide) described herein comprises the sequence set forth in SEQ ID NO: 25. In some aspects, the polynucleotide comprises one or more additional features described herein. For example, in some aspects, a polynucleotide provided herein comprises (from 5′ to 3′): (1) a 5′-cap (or cap analog), (2) a first nucleotide sequence comprising the sequence set forth in SEQ ID NO: 50 (i.e., leader sequence), (3) a second nucleotide sequence comprising the sequence set forth in SEQ ID NO: 75 (i.e., IL-12β subunit), (4) a third nucleotide sequence comprising the sequence set forth in SEQ ID NO: 100 (i.e., first GS linker), (5) a fourth nucleotide sequence comprising the sequence set forth in SEQ ID NO: 125 (i.e., IL-12α subunit), (6) a fifth nucleotide sequence comprising the sequence set forth in SEQ ID NO: 146 (i.e., second GS linker), (7) a sixth nucleotide sequence comprising the sequence set forth in SEQ ID NO: 167 (i.e., human serum albumin), (8) a seventh nucleotide sequence comprising the sequence set forth in SEQ ID NO: 170 (i.e., third GS linker), (9) an eighth nucleotide sequence comprising the sequence set forth in SEQ ID NO: 173 (i.e., lumican), and (10) a poly(A) tail. An example of such a polynucleotide is described herein as the “C3 Construct.”

### Lipid Nanoparticle and Delivery System

In some aspects, the present disclosure relates to the delivery of biologically active molecules (e.g., an IL-12 protein) to cells. In certain aspects, the delivery can occur in vivo (e.g., by administering a polynucleotide described herein to a subject) or ex vivo (e.g., by culturing a polynucleotide described herein with the cells in vitro). In some aspects, delivery of a polynucleotide (e.g., isolated polynucleotide) described herein can be performed using any suitable delivery system known in the art. In certain aspects, the delivery system is a vector. Accordingly, in some aspects, the present disclosure provides a vector comprising a polynucleotide of the present disclosure. Suitable vectors that can be used are known in the art. See, e.g., Sung et al., Biomater Res 23(8) (2019).

In some aspects, a polynucleotide described herein (e.g., an isolated polynucleotide comprising a nucleic acid molecule encoding an IL-12 protein) is delivered using lipid nanoparticles. Accordingly, in some aspects, the present disclosure relates to an IL-12-expressing polynucleotide (e.g., RNA) encapsulated by lipid nanoparticles, the composition thereof, and use of the composition thereof to treat a subject having cancer or suspected of having cancer.

A “lipid nanoparticle” (LNP), as used herein, refers to a vesicle, such as a spherical vesicle, having a contiguous lipid bilayer. Lipid nanoparticles can be used in methods by which pharmaceutical therapies are delivered to targeted locations. Non-limiting examples of LNPs include liposomes, bolaamphihiles, solid lipid nanoparticles (SLN), nanostructured lipid carriers (NLC), and monolayer membrane structures (e.g., archaeosomes and micelles).

In some aspects, the lipid nanoparticle comprises one or more types of lipids. A lipid, as used herein, refers to a group of organic compounds that include, but are not limited to, esters of fatty acids and in some aspects are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. Non-limiting examples of lipids include triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). In some aspects, the one or more types of lipids in the LNP comprises a cationic lipid. In some aspects, the one or more types of lipids in the LNP comprises a lipidoid, e.g., TT3. Accordingly, in some aspects, any of the polynucleotides described herein (e.g., comprising the sequence set forth in any one of SEQ ID Nos: 1-25, a 5′-cap (or a cap analog), and a poly(A) tail) can be encapsulated in a lipidoid nanoparticle (e.g., TT3). In some aspects, a polynucleotide comprises the sequence set forth in SEQ ID NO: 1, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 2, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 3, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 4, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 5, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 6, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 7, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 8, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 9, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 10, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 11, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 12, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 13, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 14, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 15, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 16, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 17, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 18, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 19, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 20, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 21, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 22, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 23, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 24, 5′-cap (or a cap analog), and a poly(A) tail, wherein the polynucleotide is encapsulated in a lipidoid nanoparticle (e.g., TT3). In some aspects, a polynucleotide comprises the sequence set forth in SEQ ID NO: 25, 5′-cap (or a cap analog), and a poly(A) tail, wherein the polynucleotide is encapsulated in a lipidoid nanoparticle (e.g., TT3).

Such lipids useful for the present disclosure include, but are not limited to N1,N3,N5-tris(3-(didodecylamino)propyl)benzene-1,3,5-tricarboxamide (TT3), N-(2,3-dioleoyloxy)propyl)-N,N,N-trimethylammonium chloride (DOTAP); lipofectamine; 1,2-DiLinoleyloxy-N,N-dimethylaminopropane (DLinDMA), 1,2-Dilinolenyloxy-N,N-dimethylaminopropane (DLenDMA); dioctadecyldimethylammonium (DODMA), Distearyldimethylammonium (DSDMA), N,N-dioleyl-N,N,-dimethylammonium chloride (DODAC); N-(2,3-dioleyloxy)propyl)-N,N,N-trimethylammonium chloride (DOTMA); N—N-distearyl-N,N-dimethylammonium bromide (DDAB); 3-(N—(N′,N′-dimethylaminoethane)-carbamoyl)cholesterol (DC-Chol) and N-(1,2-dimyristyloxprop-3-yl)-N,N-dimethyl-N-hydroxyethyl ammonium bromide (DMRIE).

In some aspects of the disclosure, the lipids, e.g., lipidoid, is TT3. TT3, as used herein, is capable of forming lipid nanoparticles for delivery of various biologic active agents into the cells. In addition, the present disclosure also demonstrates that an unloaded TT3-LNP can induce immunogenic cell death (ICD) in cancer cells in vivo and in vitro. Immunogenic cell death, as described herein, refers to a form of cell death that can induce an effective immune response through activation of dendritic cells (DCs) and consequent activation of specific T cell response. In some aspects of the disclosure, the cells that undergo immunogenic cell death are tumor cells. Immunogenic tumor cell death can trigger an effective anti-tumor immune response. In some aspects of the disclosure, the lipid nanoparticle comprises TT3-LNP encapsulating a nucleotide sequence encoding IL-12 (modRNA) encoding only a reporter gene (TT3-LNP-modRNA). The nucleotide sequence encoding IL-12 can work synergistically with the TT3-LNP to induce higher level of ICD in tumor cells compared to TT3-LNP alone. In some aspects of the disclosure, the lipid nanoparticle comprises a TT3-LNP encapsulating a modRNA encoding an IL-12 molecule. IL-12, which is an immunoregulatory cytokine, elicits a potent immune response against the local tumor. The combination of TT3-LNP, modRNA, and IL-12 expression, not only is effective in synergistic inhibition of tumor cells on site, but also elicits a systemic anti-tumor immune response to kill distal tumor cells and prevent the recurrence of tumors.

In some aspects of the disclosure, the cationic lipid is DOTAP. DOTAP, as used herein, is also capable of forming lipid nanoparticles. DOTAP can be used for the highly efficient transfection of DNA including yeast artificial chromosomes (YACs) into eukaryotic cells for transient or stable gene expression, and is also suitable for the efficient transfer of other negatively charged molecules, such as RNA, oligonucleotides, nucleotides, ribonucleoprotein (RNP) complexes, and proteins into research samples of mammalian cells.

In some aspects of the disclosure, the cationic lipid is lipofectamine. Lipfectamine, as used herein, is a common transfection reagent, produced and sold by Invitrogen, used in molecular and cellular biology. It is used to increase the transfection efficiency of RNA (including mRNA and siRNA) or plasmid DNA into in vitro cell cultures by lipofection. Lipofectamine contains lipid subunits that can form liposomes or lipid nanoparticles in an aqueous environment, which entrap the transfection payload, e.g., modRNA. The RNA-containing liposomes (positively charged on their surface) can fuse with the negatively charged plasma membrane of living cells, due to the neutral co-lipid mediating fusion of the liposome with the cell membrane, allowing nucleic acid cargo molecules to cross into the cytoplasm for replication or expression.

In some aspects of the disclosure, LNPs are composed primarily of cationic lipids along with other lipid ingredients. These typically include other lipid molecules belonging but not limited to the phophatidylcholine (PC) class (e.g., 1,s-Distearoyl-sn-glycero-3-phophocholine (DSPC), and 1,2-Dioleoyl-sn-glycero-3-phophoethanolamine (DOPE), sterols (e.g., cholesterol) and Polyethylene glycol (PEG)-lipid conjugates (e.g., 1,2-distearoyl-sn-glycero-3-phosphoethanolamine-N-[folate(polyethylene glycol)-2000 (DSPE-PEG2000), and C14-PEG2000. Table 2 shows the formulation of exemplary LNPs, TT3-LNP and DOTAP-LNP.

TABLE 2

| DOTAP-LNP | DOTAP | DSPC | Cholesterol | DSPE-PEG2000 |
|---|---|---|---|---|
| Molar ratio | 40 | 10 | 48 | 2 |
| TT3-LNP | TT3 | DOPE | Cholesterol | C14-PEG2000 |
| Molar Ratio | 20 | 30 | 40 | 0.75 |

In some aspects, the LNP comprises C14-PEG2000. In certain aspects, C14-PEG2000 comprises 1,2-dimyristoyl-rac-glycero-3-methoxypolyethylene glycol-2000 (DMG-PEG2000), 1,2-dimyristoyl-sn-glycero-3-phosphoethanolamine-N-[methoxy(polyethylene glycol)-2000] (DMPE-PEG2000), or both. As described herein (see, e.g., Example 3), in some aspects, the C14-PEG2000 (or other lipid ingredients disclosed herein) can be embedded in the LNP prior to the encapsulation of the polynucleotide. In some aspects, the C14-PEG2000 (or other lipid ingredients disclosed herein) can be added to the LNP after the encapsulation of the polynucleotide. For example, in certain aspects, a polynucleotide (e.g., isolated polynucleotide) comprising a nucleic acid molecule encoding an IL-12 protein (e.g., IL-12α and/or IL-12β subunit) is encapsulated in the LNP, and then the C14-PEG2000 (other lipid ingredients disclosed herein) is attached to the LNP using, e.g., micelles.

Particle size of lipid nanoparticles can affect drug release rate, bio-distribution, mucoadhesion, cellular uptake of water and buffer exchange to the interior of the nanoparticles, and protein diffusion. In some aspects of the disclosure, the diameter of the LNPs ranges from about 30 to about 500 nm. In some aspects of the disclosure, the diameter of the LNPs ranges from about 30 to about 500 nm, about 50 to about 400 nm, about 70 to about 300 nm, about 100 to about 200 nm, about 100 to about 175 nm, or about 100 to about 160 nm. In some aspects of the disclosure, the diameter of the LNPs ranges from 100-160 nm. In some aspects of the disclosure, the diameter of the LNPs can be about 30 nm, about 40 nm, about 50 nm, about 60 nm, about 70 nm, about 80 nm, about 90 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm., about 130 nm, about 140 nm, about 150 nm, or about 160 nm. In certain aspects, the lipid nanoparticle has a diameter of about 140 nm.

Zeta potential is a measure of the effective electric charge on the lipid nanoparticle surface. The magnitude of the zeta potential provides information about particle stability. In some aspects of the disclosure, the zeta potential of the LNPs ranges from about 3 to about 6 my. In some aspects of the disclosure, the zeta potential of the LNPs can be about 3 my, about 3.1 my, about 3.2 my, about 3.3 my, about 3.4 my, about 3.5 my, about 3.6 my, about 3.7 my, about 3.8 my, about 3.9 my, about 4 my, about 4.1 my, about 4.2 my, about 4.3 my, about 4.4 my, about 4.5 my, about 4.6 my, about 4.7 my, about 4.8 my, about 4.9 my, about 5 my, about 5.1 my, about 5.2 my, about 5.3 my, about 5.4 my, about 5.5 my, about 5.6 my, about 5.7 my, about 5.8 my, about 5.9 my, or about 6 my.

In some aspects, the disclosure is related to encapsulated polynucleotide (e.g., mRNA) with lipid nanoparticles (LNPs). In some aspects of the disclosure, the mass ratio between the lipid of LNPs and the polynucleotide (e.g., mRNA) ranges from about 1:2 to about 15:1. In some aspects, the mass ratio between the lipid and the polynucleotide (e.g., mRNA) can be about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, or about 15:1. In some aspects of the disclosure, the mass ratio between the lipid and the polynucleotide (e.g., mRNA) is about 10:1.

### Pharmaceutical Compositions

In some aspects, the disclosure relates to a pharmaceutical composition comprising the polynucleotide, vector, and/or lipid nanoparticle described herein. In some aspects of the disclosure, the pharmaceutical composition further comprises a pharmaceutically acceptable carrier (excipient). “Acceptable”, as used herein, means that the carrier must be compatible with the active ingredient of the composition and not deleterious to the subject to be treated. In some aspects, the carrier is capable of stabilizing the active ingredient. Pharmaceutically acceptable excipients (carriers) include buffers, which are well known in the art. See, e.g., Remington: The Science and Practice of Pharmacy 20th Ed. (2000) Lippincott Williams and Wilkoins, Ed. K. E. Hoover.

The pharmaceutical compositions to be used for in vivo administration must be sterile. This is readily accomplished by, for example, filtration through sterile filtration membranes. The lipid nanoparticles can be placed into a container having a sterile access port, for example, an intravenous solution bag or vial having a stopper pierceable by a hypodermic injection needle.

In some aspects of the disclosure, the pharmaceutical composition can be formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration. In some aspects of the disclosure, the pharmaceutical composition can be formulated for intratumoral injection. Intratumoral injection, as used herein, refers to direct injections into the tumor. A high concentration of composition can be achieved in situ, while using small amounts of drugs. Local delivery of immunotherapies allows multiple combination therapies, while preventing significant system exposure and off-target toxicities.

In some aspects of the disclosure, the pharmaceutical composition can be formulated for intramuscular injection, intravenous injection, or subcutaneous injection.

In some aspects of the disclosure, the pharmaceutical composition comprises pharmaceutically acceptable carriers, buffer agents, excipients, salts, or stabilizers in the form of lyophilized formulations or aqueous solutions. See, e.g., Remington: The Science and Practice of Pharmacy 20^{th} Ed. (2000) Lippincott Williams and Wilkins, Ed. K. E. Hoover. Acceptable carriers and excipients, or stabilizers are nontoxic to recipients at the dosages and concentrations used, and comprises buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In some aspects, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545, which are hereby incorporated by reference in their entirety. Liposomes with enhanced circulation time are disclosed in U.S. Pat. No. 5,013,556, which is hereby incorporated by reference in its entirety. In some aspects, liposomes can be generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some aspects of the disclosure, the pharmaceutical composition is formulated in sustained-release format. Suitable examples of sustained-release preparations include semipermeable matrices of solid hydrophobic polymers containing the lipid nanoparticles which matrices are in the form of shaped articles, e.g., films or microcapsules. Examples of sustained-release matrices include, but are not limited to, polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPROM DEPOT™ (injectable microspheres composed of lactic acid-glycolic acid copolymer and leuprolide acetate), sucrose acetate isobutyrate, and poly-D-(−)-3-hydroxybutyric acid.

In some aspects, suitable surface-active agents include, but are not limited to, non-ionic agents, such as polyoxyethylenesorbitans (e.g., TWEEN™ 20, 40, 60, 80 or 85) and other sorbitans (e.g., SPAN™ 20, 30, 60, 80, or 85). In some aspects, compositions with a surface-active agent comprise between 0.05 and 5% surface-active agent. In some aspects the composition comprises 0.1 and 2.5%. It will be appreciated that other ingredients can be added, for example mannitol or other pharmaceutically acceptable vehicles, if necessary.

In some aspects, the pharmaceutical composition is in unit dosage forms such as tablets, pills, capsules, powders, granules, solutions or suspensions, or suppositories, for oral, parenteral, or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g, water, to form a solid preformulation composition containing a homogenous mixture of a compound of the present disclosure, or a non-toxic pharmaceutically acceptable salt thereof. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from about 0.1 to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

Suitable emulsions can be prepared using commercially available fat emulsions, such as INTRALIPID™, LIPOSYN™, INFONUTROL™, LIPOFUNDIN™, and LIPIPHYSAN™. The active ingredient can be either dissolved in a pre-mixed emulsion composition or alternatively it can be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids, or soybean lecithin) and water. It will be appreciated that other ingredients can be added, for example glycerol or glucose, to adjust tonicity of the emulsion. Suitable emulsions will typically contain up to about 20% oil, for example, between about 5 and about 20%. The fat emulsion can comprise fat droplets having a suitable size and can have a pH in the range of about 5.5 to about 8.0.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some aspects, the composition is administered by the oral or nasal respiratory route for local or systemic effect.

Compositions in pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, or powder compositions can be administered from devices which deliver the formulation in an appropriate manner.

### Therapeutic Applications

In some aspects of the disclosure, the polynucleotides, vectors, lipid nanoparticles, and/or pharmaceutical compositions described herein (also collectively referred to herein as “compositions”) are used to treat a disease or disorder. In certain aspects, the disease or disorder comprises a cancer. Non-limiting examples of cancers that can be treated are provided elsewhere in the present disclosure.

In some aspects, an effective amount of any of the compositions described herein is administered to a subject in need thereof via a suitable route, such as intratumoral administration, intravenous administration (e.g, as a bolus or by continuous infusion over a period of time), by intramuscular, intraperitoneal, intracerebrospinal, subcutaneous, intra-articular, intrasynovial, intrathecal, oral, inhalation, or topical routes. Commercially available nebulizers for liquid formulations, including jet nebulizers and ultrasonic nebulizers are useful for administration. Liquid formulations can be nebulized and lyophilized powder can be nebulized after reconstitution. In some aspects, the pharmaceutical composition described herein is aerolized using a fluorocarbon formulation and a metered dose inhaler, or inhaled as a lyophilized and milled powder. In some aspects, the pharmaceutical composition described herein is formulated for intratumoral injection. In some aspects, the pharmaceutical composition described herein is administered to a subject via a local route, for example, injected to a local site such as a tumor site or an infectious site. In some aspects, the subject is a human.

As will be apparent from the present disclosure, in some aspects, the compositions described herein are administered to a subject in an effective amount to confer a therapeutic effect, either alone or in combination with one or more other active agents. In some aspects, the compositions are administered to a subject suffering from a cancer, and the therapeutic effect comprises reduced tumor burden, reduction of cancer cells, increased immune activity, or combinations thereof. Whether the administered composition (e.g., a lipid nanoparticle) achieved the therapeutic effect can be determined using any suitable methods known in the art (e.g., measuring tumor volume and/or T cell activity). Effective amounts vary, as recognized by those skilled in the art, depending on the particular condition being treated, the severity of the condition, the individual patient parameters including age, physical condition, size, gender and weight, the duration of the treatment, the nature of the concurrent therapy (if any), the specific route of administration and like factors within the knowledge of expertise of the health practitioner.

Empirical considerations, such as the half-life, generally will contribute to the determination of the dosage. Frequency of administration can be determined and adjusted over the course of therapy, and is generally, but not necessarily, based on treatment and/or suppression and/or amelioration and/or delay of a target disease/disorder. Alternatively, sustained continuous release formulations of a composition described herein (e.g., lipid nanoparticle) can be appropriate. Various formulations and devices for achieving sustained release are known in the art.

In some aspects of the disclosure, the treatment is a single injection of the composition disclosed herein. In some aspects, the single injection is administered intratumorally to the subject in need thereof.

In some aspects of the disclosure, dosages for a composition described herein can be determined empirically in individuals who have been given one or more administration(s) of the composition (e.g., lipid nanoparticle described herein). In some aspects, the individuals are given incremental dosages of the composition described herein. To assess efficacy of the composition herein, an indicator of disease/disorder can be followed. For repeated administrations over several days or longer, depending on the condition, in some aspects, the treatment is sustained until a desired suppression of symptoms occurs or until sufficient therapeutic levels are achieved to alleviate a target disease or disorder, or symptom thereof.

In some aspects of the disclosure, dosing frequency is about once every week, about once every 2 weeks, about once every 3 weeks, about once every 4 weeks, about once every 5 weeks, about once every 6 weeks, about once every 7 weeks, about once every 8 weeks, about once every 9 weeks, or about once every 10 weeks; or about once a month, about every 2 months, or about every 3 months, or longer. The dosing regimen (e.g., dosage and/or dosing frequency) of the composition described herein (e.g., lipid nanoparticle) used can vary over time.

In some aspects of the disclosure, the method comprises administering to a subject in need thereof one or multiple doses of a composition described herein.

The appropriate dosage of the composition (e.g., lipid nanoparticle described herein) will depend on the specific composition (e.g., lipid nanoparticle), the type and severity of the disease/disorder (e.g., cancer), whether the composition (e.g., lipid nanoparticle) is administered for preventive or therapeutic purposes, previous therapy, the subject's clinical history and response to the composition (e.g., lipid nanoparticle), and the discretion of the attending physician. In some aspects, a clinician can administer a composition disclosed herein until a dosage is reached that achieves the desired result. In some aspects, the desired result is a decrease in tumor burden, a decrease in cancer cells, or increased immune activity. Administration of one or more compositions described herein can be continuous or intermittent, depending, for example, upon the recipient's physiological condition, whether the purpose of the administration is therapeutic or prophylactic, and other factors known to skilled practitioners. The administration of the composition described herein can be essentially continuous over a preselected period of time or can be in a series of spaced doses, e.g., either before, during, or after developing a target disease or disorder.

As used herein, alleviating a target disease/disorder includes delaying the development or progression of the disease, or reducing disease severity. Alleviating the disease does not necessarily require curative results. As used herein, “delaying” the development of a target disease or disorder means to defer, hinder, slow, retard, stabilize, and/or postpone progression of the disease. This delay can be for varying lengths of time, depending on the history of the disease and/or subject being treated. A method that delays or alleviates the development of a disease, or delays the onset of the disease, is a method that reduces probability of developing one or more symptoms of the disease in a given time frame and/or reduces the extent of the symptoms in a given time frame, when compared to not using the method. Such comparisons are typically based on clinical studies, using a number of subjects sufficient to give a statistically significant result.

In some aspects, a composition described herein is administered to a subject in need thereof at an amount sufficient to reduce tumor burden or cancer cell growth in vivo by at least about 5%, at least about 10%, at least about 20%, at least about 30%, at least about 40%, at least about 50%, at least about 60%, at least about 70%, at least about 80%, at least about 90%, or greater. In some aspects, the composition described herein is administered in an amount effective in increasing immune activity by at least about 5%, at least about 10%, at least about 20%, at least about 30%, at least about 40%, at least about 50%, at least about 60%, at least about 70%, at least about 80%, at least about 90%, or greater.

In some aspects, administering the composition (e.g., the polynucleotide, vector, lipid nanoparticle, or pharmaceutical composition described herein) to a subject enhances immune activity, such as T cell activity, in the subject. In certain aspects, the immune activity is enhanced or increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold or more, compared to the immune activity of a reference subject (e.g., the subject prior to the administration of the composition or a corresponding subject that did not reactive an administration of the composition).

In some aspects, the subject is a human having, suspected of having, or at risk for a cancer. In some aspects the cancer is selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and various types of head and neck cancer, including squamous cell head and neck cancer. In some aspects, the cancer can be melanoma, lung cancer, colorectal cancer, renal-cell cancer, urothelial carcinoma, or Hodgkin's lymphoma.

A subject having a target disease or disorder can be identified by routine medical examination, e.g., laboratory tests, organ functional tests, CT scans, or ultrasounds. A subject suspected of having a target disease or disorder might show one or more symptoms of the disease or disorder. A subject at risk for the disease or disorder can be a subject having one or more of the risk factors associated with that disease or disorder. A subject at risk for a disease or disorder can also be identified by routine medical practices.

In some aspects, the composition described herein is co-administered with at least one additional suitable therapeutic agent. In some aspects, the at least one additional suitable therapeutic agent comprises an anti-cancer agent, an anti-viral agent, an anti-bacterial agent, or other agents that serve to enhance and/or complement the immunostimulatory effect of the composition (e.g., lipid nanoparticle) described herein. Further examples of additional therapeutic agents that can be used in combination with the compositions described herein include: a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof. In some aspects, the composition described herein and the at least one additional therapeutic agent are administered to the subject in a sequential manner, i.e, each therapeutic agent is administered at a different time. In some aspects, the composition described herein and the at least one additional therapeutic agent are administered to the subject in a substantially simultaneous manner.

It will be appreciated by one of skill in the art that any combination of the composition described herein and another anti-cancer agent (e.g., a chemotherapeutic agent) can be used in any sequence for treating a cancer. The combinations described herein can be selected on the basis of a number of factors, which include, but are not limited to, the effectiveness or reducing tumor formation or tumor growth, reducing cancer cells, increasing immune activity, and/or alleviating at least one symptom associated with the cancer, or the effectiveness for mitigating the side effects of another agent of the combination. For example, a combined therapy described herein can reduce any of the side effects associated with each individual members of the combination, for example, a side effect associated with the anti-cancer agent.

In some aspects, the other anti-cancer therapeutic agent is a chemotherapy, a radiation therapy, a surgical therapy, an immunotherapy, or combinations thereof. In some aspects, the chemotherapeutic agent is carboplatin, cisplatin, docetaxel, gemcitabine, nab-paclitaxel, pemetrexed, vinorelbine, or combinations thereof. In some aspects, the radiation therapy is ionizing radiation, gamma-radiation, neutron beam radiotherapy, electron beam radiotherapy, proton therapy, brachytherapy, systemic radioactive isotopes, radiosensitizers, or combinations thereof. In some aspects, the surgical therapy is a curative surgery (e.g., tumor removal surgery), a preventative surgery, a laparoscopic surgery, a laser surgery, or combinations thereof. In some aspects, the immunotherapy is adoptive cell transfer, therapeutic cancer vaccines, or combinations thereof.

In some aspects, the chemotherapeutic agent is platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives); Purine antagonists (Thioguanine, Fludarabine, Cladribine, 6-Mercaptopurine, Pentostatin, clofarabine and relatives) and Pyrimidine antagonists (Cytarabine, Floxuridine, Azacitidine, Tegafur, Carmofur, Capecitabine, Gemcitabine, hydroxyurea, 5-Fluorouracil (5FU), and relatives); Alkylating agents, such as Nitrogen mustards (e.g., Cyclophosphamide, Melphalan, Chlorambucil, mechlorethamine, Ifosfamide, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); Triazenes (e.g., Dacarbazine, Altretamine, Temozolomide, and relatives); Alkyl sulphonates (e.g., Busulfan, Mannosulfan, Treosulfan, and relatives); Procarbazine; Mitobronitol, and Aziridines (e.g., Carboquone, Triaziquone, ThioTEPA, triethylenemelamine, and relatives); Antibiotics, such as Hydroxyurea, Anthracyclines (e.g., doxorubicin agent, daunorubicin, epirubicin and other derivatives); Anthracenediones (e.g., Mitoxantrone and relatives); Streptomyces family (e.g., Bleomycin, Mitomycin C, Actinomycin, Plicamycin); Ultraviolet light; and combinations thereof.

In some aspects, the other anti-cancer therapeutic agent is an antibody. Antibodies (preferably monoclonal antibodies) achieve their therapeutic effect against cancer cells through various mechanisms. They can have direct effects in producing apoptosis or programmed cell death. They can block components of signal transduction pathways such as e.g., growth factor receptors, effectively arresting proliferation of tumor cells. In cells that express monoclonal antibodies, they can bring about anti-idiotype antibody formation. Indirect effects include recruiting cells that have cytotoxicity, such as monocytes and macrophages. This type of antibody-mediated cell kill is called antibody-dependent cell mediated cytotoxicity (ADCC). Antibodies also bind complement, leading to direct cell toxicity, known as complement dependent cytotoxicity (CDC). Combining surgical methods with immunotherapeutic drugs or methods is an successful approach, as e.g., demonstrated in Gadri et al. 2009: Synergistic effect of dendritic cell vaccination and anti-CD20 antibody treatment in the therapy of murine lymphoma. J Immunother. 32(4): 333-40. The following list provides some non-limiting examples of anti-cancer antibodies and potential antibody targets (in brackets) which can be used in combination with the present disclosure: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Bavituximab (phosphatidylserine), Bectumomab (CD22), Belimumab (BAFF), Bevacizumab (VEGF-A), Bivatuzumab mertansine (CD44 v6), Blinatumomab (CD19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin av(33), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (IL-1β), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL-5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Narnatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL-6), Tabalumab (BAFF), Tacatuzumab tetraxetan (alpha-fetoprotein), Taplitumomab paptox (CD19), Tenatumomab (tenascin C), Teprotumumab (CD221), Ticilimumab (CTLA-4), Tigatuzumab (TRAIL-R2), TNX-650 (IL-13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1BB), Volociximab (integrin α5β1), Votumumab (tumor antigen CTAA16.88), Zalutumumab (EGFR), Zanolimumab (CD4).

In some aspects, the other anti-cancer therapeutic agent is a cytokine, chemokine, costimulatory molecule, fusion protein, or combinations thereof. Examples of chemokines include, but are not limited to, CCR7 and its ligands CCL19 and CCL21, furthermore CCL2, CCL3, CCL5, and CCL16. Other examples are CXCR4, CXCR7 and CXCL12. Furthermore, costimulatory or regulatory molecules such as e.g., B7 ligands (B7.1 and B7.2) are useful. Also useful are other cytokines such as e.g., interleukins especially (e.g., IL-1 to IL17), interferons (e.g., IFNalpha1 to IFNalpha8, IFNalpha10, IFNalpha13, IFNalpha14, IFNalpha16, IFNalpha17, IFNalpha21, IFNbeta1, IFNW, IFNE1 and IFNK), hematopoietic factors, TGFs (e.g., TGF-α, TGF-β, and other members of the TGF family), finally members of the tumor necrosis factor family of receptors and their ligands as well as other stimulatory molecules, comprising but not limited to 41BB, 41BB-L, CD137, CD137L, CTLA-4GITR, GITRL, Fas, Fas-L, TNFR1, TRAIL-R1, TRAIL-R2, p75NGF-R, DR6, LT.beta.R, RANK, EDAR1, XEDAR, Fn114, Troy/Trade, TAJ, TNFRII, HVEM, CD27, CD30, CD40, 4-1BB, OX40, GITR, GITRL, TACI, BAFF-R, BCMA, RELT, and CD95 (Fas/APO-1), glucocorticoid-induced TNFR-related protein, TNF receptor-related apoptosis-mediating protein (TRAMP) and death receptor-6 (DR6). Especially CD40/CD40L and OX40/OX40L are important targets for combined immunotherapy because of their direct impact on T cell survival and proliferation. For a review see Lechner et al. 2011: Chemokines, costimulatory molecules and fusion proteins for the immunotherapy of solid tumors. Immunotherapy 3 (11), 1317-1340.

In some aspects, the other anti-cancer therapeutic is a bacterial treatment. Researchers have been using anaerobic bacteria, such as Clostridium novyi, to consume the interior of oxygen-poor tumors. These should then die when they come in contact with the tumor's oxygenated sides, meaning they would be harmless to the rest of the body. Another strategy is to use anaerobic bacteria that have been transformed with an enzyme that can convert a non-toxic prodrug into a toxic drug. With the proliferation of the bacteria in the necrotic and hypoxic areas of the tumor, the enzyme is expressed solely in the tumor. Thus, a systemically applied prodrug is metabolized to the toxic drug only in the tumor. This has been demonstrated to be effective with the nonpathogenic anaerobe Clostridium sporogenes.

In some aspects, the other anti-cancer therapeutic agent is a kinase inhibitor. The growth and survival of cancer cells is closely interlocked with the deregulation of kinase activity. To restore normal kinase activity and therefor reduce tumor growth a broad range of inhibitors is in used. The group of targeted kinases comprises receptor tyrosine kinases e.g., BCR-ABL, B-Raf, EGFR, HER-2/ErbB2, IGF-IR, PDGFR-α, PDGFR-β, c-Kit, Flt-4, Flt3, FGFR1, FGFR3, FGFR4, CSF1R, c-Met, RON, c-Ret, ALK, cytoplasmic tyrosine kinases e.g., c-SRC, c-YES, Abl, JAK-2, serine/threonine kinases e.g., ATM, Aurora A & B, CDKs, mTOR, PKCi, PLKs, b-Raf, S6K, STK11/LKB1 and lipid kinases e.g., PI3K, SK1. Small molecule kinase inhibitors are e.g., PHA-739358, Nilotinib, Dasatinib, and PD166326, NSC 743411, Lapatinib (GW-572016), Canertinib (CI-1033), Semaxinib (SU5416), Vatalanib (PTK787/ZK222584), Sutent (SU11248), Sorafenib (BAY 43-9006) and Leflunomide (SU101). For more information see e.g., Zhang et al. 2009: Targeting cancer with small molecule kinase inhibitors. Nature Reviews Cancer 9, 28-39.

In some aspects, the other anti-cancer therapeutic agent is a toll-like receptor. The members of the Toll-like receptor (TLRs) family are an important link between innate and adaptive immunity and the effect of many adjuvants rely on the activation of TLRs. A large number of established vaccines against cancer incorporate ligands for TLRs for boosting vaccine responses. Besides TLR2, TLR3, TLR4 especially TLR7 and TLR8 have been examined for cancer therapy in passive immunotherapy approaches. The closely related TLR7 and TLR8 contribute to antitumor responses by affecting immune cells, tumor cells, and the tumor microenvironment and can be activated by nucleoside analogue structures. All TLR's have been used as stand-alone immunotherapeutics or cancer vaccine adjuvants and can be synergistically combined with the formulations and methods of the present disclosure. For more information see van Duin et al. 2005: Triggering TLR signaling in vaccination. Trends in Immunology, 27(1):49-55.

In some aspects, the other anti-cancer therapeutic agent is an angiogenesis inhibitor. Angiogenesis inhibitors prevent the extensive growth of blood vessels (angiogenesis) that tumors require to survive. The angiogenesis promoted by tumor cells to meet their increasing nutrient and oxygen demands for example can be blocked by targeting different molecules. Non-limiting examples of angiogenesis-mediating molecules or angiogenesis inhibitors which can be combined with the present disclosure are soluble VEGF (VEGF isoforms VEGF121 and VEGF165, receptors VEGFR1, VEGFR2 and co-receptors Neuropilin-1 and Neuropilin-2) 1 and NRP-1, angiopoietin 2, TSP-1 and TSP-2, angiostatin and related molecules, endostatin, vasostatin, calreticulin, platelet factor-4, TIMP and CDAI, Meth-1 and Meth-2, IFN-α, -β and -γ, CXCL10, IL-4, -12 and -18, prothrombin (kringle domain-2), antithrombin III fragment, prolactin, VEGI, SPARC, osteopontin, maspin, canstatin, proliferin-related protein, restin and drugs like e.g., bevacizumab, itraconazole, carboxyamidotriazole, TNP-470, CM101, IFN-α, platelet factor-4, suramin, SU5416, thrombospondin, VEGFR antagonists, angiostatic steroids+heparin, cartilage-derived angiogenesis Inhibitory factor, matrix metalloproteinase inhibitors, 2-methoxyestradiol, tecogalan, tetrathiomolybdate, thalidomide, thrombospondin, prolactina Vβ3 inhibitors, linomide, tasquinimod, For review see Schoenfeld and Dranoff 2011: Anti-angiogenesis immunotherapy. Hum Vaccin. (9):976-81.

In some aspects, the other anti-cancer therapeutic agent is a virus-based vaccine. There are a number of virus-based cancer vaccines available or under development which can be used in a combined therapeutic approach together with the formulations of the present disclosure. One advantage of the use of such viral vectors is their intrinsic ability to initiate immune responses, with inflammatory reactions occurring as a result of the viral infection creating the danger signal necessary for immune activation. An ideal viral vector should be safe and should not introduce an anti-vector immune response to allow for boosting anti-tumor specific responses. Recombinant viruses such as vaccinia viruses, herpes simplex viruses, adenoviruses, adeno-associated viruses, retroviruses and avipox viruses have been used in animal tumor models and based on their encouraging results, human clinical trials have been initiated. Especially important virus-based vaccines are virus-like particles (VLPs), small particles that contain certain proteins from the outer coat of a virus. Virus-like particles do not contain any genetic material from the virus and cannot cause an infection but they can be constructed to present tumor antigens on their coat. VLPs can be derived from various viruses such as e.g., the hepatitis B virus or other virus families including Parvoviridae (e.g., adeno-associated virus), Retroviridae (e.g., HIV), and Flaviviridae (e.g., Hepatitis C virus). For a general review see Sorensen and Thompsen 2007: “Virus-based immunotherapy of cancer: what do we know and where are we going?” APMIS 115(11):1177-93; virus-like particles against cancer are reviewed in Buonaguro et al. 2011: Developments in virus-like particle-based vaccines for infectious diseases and cancer. Expert Rev Vaccines 10(11):1569-83; and in Guillen et al. 2010: Virus-like particles as vaccine antigens and adjuvants: application to chronic disease, cancer immunotherapy and infectious disease preventive strategies. Procedia in Vaccinology 2 (2), 128-133.

In some aspects, the other anti-cancer therapeutic agent is a peptide-based target therapy. Peptides can bind to cell surface receptors or affected extracellular matrix surrounding the tumor. Radionuclides which are attached to these peptides (e.g., RGDs) eventually kill the cancer cell if the nuclide decays in the vicinity of the cell. Especially oligo- or multimers of these binding motifs are of great interest, since this can lead to enhanced tumor specificity and avidity. For non-limiting examples see Yamada 2011: Peptide-based cancer vaccine therapy for prostate cancer, bladder cancer, and malignant glioma. Nihon Rinsho 69(9): 1657-61.

In some aspects, a therapeutic application of a polynucleotide (e.g., isolated polynucleotide) described herein comprises producing the encoded IL-12 protein. Accordingly, in some aspects, the present disclosure relates to a method of producing an IL-12 protein. In certain aspects, the method comprises contacting a cell with any of the compositions described herein (e.g., polynucleotides, vectors, and/or lipid nanoparticles) under conditions suitable for producing the encoded IL-12 protein. In some aspects, the method further comprises purifying the produced IL-12 protein. In some aspects, the contacting occurs in vivo (e.g., by administering the polynucleotide, vector, and/or lipid nanoparticle to a subject). In some aspects, the contacting occurs ex vivo (e.g., by culturing cells with the polynucleotide, vector, and/or lipid nanoparticles in vitro). Cells (e.g., host cells) comprising the polynucleotide, vector, and/or lipid nanoparticle are encompassed herein. Non-limiting examples of cells that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### Kits for Use in Therapy

The present disclosure also provides kits for use in immunotherapy against a disease or disorder, such as a cancer (e.g., melanoma, lung cancer, colorectal cancer, or renal-cell cancer), and/or treating or reducing the risk for the disease or disorder (e.g., cancer). In some aspects, the kit includes one or more containers comprising a composition described herein.

In some aspects, the kit comprises instructions for use in accordance with any of the methods described herein. For example, the included instructions can comprise a description of administration of the pharmaceutical composition described herein to treat, delay the onset, or alleviate a target disease. In some aspects, the instructions comprise a description of administering the composition described herein to a subject at risk of the target disease/disorder (e.g., cancer).

In some aspects, the instructions comprise dosage information, dosing schedule, and route of administration. In some aspects, the containers are unit doses, bulk packages (e.g., multi-dose packages) or sub-unit doses. In some aspects, the instructions are written instructions on a label or package insert (e.g., a paper sheet included in the kit). In some aspects, the instructions are machine-readable instructions (e.g., instructions carried on a magnetic or optical storage disk).

In some aspects, the label or package insert indicates that the composition disclosed herein is used for treating, delaying the onset, and/or alleviating a disease or disorder associated with cancer, such as those described herein. Instructions can be provided for practicing any of the methods described herein.

In some aspects, the kits described herein are in suitable packaging. In some aspects, suitable packing comprises vials, bottles, jars, flexible packaging (e.g., seal Mylar or plastic bags), or combinations thereof. In some aspects, the packaging comprises packages for use in combination with a specific device such as an inhaler, nasal administration device (e.g., an atomizer), or an infusion device such as a minipump. In some aspects, the kit comprises a sterile access port (for example the container can be an intravenous solution bag or a vial having a stopper pierceable by a hypodermic injection needle). In some aspects, the container can also have a sterile access port (for example the container can be an intravenous solution bag or a vial having a stopper pierceable by a hypodermic injection needle). In some aspects, at least one active agent is a composition as described herein.

In some aspects, the kits further comprise additional components such as buffers and interpretive information. In some aspects, the kit comprises a container and a label or package insert(s) on or associated with the container. In some aspects, the disclosure provides articles of manufacture comprising the contents of the kits described herein.

### General Techniques

The practice of the present disclosure will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Giffiths, and D. G. Newell, eds., 1993-8) J. Wiley and Sons; Method of Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987): PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanette and J. D. Capra, eds., Harwood Academic Publishers, 1995). Without further elaboration, it is believed that one skilled in the art can, based on the above description, utilize the present disclosure to its fullest extent. All publications cited herein (including those listed above and elsewhere in the present disclosure) are incorporated by reference in their entirety.

### EXAMPLES

### Example 1: Construction of Nucleotide Sequence Encoding IL-12S

To construct the polynucleotides disclosed herein (i.e., comprising a nucleic acid molecule encoding an IL-12 protein), the following materials and methods were used:

### Template Preparation

For replicon RNA, a VEE replicon vector containing the payload was prepared. VEE replicon vector backbones used included one or more of the following modifications: (i) “A3G”: represents a change in the 5′ UTR of the TC-83 VEE backbone that enhances expression (see, e.g., Kulasegaran-Shylini, R., et al., Virology 287: 211-221 (2009)); (ii) “+E1”: indicates that the 3′-end of the VEE “E1” coding region was included; (iii) “−E1”: indicates that the 3′-end of the VEE “E1” coding region was not included; (iv) “Alternative”: represents a VEE replicon backbone sequence described in, e.g., AddGene catalog number 58977; and Yoshioka, N., et al., Cell Stem Cell. 13(2):246-54 (2013); (v) “Evolved”: indicates that a set of mutations that were identified as enhancing VEE replicon expression was included (see, e.g., Li, Y., et al., Scientific Reports 9:6932 (2019)).

Methods of preparing such vectors are known in the art. The vector plasmid was further linearized using I-SceI as follows. Briefly, 1 μg of replicon plasmid vector was treated with I-SceI in CutSmart buffer for 1 hour at 37° C. Then, the enzyme was heat inactivated at 65° C. for 20 minutes. The concentration and volume of the different components are provided in Table 3 (below).

Additional vectors used in the examples were prepared as follows. Alternative Evolved −E1 SGP scar vector was linearized using MIuI by following a similar procedure and conditions as were used for the I-SceI treatment of the VEE vector. A3G +E1 (Clean end repRNA) was digested using SapI by following a similar procedure and conditions as were used for the I-SceI treatment of the VEE vector.

TABLE 3
Vector Plasmid Linearization

| Component | Volume (μL) | Concentration | |
|---|---|---|---|
| DNA template | | 1 | μg |
| Cutsmart buffer | 5 | 1x | |
| I-SceI | 1 | 5 | units |

| | |
|---|---|
| Water | 44 |

For modified RNA (modRNA) template, the DNA vector was generated using a replicon plasmid with forward primer containing T7 promoter (TAA TAC GAC TCA CTA TA ATG GAC TAC GAC ATA GT; SEQ ID NO: 181) and SGP and a reverse primer in the 3′-UTR (GAA ATA TTA AAA ACA AAA TCC GAT TCG GAA AAG AA; SEQ ID NO: 185). The Tₘ for the forward and reverse primers were 68° C. and 64° C., respectively. Tables 4 and 5 (below) provide additional information relating to the PCR reaction.

TABLE 4
PCR Setup

| | 25 μl | | FINAL | |
|---|---|---|---|---|
| COMPONENT | REACTION | | CONCENTRATION | |
| 10 μM Forward Primer | 1.25 | μl | 0.5 | μM |
| 10 μM Reverse Primer | 1.25 | μl | 0.5 | μM |

| | | | |
|---|---|---|---|
| Template DNA | variable | 10 | ng |

| | | |
|---|---|---|
| 2X Q5 Hot Start Master mix | 12.5 | μl |
| Nuclease-Free Water | to 25 | μl |

TABLE 5
PCR Cycling Conditions

| | | |
|---|---|---|
| STEP | TEMP | TIME |

| | | | | |
|---|---|---|---|---|
| Initial Denaturation | 98° | C. | 30 | seconds |
| 30 Cycles | 98° | C. | 10 | seconds |
| | 65° | C. | 15 | seconds |
| | 72° | C. | 4 | minutes |
| Final Extension | 72° | C. | 4 | minutes |

| | | |
|---|---|---|
| Hold | 4-10° | C. |

Plasmid DNA (template) in the PCR reaction was digested by DpnI. More specifically, 1 μL DpnI per μg of initial plasmid was added to the PCR sample and incubated for 1 hour at 37° C.

The PCR (modRNA template) and I-SceI treated replicon DNA (repRNA template) was checked on pre-cast gels to confirm the purity (PCR) and integrity (replicon template) of the replicated construct. Specifically, 20 ng of the DNA was loaded onto a 1.2% DNA gel and ran at 275V for 7-10 minutes. Once confirmed, the DNA was eluted in 20 μL of water.

### In Vitro Transcription

To transcribe the above DNA to RNA, a HiScribe High yield T7 kit (New England Biolabs) was used with the modifications described herein. For modified RNA (modRNA) synthesis, the UTP component of the kit was replaced with N1-methylpseudouridine-5′-triphosphate. To begin the in vitro transcription process, the kit components were thawed on ice, mixed, and pulse-spinned in a microfuge. The sample was placed on ice until further use.

Co-transcriptional capping method: For production using cap analog replicon plasmids and modRNA templates, the components shown in Table 6 (below) were mixed, pulse-spun in a microfuge, and then incubated at 37° C. for three hours in the Thermomixer at 400 rpm. A 1 μL aliquot was taken for quality control purposes.

TABLE 6
Co-Transcriptional Capping Components

| Component | Volume | | Final Conc. |
|---|---|---|---|
| Nuclease-free water | X | μl | |
| 10X Reaction Buffer (NEB | 2 | μl | 1X |
| ATP (100 mM) | 2 | μl | 10 mM final |
| GTP (100 mM) | 2 | μl | 10 mM final |
| UTP or N1-methylpseudoUTP (100 mM) | 2 | μl | 10 mM final |
| CTP (100 mM) | 2 | μl | 10 mM final |
| Cap Analog | 1 | μl | |
| Template DNA | X | μl | 1 μg |
| T7 RNA Polymerase Mix | 2 | μl | |
| SUPERase Inh. | 1 | μl | |
| Total reaction volume | 20 | μl | |

Post-transcriptional enzymatic capping method: In addition to the co-transcriptional capping method that was performed after IVT, a post-transcriptional enzymatic capping method was used for vectors that contained a terminal ‘G’ in the T7 promoter. This method comprised enzymatic production of a ‘Cap 1’ mRNA following in vitro transcription. For production using enzymatic replicon plasmids, the reaction was assembled at room temperature in the order shown in Table 7.

TABLE 7
Post-Transcriptional Enzymatic Capping Components

| Component | Volume | | Final Conc. |
|---|---|---|---|
| Nuclease-free water | X | μl | |
| 10X Reaction Buffer | 2 | μl | 1X |
| ATP (100 mM) | 2 | μl | 10 mM final |
| GTP (100 mM) | 2 | μl | 10 mM final |
| UTP or N1-methylpseudoUTP (100 mM) | 2 | μl | 10 mM final |
| CTP (100 mM) | 2 | μl | 10 mM final |
| Template DNA | X | μl | 1 μg |
| T7 RNA Polymerase Mix | 2 | μl | |
| SUPERase Inh. | 1 | μl | |
| Total reaction volume | 20 | μl | |

DNAse treatment: After capping with either the co-transcriptional capping method or the post-transcriptional enzymatic capping method, Turbo DNase enzyme was used. There was no need to add the 10× buffer since the enzyme was active in the IVT reactions. The reaction was diluted to 50 μL with nuclease free water. Then, 5 μL of the enzyme (2 U/μL) was added to 20 μL IVT reaction. Then, the mixture was incubated for 30 minutes at 37° C. Afterwards, the RNA was purified using Monarch RNA cleanup kit. A 1 μL aliquot was taken for quality control purposes

Capping and 2′-O-methylation: To prepare a methylated guanine-cap with 2′-O-methylation (Cap 1) structure on the 5′-ends of the IVT mRNAs made using the above-described post-transcriptional capping method, the following method was used. First, the uncapped RNA and nuclease-free water were mixed to a final volume of 13 μL. Then, the mixture was heated at 65° C. for 5 minutes. The mixture was then placed on ice for 5 additional minutes. Then, the components provided in Table 8 (below) were added to the mixture and incubated for 60 minutes at 37° C. Next, the RNA was purified using the small Monarch RNA cleanup kit.

TABLE 8
Capping and 2′-O-Methylation Components

| Component | Volume | |
|---|---|---|
| Denatured uncapped RNA (from above) | 13.0 | μl |
| 10X Capping Buffer | 2.0 | μl |
| GTP (10 mM) | 1.0 | μl |
| SAM (4 mM, dilute 32 mM stock to 4 mM) | 1.0 | μl |
| Vaccinia Capping Enzyme (10 U/μl) | 1.0 | μl |
| mRNA Cap 2′-O-Methyltransferase (50 U/μl) | 1.0 | μl |
| SUPERase Inh | 1.0 | μl |
| Total | 20 | μL |

Poly(A) tail synthesis: To add the poly(A) tails to the modified RNAs, the components provided in Table 9 (below) were added to a reaction tube. Then, the reaction was incubated for 30 minutes at 37° C. Then, the reaction was stopped by directly purifying the RNA with the small Monarch cleanup kit. As a quality control, 200 ng of the RNA was run on a 1.2% RNA gel to confirm the size of the RNA. To do so, RNA was denatured with 50% formaldehyde sample buffer for 5 minutes at 65° C., and then, immediately placed on ice for at least one minute. Then, the denatured RNA was loaded onto a gel and visualized using a Transilluminator.

RNA purification: A poly A containing RNA transcript was purified from IVT reaction impurities.

Table 9 (below) provides a summary of the different IL-12-expressing RNA constructs produced and analyzed in the following examples. Constructs #1-#9 all have a 3′-end scar following the polyA tail (i.e., 3′-end termination with a restriction enzyme after SGP).

TABLE 9

| Name of Construct | Description of Construct |
|---|---|
| repRNA | (i) Self-replicating mRNA + (ii) Cap Analog |
| (Cap Analog) | co-transcriptional capping method |
| (“Construct #1”) | |
| repRNA | (i) Self-replicating mRNA + (ii) enzymatic |
| (Enzymatic) | post-translational capping method |
| (“Construct #2”) | |
| repRNA | (i) Self-replicating mRNA + (ii) Cap Analog |
| (Cap Analog) | co-transcriptional capping method + (iii) VEE |
| A3G + E1 | replicon vector comprising A3G and + E1 |
| (“Construct #3”) | modifications |
| repRNA | (i) Self-replicating mRNA + (ii) enzymatic |
| (Enzymatic) | post-translational capping method + (iii) VEE |
| A3G + E1 | replicon vector comprising A3G and +El |
| (“Construct #4”) | modifications |
| repRNA | (i) Self-replicating mRNA + (ii) Cap Analog |
| (Cap Analog) | co-transcriptional capping method + (iii) VEE |
| Alternative | replicon vector comprising Alternative, Evolved, |
| Evolved + E1 | and + E1 modifications |
| (“Construct #5”) | |
| repRNA | (i) Self-replicating mRNA + (ii) enzymatic |
| (Enzymatic) | post-translational capping method + (iii) VEE |
| Alternative | replicon vector comprising Alternative, Evolved, |
| Evolved + E1 | and + E1 modifications |
| (“Construct #6”) | |
| repRNA | (i) Self-replicating mRNA + (ii) Cap Analog |
| (Cap Analog) | co-transcriptional capping method + (iii) VEE |
| Alternative + E1 | replicon vector comprising Alternative and + |
| (“Construct #7”) | E1 modifications |
| repRNA | (i) Self-replicating mRNA + (ii) Cap Analog |
| (Cap Analog) | co-transcriptional capping method + (iii) VEE |
| A3G + E1 Evolved | replicon vector comprising A3G, +E1, and |
| (“Construct #8”) | Evolved modifications |
| repRNA | (i) Self-replicating mRNA + (ii) Cap Analog |
| (Cap Analog) | co-transcriptional capping method + (iii) VEE |
| A3G − E1 | replicon vector comprising A3G and − El |
| (“Construct #9) | modifications |
| repRNA | (i) Self-replicating mRNA + (ii) enzymatic |
| (Enzymatic) | post-translational capping method + (iii) VEE |
| Alternative Evolved − | replicon vector comprising Alternative, Evolved, |
| E1 SGP scar | and − E1 modifications + (iv) 3′-end |
| (“Construct #10”) | termination with a restriction enzyme scar |
| | after SGP |
| modRNA | Non-self-replicating modified mRNA + Cap |
| (Cap Analog) | Analog co-transcriptional capping method |
| (“Construct #11”) | |
| repRNA | (i) Self-replicating mRNA + (ii) Cap Analog |
| (Cap Analog) | co-transcriptional capping method + (iii) VEE |
| A3G + E1 | replicon vector comprising A3G and +E1 |
| SGP scar | modifications + (iv) 3′-end termination |
| (“Construct #12”) | with a restriction enzyme scar after SGP |
| repRNA | (i) Self-replicating mRNA + (ii) Cap Analog |
| (Cap Analog) | co-transcriptional capping method + (iii) VEE |
| A3G + E1 | replicon vector comprising A3G and + E1 |
| clean 3′-end | modifications + (iv) 3′-end termination |
| (“Construct #13”) | with a clean polyA sequence |

### Example 2: mRNA Composition Analysis

### Comparison of Capping Methods on Anti-Tumor Efficacy

To begin analyzing the different RNA constructs produced in Example 1 above, a mouse melanoma model was used to compare the anti-tumor efficacy of (i) repRNA co-transcriptionally capped with a 5′ cap analog (i.e., Construct #1 in Table 9) to that of (ii) repRNA post-transcriptionally capped by enzymatic addition of 5′ cap (i.e., Construct #2 in Table 9). Briefly, melanoma was induced by inoculating the animals with B6-F10 cells (via subcutaneous administration). B16-F10 cell lines were obtained from American Type Culture Collection (ATCC) and grown in DMEM supplemented with 10% fetal bovine serum, 50U/ml Penicillin-Streptomycin, and 2 mM L-Glutamine in a humidified incubator (37° C. and 5% CO2). At ˜80% confluence cells were washed in phosphate-buffered saline solution (PBS), harvested from tissue culture flasks by incubating cells in 0.25% Trypsin-EDTA until detached, washed twice in PBS and resuspended PBS at a concentration of 2 million cells per ml. 1 million cells were implanted subcutaneously under the left hind-flank of female 6-8 week old C57BL/6J mice obtained from The Jackson Laboratory. Tumor-bearing mice were monitored until the tumors reached a mean volume of 350 mm³, then mice were randomized and then either of the above-described constructs were delivered by intratumoral injection using a 27 g syringe. Control animals were treated with PBS. Tumor sizes were recorded thrice weekly with a digital caliper and volumes were estimated using the following formula: (l×w\hat{ }2)×0.5, where 1=length (longest measurement) and w=widest diameter of the tumor, axis perpendicular to length.

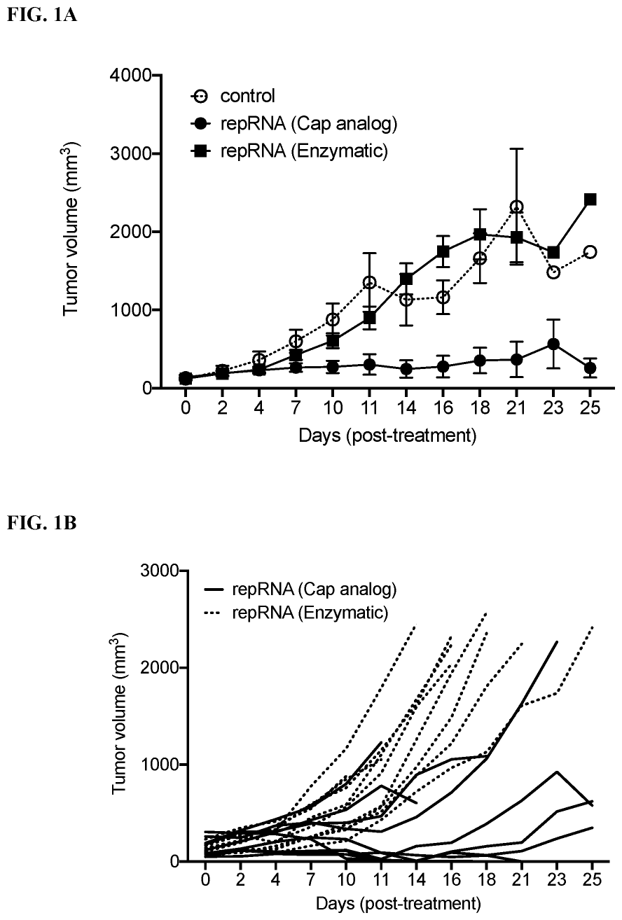

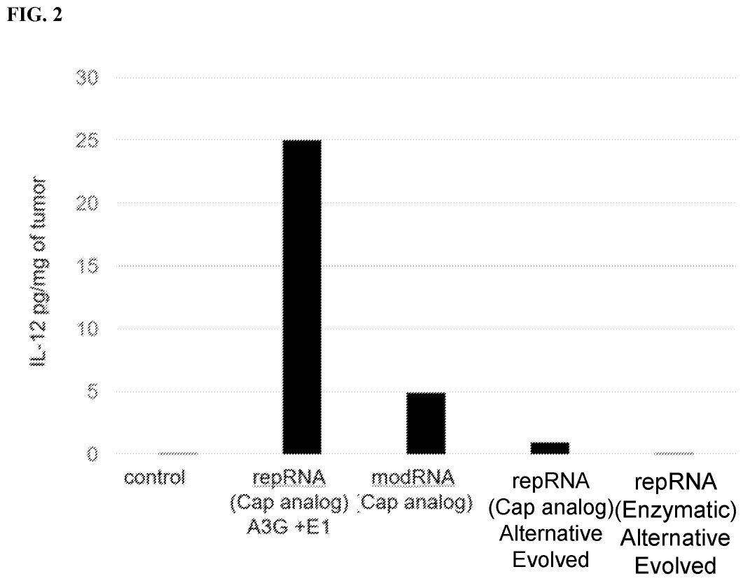

As shown in FIG. 1A and FIG. 1B, animals treated with Construct #1 (i.e., the repRNA made using Cap Analog co-transcriptional capping) had significantly reduced tumor volume compared to animals from the other treatment groups. Similar to the control group, animals treated with Construct #2 (i.e., the repRNA made using enzymatic post-translational capping method) failed to control the growth of the tumor. This data suggested that the Cap Analog co-transcriptional capping method was much more favorable compared to the enzymatic post-translational capping method in constructing the IL-12 expressing self-replicating constructs described herein.

### Comparison of VEE Replicon Backbones on Anti-Tumor Efficacy

Next, to assess whether the different VEE replicon backbones described in Example 1 had any effect on the therapeutic efficacy of the mRNA constructs, the above-described melanoma mouse model was used again. Once optimal tumor size was reached, the animals received a single intratumoral injection of one of the following: (i) PBS; (ii) repRNA made using the A3G +E1 vector and Cap Analog co-transcriptional capping (“Construct #3” in Table 9); (iii) modified mRNA (non-self-replicating) made using Cap Analog co-transcriptional capping (“Construct #5” in Table 9); (iv) repRNA made using the Alternative Evolved vector and Cap Analog co-transcriptional capping (“Construct #6” in Table 9); and (v) repRNA made using the Alternative Evolved vector and enzymatic post-translational capping (“Construct #7” in Table 9). Then, the expression of the IL-12 protein in the tumors was assessed at day 3 post-administration. Survival of the treated animals was also assessed.

Quantification of the IL-12 protein in the tissues (e.g., tumors) was performed as follows. Tissues were dissected out (e.g., at 72 hours post-administration), weighed, snap frozen, and subsequently lysed in radioimmunoprecipitation assay buffer. The concentration of the IL-12 protein in tissue lysates was determined using a commercially available sandwich enzyme-linked immunosorbent assay (ELISA) against the p70 subunit of murine IL-12, according to the manufacturer's (BioLegend) protocol.

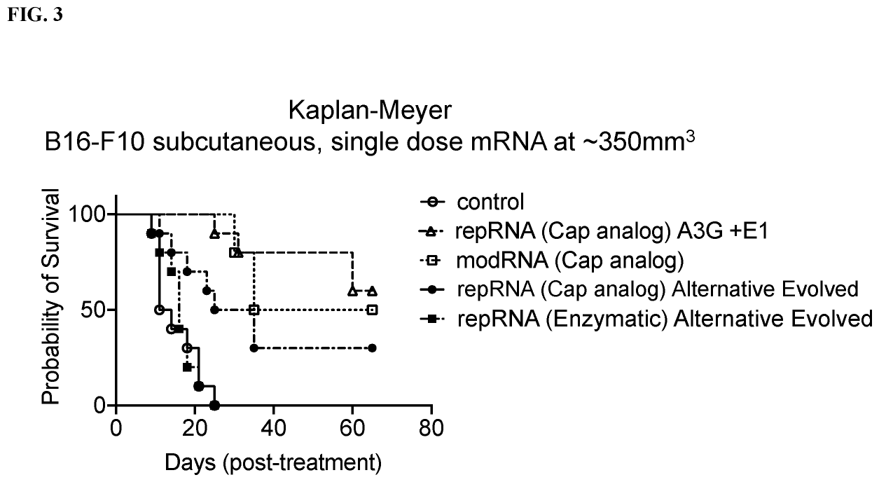

As shown in FIG. 2, IL-12 expression in the tumor delivery site was higher in animals that received Construct #3 (i.e., repRNA made using the A3G +E1 vector and Cap Analog co-transcriptional capping) as compared to animals that received either Construct #5 or Construct #6. In agreement with the expression data, animals treated with Construct #3 (i.e., repRNA made using the A3G +E1 vector and Cap Analog co-transcriptional capping) also exhibited the greatest survival (see FIG. 3).

### Comparison of Transfection Efficiency and IL-12 Secretion

To further analyze the different RNA constructs described in Example 1, both the transfection efficiency and IL-12 secretion were assessed in vitro. Briefly, B16.F10 cells (100,000 cells per well) were split into 24-well plates one day before transfection. The cells were then transfected using Lipofectamine messengerMAX according to the manufacturer's instructions. Briefly, 100 ng of total RNA was transfected with 1.5 uL Lipofectamine reagent per well and incubated until desired timepoints (i.e., 24 and 48 hours post-transfection). The specific RNA constructs tested included the following: (i) repRNA made using the A3G +E1 vector and Cap Analog co-transcriptional capping (“Construct 3” in Table 9); (ii) repRNA made using the A3G +E1 vector and enzymatic post-translational capping (“Construct #4” in Table 9); (iii) repRNA made using the Alternative Evolved +E1 vector and Cap Analog co-transcriptional capping (“Construct #5” in Table 9); (iv) repRNA made using the Alternative Evolved +E1 vector and enzymatic post-translational capping (“Construct #6” in Table 9); (v) repRNA made using the Alternative +E1 vector and Cap Analog co-transcriptional capping (“Construct #7” in Table 9); (vi) repRNA made using the A3G +E1 Evolved vector and Cap Analog co-transcriptional capping (“Construct #8” in Table 9); (vii) repRNA made using the A3G −E1 vector and Cap Analog co-transcriptional capping (“Construct #9” in Table 9); and (viii) repRNA made using the Alternative Evolved −E1 SGP scar end vector (“Construct #10” in Table 9).

To assess the transfection efficiency of the RNA constructs, FACS analysis was used to measure IL-12 expression in the cells. Briefly, Golgi transport blocking incubation was set up by trypsinizing cells using 100 uL trypsin. Next, complete media with Brefeldin A was added, and the cells then transferred to a 96-well deep well assay plate. The plate was then covered with parafilm and the cells incubated in complete media with Brefeldin A for 4 hours in the cell culture incubator. Staining for dead cell discrimination was performed by spinning down the cells at 5′, 400 g. Cells were washed in PBS and subsequently transferred to V-bottom 96-well plates, which were then centrifuged at 400×g. Cells were then resuspended in Zombie Live/Dead Green dye diluted in PBS and incubated for 10 minutes at room temperature in the dark. The cells were then washed in FACS buffer, which comprised 1×PBS (Ca²⁺ and Mg²⁺ free), 2 mM EDTA, 2% BSA, and 0.1% Sodium Azide. Cells were then fixated by resuspending the cells in fixation buffer and incubated for 30 minutes at room temperature in the dark.

Cells were permeabilized and cytoplasmic proteins stained as follows. Cells were washed in permeabilization/wash buffer and centrifuged at 800×g. Cells were resuspended in cytoplasmic antibody cocktail and incubated at room temperature in the dark. Cells were washed in permeabilization/wash buffer and centrifuged at 800×g, which was followed by a wash with FACS buffer and centrifugation at 800×g. Samples were then prepared for flow cytometry by resuspending the cells in FACS buffer. FACS analysis of the cells was performed by using the red and the blue laser on the Attune Flow cytometer. After gating out cell debris and doublets, the single cells were analyzed on the 2 channels of the cytometer and gated to quantify the percent of cells positive for IL-12. The results were subsequently plotted in Prism.

To measure IL-12 secretion, an ELISA assay was used. Briefly, supernatant for cell cultures was collected for subsequent ELISA-based analysis for various different proteins of interest. For each supernatant sample timepoint, the cell culture supernatant from the appropriate well was aspirated into a 96-well deep well plate, which could be stored at −80° C. for future use. After collection of all desired timepoints, the 96-well plate containing the supernatant samples was centrifuged at 500 g for 5 minutes to pellet any remaining cells. Following centrifugation, ˜350-400 uL supernatant was aspirated from the plate into a fresh deep well plate without disturbing the cell pellet. The supernatant was then diluted and tested in ELISA assays against mouse IL-12 or human IL-12 using Invitrogen ELISA kits according to the manufacturer's instructions, i.e., Invitrogen Mouse IL-12 p70 uncoated ELISA kit; Invitrogen Human IL-12 p70 Uncoated ELISA kit. Following ELISA assay data acquisition, a standard curve was drawn in Prism using 4PL method, and the concentration data was interpolated for all samples.

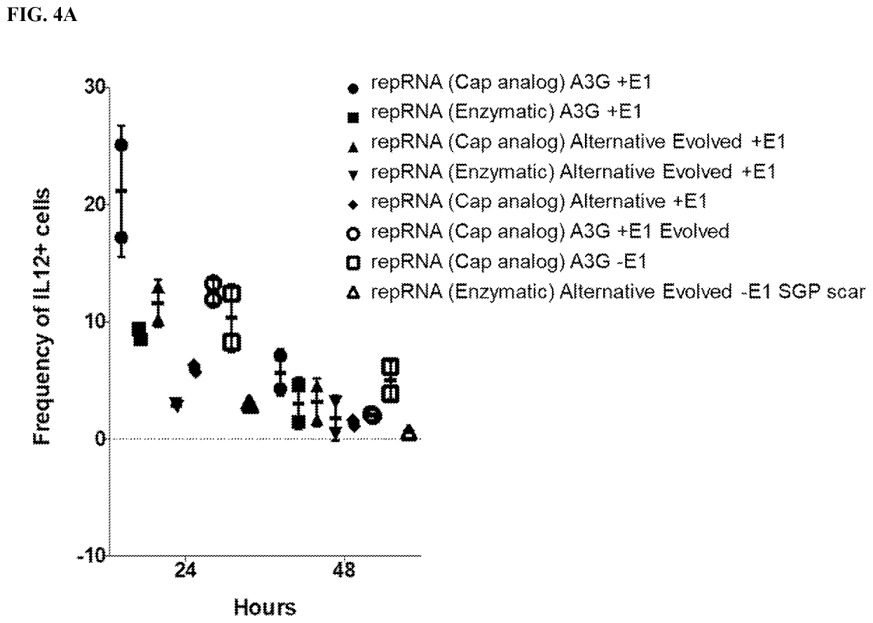

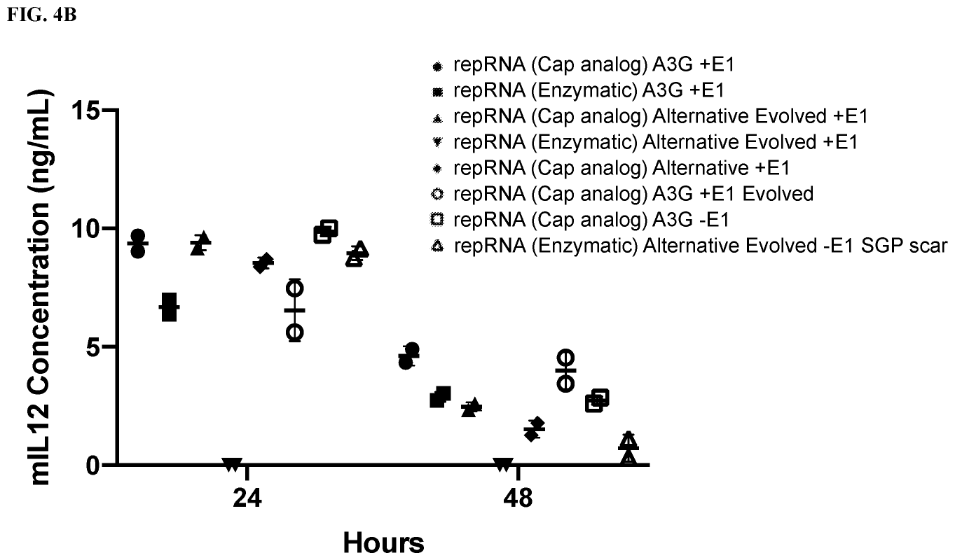

As shown in FIGS. 4A and 4B, the A3G and +E1 vector constructs generally performed better as compared to the Alternative, Evolved, −E1 constructs, or combinations. Moreover, in agreement with the earlier data, Cap Analog constructs generally performed better as compared to enzymatic capped constructs. Furthermore, the A3G +E1 repRNA (Construct #3) performed better than the other tested repRNA constructs in IL12 expression and total protein secretion at 24 and 48 hrs (see FIGS. 4A and 4B, respectively).

### Comparison of “Clean” and “Scar” 3′-End Termination

Next, the transfection efficiency of self-replicating mRNA with the 3′ end terminating with a “clean” polyA sequence was compared to a 3′ end terminating with a restriction enzyme “scar.” Briefly, the following constructs were used to transfect cells as described above: (i) repRNA made using the Cap Analog co-transcriptional capping method and A3G +E1 vector with a 3′-end terminating with a restriction enzyme scar (“Construct #12” in Table 10; circle and square); (ii) repRNA made using the Cap Analog co-transcriptional capping method and A3G+E1 vector with a 3′-end terminating with a clean polyA sequence (“Construct #13” in Table 10; triangle and inverted triangle). Then, IL-12 expression was assessed in the cells using FACS analysis as described above.

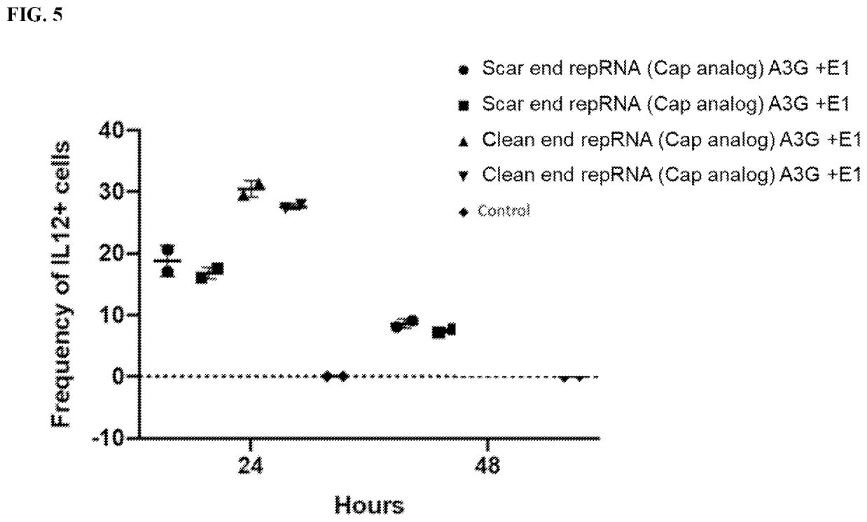

As shown in FIG. 5, 3′-end termination with a clean polyA sequence improved expression in vitro.

### Example 3: Lipid Nanoparticle (LNP) Formulation

In the present example, replicating mRNA encapsulated in an LNP formulations comprising cationic, phospho, and PEG lipids and cholesterol and LNP formulations comprising cationic and phospho lipids and cholesterol (with PEG-lipid micelles added to the solution as an independent component rather than part of the LNP per se) were prepared and analyzed. The mRNAs evaluated included mCherry replicating mRNAs as follows: 1. TT3-DMG (A3G +E1 backbone); and 2. TT3-post PEG micelles (A3G +E1 backbone).

To prepare the conventional TT3 LNP formulations, the following procedure was used. The lipid materials were each weighed out and dissolved in ethanol. The ethanol phase was prepared by mixing all the lipid materials according to composition weight ratio in Table 10 below. The aqueous phase was prepared by diluting purified JK001 mCherry repRNA with 20 mM Citrate Buffer (pH 4.0), 300 mM NaCl and water so that the final composition of the salt was 10 mM citrate Buffer (pH 4.0, 150 mM NaCl). The conventional TT3 LNPs were afforded by mixing the ethanol phase and aqueous phase of the LNPs through T-junction mixing at the flow rate ratio of 3:1 (aqueous phase: ethanol phase).

TABLE 10

| TT3 LNP Composition Component | Weight Ratio |
|---|---|
| mRNA | 1.0 |
| TT3 | 10.0 |
| DOPE | 8.0 |
| Cholesterol | 5.6 |
| DMG-PEG-2K | 0.7 |

To prepare the post-PEG micelles TT3 LNPs formulations, the following procedure was used. The lipid materials were each weighed out and dissolved in ethanol. The ethanol phase was prepared by mixing all the lipid materials (i.e., TT3, DOPE, and cholesterol) except from DMG-PEG-2K, according to composition weight ratio described below (see, e.g., Table 11a). The aqueous phase was prepared by diluting purified JK001 mCherry repRNA (i.e., mRNA) with 20 mM Citrate Buffer (pH 4.0), 300 mM NaCl and water so that the final composition of the salt was 10 mM citrate Buffer (pH 4.0, 150 mM NaCl). PEG micelle phase was prepared by adding the corresponding volume of DMG-PEG-2K into TBS buffer and mixing thoroughly via vortex. Finally, the post-PEG micelles TT3 LNPs were afforded by first mixing the ethanol phase and aqueous phase of the LNPs through a T-junction mixing at the flow rate ratio of 3:1 (aqueous phase: ethanol phase), and followed by an immediate in-line dilution with the PEG micelle phase via T-junction mixing at the flow rate ratio of 1:1 (LNP phase:PEG phase). The final lipid composition of Post-PEG micelle TT3 LNP is described in Table 11b.

TABLE 11a

| TT3 LNP Composition Component | Weight Ratio |
|---|---|
| mRNA | 1.0 |
| TT3 | 10.0 |
| DOPE | 8.0 |
| Cholesterol | 5.6 |
| PEG-DMG-2k Micelle | Weight Ratio |
| DMG-PEG-2K | 4.2 (relative to mRNA above, which |
| | is not part of the micelle) |

TABLE 11b

| TT3 LNP Composition Component | Weight Ratio |
|---|---|
| mRNA | 1.0 |
| TT3 | 10.0 |
| DOPE | 8.0 |
| Cholesterol | 5.6 |
| DMG-PEG-2K | 4.2 |

The TT3 LNP formulations above were buffer exchanged and freeze/thawed as follows. The afforded TT3 LNPs were transferred to the dialysis cassettes and dialyzed in TBS buffer for 2 hours. 40% sucrose (W/V) in TBS stock solution was added into all the prepared TT3 LNPs to make a final solution of TT3 LNPs in 10% sucrose. The final RNA concentrations of LNPs were measured by dissociating the LNPs with 2% TE+Triton and further detected with Qubit assay. TT3 LNPs were aliquot into 50 μl/tube aliquots and put the at −80° C. for freezing. Before treating cells with LNPs, TT3 LNPs were thawed at room temperature.

### Example 4: In Vivo Mouse IL-12 Variant Analysis

In the present example, assays were performed to evaluate various different mouse IL-12 variants in a B16.F10 mouse syngeneic cancer model. Briefly, the animals were treated via intratumoral administration with RNA constructs encoding one of the following mouse IL-12 proteins: (i) mIL-12 alone; (ii) mIL-12 conjugated to albumin; and (iii) mIL-12 conjugated to albumin and lumican. The RNA constructs were administered to the animals at a dose of either 0.25 μg or 2.5 μg. Then, the concentration of IL-12 and/or IFN-γ was measured using ELISA-based assays at days 1, 4, and/or 7 post-administration in one or more of the following tissues: tumor, serum, spleen, draining lymph nodes, and non-draining lymph nodes.

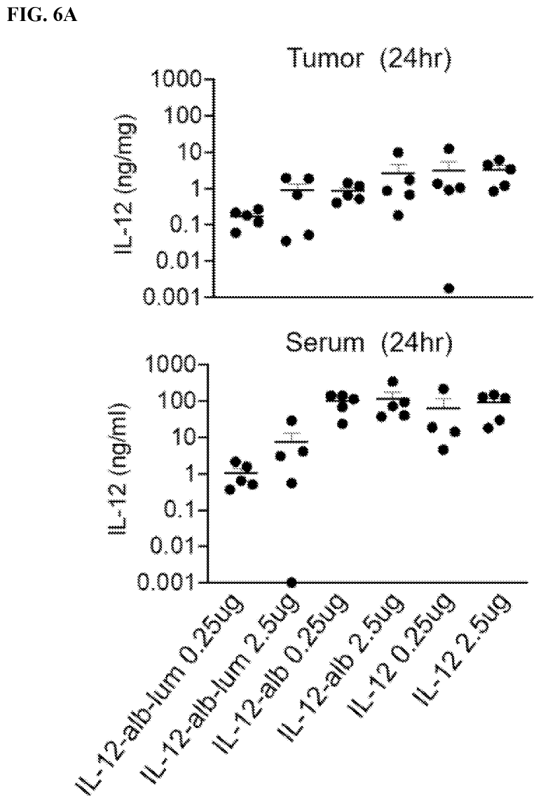

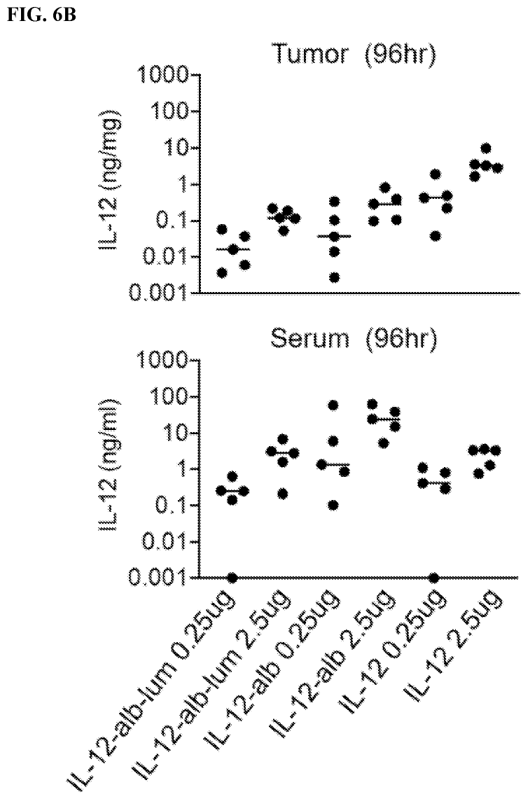

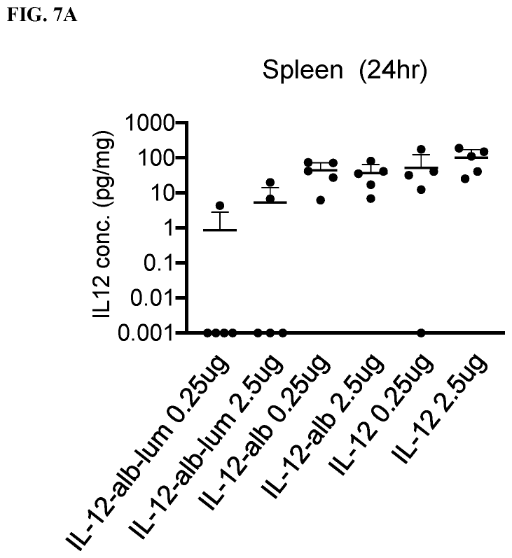

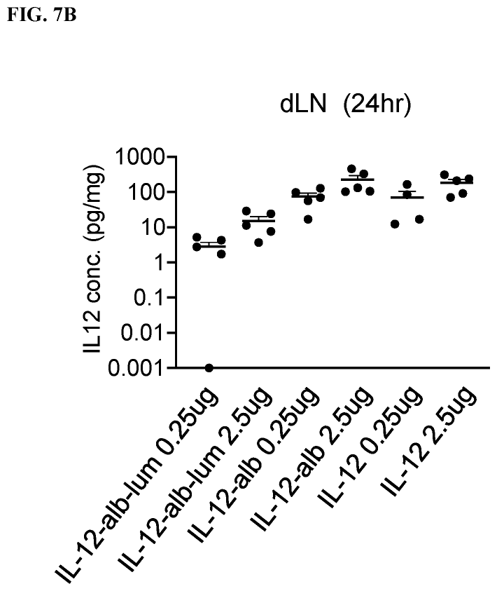

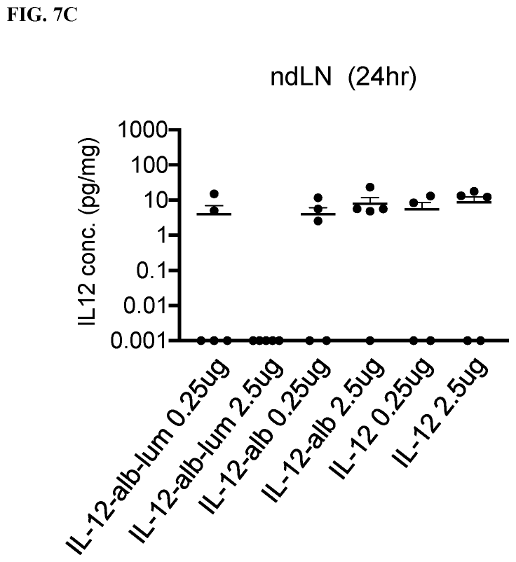

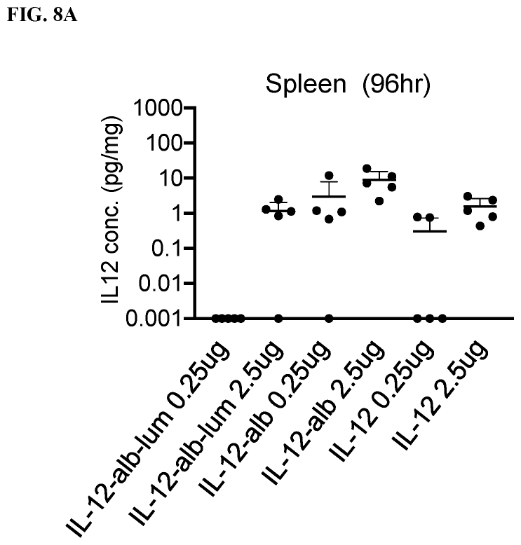

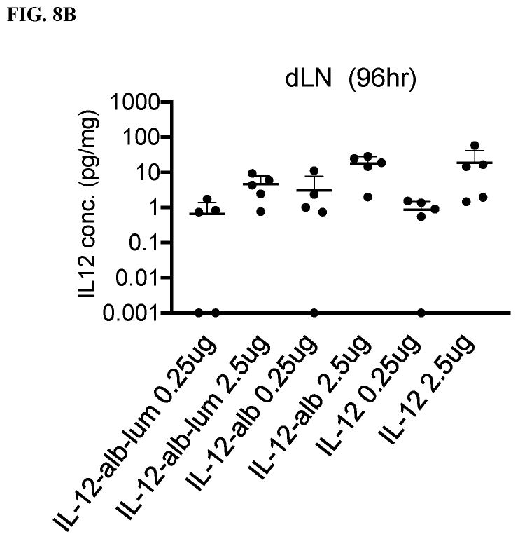

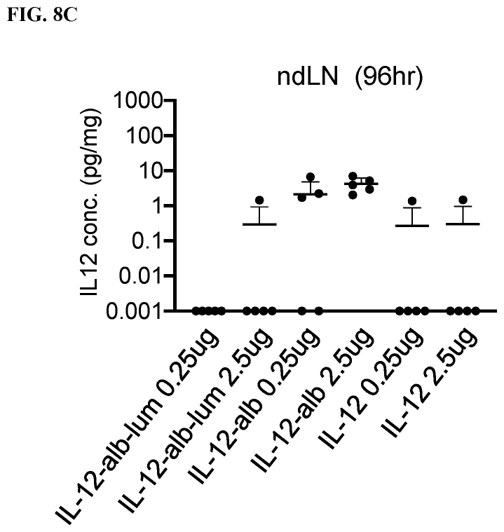

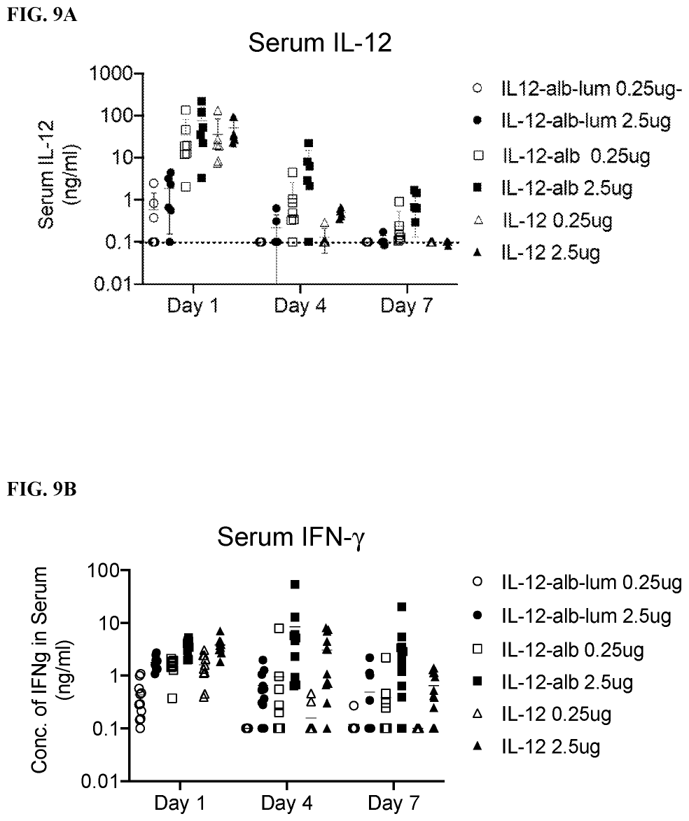

As shown in FIGS. 6A and 6C, at day 1 post-administration, the concentration of the IL-12 protein in the tumors was comparable among the different animals, with modest decrease in animals treated with the RNA construct encoding mIL-12 conjugated to albumin and lumican. Similar results were observed in the serum (FIG. 6B), spleen (FIG. 7A), draining lymph nodes (FIG. 7B), and non-draining lymph nodes (FIG. 7C). At day 4 post-administration, animals treated with 2.5 μg of the RNA construct encoding the mIL-12 alone had the highest IL-12 expression level in the tumor (see FIG. 6C). In the other tissues analyzed, the IL-12 expression levels were more comparable, with slightly higher levels observed in animals treated with the RNA construct encoding mIL-12 conjugated to albumin (see FIGS. 6D, 8A, 8B, 8C, and 9A). IFN-γ expression level was also the highest in animals treated with the RNA construct encoding mIL-12 conjugated to albumin (FIG. 9B).

### Example 5: Human IL-12 Codon Optimization

In the present example, human IL-12 codon was optimized as follows. 20301 diverse sequences encoding the fusion protein human light chain leader—hIL12p40—GGS(GGGS)₃ linker—hIL12p35—GSGGGS linker—Human serum albumin were generated algorithmically. Codon optimality was calculated as the average frequency with which each codon in a given coding sequence is used to encode a given amino acid in the standard human transcriptome. 1137 representative sequences were identified, and their minimum free energy of folding (MFE) was calculated by ViennaRNA-2.4.14. Representative sequences with low, mid, and high codon optimality and MFE (L1, L2, L3; M1, M2, M3; H1, H2, H3) were modified to contain an identical light chain linker and enable efficient commercial synthesis and assembly and tested experimentally. A sequence (CO) containing the most frequently employed codon for each amino acid was also generated by this algorithm. A separate sequence (CP), containing an optimally frequent set of codon pairs, was generated by a related algorithm.

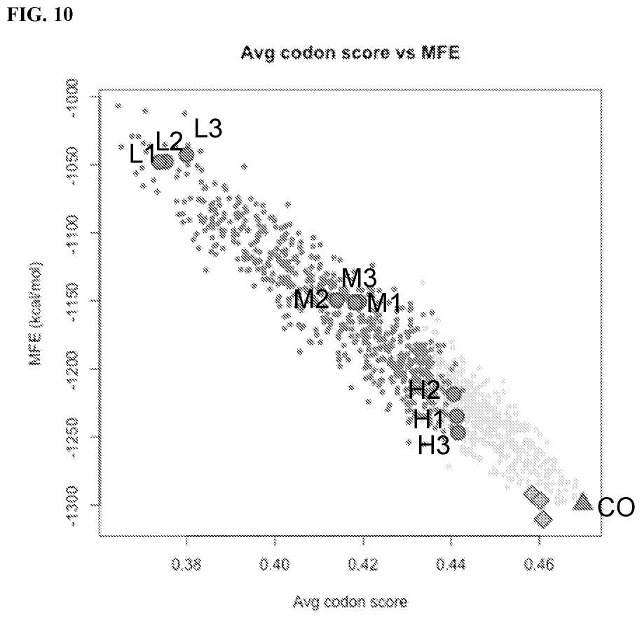

FIG. 10 presents data related to optimality (avg_codon_score) vs minimum folding free energy in kcal/mol (MFE) for 1137 distinct sequences encoding the fusion protein human light chain leader—hIL12p40—GGS(GGGS)3 linker—hIL12p35—GSGGGS linker—Human serum albumin. The codon optimal (“CO”) sequence containing the most frequently used triplet at each position is shown as the triangle. Sequences with >90% and >90% identity to CO are shown as dark gray and light gray dots, respectively. The representative sequences with low, mid, and high codon optimality and MFE (L1, L2, L3; M1, M2, M3; H1, H2, H3), are shown using a circle symbol. Representative sequences with very high codon optimality and MFE are shown as diamonds and were not experimentally tested. Initial screening demonstrated that highly structured and frequently used codons gave high expression from repRNA.

IL-12 expression levels of variant self-replicating mRNAs encoding hIL-12 (A1-A4), hIL12-albumin (B1-B4), and hIL12-albumin-lumican (C1-C3) were measured in an in vitro expression assay in human TNBC cell line. IL-12, IL-12-alb, and IL-12-alb-lum versions of each of these constructs were tested. Human TNBC BT20 cells were transfected via TT3-LNP and incubated for 20 hours prior to expression level measurements by ELISA-based assays.

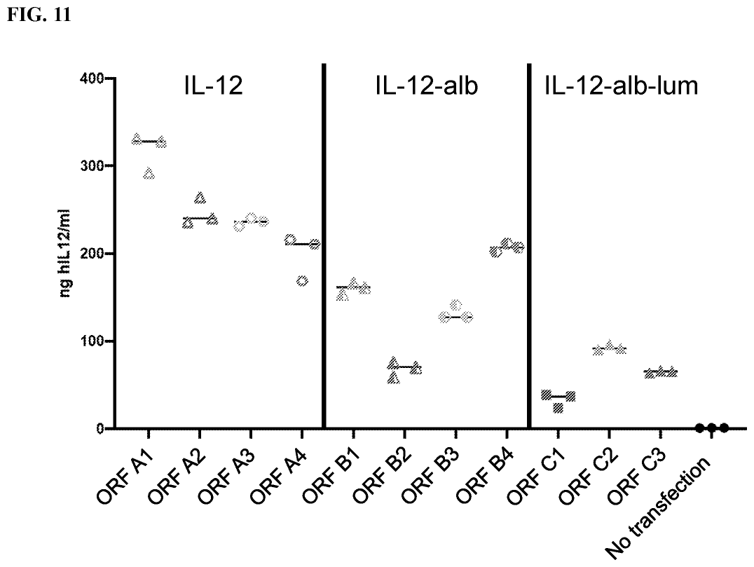

FIG. 11 presents data related to IL-12 expression level of variant self-replicating mRNAs encoding hIL-12 (A1-A4), hIL12-albumin (B1-B4), and hIL12-albumin-lumican (C1-C3) 20 hours after transfection via TT3-LNP into BT-20 cells. Individual triplicate measurements are shown as triangular, square, octagonal, or circular points, and horizontal bars show the average of triplicate measurements. The best expressing constructs were used in subsequent TNBC PDX mouse experiments. Briefly, patient-derived xenograft (PDX) experiments were performed as follows: PDXs were initially established from fresh surgically resected tumors from TNBC (ER,PR,HER2-negative breast cancer) patients. Tumors specimens were mechanically dissociated into small fragments, mixed with Matrigel solution, and surgically implanted as solid fragments under the skin of NOD.Cg-Prkdc-^{scid}Il2rg-^{tm1Wjl}/SzJ (NSG) mice using a trocar. PDX tissue was serially passaged in vivo prior to initiation of the experiment. For the experiments described, PDX tumor fragments were engrafted subcutaneously into NSG mice and randomized upon tumors reaching a mean size of 200-350 mm3. Immediately after randomization mice were treated with indicated agents and serum, spleen, and tumors were collected 24 hours later. Human IL-12 concentrations in the serum, spleen lysates, and tumor lysates were determined by ELISA-based assays.

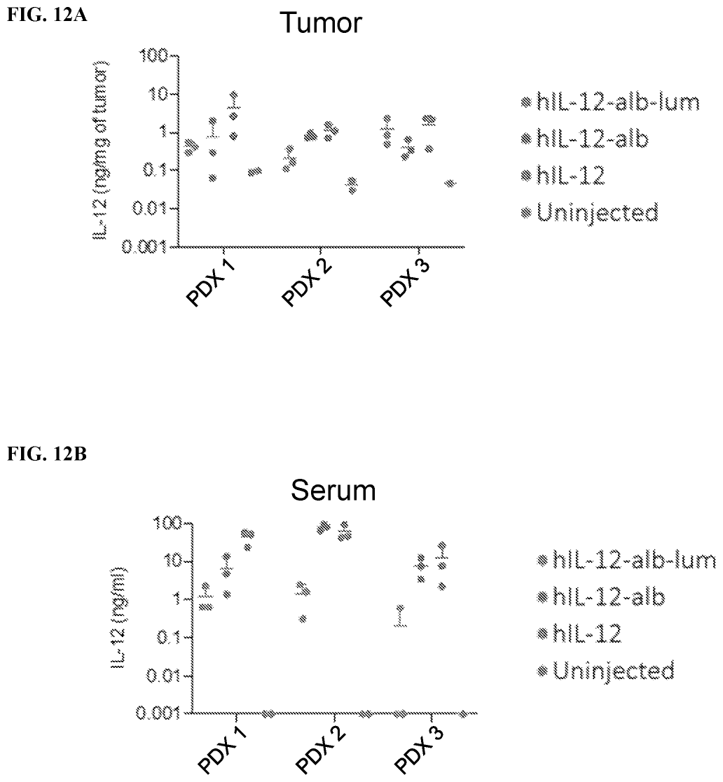

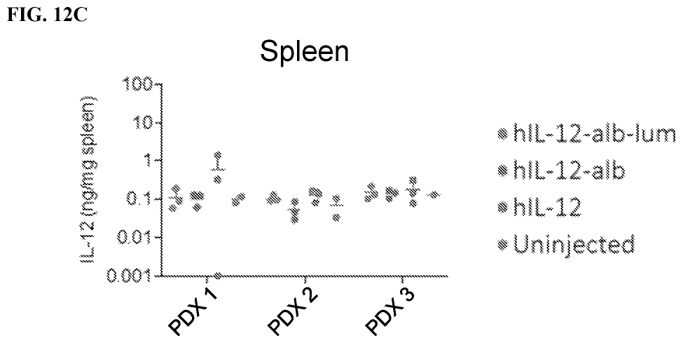

As shown in FIGS. 12A-12C, in the different tissues analyzed (tumor, serum, and spleen), animals treated with the mRNA construct encoding the human IL-12 alone (i.e., not conjugated to albumin and/or lumican) exhibited the highest IL-12 expression levels. Such results demonstrate the effectiveness of the codon-optimization strategy described above.

### Example 6: Analysis of Dosage Effects in Breast Cancer Mouse Model

Further to Example 4 provided above, the anti-tumor effects of the IL-12 constructs provided herein were assessed in a breast cancer animal model. Briefly, triple-negative breast cancer (TNBC) was induced by inoculating the animals with 4T1 cells (via administration in the mammary fat pad). Once the tumors reached an optimal size (˜150 mm³), one of the following was injected into the primary tumors of the animals: (i) vehicle control; (ii) 5 ug of self-replicating mRNA encoding IL-12 (repRNA-IL12); (iii) 0.5 ug of self-replicating mRNA encoding IL-12 (repRNA-IL12); (iv) 0.05 ug of self-replicating mRNA encoding IL-12 (repRNA-IL12). Animals received a total of 4 doses on a weekly schedule. Then, tumor volume was assessed at various time points post-treatment.

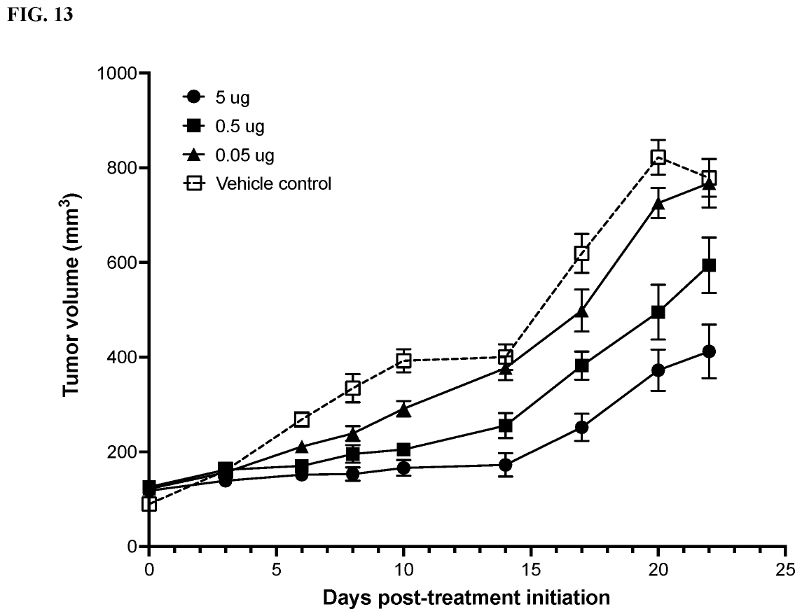

As shown in FIG. 13, there was a dose dependent inhibition of primary tumor growth over the duration of the study in the repRNA-IL12 treated animals, resulting in an approximate 50% reduction in primary tumor size at the time the study was concluded. These results confirm that the self-replicating mRNA constructs disclosed herein (e.g., encoding IL-12) are effective in treating various cancers, including aggressive and immunotherapy resistant breast cancer.

### Example 7: Analysis of the Anti-Tumor Effects of IL-12 Constructs Comprising Modified Nucleoside Triphosphates

Next, to assess whether incorporation of different proportions of modified nucleoside triphosphates (modNTPs) into self-replicating mRNAs described herein had any effect on the therapeutic efficacy of the mRNA constructs, the above-described TNBC mouse model (see Example 6) was used. Once optimal tumor size was reached, the animals received weekly intratumoral injections of one of the following: (i) PBS (vehicle control); (ii) 5 ug repRNA made with 0% modNTPs; (iii) 5 ug repRNA made with 25% modNTPs; (iv) 5 ug repRNA made with 37.5% modNTPs; and (v) 5 ug repRNA made with 50% modNTPs. Then tumor volume was assessed at various time points post-treatment.

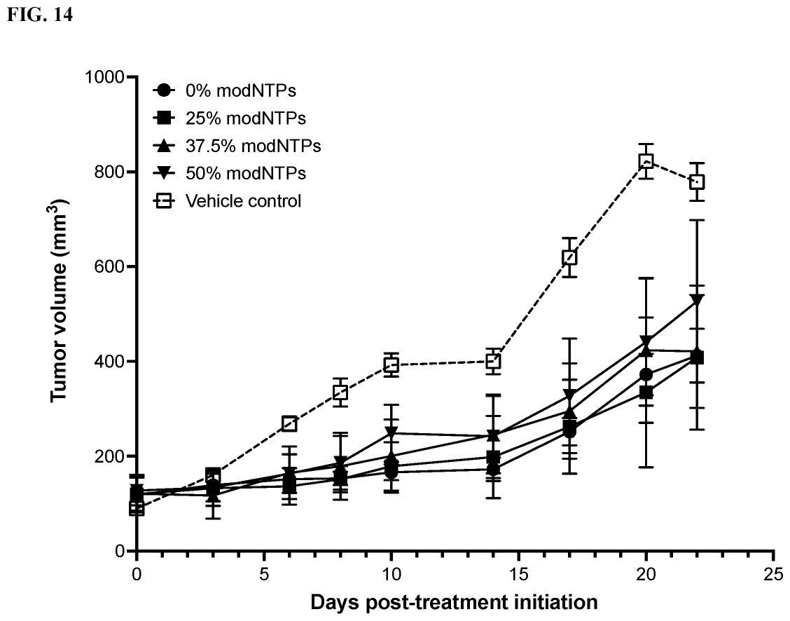

As shown in FIG. 14, animals that were treated with the different modified repRNA IL-12 constructs behaved similarly (regardless of the amount) to those treated with the non-modified repRNA construct. This data suggest that the addition of modNTPs had limited impact on the efficacy of the repRNA constructs to control tumor growth.

### Example 8: Analysis of the Abscopal Effect in Mouse Model of Melanoma

To assess the ability of repRNA encoding IL-12 (e.g., described herein) to induce systemic anti-tumor immune responses that inhibit the growth of distal untreated tumor lesions, a modified version of the mouse melanoma model described in Example 4 was used. Briefly, to induce formation of two separate primary melanoma tumors, 1×10⁶ B16-F10 cells were subcutaneously implanted on the left hind flank of the animal, whereas at the same time the 2×10⁵ B16-F10 cells were implanted on the right hind flank. Once optimal tumor size was reached the animals received a single intratumoral injection in only the left flank tumor of the following: (i) PBS (vehicle control); and (ii) 2.5 ug of repRNA encoding IL-12. Tumor growth was assessed by measuring tumor volumes of both the treated (left flank) and untreated (right flank) tumors at various time points post-treatment.

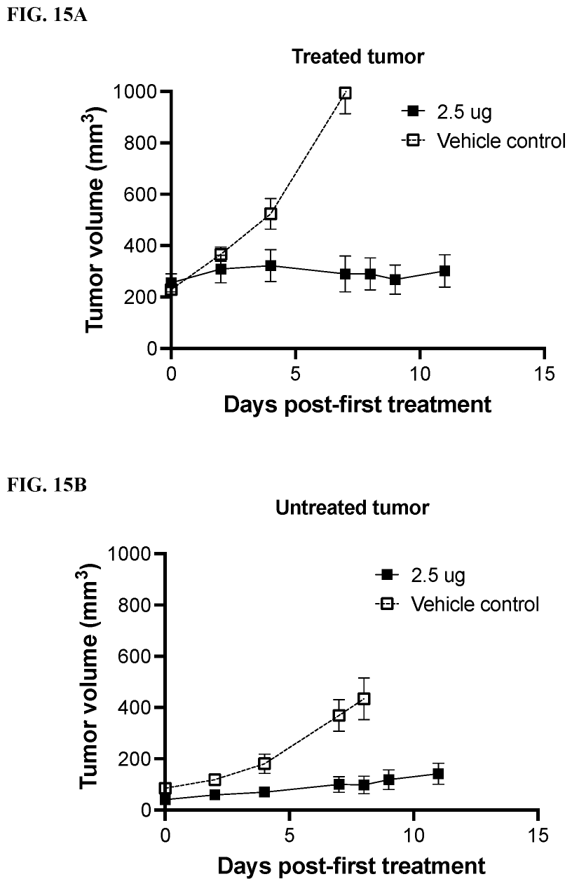

As shown in FIGS. 15A and 15B, repRNA encoding IL-12 delivered intratumorally was able to control the growth of both the treated tumor and the untreated distal tumors. These data suggest that local delivery of repRNA encoding IL-12 could be an effective therapy in metastatic cancer.

### Example 9: Analysis of Payload Expression after Re-Dosing

To evaluate the re-dosability of the mRNA constructs described herein, the TNBC mouse model described in Example 6 was used again. Briefly, mRNA constructs encoding the reporter gene firefly luciferase were delivered by intratumoral injection, 5 ug once weekly for two doses. In some groups, animals were co-administered twice weekly with intraperitoneal injections of 10 mg/kg mouse anti-IFNAR1 antibodies (Clone MAR1-5A3) to inhibit activity of IFN-α. 24 hours after each dose of mRNA constructs, animals received 150 mg/kg D-Luciferin (the substrate that is converted by firefly luciferase into luminescent signal) by intraperitoneal injection and tumors were live imaged using an in vivo imaging system. Relative bioluminescence was quantified 10 minutes after substrate administration.

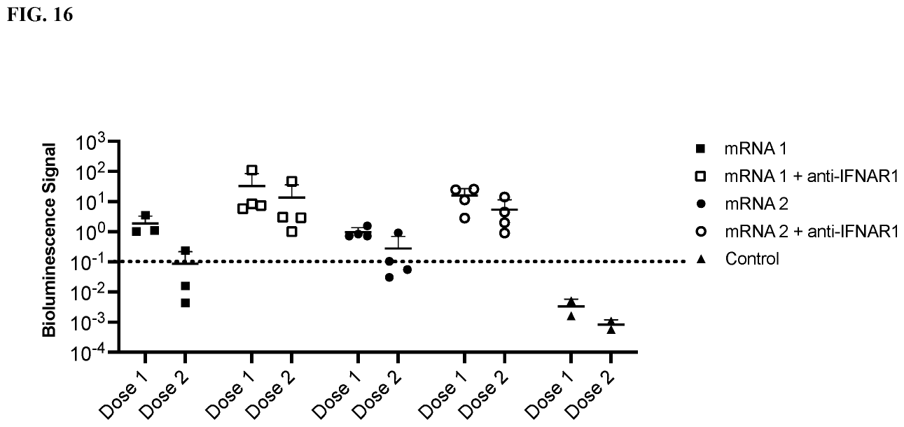

As shown in FIG. 16, some decrease in payload expression was observed upon second dose compared to the first dose. The payload expression after the second dose was rescued when the IFN-α receptor activity was inhibited by antibody blockade. These data suggest that mRNA constructs described herein can be dosed repeatedly and decreased payload expression observed upon re-dosing is, at least in part, dependent of type interferon induction and can be overcome by IFN receptor antibody blockade.

### Example 10: Analysis of Ex Vivo Activity in Human PBMCS

To further demonstrate the activity of the IL-12 constructs provided herein, human TNBC cell line BT20 was transfected with repRNA encoding IL-12. About 24 hours later, supernatant was collected (“conditioned media”) and IL-12 level was assessed using a human IL-12 ELISA (Invitrogen). Human peripheral blood mononuclear cells (PBMCs) were isolated from leukapheresis products from healthy human donors according to the IRB approved protocol and manufacturer's recommendations (StemCell technologies). The T cells in PBMCs were activated with IL-2 (10 ng/mL) and anti-CD3/CD28/CD2 antibody cocktail (StemCell technologies) for 2 days. Following the activation, the media was washed and cells were treated with the conditioned media (comprising known concentrations of hIL12 (1 or 10 ng/mL)) for 24 hrs. As a positive control, PBMCs were treated with indicated concentrations of recombinant hIL12 (rhIL12) (StemCell technologies) at various doses (0.01, 0.1, 1. 10, or 100 ng/mL) for the same duration. IL-12 is known to activate T and NK cells within the PBMCs, which leads to the production of Interferon-gamma (IFN-g) which can be tested from the supernatant. The supernatant was collected from the PBMCs and subjected to IFN-g ELISA (Invitrogen) to assay for its levels.

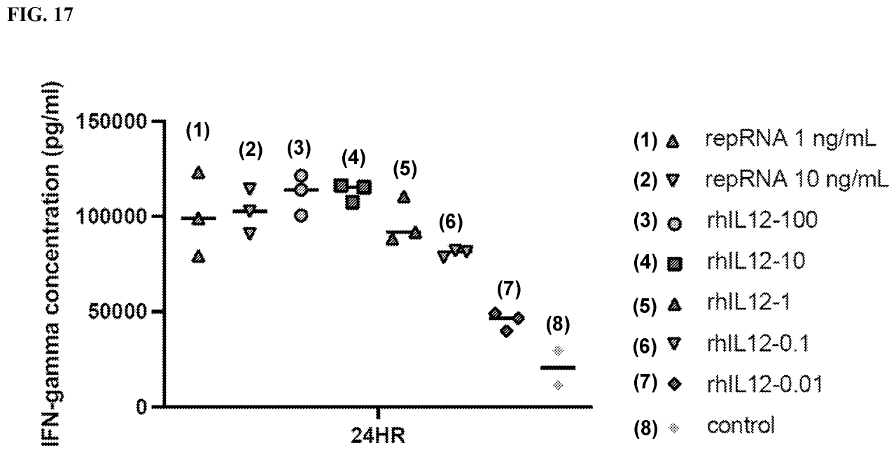

As shown in FIG. 17, the repRNA constructs described herein were able to induce potent IL-12 production, which in turn was able to induce the activation of T and NK cells, and the subsequent production of IFN-γ. The above results further confirm the activity of the IL-12 constructions described herein.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. An isolated polynucleotide comprising a nucleic acid molecule encoding a beta subunit of an IL-12 protein (“IL-12β”), wherein the nucleic acid molecule comprises a nucleotide sequence that has at least about 75% sequence identity to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75.

2. The isolated polynucleotide of claim 1, wherein the nucleic acid molecule encoding the IL-12β comprises:
(a) a nucleotide sequence that has at least 76% sequence identity to the sequence set forth in SEQ ID NO: 51;
(b) a nucleotide sequence that has at least 78% sequence identity to the sequence set forth in SEQ ID NO: 52;
(c) a nucleotide sequence that has at least 77% sequence identity to the sequence set forth in SEQ ID NO: 53;
(d) a nucleotide sequence that has at least 88% sequence identity to the sequence set forth in SEQ ID NO: 54;
(e) a nucleotide sequence that has at least 88% sequence identity to the sequence set forth in SEQ ID NO: 55;
(f) a nucleotide sequence that has at least 87% sequence identity to the sequence set forth in SEQ ID NO: 56;
(g) a nucleotide sequence that has at least 91% sequence identity to the sequence set forth in SEQ ID NO: 57;
(h) a nucleotide sequence that has at least 92% sequence identity to the sequence set forth in SEQ ID NO: 58;
(i) a nucleotide sequence that has at least 91% sequence identity to the sequence set forth in SEQ ID NO: 59;
(j) a nucleotide sequence that has at least 91% sequence identity to the sequence set forth in SEQ ID NO: 65, 69, or 74;
(k) a nucleotide sequence that has at least 91% sequence identity to the sequence set forth in SEQ ID NO: 66, 70, or 75;
(l) a nucleotide sequence that has at least 97% sequence identity to the sequence set forth in SEQ ID NO: 62;
(m) a nucleotide sequence that has at least 99% sequence identity to the sequence set forth in SEQ ID NO: 63; or
(n) a nucleotide sequence that has at least 98% sequence identity to the sequence set forth in SEQ ID NO: 64.

3.-15. (canceled)

16. An isolated polynucleotide comprising a nucleic acid molecule encoding an alpha subunit of an IL-12 protein (“IL-12α”), wherein the nucleic acid molecule comprises a nucleotide sequence that has at least about 77% sequence identity to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

17. The isolated polynucleotide of claim 16, wherein the nucleic acid molecule encoding the IL-12α comprises:
(A) a nucleotide sequence that has at least 79% sequence identity to the sequence set forth in SEQ ID NO: 101;
(b) a nucleotide sequence that has at least 78% sequence identity to the sequence set forth in SEQ ID NO: 102;
(c) a nucleotide sequence that has at least 77% sequence identity to the sequence set forth in SEQ ID NO: 103;
(d) a nucleotide sequence that has at least 86% sequence identity to the sequence set forth in SEQ ID NO: 104;
(e) a nucleotide sequence that has at least 88% sequence identity to the sequence set forth in SEQ ID NO: 105;
(f) a nucleotide sequence that has at least 88% sequence identity to the sequence set forth in SEQ ID NO: 106;
(g) a nucleotide sequence that has at least 91% sequence identity to the sequence set forth in SEQ ID NO: 107;
(h) a nucleotide sequence that has at least 91% sequence identity to the sequence set forth in SEQ ID NO: 108;
(i) a nucleotide sequence that has at least 92% sequence identity to the sequence set forth in SEQ ID NO: 109;
(j) a nucleotide sequence that has at least 91% sequence identity to the sequence set forth in SEQ ID NO: 115, 119, or 124;
(k) a nucleotide sequence that has at least 92% sequence identity to the sequence set forth in SEQ ID NO: 116, 120, or 125;
(l) a nucleotide sequence that has at least 98% sequence identity to the sequence set forth in SEQ ID NO: 112;
(m) a nucleotide sequence that has at least 98% sequence identity to the sequence set forth in SEQ ID NO: 113; or
(n) a nucleotide sequence that has at least 98% sequence identity to the sequence set forth in SEQ ID NO: 114.

18.-30. (canceled)

31. An isolated polynucleotide comprising a first nucleic acid molecule and a second nucleic acid molecule,
wherein the first nucleic acid molecule encodes a beta subunit of an IL-12 protein (“IL-12β”) and comprises a nucleotide sequence that has at least about 75% sequence identity to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75; and
wherein the second nucleic acid molecule encodes an alpha subunit of an IL-12 protein (“IL-12α”) and comprises a nucleotide sequence that has at least about 77% sequence identity to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

32. The isolated polynucleotide of claim 31, wherein:
(a) the first nucleic acid molecule comprises a nucleotide sequence that has at least 76% to the sequence set forth in SEQ ID NO: 51 and/or the second nucleic acid molecule comprises a nucleotide sequence that has at least 79% sequence identity to the sequence set forth in SEQ ID NO: 101;
(b) the first nucleic acid molecule comprises a nucleotide sequence that has at least 78% sequence identity to the sequence set forth in SEQ ID NO: 52 and/or the second nucleic acid molecule comprises a nucleotide sequence that has at least 78% sequence identity to the sequence set forth in SEQ ID NO: 102;
(c) the first nucleic acid molecule comprises a nucleotide sequence that has at least 77% sequence identity to the sequence set forth in SEQ ID NO: 53 and/or the second nucleic acid molecule comprises a nucleotide sequence that has at least 77% sequence identity to the sequence set forth in SEQ ID NO: 103;
(d) the first nucleic acid molecule comprises a nucleotide sequence that has at least 88% sequence identity to the sequence set forth in SEQ ID NO: 54 and/or the second nucleic acid molecule comprises a nucleotide sequence that has at least 86% sequence identity to the sequence set forth in SEQ ID NO: 104;
(e) the first nucleic acid molecule comprises a nucleotide sequence that has at least 88% sequence identity to the sequence set forth in SEQ ID NO: 55 and/or the second nucleic acid molecule comprises a nucleotide sequence that has at least 88% sequence identity to the sequence set forth in SEQ ID NO: 105;
(f) the first nucleic acid molecule comprises a nucleotide sequence that has at least 87% sequence identity to the sequence set forth in SEQ ID NO: 56 and/or the second nucleic acid molecule comprises a nucleotide sequence that has at least 88% sequence identity to the sequence set forth in SEQ ID NO: 106;
(g) the first nucleic acid molecule comprises a nucleotide sequence that has at least 91% sequence identity to the sequence set forth in SEQ ID NO: 57 and/or the second nucleic acid molecule comprises a nucleotide sequence that has at least 91% sequence identity to the sequence set forth in SEQ ID NO: 107;
(h) the first nucleic acid molecule comprises a nucleotide sequence that has at least 92% sequence identity to the sequence set forth in SEQ ID NO: 58 and/or the second nucleic acid molecule comprises a nucleotide sequence that has at least 91% sequence identity to the sequence set forth in SEQ ID NO: 108;
(i) the first nucleic acid molecule comprises a nucleotide sequence that has at least 91% sequence identity to the sequence set forth in SEQ ID NO: 59 and/or the second nucleic acid molecule comprises a nucleotide sequence that has at least 92% sequence identity to the sequence set forth in 109;
(j) the first nucleic acid molecule comprises a nucleotide sequence that has at least 91% sequence identity to the sequence set forth in SEQ ID NO: 65, 69, or 74 and/or the second nucleic acid molecule comprises a nucleotide sequence that has at least 91% sequence identity to the sequence set forth in SEQ ID NO: 115, 119, or 124;
(k) the first nucleic acid molecule comprises a nucleotide sequence that has at least 91% sequence identity to the sequence set forth in SEQ ID NO: 66, 70, or 75 and/or the second nucleic acid molecule comprises a nucleotide sequence that has at least 92% sequence identity to the sequence set forth in SEQ ID NO: 116, 120, or 125;
(l) the first nucleic acid molecule comprises a nucleotide sequence that has at least 97% sequence identity to the sequence set forth in SEQ ID NO: 62 and/or the second nucleic acid molecule comprises a nucleotide sequence that has at least 98% sequence identity to the sequence set forth in SEQ ID NO: 112;
(m) the first nucleic acid molecule comprises a nucleotide sequence that has at least 99% sequence identity to the sequence set forth in SEQ ID NO: 63 and/or the second nucleic acid molecule comprises a nucleotide sequence that has at least 98% sequence identity to the sequence set forth in SEQ ID NO: 113; or
(n) the first nucleic acid molecule comprises a nucleotide sequence that has at least 98% sequence identity to the sequence set forth in SEQ ID NO: 64 and/or the second nucleic acid molecule comprises a nucleotide sequence that has at least 98% sequence identity to the sequence set forth in SEQ ID NO: 114.

33.-45. (canceled)

46. The isolated polynucleotide of claim 31, which further comprises: (a) a third nucleic acid molecule encoding a linker that joins the first nucleic acid molecule and the second nucleic acid molecule, (b) an additional nucleic acid molecule encoding a half-life extending moiety, (c) an additional nucleic acid molecule encoding a leader sequence, or (d) any combination of (a) to (c).

47.-50. (canceled)

51. The isolated polynucleotide of claim 46, wherein the third nucleic acid molecule encoding the linker comprises the sequence set forth in any one of SEQ ID NOs: 168 to 170, and/or wherein the additional nucleic acid molecule encoding a leader sequence comprises the sequence set forth in any one of SEQ ID NO: 26 to 50.

52.-75. (canceled)

76. An isolated polynucleotide comprising (from 5′ to 3′) (i) a first nucleic acid molecule encoding a leader sequence, (ii) a second nucleic acid molecule encoding a beta subunit of an IL-12 protein (“IL-12β”), (iii) a third nucleic acid molecule encoding a first linker, and (iv) a fourth nucleic acid molecule encoding an alpha subunit of an IL-12 protein (“IL-12α”), wherein:
a. the first nucleic acid molecule comprises the sequence set forth in SEQ ID NO: 40;
b. the second nucleic acid molecule comprises the sequence set forth in SEQ ID NO: 65;
c. the third nucleic acid molecule comprises the sequence set forth in SEQ ID NO: 90; and
d. the fourth nucleic acid molecule comprises the sequence set forth in SEQ ID NO: 115.

77.-79. (canceled)

80. The isolated polynucleotide of claim 31, which further comprises:
(a) a 5′-cap,
(b) a regulatory element,
(c) a 3′ tailing region of linked nucleosides, or
(d) any combination of (a) to (c).

81.-85. (canceled)

86. The isolated polynucleotide of claim 31, which comprises at least one modified nucleoside.

87. (canceled)

88. The isolated polynucleotide of claim 31, which is capable of self-replicating.

89-91. (canceled)

92. A vector comprising the isolated polynucleotide of claim 31.

93. A lipid nanoparticle comprising (i) the isolated polynucleotide of claim 31, and (ii) one or more types of lipids.

94-108. (canceled)

109. A pharmaceutical composition, comprising the isolated polynucleotide of claim 31 and a pharmaceutically acceptable carrier.

110. (canceled)

111. A cell comprising the isolated polynucleotide of claim 31.

112. (canceled)

113. A method of making a polynucleotide comprising enzymatically or chemically synthesizing the isolated polynucleotide of claim 31.

114. A method of producing an IL-12 protein, comprising contacting a cell with the isolated polynucleotide of claim 31.

115. (canceled)

116. A method of treating a disease or disorder in a subject in need thereof, comprising administering to the subject the isolated polynucleotide of claim 31.

117. The method of claim 116, wherein the disease or disorder comprises a cancer.

118.-121. (canceled)
